(19) United States
(12) Patent Application Publication
(10) Pub. No.: US 2024/0103364 A1
(43) Pub. Date: Mar. 28, 2024

(54) ONIUM SALT, CHEMICALLY AMPLIFIED RESIST COMPOSITION, AND PATTERNING PROCESS

(71) Applicant: Shin-Etsu Chemical Co., Ltd., Tokyo (JP)

(72) Inventors: Masahiro Fukushima, Joetsu-shi (JP); Tomomi Watanabe, Joetsu-shi (JP); Kenji Yamada, Joetsu-shi (JP)

(73) Assignee: Shin-Etsu Chemical Co., Ltd., Tokyo (JP)

(21) Appl. No.: 18/229,863

(22) Filed: Aug. 3, 2023

(30) Foreign Application Priority Data
Aug. 16, 2022 (JP) 2022-129450

(51) Int. Cl. G03F 7/004; G03F 7/039; G03F 7/20

(52) U.S. Cl. CPC G03F 7/0045; G03F 7/0392; G03F 7/2006

(57) ABSTRACT

An onium salt having formula (1) is provided. A chemically amplified resist composition comprising the onium salt as a PAG has advantages including solvent solubility and improved lithography properties such as high sensitivity, high contrast, EL, and LWR when processed by photolithography using high-energy radiation.

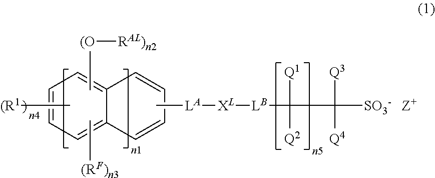

### CROSS-REFERENCE TO RELATED APPLICATIONS

This non-provisional application claims priority under 35 U.S.C. § 119(a) on Patent Application No. 2022-129450 filed in Japan on Aug. 16, 2022, the entire contents of which are hereby incorporated by reference.

### TECHNICAL FIELD

This invention relates to an onium salt, a chemically amplified resist composition, and a pattern forming process.

### BACKGROUND ART

While a number of recent efforts are being made to achieve a finer pattern rule in the drive for higher integration and operating speeds in LSIs, DUV and EUV lithography processes are thought to hold particular promise as the next generation in microfabrication technology. In particular, photolithography using an ArF excimer laser is requisite to the micropatterning technique capable of achieving a feature size of 0.13 μm or less.

The ArF lithography started partial use from the fabrication of 130-nm node devices and became the main lithography since 90-nm node devices. Although lithography using F2 laser (wavelength 157 nm) was initially thought promising as the next lithography for 45-nm node devices, its development was retarded by several problems. A highlight was suddenly placed on the ArF immersion lithography that introduces a liquid having a higher refractive index than air (e.g., water, ethylene glycol, glycerol) between the projection lens and the wafer, allowing the projection lens to be designed to a numerical aperture (NA) of 1.0 or higher and achieving a higher resolution. See Non-Patent Document 1. The ArF immersion lithography is now implemented on the commercial stage. The immersion lithography requires a resist material which is substantially insoluble in water.

In the photolithography using an ArF excimer laser (wavelength 193 nm), a high sensitivity resist material capable of achieving a high resolution at a small dose of exposure is needed to prevent the degradation of precise and expensive optical system materials. Among several measures for providing high sensitivity resist material, the most common is to select each component which is highly transparent at the wavelength of 193 nm. For example, polymers of acrylic acid and derivatives thereof, norbornene-maleic anhydride alternating copolymers, polynorbornene, ring-opening metathesis polymerization (ROMP) polymers, and hydrogenated ROMP polymers have been proposed as the base resin. This choice is effective to some extent in that the transparency of a resin alone is increased.

Recently a highlight is put on the negative tone resist adapted for organic solvent development as well as the positive tone resist adapted for alkaline development. It would be desirable if a very fine hole pattern, which is not achievable with the positive tone, is resolvable through negative tone exposure. To this end, a positive resist material featuring a high resolution is subjected to organic solvent development to form a negative pattern. An attempt to double a resolution by combining two developments, alkali development and organic solvent development is under study. As the ArF resist material for negative tone development with organic solvent, positive ArF resist compositions of the prior art design may be used. Such pattern forming processes are described in Patent Documents 1 to 3.

To meet the current rapid progress of microfabrication technology, development efforts are put on not only the process, but also the resist material. Studies have also been made on photoacid generators (PAGs). Commonly used are sulfonium salts of triphenylsulfonium cations with perfluoroalkanesulfonic acid anions. These salts generate perfluoroalkanesulfonic acids, especially perfluorooctanesulfonic acid (PFOS), which are considered problematic with respect to their non-degradability, biological concentration and toxicity. It is rather restricted to apply these salts to the resist material. Instead, PAGs capable of generating perfluorobutanesulfonic acid are currently used, but are awkward to achieve a high resolution because of substantial diffusion of the generated acid in the resist material. To address the problem, partially fluorinated alkane sulfonic acids and salts thereof are developed. For instance, Patent Document 1 describes the prior art PAGs capable of generating α,α-difluoroalkanesulfonic acid, such as di(4-tert-butylphenyl)iodonitm 1,1-difluoro-2-(1-naphthyl)ethanesulfonate and PAGs capable of generating α,α,β,β-tetrafluoroalkanesulfonic acid. Despite a reduced degree of fluorine substitution, these PAGs still have the following problems. Since they do not have a decomposable substituent group such as ester structure, they are unsatisfactory from the aspect of environmental safety or ease of decomposition. The molecular design to change the size of alkanesulfonic acid is limited. Fluorine-containing starting reactants are expensive.

As the circuit line width is reduced, the degradation of contrast by acid diffusion becomes more serious for the resist material. The reason is that the pattern feature size is approaching the diffusion length of acid. This invites a lowering of mask fidelity and a degradation of pattern rectangularity because a dimensional shift on wafer (known as mask error factor (MEF)) relative to a dimensional shift on mask is exaggerated. Accordingly, to gain more benefits from a reduction of exposure light wavelength and an increase of lens NA, the resist material is required to increase a dissolution contrast or restrain acid diffusion, as compared with the prior art materials. One approach is to lower the bake temperature for suppressing acid diffusion and hence, improving MEF. A low bake temperature, however, inevitably leads to a low sensitivity.

Incorporating a bulky substituent or polar group into PAG is effective for suppressing acid diffusion. Patent Document 4 discloses a PAG capable of generating 2-acyloxy-1,1,3,3,3-pentafluoropropane-1-sulfonic acid which is fully soluble and stable in resist solvents and allows for a wide span of molecular design. In particular, a PAG having a bulky substituent incorporated therein or capable of generating 2-(1-adamantyloxy)-1,1,3,3,3-pentafluoropropane-1-sulfonic acid is characterized by slow acid diffusion. Patent Documents 5 to 7 describe PAGs having fused ring lactone, sultone or thiolactone incorporated as the polar group. Although some improvement in performance is observed due to the acid diffusion suppressing effect of the polar group incorporated, they are still insufficient in precise control of acid diffusion. Their lithography performance is unsatisfactory when evaluated totally in terms of MEF, pattern profile and sensitivity.

Incorporating a polar group into an anion of PAG is effective for suppressing acid diffusion, but disadvantageous from the standpoint of solvent solubility. Attempting to improve solvent solubility, Patent Documents 8 and 9 propose to incorporate an alicyclic group into a cation moiety of a PAG. Specifically, a cyclohexane ring or adamantane ring is incorporated. While incorporating such an alicyclic group achieves an improvement in solubility, a relatively large number of carbon atoms is necessary to insure a satisfactory solubility. This means that the molecular structure of PAG becomes bulky, causing to degrade lithography performance factors such as LWR and CDU in forming small-size patterns.

With the aim to enhance dissolution contrast, it is also in practice to incorporate an acid labile group into an anion or cation of PAG as disclosed in Patent Documents 10 and 11. Many of these PAGs have the structure having a carboxy group protected with an acid labile group. Upon light exposure, elimination reaction of the acid labile group takes place under the catalysis of generated acid. Since the resulting polar group is a carboxy group, the resist film is swollen with the developer during alkaline development and pattern collapse occurs in forming small-size patterns. To meet the demand for further miniaturization, it is crucial to develop a novel PAG. There is the desire to have a PAG capable of fully suppressing acid diffusion, achieving high solvent solubility, and restraining pattern collapse.

### CITATION LIST

Patent Document 1: JP-A 2008-281974
Patent Document 2: JP-A 2008-281975
Patent Document 3: JP 4554665
Patent Document 4: JP-A 2007-145797
Patent Document 5: JP 5061484
Patent Document 6: JP-A 2016-147879
Patent Document 7: JP-A 2015-063472
Patent Document 8: JP 5573098
Patent Document 9: JP 6461919 Patent Document 10: JP 5544078 Patent Document 11: JP 5609569
Non-Patent Document 1: Journal of Photopolymer Science and Technology, Vol. 17, No. 4, p. 587-601 (2004)

### SUMMARY OF THE INVENTION

While it is recently demanded to form resist patterns at a high resolution, a resist composition using a conventional PAG of sulfonium salt type fails to fully suppress acid diffusion. As a result, lithography properties such as contrast, MEF and LWR are degraded. Also, a problem of pattern collapse arises due to swell in forming a small-size pattern.

An object of the invention is to provide an onium salt and a chemically amplified resist composition comprising the same as a photoacid generator, the resist composition having a high solvent solubility and a high sensitivity and being improved in lithography properties such as EL and LWR when processed by photolithography using high-energy radiation such as KrF or ArF excimer laser, EB or EUV; and a pattern forming process using the resist composition.

The inventors have found that an onium salt of specific structure has a high solvent solubility and that a chemically amplified resist composition comprising the onium salt as a photoacid generator exhibits a high sensitivity, high contrast, and improved lithography properties such as EL and LWR, and minimizes the risk of pattern collapse during small-size pattern formation.

In one aspect, the invention provides an onium salt having the formula (1).

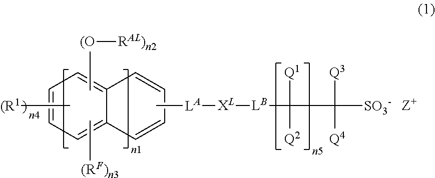

Herein n1 is 0 or 1, n2 is an integer of 1 to 3, n3 is an integer of 1 to 4, n4 is an integer of 0 to 4, meeting n2+n3+n4≤5 in case of n1=0 and n2+n3+n4≤7 in case of n1=1, n5 is an integer of 0 to 4,
R^{AL} forms an acid labile group with the adjoining oxygen atom.
R^{F} is fluorine, a C₁-C₆ fluorinated saturated hydrocarbyl group, C₁-C₆ fluorinated saturated hydrocarbyloxy group, or C₁-C₆ fluorinated saturated hydrocarbylthio group, a plurality of R^{F} may be identical or different in case of n3≥2,
R^{F} and —O—R^{AL} are attached to adjoining carbon atoms,
R¹ is a C₁-C₂₀ hydrocarbyl group which may contain a heteroatom,
L^{A} and L^{B} are each independently a single bond, ether bond, ester bond, sulfonic ester bond, carbonate bond or carbamate bond,
X^{L} is a single bond or a C₁-C₄₀ hydrocarbylene group which may contain a heteroatom,
Q¹ and Q² are each independently hydrogen, fluorine or a C₁-C₆ fluorinated saturated hydrocarbyl group,
Q³ and Q⁴ are each independently fluorine or a C₁-C₆ fluorinated saturated hydrocarbyl group, and
Z⁺ is an onium cation.

In one preferred embodiment, R^{AL} is a group having the formula (AL-1) or (AL-2).

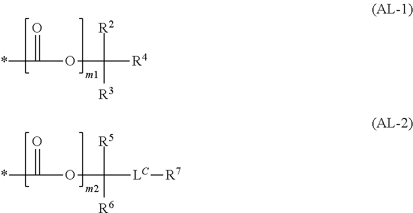

Herein R², R³ and R⁴ are each independently a C₁-C₁₂ hydrocarbyl group in which some —CH₂— may be replaced by —O— or —S—, with the proviso that when the hydrocarbyl group contains an aromatic ring, some or all of the hydrogen atoms on the aromatic ring may be substituted by halogen, cyano, nitro, optionally halogenated C₁-C₄ alkyl moiety or optionally halogenated C₁-C₄ alkoxy moiety, and R² and R³ may bond together to form a ring with the carbon atom to which they are attached, some —CH₂— in the ring may be replaced by —O— or —S—,
R⁵ and R⁶ are each independently hydrogen or a C₁-C₁₀ hydrocarbyl group, R⁷ is a C₁-C₂₀ hydrocarbyl group in which some —CH₂— may be replaced by —O— or —S—, and R⁶ and R⁷ may bond together to form a C₃-C₂₀ heterocyclic group with the carbon atom and L^{C} to which they are attached, some —CH₂— in the heterocyclic group may be replaced by —O— or —S—,
L^{C} is —O— or —S—,
m1 is 0 or 1, m2 is 0 or 1,
designates a point of attachment to the adjoining —O—.

Preferably the onium salt has the formula (1A):

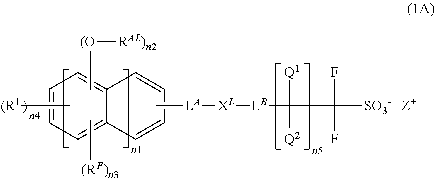

wherein R^{AL}, R^{F}, R¹, L^{A}, L^{B}, X^{L}, Q¹, Q², n1 to n5, and Z are as defined above.

More preferably, the onium salt has the formula (1B):

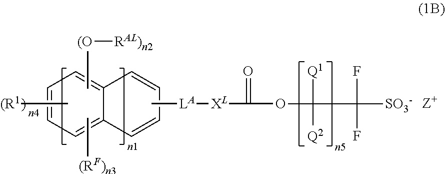

wherein R^{AL}, R^{F}, R¹, L^{A}. X^{L}, Q¹, Q² n1 to n5, and Z⁺ are as defined above.

In one preferred embodiment, Z⁺ is an onium cation having the formula (cation-1) or (cation-2).

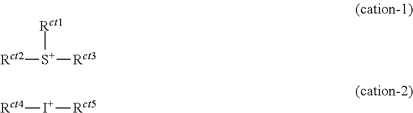

Herein R^{ct1} to R^{ct5} are each independently a C₁-C₃₀ hydrocarbyl group which may contain a heteroatom, and R^{ct1} and R^{ct2} may bond together to form a ring with the sulfur atom to which they are attached.

The invention also provides a photoacid generator comprising the onium salt defined herein.

A chemically amplified resist composition comprising the photoacid generator is also contemplated herein.

Often, the resist composition further comprises a base polymer comprising repeat units having the formula (a1).

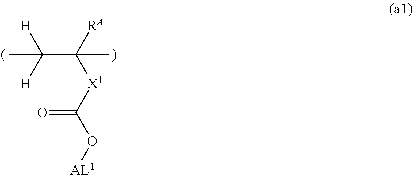

Herein R^{A} is hydrogen, fluorine, methyl or trifluoromethyl,
X¹ is a single bond, phenylene group, naphthylene group or *—C(═O)—O—X¹¹—, the phenylene or naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, or phenylene or naphthylene group, * designates a point of attachment to the carbon atom in the backbone, and
AL¹ is an acid labile group.

In a preferred embodiment, the base polymer further comprises repeat units having the formula (a2).

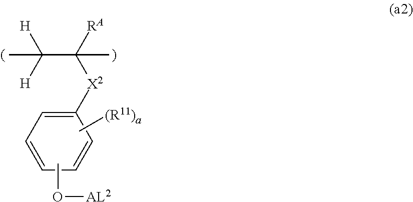

Herein R^{A} is hydrogen, fluorine, methyl or trifluoromethyl,
X² is a single bond or *—C(═O)—O—, * designates a point of attachment to the carbon atom in the backbone,
R²¹ is halogen, cyano, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
AL² is an acid labile group, and
a is an integer of 0 to 4.

In a more preferred embodiment, the base polymer further comprises repeat units having the formula (b1) or (b2).

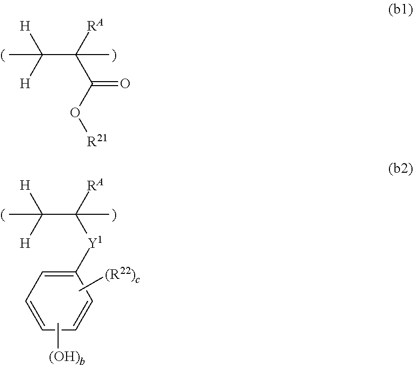

Herein R^{A} is each independently hydrogen, fluorine, methyl or trifluoromethyl,
Y¹ is a single bond or *—C(═O)—O—, * designates a point of attachment to the carbon atom in the backbone,
R¹ is hydrogen or a C₁-C₂₀ group containing at least one structure selected from the group consisting of hydroxy other than phenolic hydroxy, cyano, carbonyl, carboxy, ether bond, ester bond, sulfonic ester bond, carbonate bond, lactone ring, sultone ring and carboxylic anhydride (—C(═O)—O—C(═O)—),
R² is halogen, hydroxy, nitro, a C₁-C₂₀ hydrocarbyl group which may contain a heteroatom, a C₁-C₂ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
b is an integer of 1 to 4, c is an integer of 0 to 4, and b+c is from 1 to 5.

In a preferred embodiment, the base polymer further comprises repeat units of at least one type selected from repeat units having the formulae (c1) to (c4).

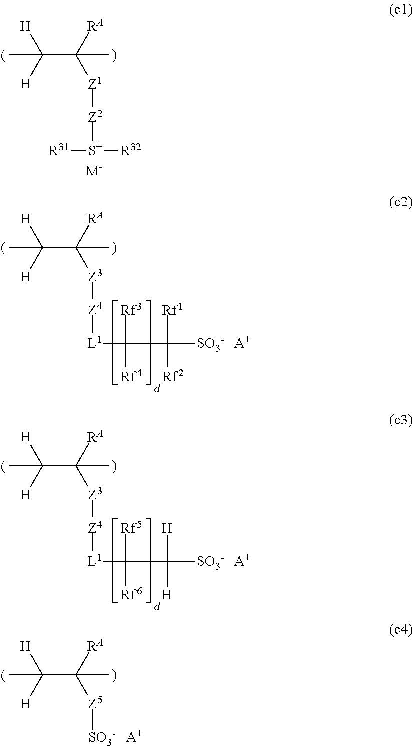

Herein R^{A} is each independently hydrogen, fluorine, methyl or trifluoromethyl,
Z¹ is a single bond or phenylene group,
Z² is *—C(═O)—O—Z²¹—, *—C(═O)—NH—Z²¹—, or *—O—Z²¹—, Z²¹ is a C₁-C₆ aliphatic hydrocarbylene group, phenylene group or a divalent group obtained by combining the foregoing, which may contain a carbonyl moiety, ester bond, ether bond or hydroxy moiety,
Z³ is a single bond, phenylene, naphthylene, or *—C(═O)—O—Z³¹—, Z³¹ is a C₁-C₁₀ aliphatic hydrocarbylene group which may contain a hydroxy moiety, ether bond, ester bond or lactone ring, or phenylene or naphthylene group.
Z⁴ is a single bond or *—Z⁴¹—C(═O)—O—, Z⁴¹ is a C₁-C₂₀ hydrocarbylene group which may contain a heteroatom,
Z⁵ is a single bond, methylene, ethylene, phenylene, fluorinated phenylene, trifluoromethyl-substituted phenylene, *—C(═O)—O—Z⁵¹—, *—C(═O)—N(H)—Z⁵¹—, or *—O—Z⁵¹—, Z⁵¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety,
* designates a point of attachment to the carbon atom in the backbone,
R³¹ and R³² are each independently a C₁-C₂₀ hydrocarbyl group which may contain a heteroatom. R³¹ and R³² may bond together to form a ring with the sulfur atom to which they are attached,
L¹ is a single bond, ether bond, ester bond, carbonyl group, sulfonic ester bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine, or a C₁-C₆ fluorinated saturated hydrocarbyl group,
Rf⁵ and Rf⁶ are each independently hydrogen, fluorine, or a C₁-C₆ fluorinated saturated hydrocarbyl group, excluding that all Rf⁵ and Rf⁶ are hydrogen at the same time,
M⁻ is a non-nucleophilic counter ion,
A⁺ is an onium cation, and
d is an integer of 0 to 3.

The resist composition may further comprise an organic solvent, a quencher, a photoacid generator other than the photoacid generator defined herein, and/or a surfactant.

In a further aspect, the invention provides a pattern forming process comprising the steps of applying the chemically amplified resist composition defined herein onto a substrate to form a resist film thereon, exposing the resist film to high-energy radiation, and developing the exposed resist film in a developer.

Typically, the high-energy radiation is KrF excimer laser radiation, ArF excimer laser radiation, EB or EUV of wavelength 3 to 15 nm.

### Advantageous Effects of Invention

When the chemically amplified resist composition comprising the onium salt as a photoacid generator is processed by lithography, patterns having a high sensitivity, high contrast, improved properties including MEF and LWR can be formed. The risk of pattern collapse is restrained.

### BRIEF DESCRIPTION OF THE DRAWINGS

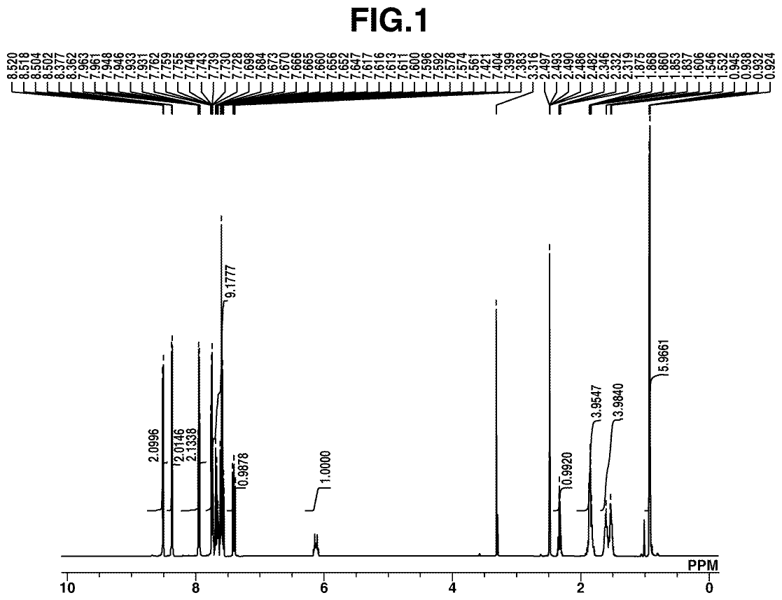

The only FIGURE, FIG. 1 is a diagram showing the ¹H-NMR spectrum of PAG-1 in Example 1-1.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, Me stands for methyl, Ac for acetyl, and the broken line designates a valence bond. Unless otherwise stated, the asterisk (*) designates a point of attachment to the carbon atom in the backbone.

The abbreviations and acronyms have the following meaning.
EB: electron beam
EUV: extreme ultraviolet
Mw: weight average molecular weight
Mn: munber average molecular weight
Mw/Mn: molecular weight dispersity
GPC: gel permeation chromatography
PEB: post-exposure bake
PAG: photoacid generator
EL: exposure latitude
LWR: line width roughness
MEF: mask error factor
CDU: critical dimension uniformity
DOF: depth of focus

Onium Salt

The invention provides an onium salt having the formula (1).

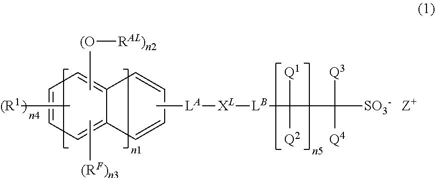

In formula (1), n1 is 0 or 1. The relevant structure is a benzene ring in case of n1=0, and a naphthalene ring in case of n1=1. Of these, a benzene ring corresponding to n1=0 is preferred from the standpoint of solvent solubility. The subscript n2 is an integer of 1 to 3. It is preferred from the standpoint of availability of reactants that n2 be 1 or 2, most preferably 1. The subscript n3 is an integer of 1 to 4. It is preferred from the standpoint of availability of reactants that n3 be 1 or 2, most preferably 1. The subscript n4 is an integer of 0 to 4. These subscripts meet n2+n3+n4≤5 in case of n1=0 and n2+n3+n4≤7 in case of n1=1. The subscript n5 is an integer of 0 to 4, preferably an integer of 0 to 3, most preferably 1.

In formula (1), R^{AL} forms an acid labile group with the adjoining oxygen atom. The preferred acid labile group has the formula (AL-1) or (AL-2).

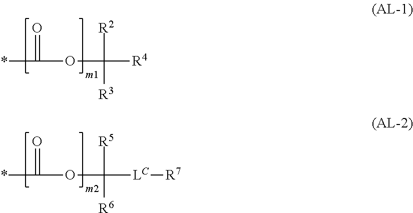

In formula (AL-1). R², R³ and R⁴ are each independently a C₁-C₁₂ hydrocarbyl group. In the hydrocarbyl group, some —CH₂— may be replaced by —O— or —S—. When the hydrocarbyl group contains an aromatic ring, some or all of the hydrogen atoms on the aromatic ring may be substituted by halogen, cyano, nitro, optionally halogenated C₁-C₄ alkyl moiety or optionally halogenated C₁-C₄ alkoxy moiety. The subscript m1 is 0 or 1, and * designates a point of attachment to the adjoining —O—.

The C₁-C₁₂ hydrocarbyl group represented by R², R³ and R⁴ may be saturated or unsaturated and straight, branched or cyclic. Examples thereof include C₁-C₁₂ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, n-decyl, n-undecyl, n-dodecyl; C₃-C₁₂ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl, tetracyclo[6.2.1.1^{3,6}.0^{2,7}]dodecyl; C₂-C₁₂ alkenyl groups such as vinyl, allyl, propenyl, butenyl, pentenyl, hexenyl; C₂-C₁₂ alkynyl groups such as ethynyl, propynyl, butynyl, pentynyl, hexynyl; C₃-C₁₂ cyclic unsaturated aliphatic hydrocarbyl groups such as cyclopentenyl and cyclohexenyl; C₆-C₁₂ aryl groups such as phenyl, naphthyl and indanyl; C₇-C₁₂ aralkyl groups such as benzyl, 1-phenylethyl, 2-phenylethyl, and combinations thereof.

Also, R² and R³ may bond together to form a ring with the carbon atom to which they are attached. Some —CH₂— in the ring may be replaced by —O— or —S—. Exemplary rings include cyclopropane, cyclobutane, cyclopentane, cyclohexane, cycloheptane, cyclooctane, norbornane, adamantane, tricyclo[5.2.1.0^{2,6}]decane, and tetracyclo[6.2.1.1^{3,6}.0^{2,7}] dodecane rings.

In formula (AL-2), R⁵ and R⁶ are each independently hydrogen or a C₁-C₁₀ hydrocarbyl group. The C₁-C₁₀ hydrocarbyl group represented by R⁵ and R⁶ may be saturated or unsaturated and straight, branched or cyclic. Examples thereof are as exemplified above for the C₁-C₁₀ hydrocarbyl groups R¹ and R².

In formula (AL-2), R⁷ is a C₁-C₂₀ hydrocarbyl group in which some —CH₂— may be replaced by —O— or —S—. The C₁-C₂₀ hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, norbornylmethyl, adamantyl, adamantylmethyl, tricyclo[5.2.1.0^{2,6}]decyl, tetracyclo[6.2.1.1^{3,6}.0^{2.7}]dodecyl; C₂-C₂₀ alkenyl groups such as vinyl, propenyl, butenyl, pentenyl, hexenyl: C₂-C₂₀ alkynyl groups such as ethynyl, propynyl, butynyl, pentynyl, hexynyl; C₃-C₂₀ cyclic unsaturated aliphatic hydrocarbyl groups such as cyclopentenyl, cyclohexenyl, norbornenyl: C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naplhthyl, methylnaphthyl, ethylnaphthyl, n-propytlnaphthyl, isopropylnaphthyl, n-butylnaphthyl, isobutylnaphthyl, sec-butylnaphthyl, tert-butylnaphthyl: C₇-C₂₀ aralkyl groups such as benzyl, phenethyl, and combinations thereof. Also, R⁶ and R⁷ may bond together to form a C₃-C₂₀ heterocyclic group with the carbon atom and L^{C} to which they are attached. Some —CH₂— in the heterocyclic group may be replaced by —O— or —S—.

In formula (AL-2), L^{C} is —O— or —S—.

In formula (AL-2), m2 is 0 or 1 and * designates a point of attachment to the adjoining —O—.

Examples of the acid labile group having formula (AL-1) are shown below, but not limited thereto. The asterisk (*) designates a point of attachment to the adjoining —O—.

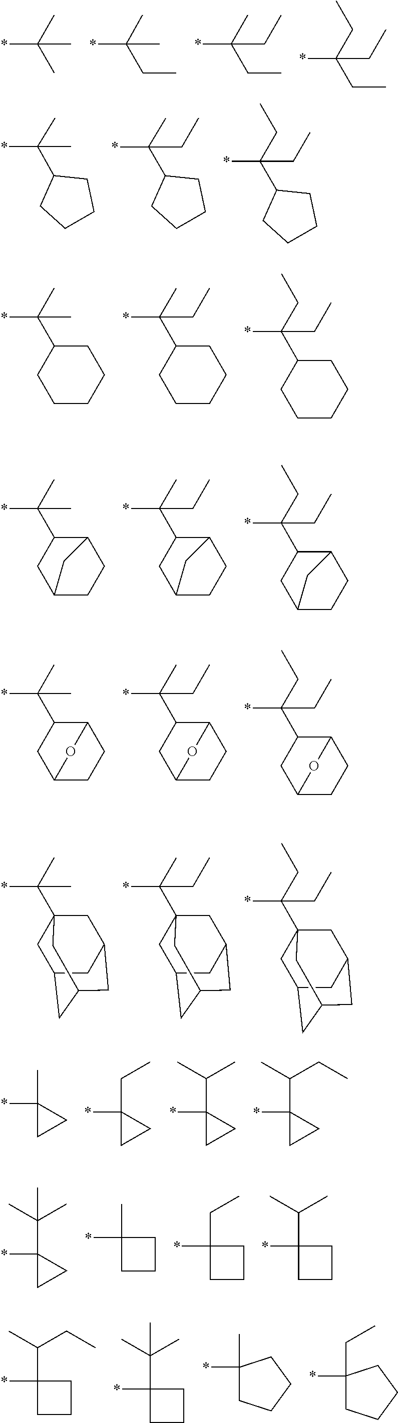

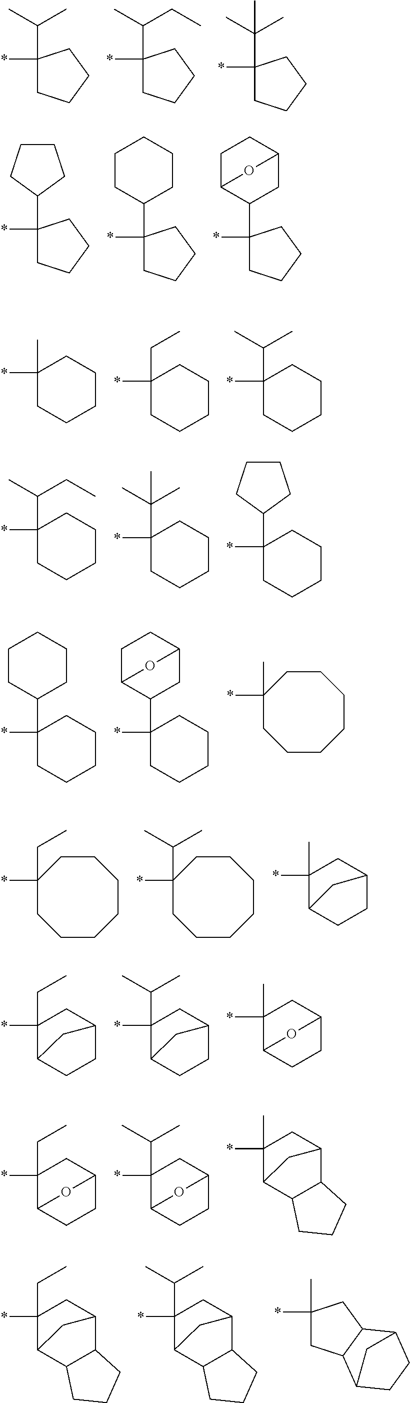

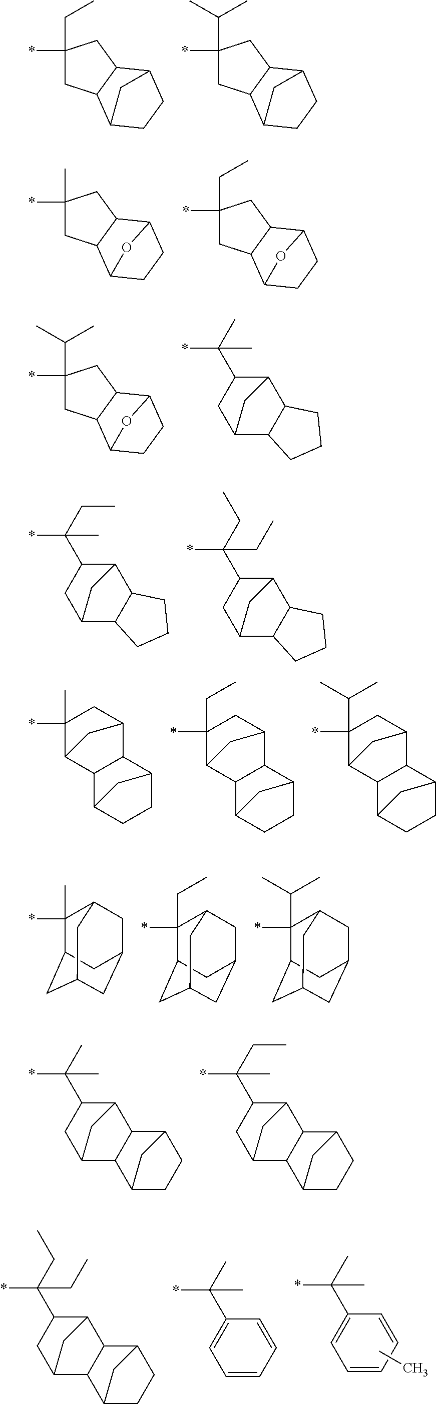

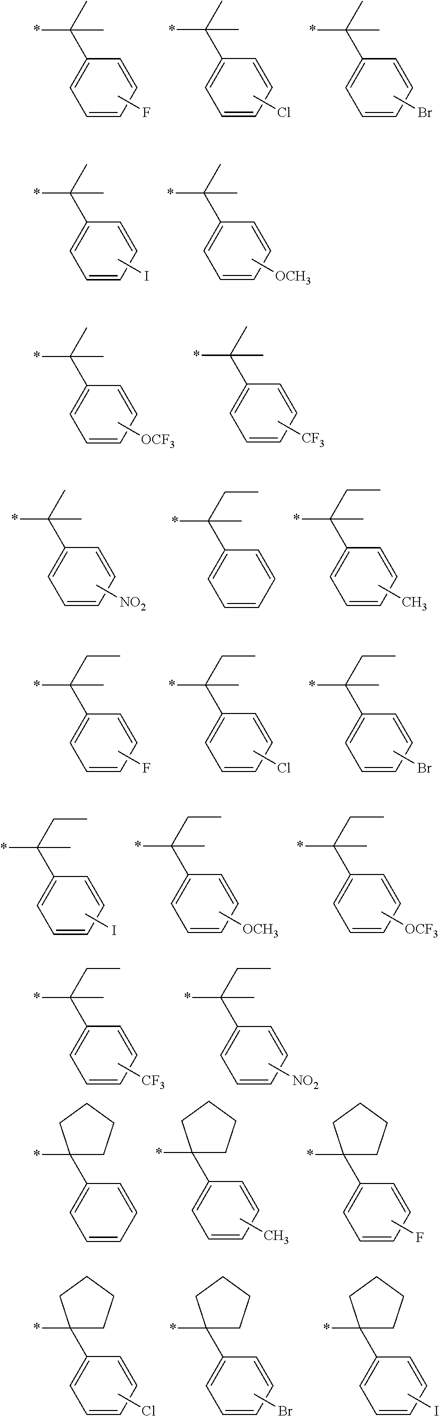

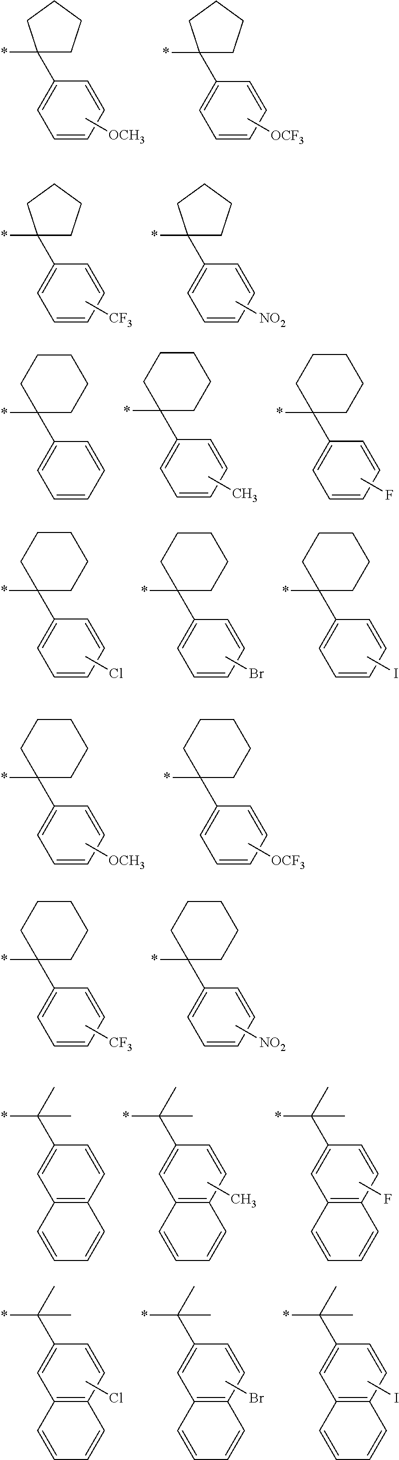

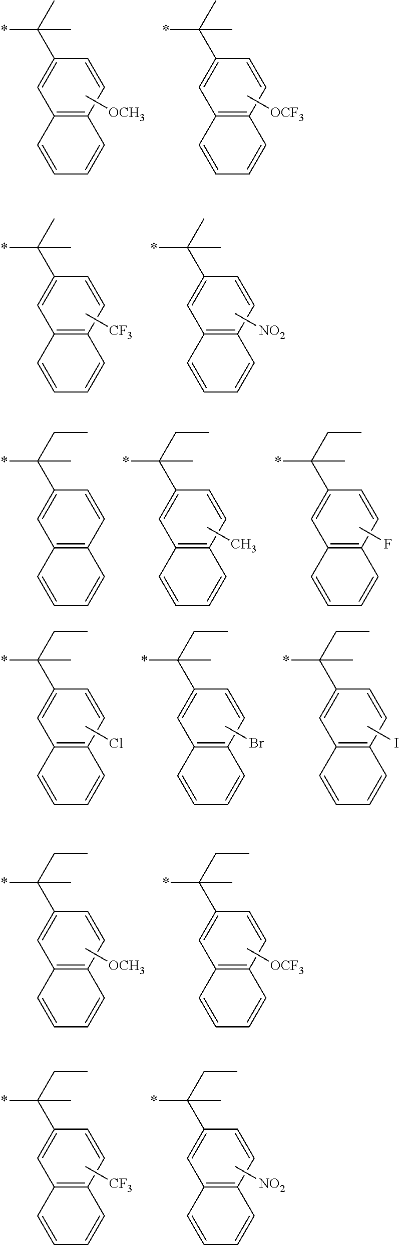

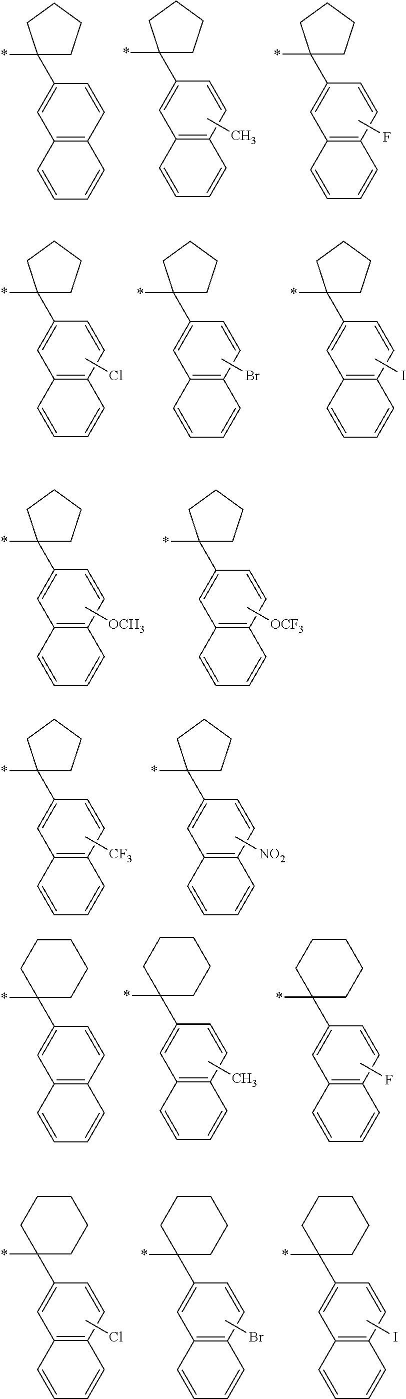

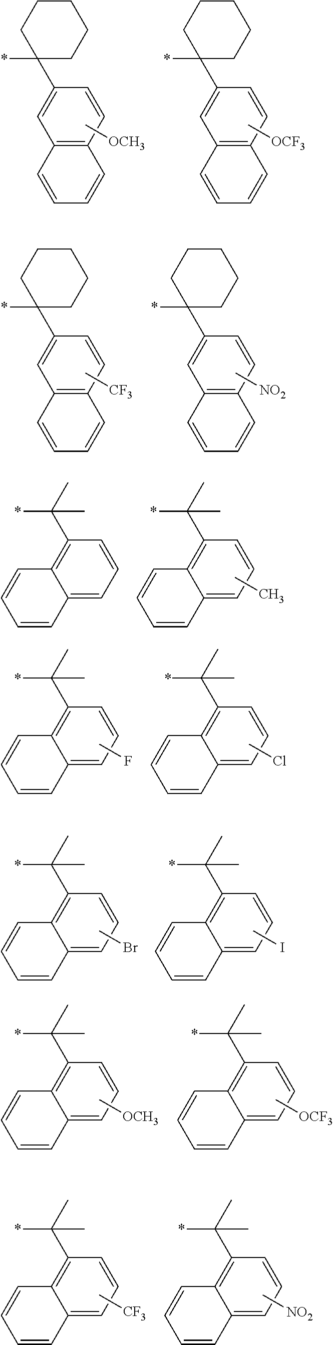

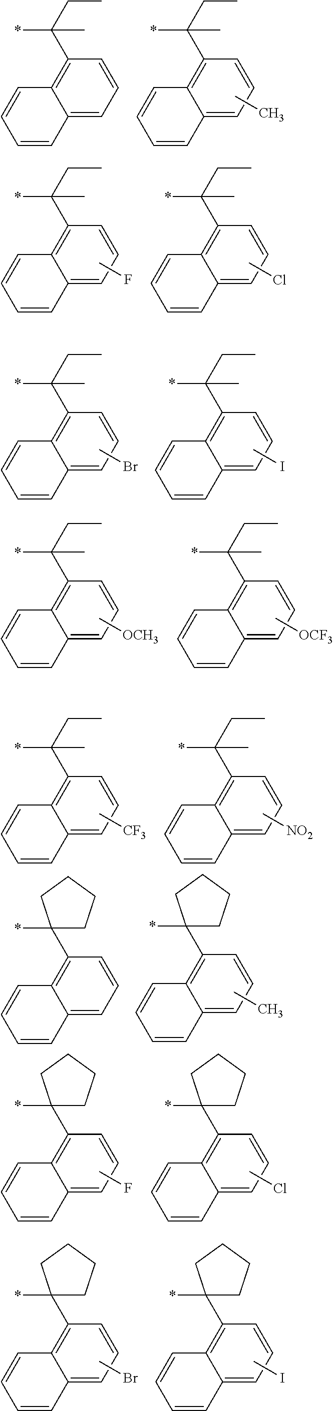

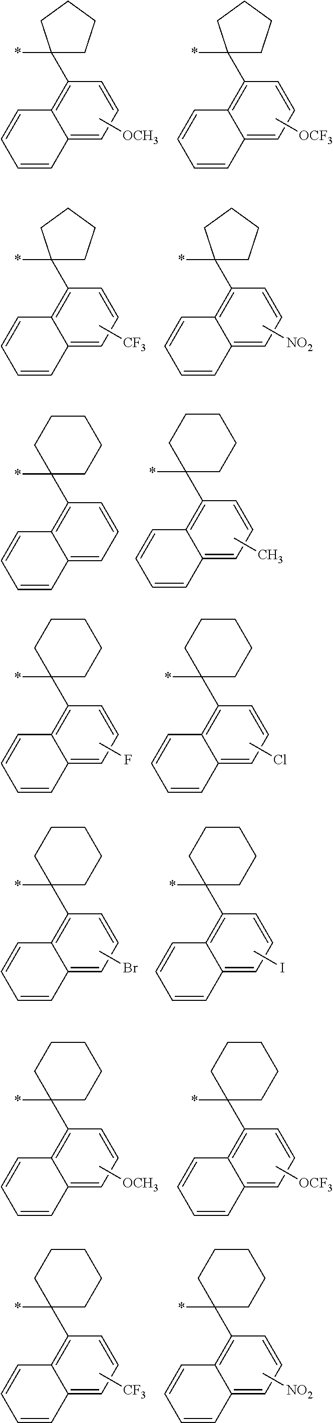

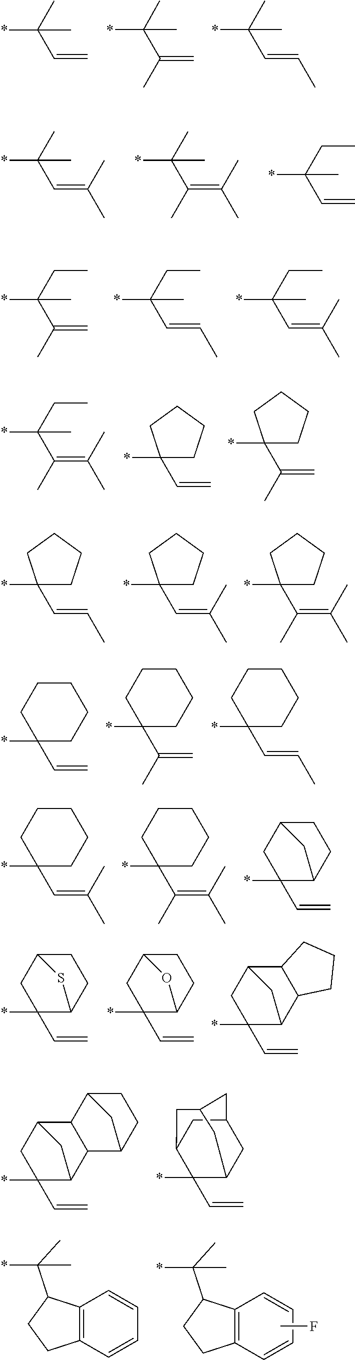

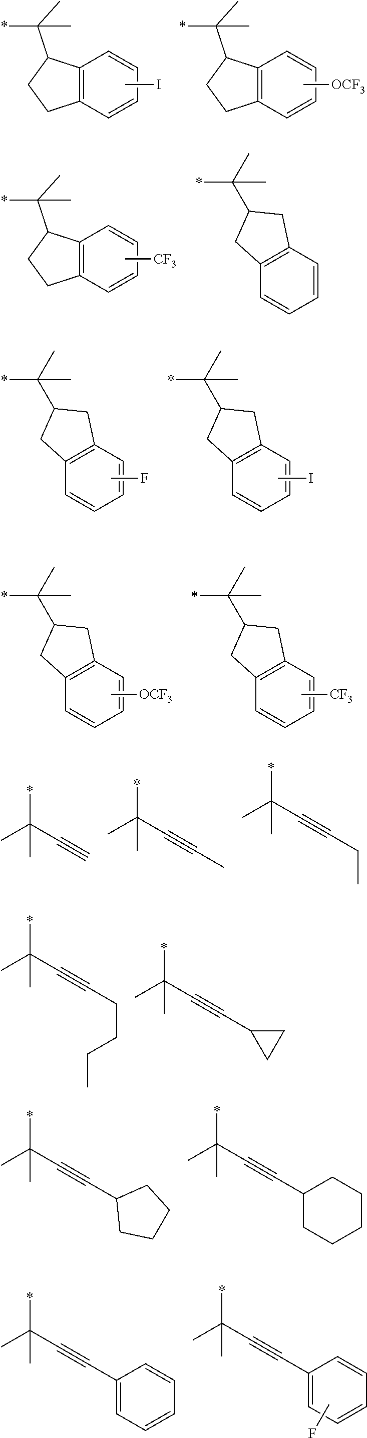

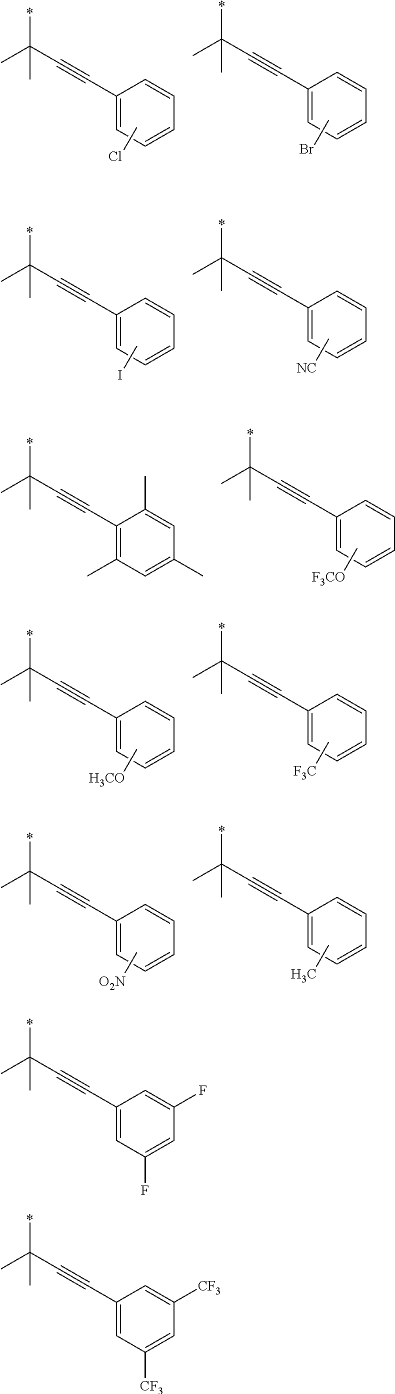

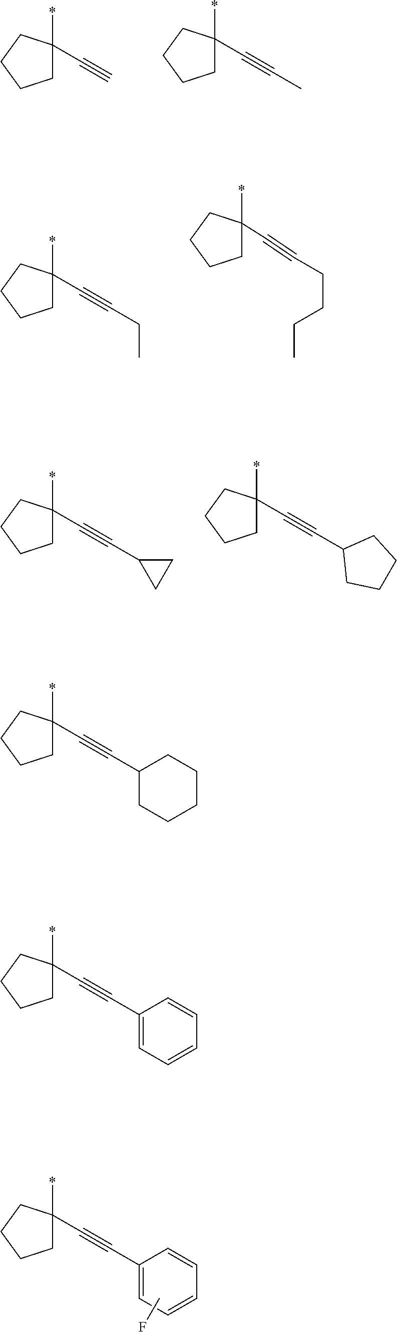

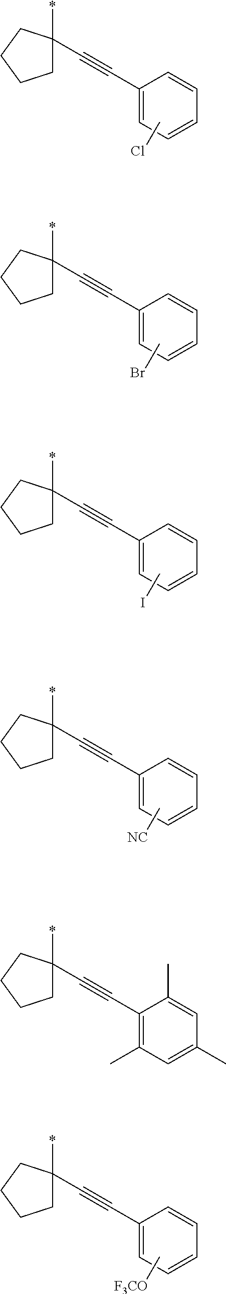

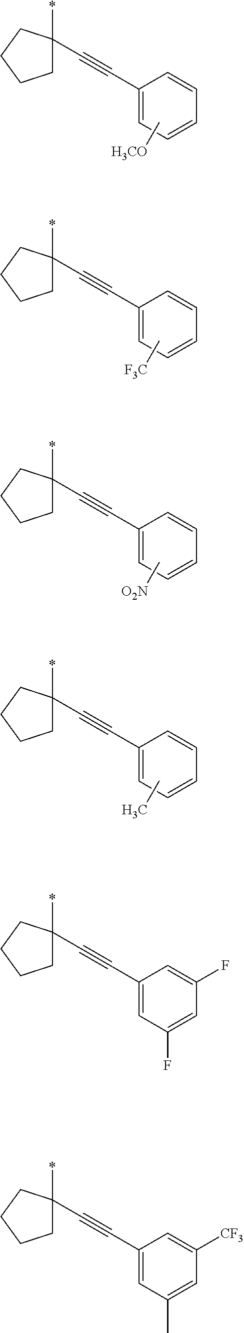

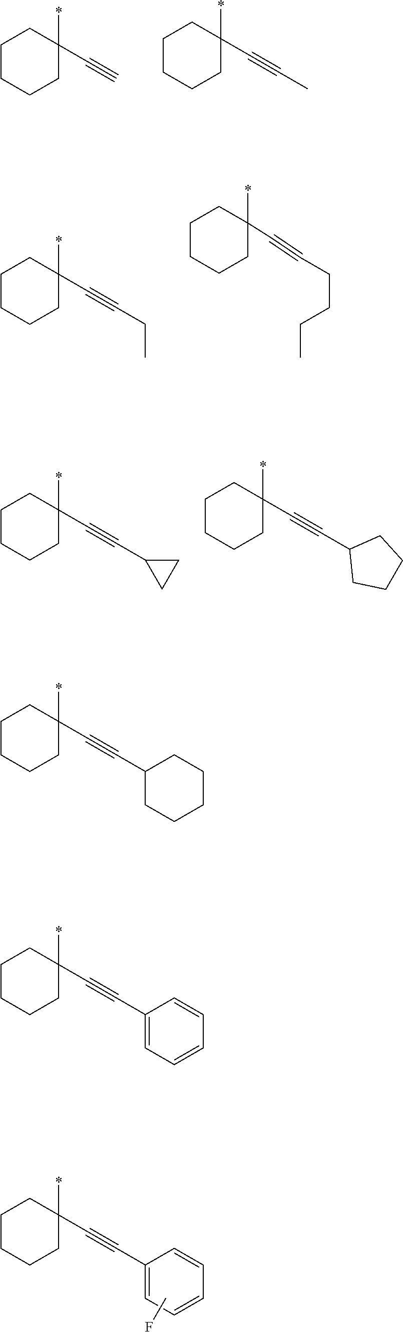

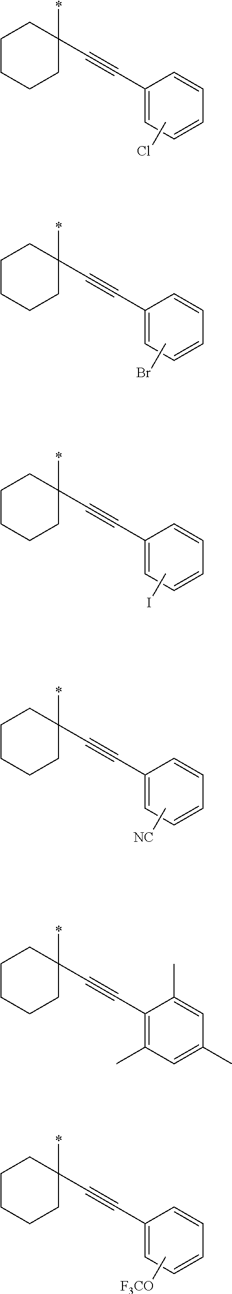

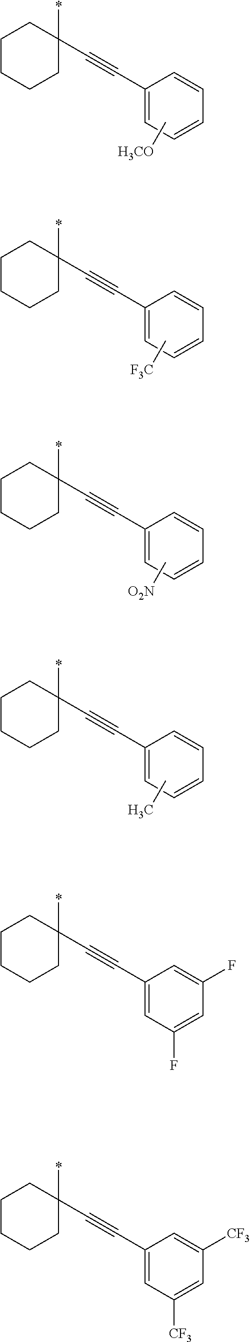

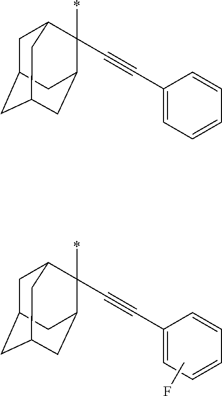

Examples of the acid labile group having formula (AL-2) are shown below, but not limited thereto. The asterisk (*) designates a point of attachment to the adjoining —O—.

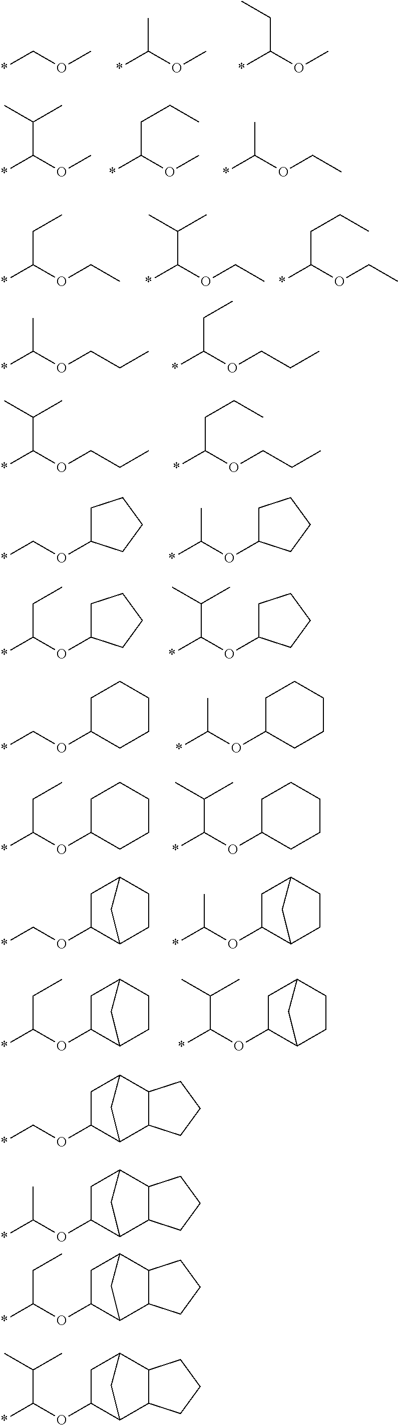

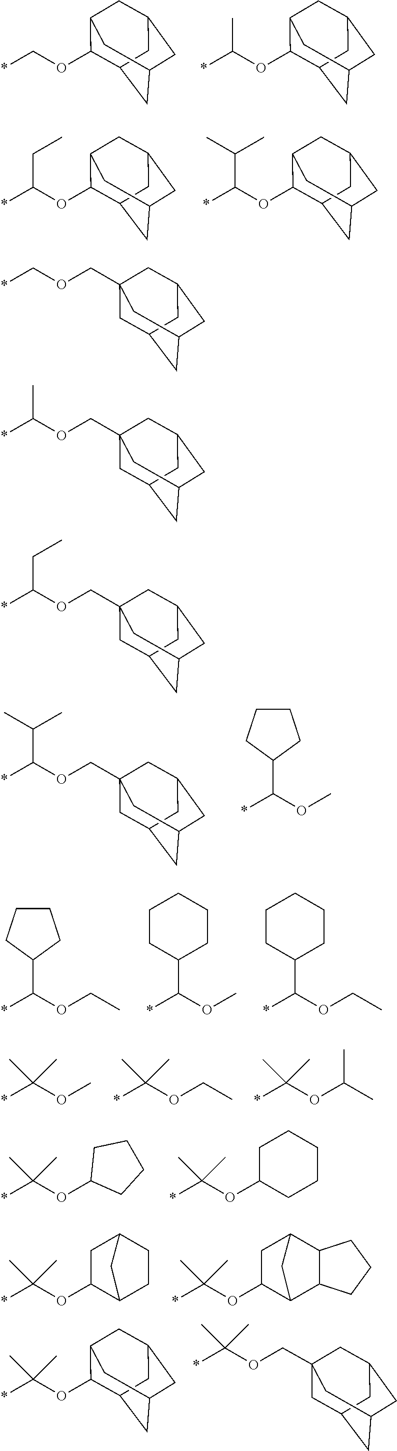

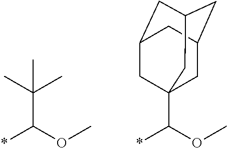

In formula (1), R^{F} is fluorine, a C₁-C₆ fluorinated saturated hydrocarbyl group. C₁-C₆ fluorinated saturated hydrocarbyloxy group, or C₁-C₆ fluorinated saturated hydrocarbylthio group. A plurality of R^{F} may be identical or different in case of n3≥22. Suitable groups include C₁-C₆ fluorinated alkyl, alkoxy, and sulfide groups, preferably trifluoromethyl, trifluoromethoxy and trifluoromethylthio.

In formula (1), R^{F} and —O—R^{AL} are attached to adjoining carbon atoms. Their adjacent arrangement leads to an improvement in the acidity of an aromatic alcohol resulting from elimination of —R^{AL}, and hence, an improvement in dissolution contrast.

In formula (1), R¹ is a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, adamantyl; C₂-C₂₀ alkenyl groups such as vinyl, allyl, propenyl, butenyl, hexenyl: C₃-C₂₀ cyclic unsaturated hydrocarbyl groups such as cyclohexenyl; C₆-C₂₀ aryl groups such as phenyl, naphthyl; C₇-C₂₀ aralkyl groups such as benzyl, 1-phenylethyl, 2-phenylethyl, and combinations thereof. Inter alia, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety.

In formula (1), L^{A} and L^{B} are each independently a single bond, ether bond, ester bond, sulfonic ester bond, carbonate bond or carbamate bond, preferably a single bond, ether bond or ester bond.

In formula (1), X^{L} is a single bond or a C₁-C₄ hydrocarbylene group which may contain a heteroatom. The hydrocarbylene group may be straight, branched or cyclic and examples thereof include alkanediyl and cyclic saturated hydrocarbylene groups. Suitable heteroatoms include oxygen, nitrogen and sulfur.

Examples of the optionally heteroatom-containing C₁-C₄ hydrocarbylene group X^{L} are shown below. The asterisk (*) designates a point of attachment to L^{A} or L^{B}.

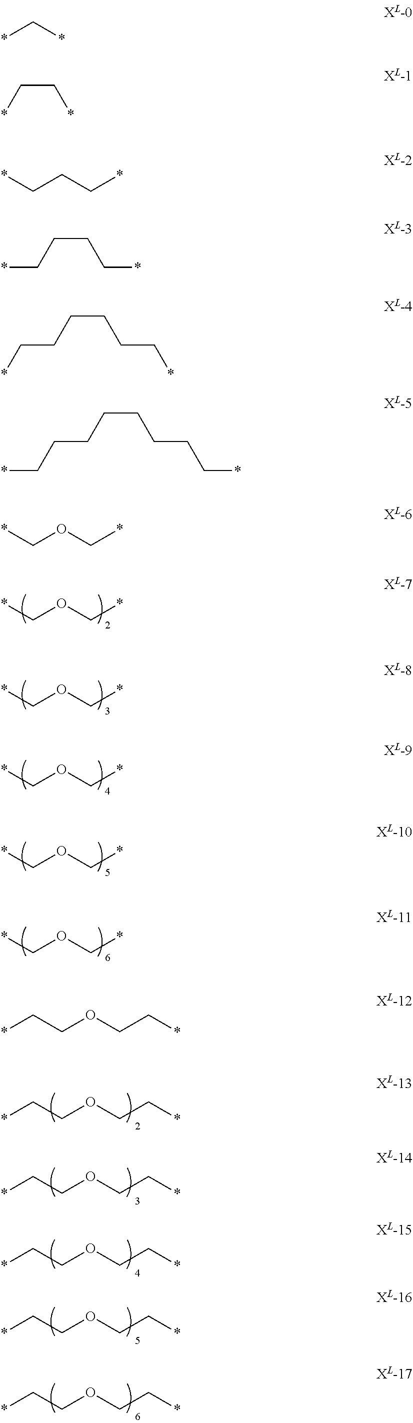

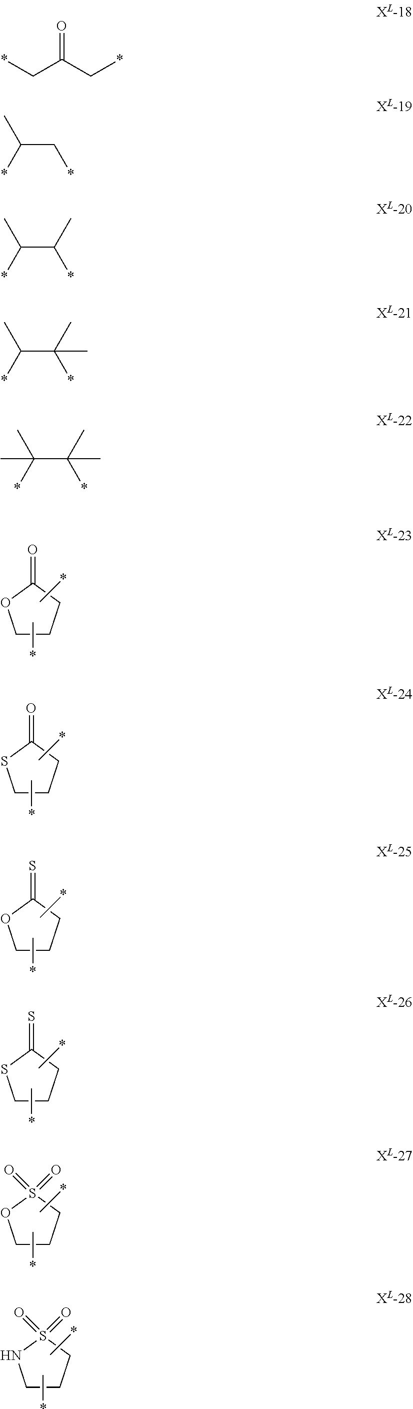

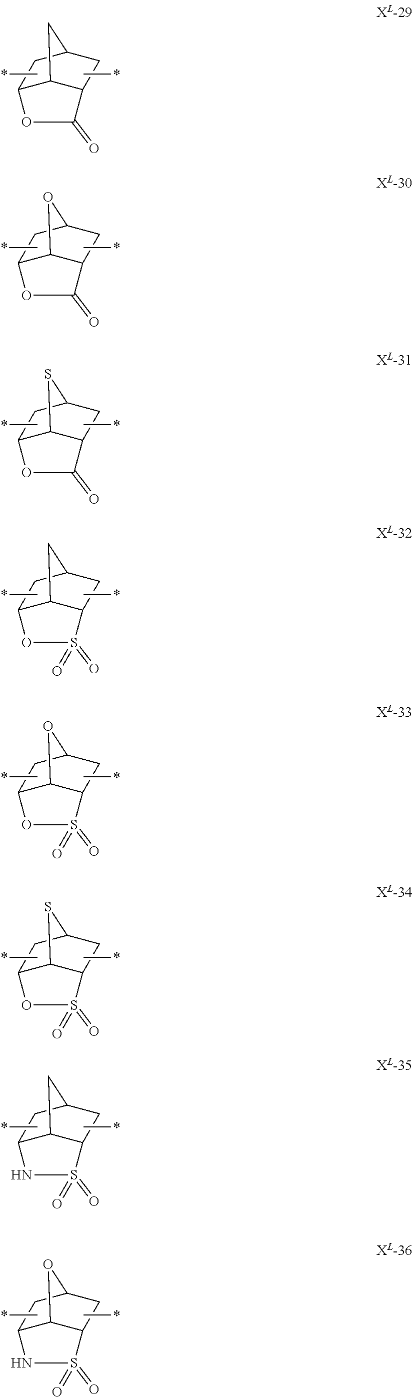

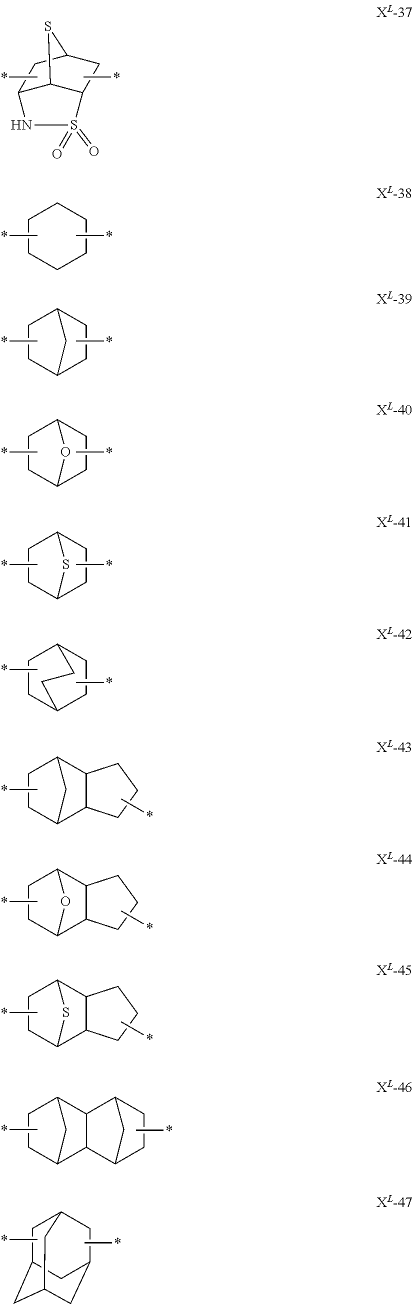

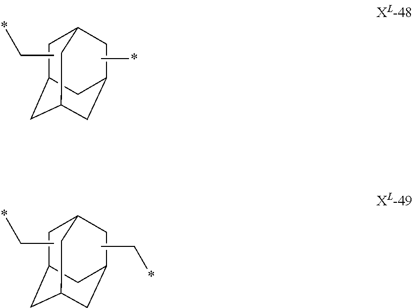

Of these, X^{L}-0 to X^{L}-3, X^{L}-29 to X^{L}-34, X^{L}-47 to X^{L}-49 are preferred, with X^{L}-0 to X^{L}-2, X^{L}-29 and X^{L}-47 being more preferred.

In formula (1). Q¹ and Q² are each independently hydrogen, fluorine or a C₁-C₆ fluorinated saturated hydrocarbyl group. Typical of the C₁-C₆ fluorinated saturated hydrocarbyl group is trifluoromethyl.

In formula (1). Q³ and Q⁴ are each independently fluorine or a C₁-C₆ fluorinated saturated hydrocarbyl group. Typical of the C₁-C₆ fluorinated saturated hydrocarbyl group is trifluoromethyl.

Preferred examples of the partial structure: —[C(Q¹)(Q²)]ₙ₅—C(Q³)(Q⁴)—SO₃— in formula (1) are shown below, but not limited thereto. Herein, * designates a point of attachment to L^{B}.

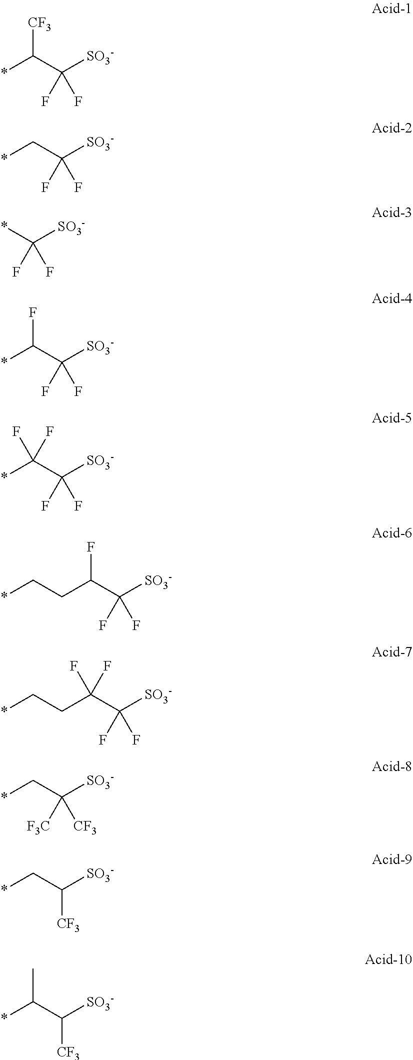

Of these, Acid-1 to Acid-7 are preferred, with Acid-1 to Acid-3, Acid-6 and Acid-7 being more preferred.

Of the onium salts having formula (1), those having the formula (1A) are preferred.

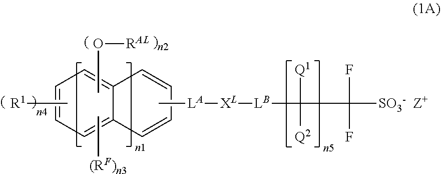

Herein R^{AL}, R^{F}, R¹, L^{A}, L^{B}, X^{L}, Q¹, Q², n1 to n5, and Z⁺ are as defined above.

Of the onium salts having formula (1A), those having the formula (1B) are more preferred.

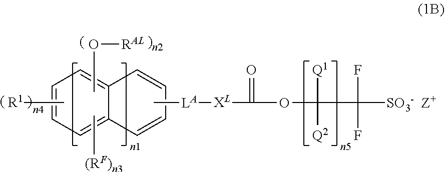

Herein R^{AL}, R^{F}, R¹, L^{A}, X^{L}, Q¹, Q², n1 to n5, and Z⁺ are as defined above.

Examples of the anion in the onium salt having formula (1) are shown below, but not limited thereto. The position of a substituent on the aromatic ring is not limited to the illustrated one as long as —O—R^{L} and R^{F} are arranged adjacent. Herein Q¹ is as defined above.

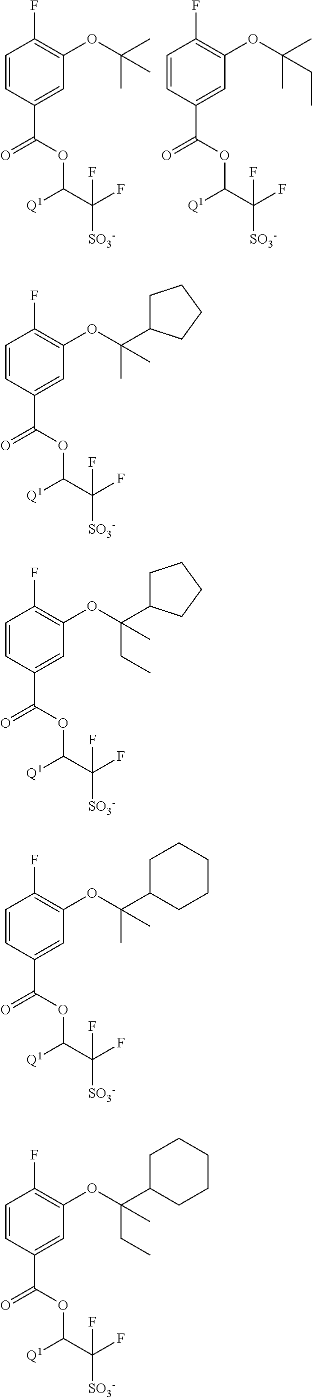

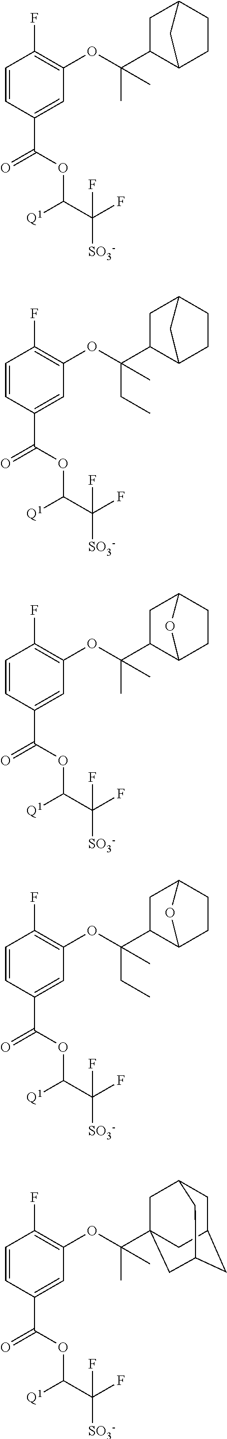

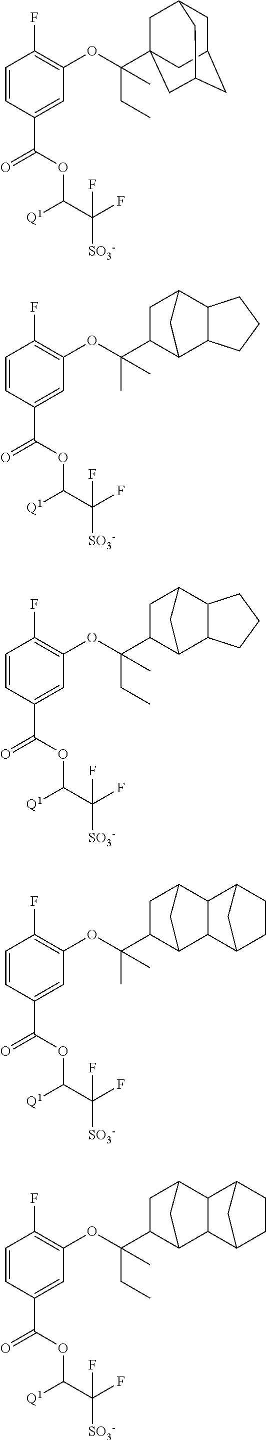

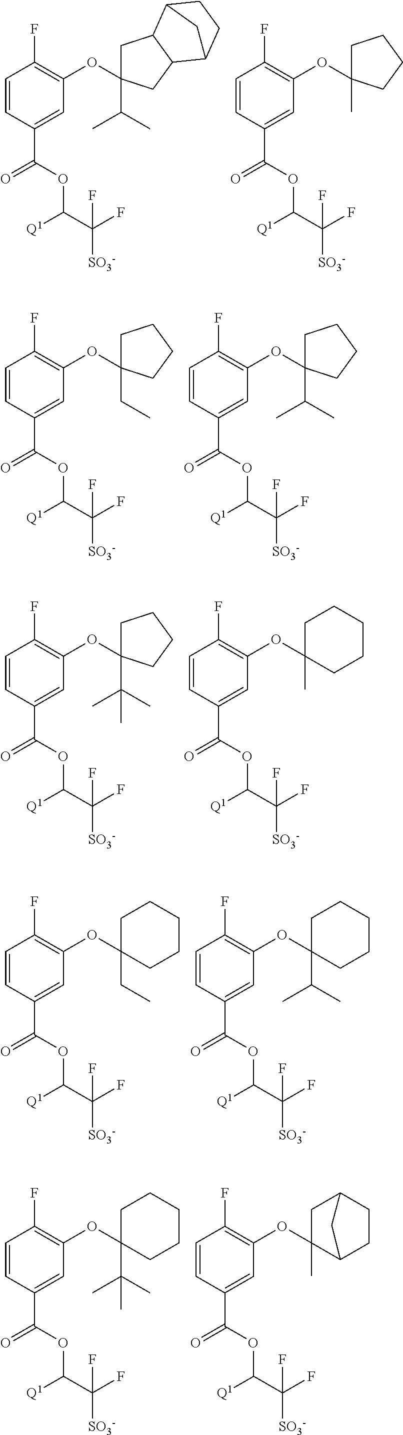

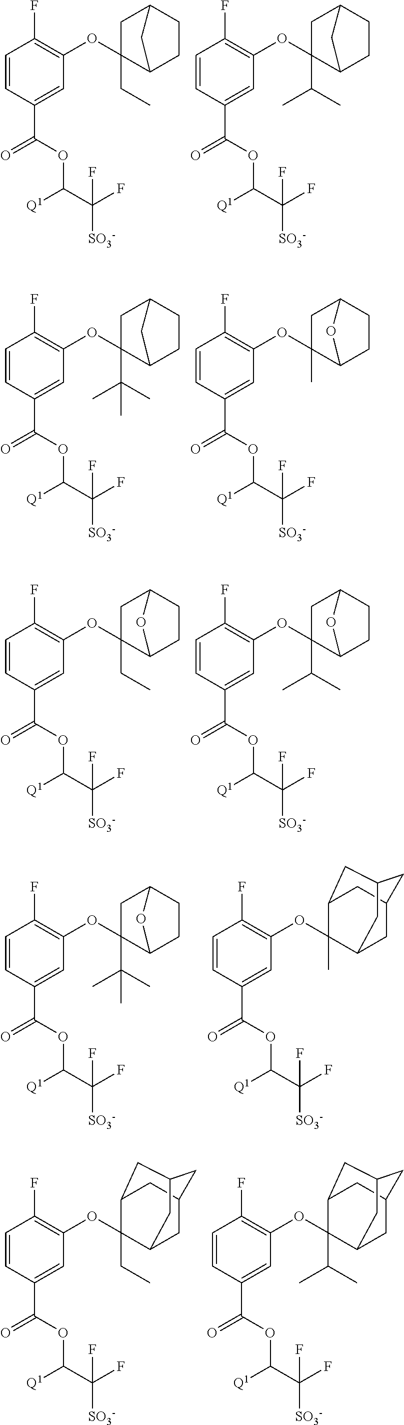

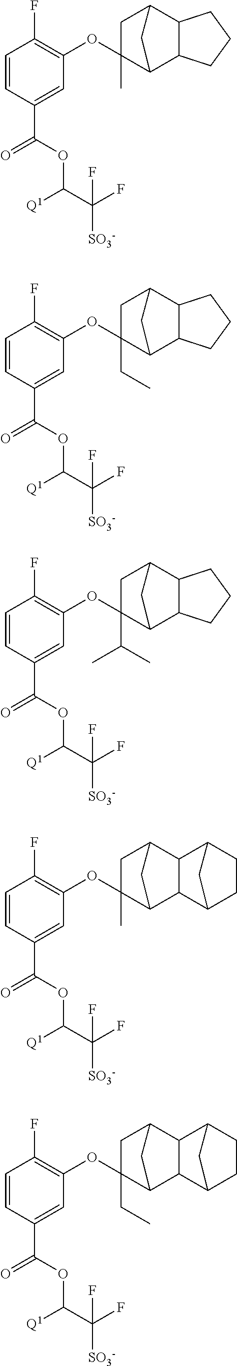

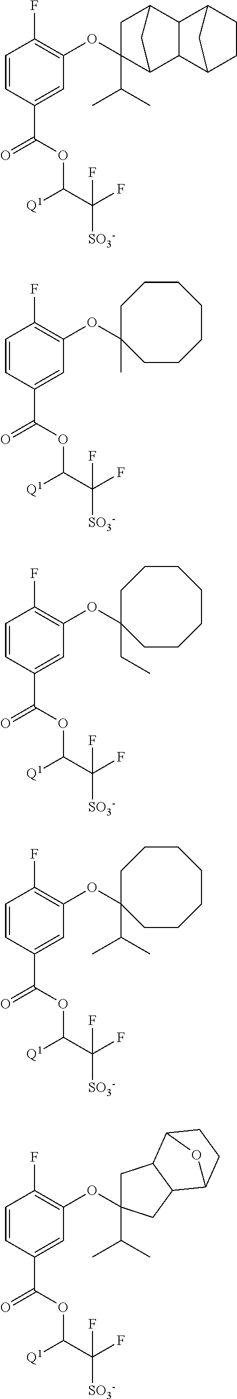

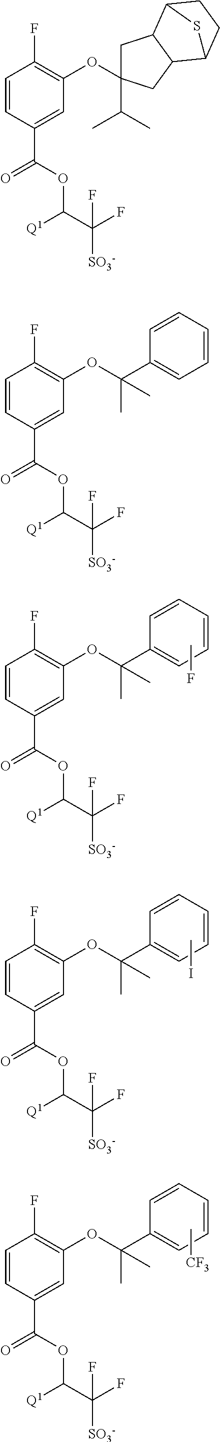

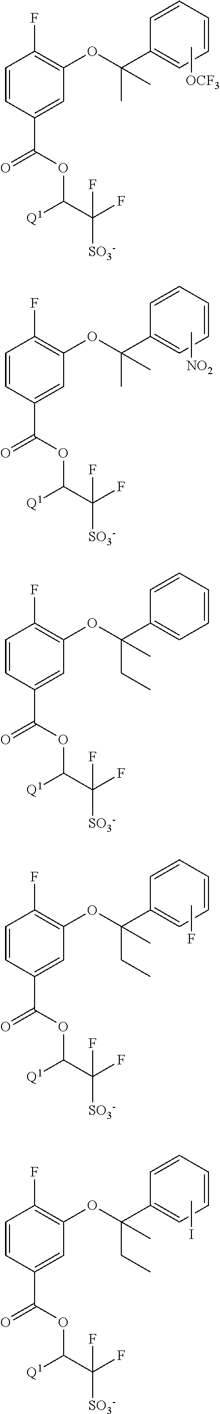

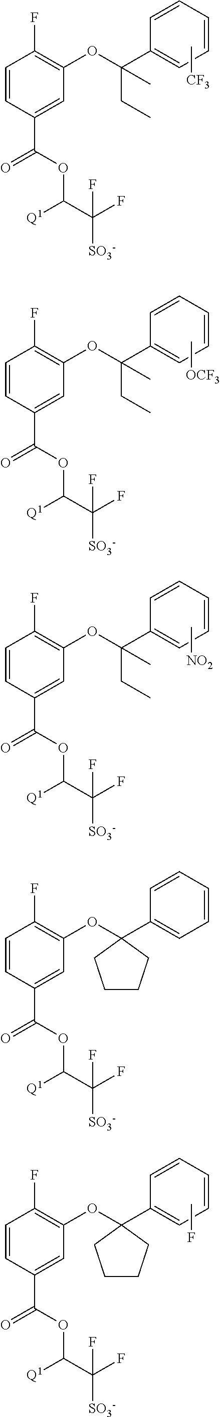

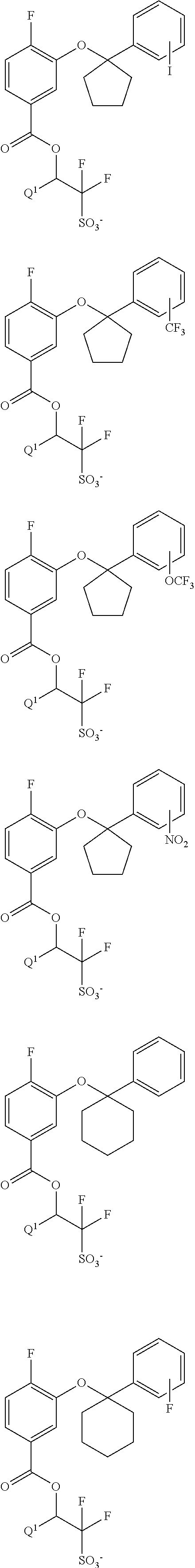

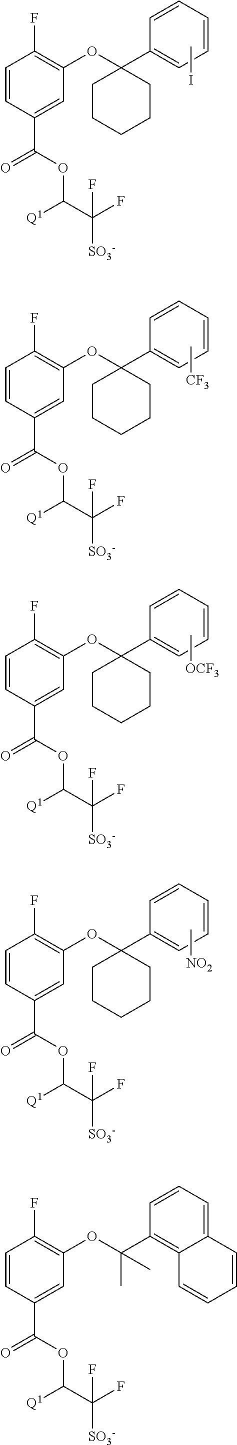

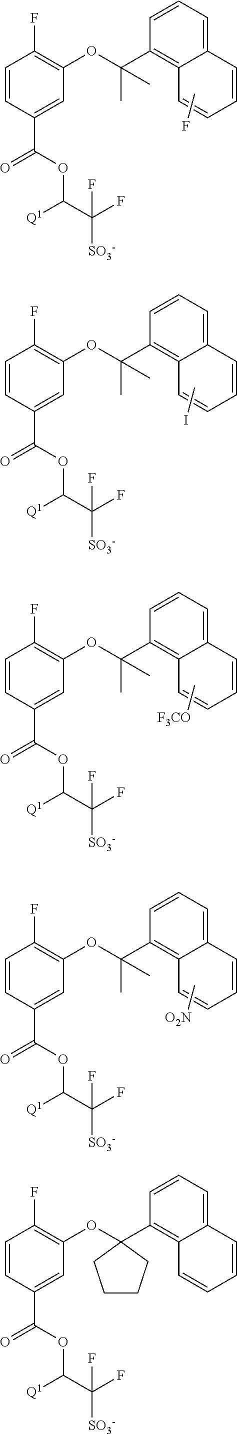

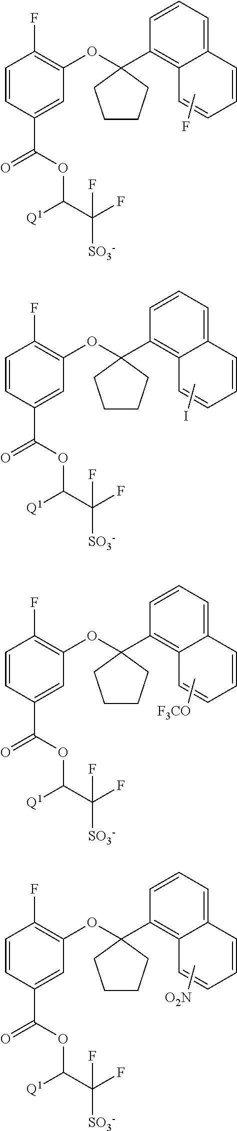

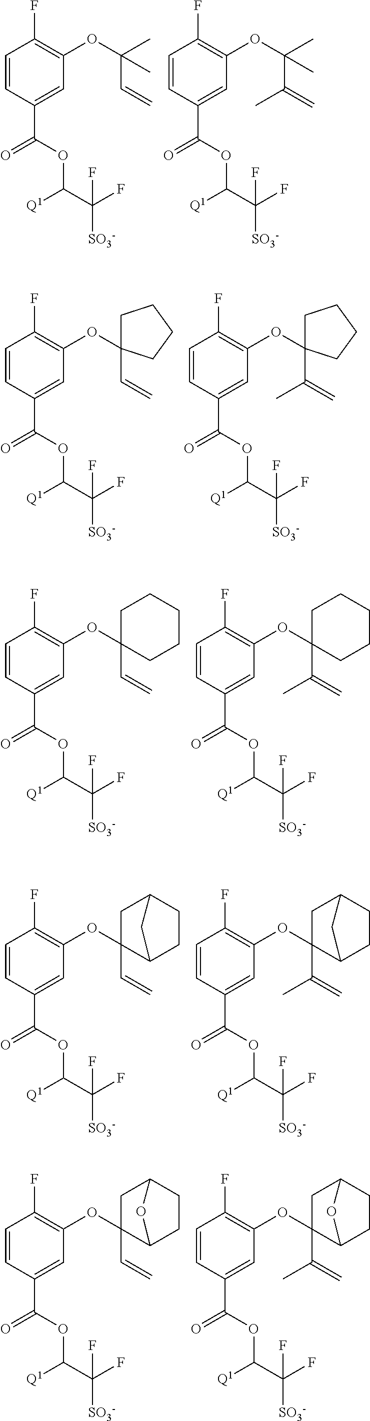

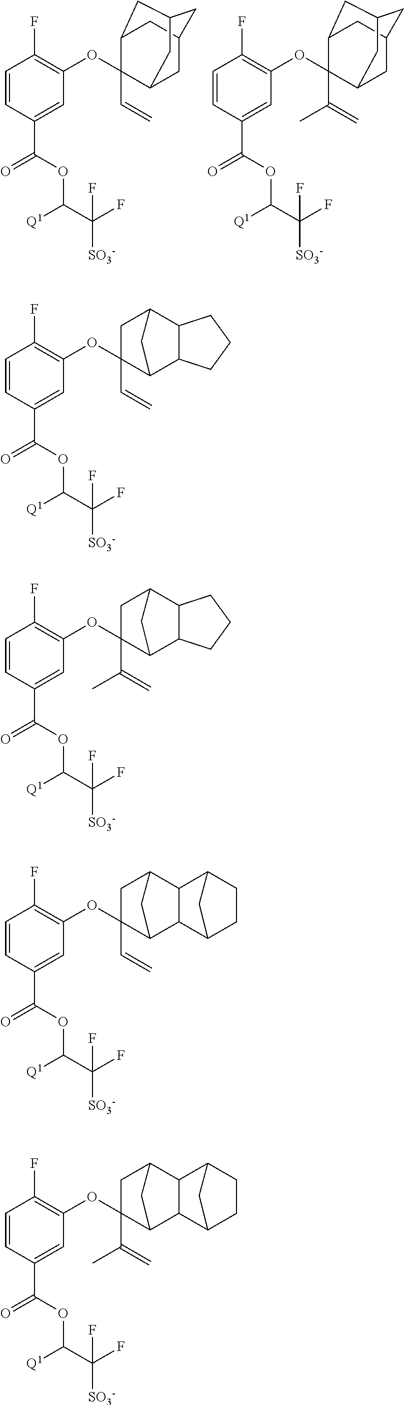

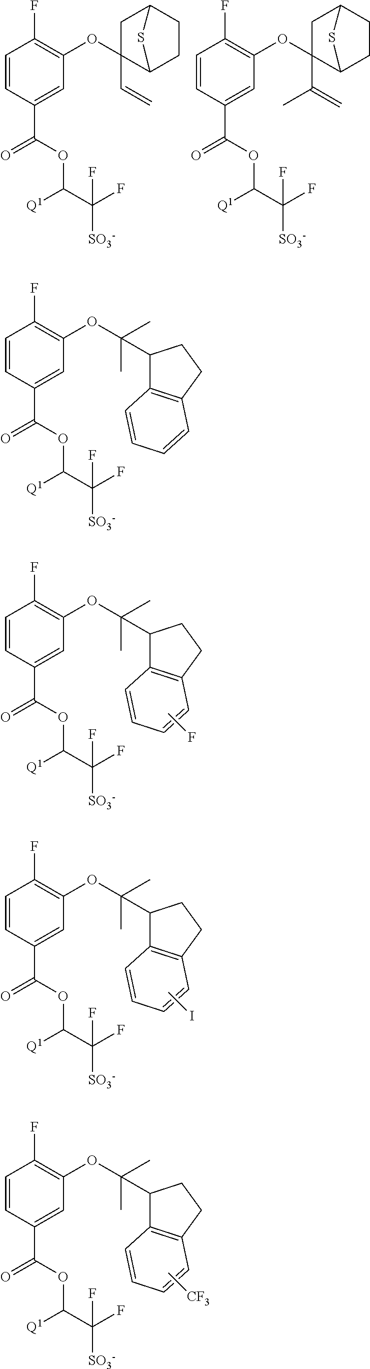

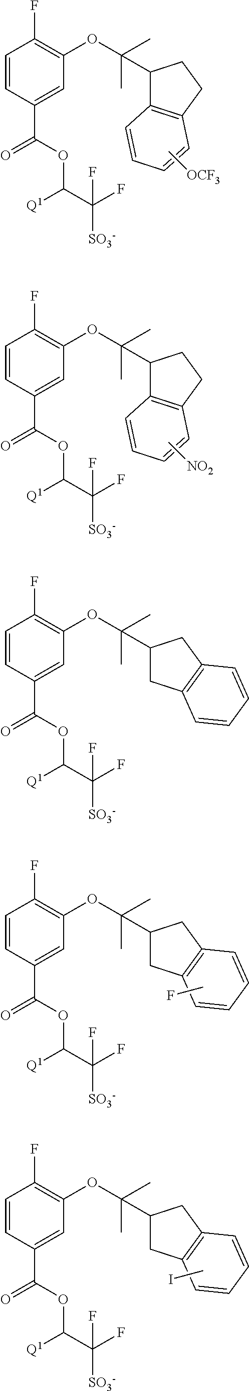

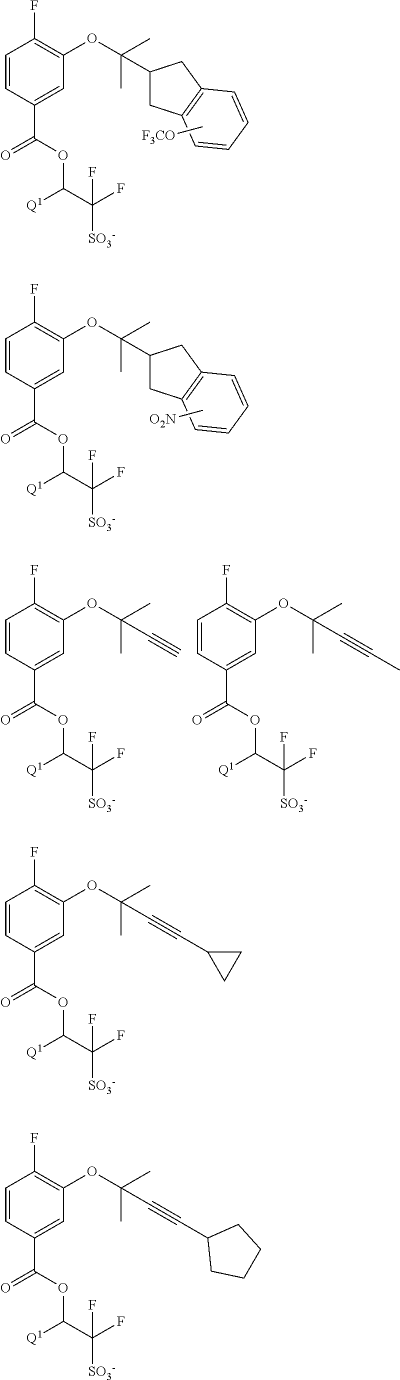

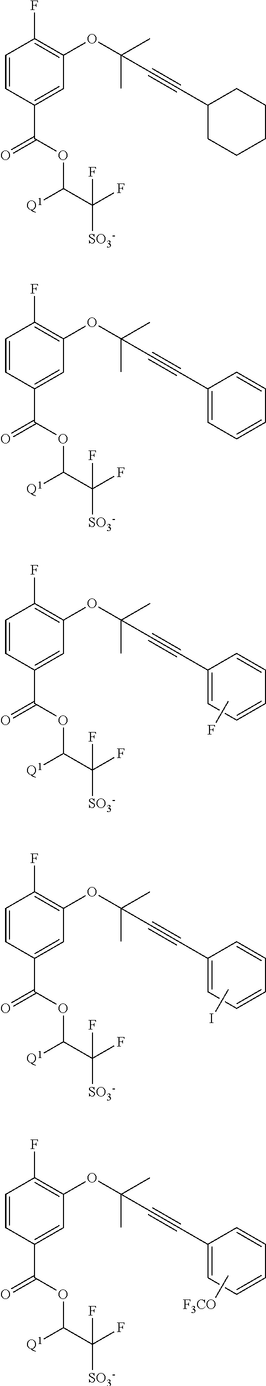

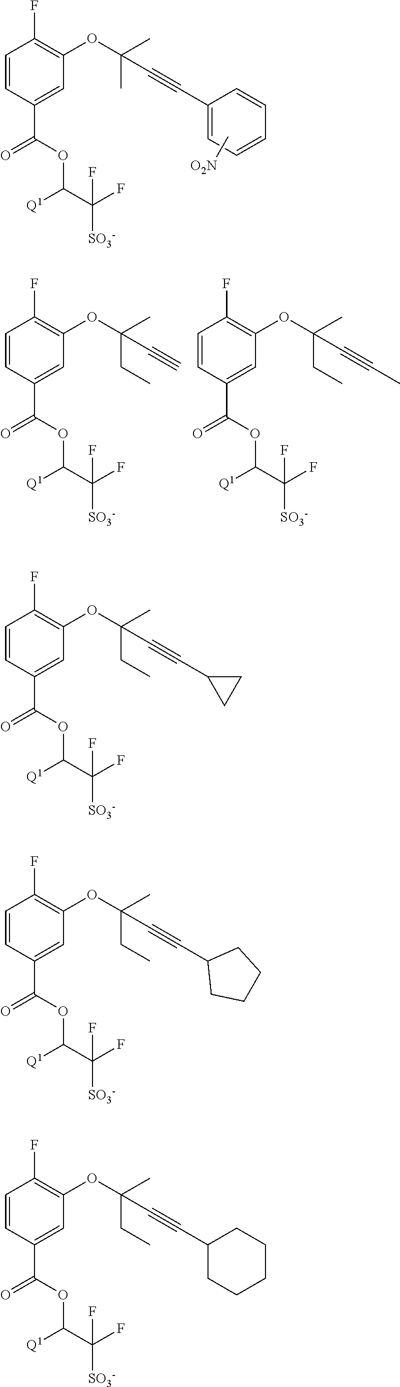

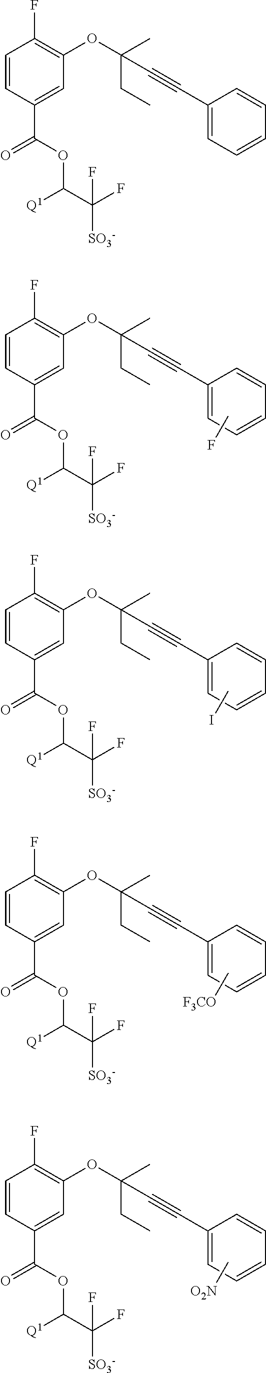

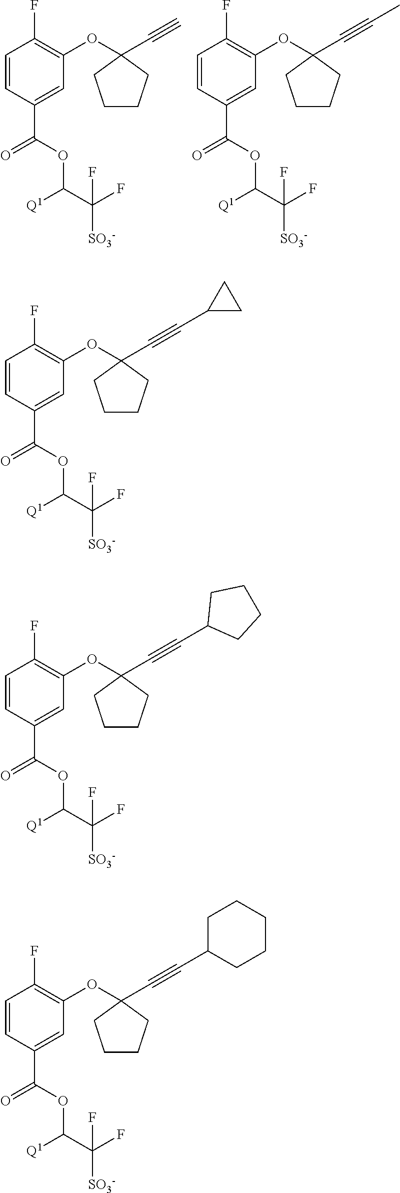

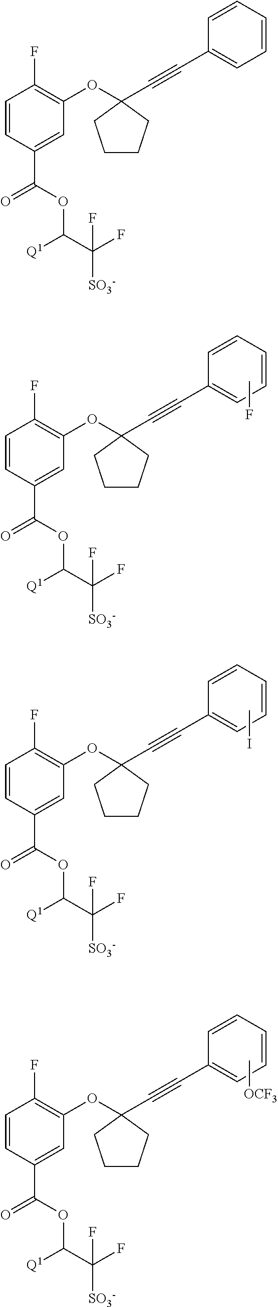

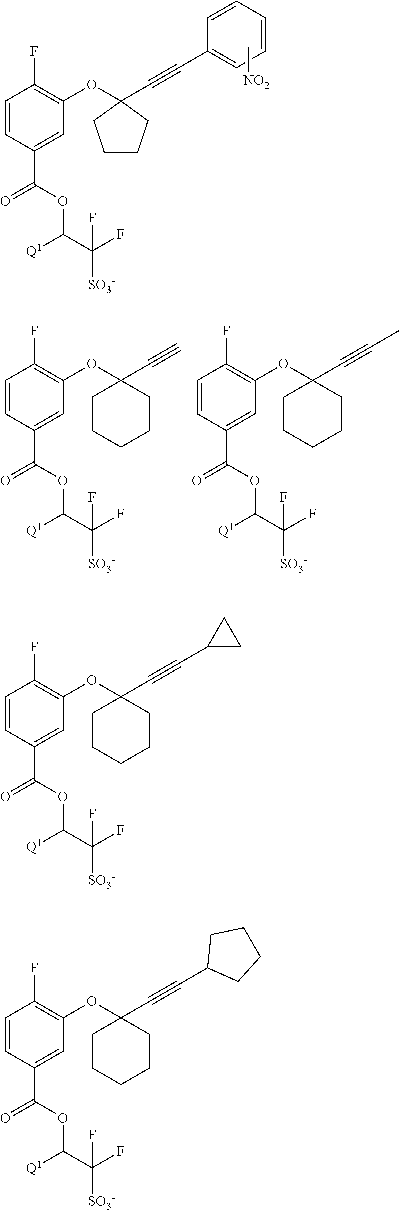

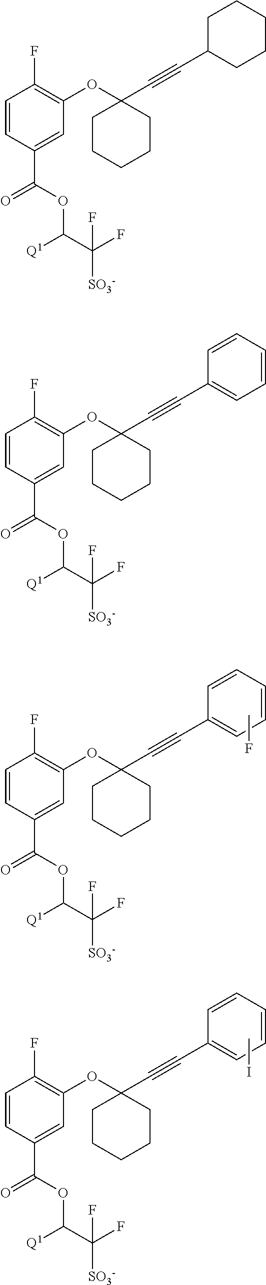

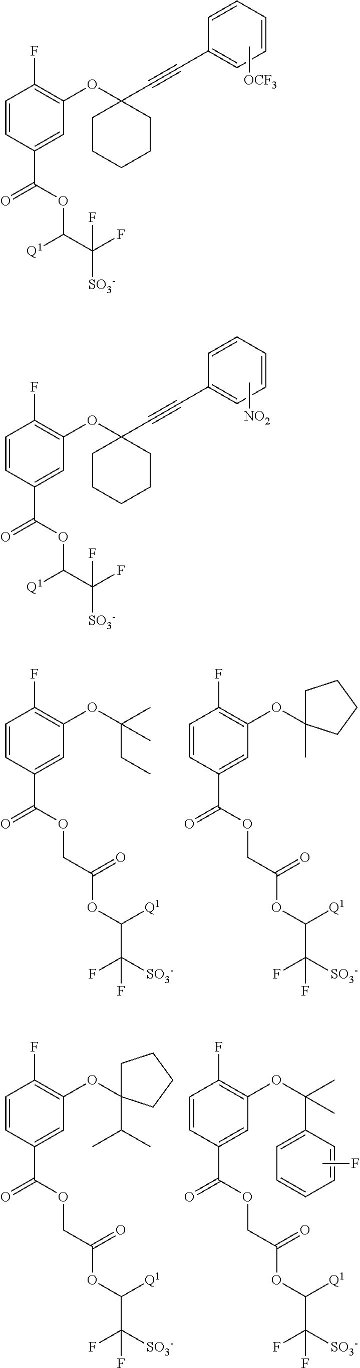

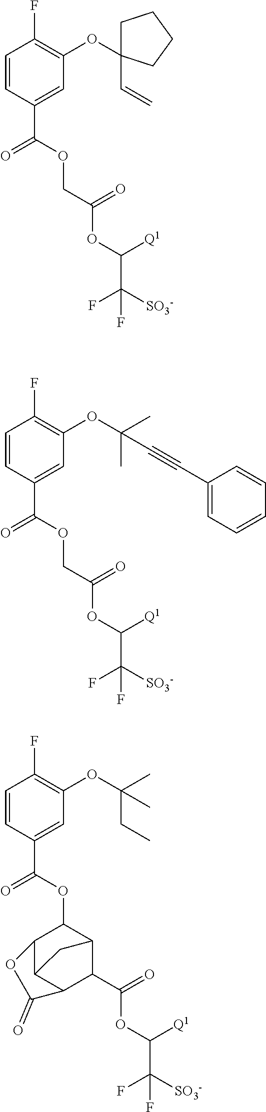

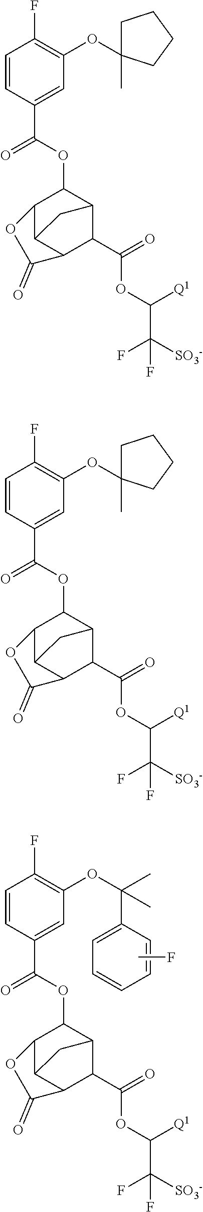

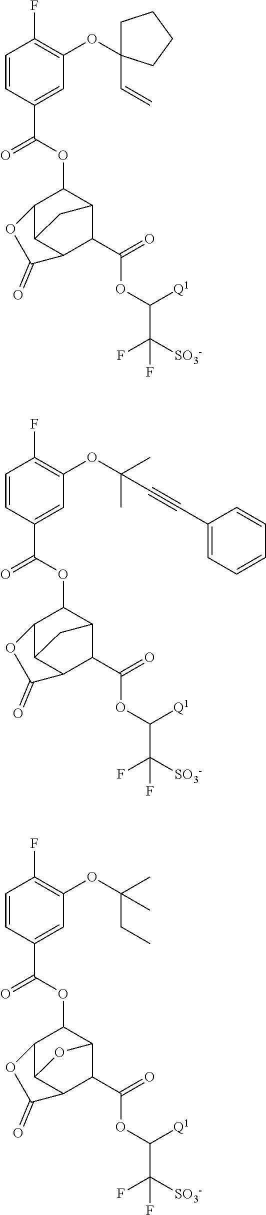

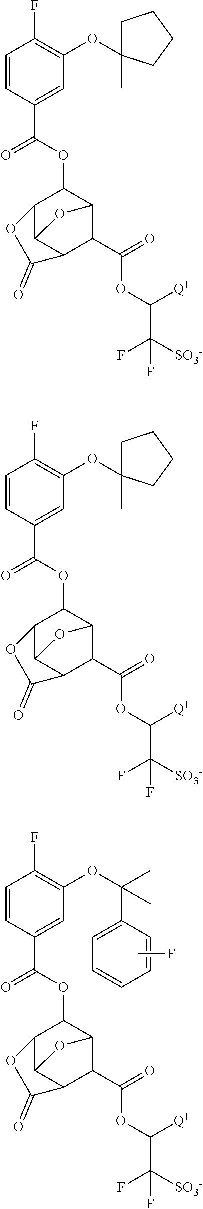

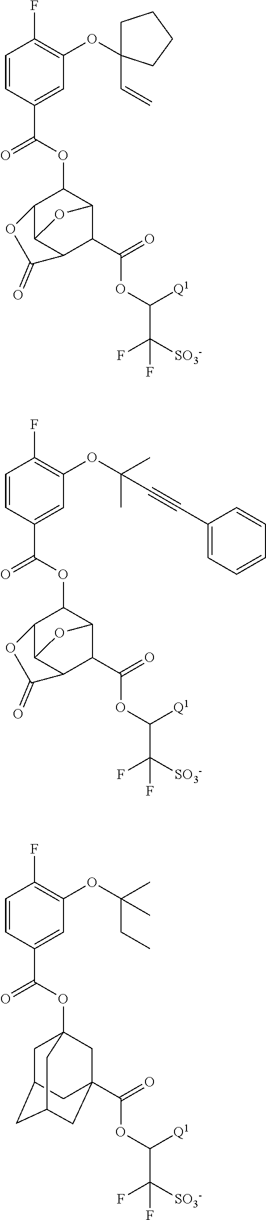

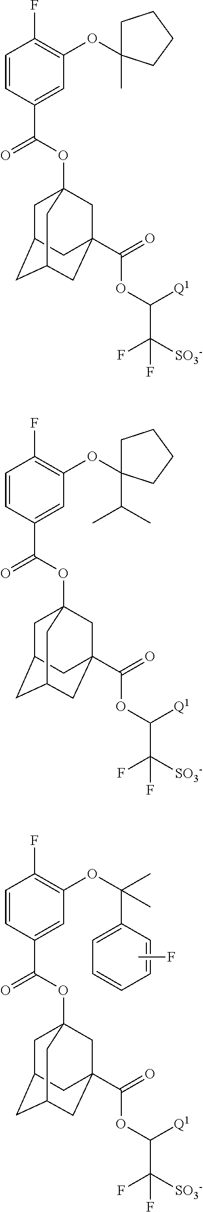

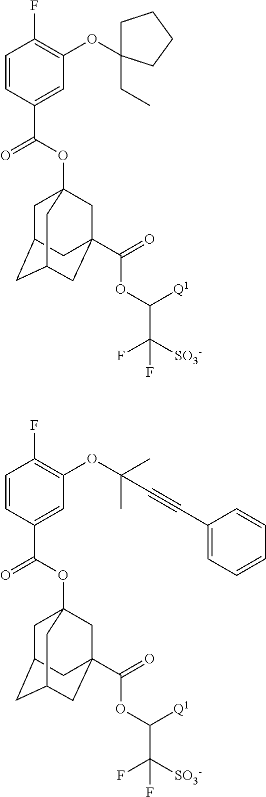

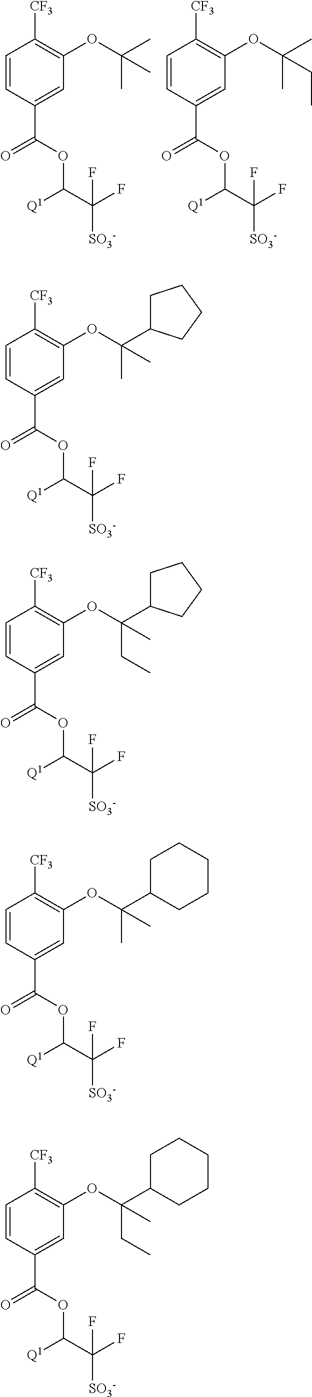

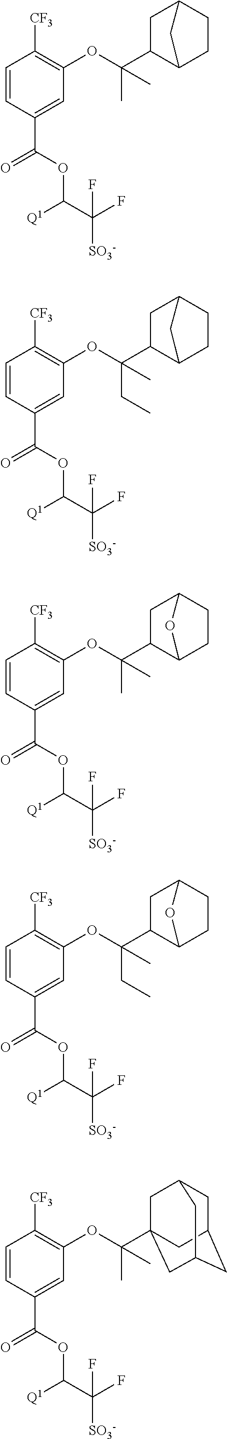

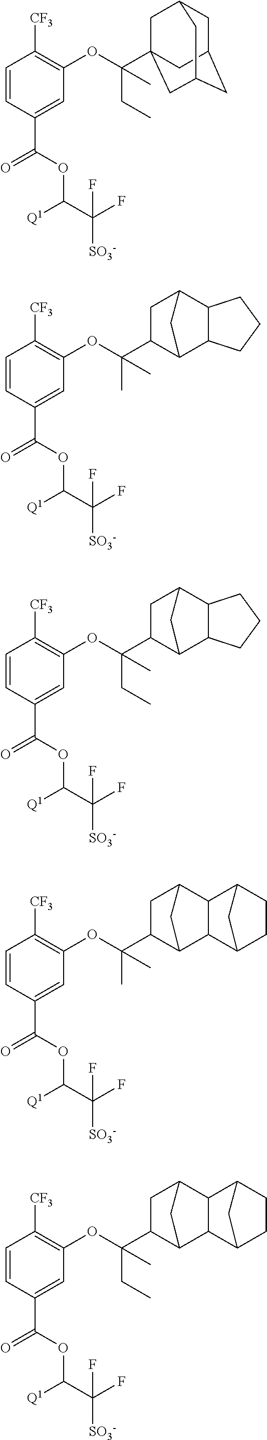

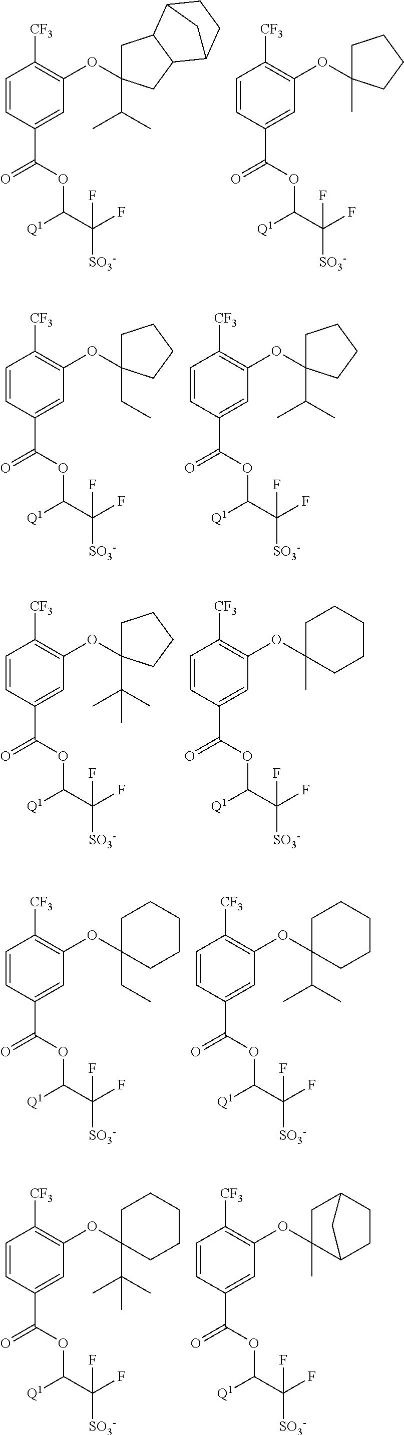

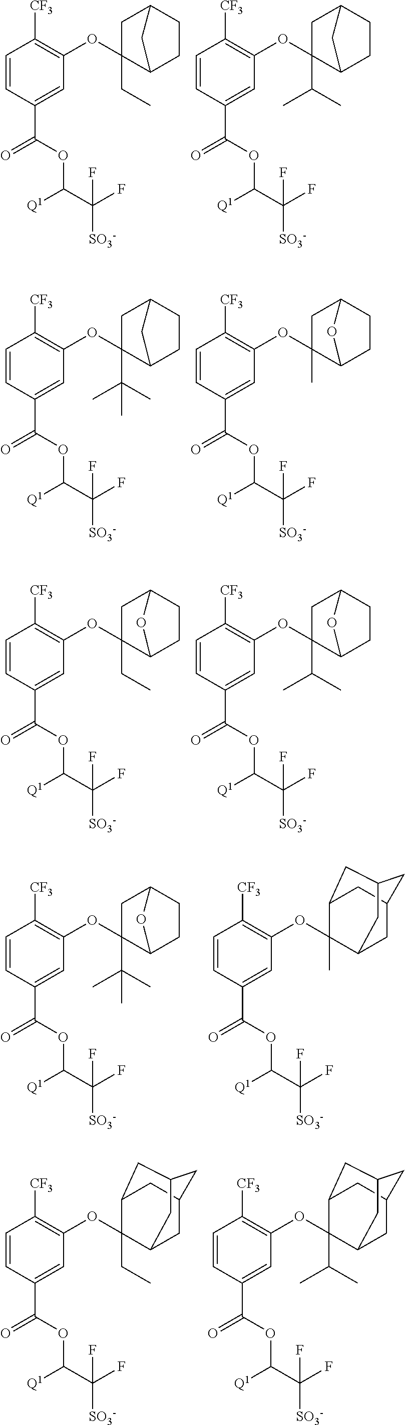

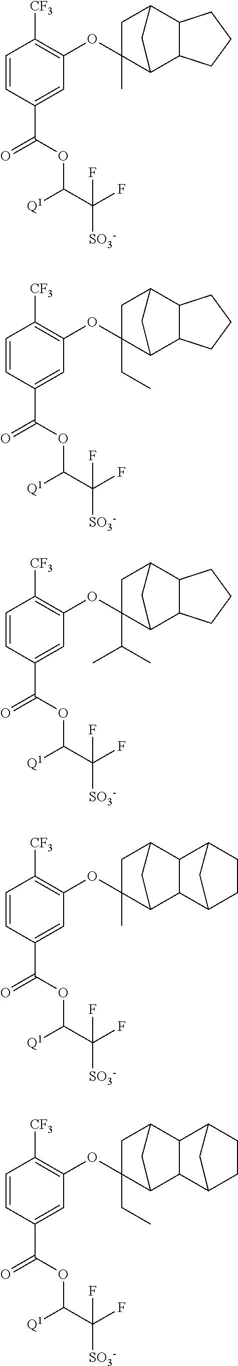

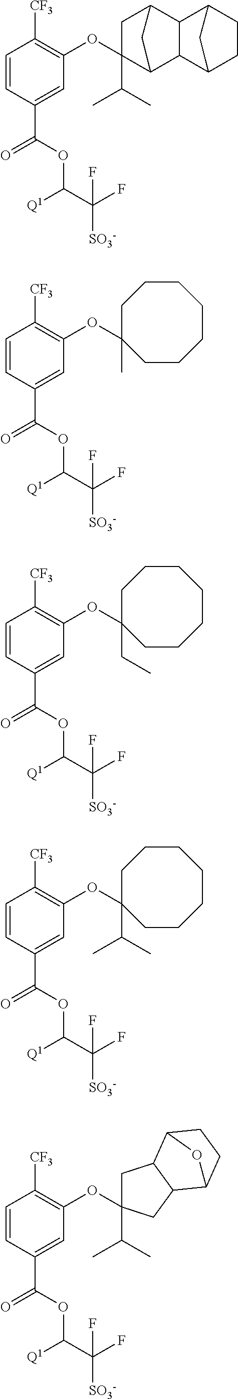

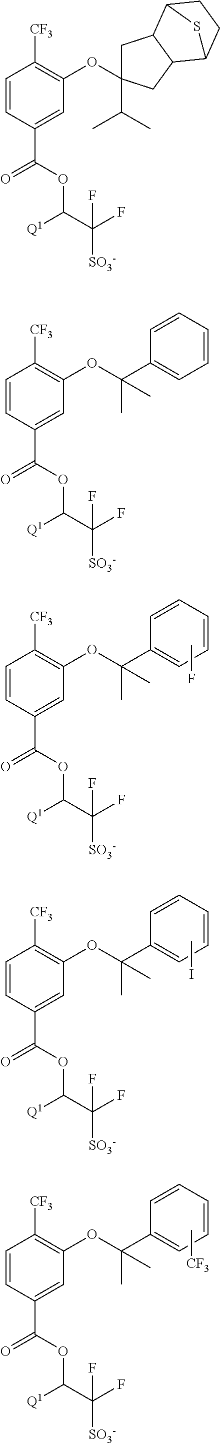

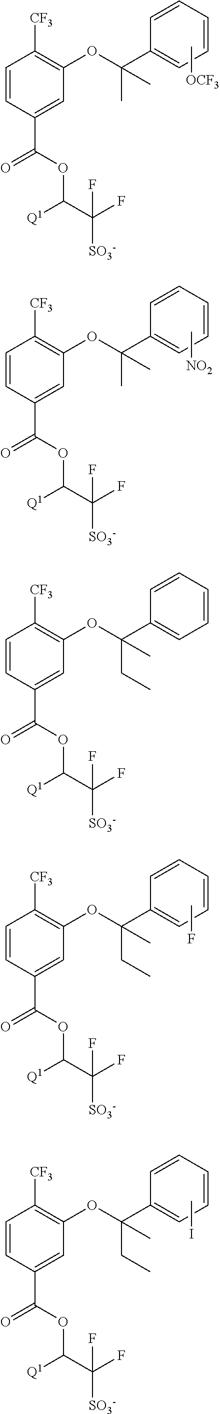

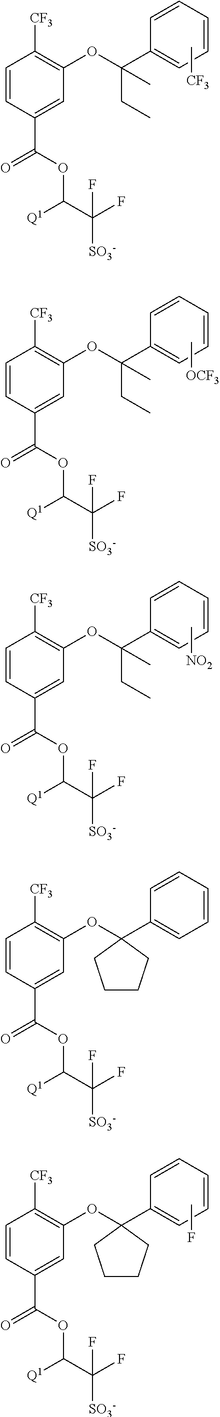

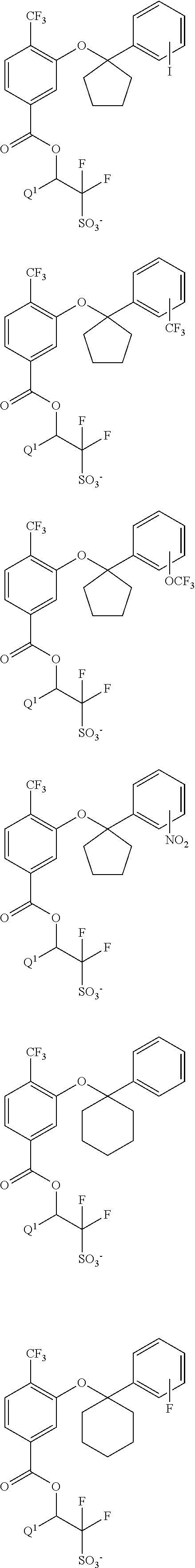

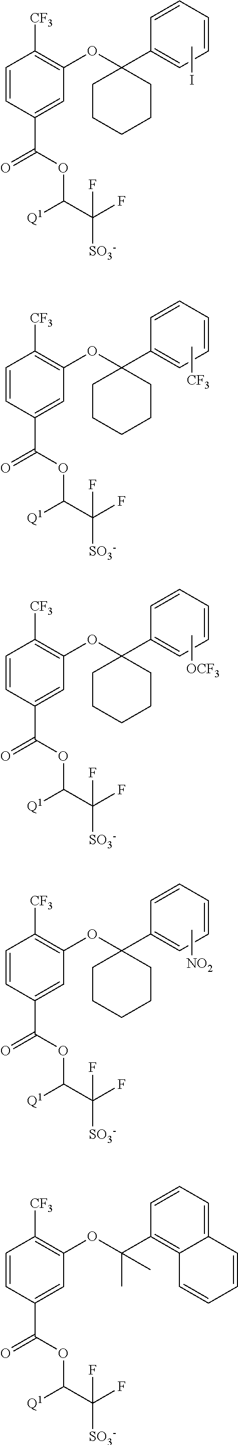

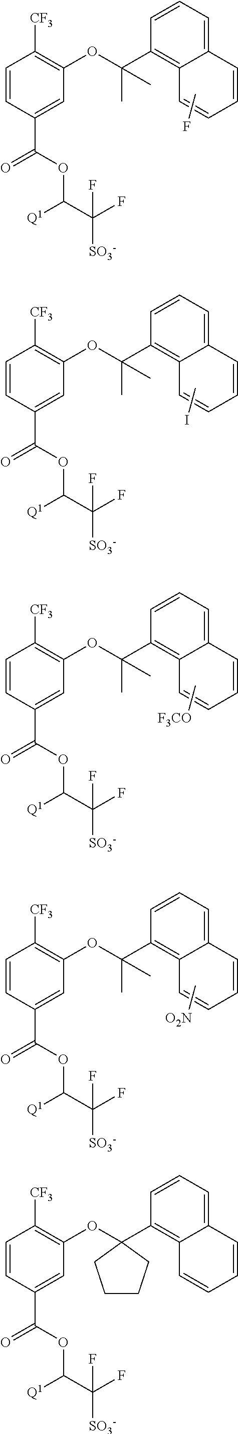

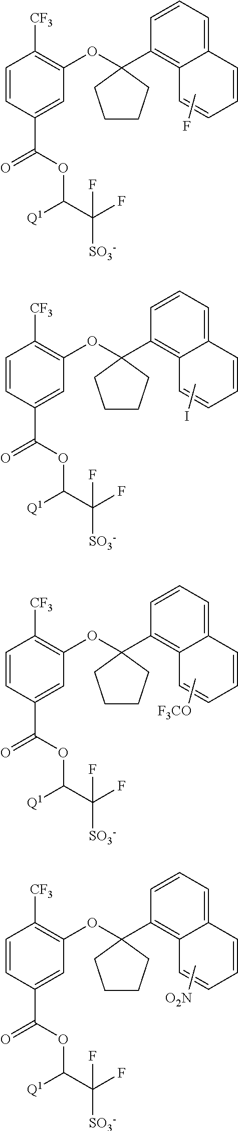

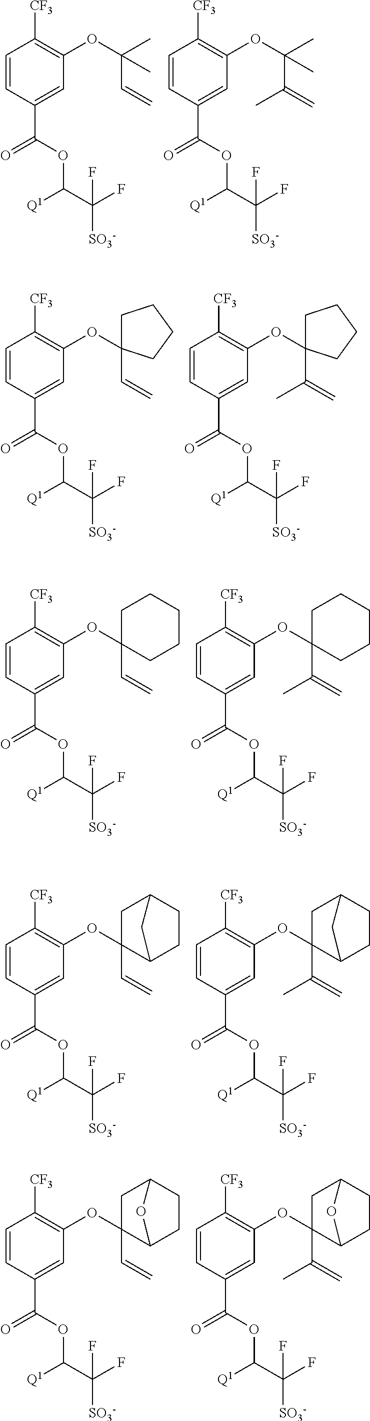

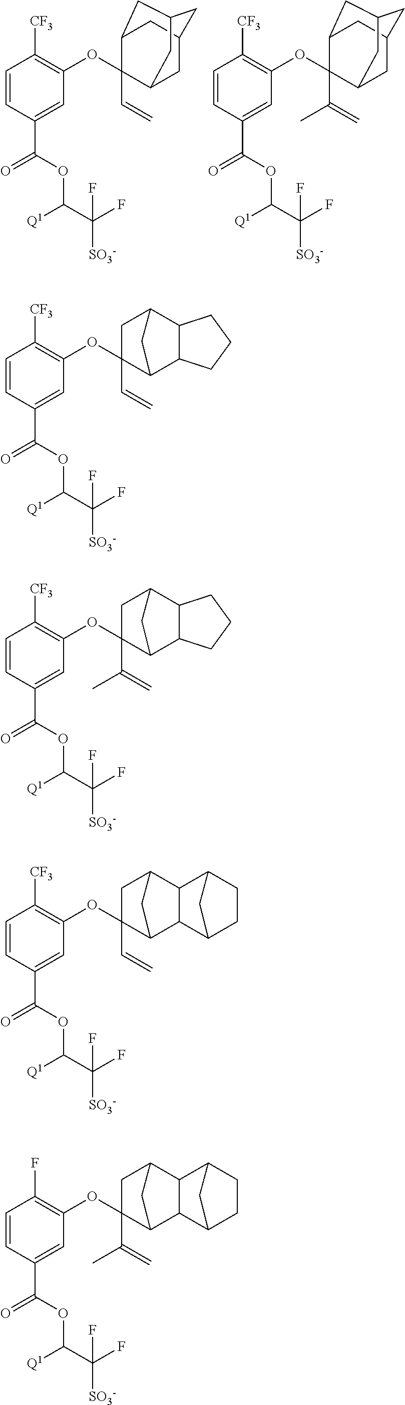

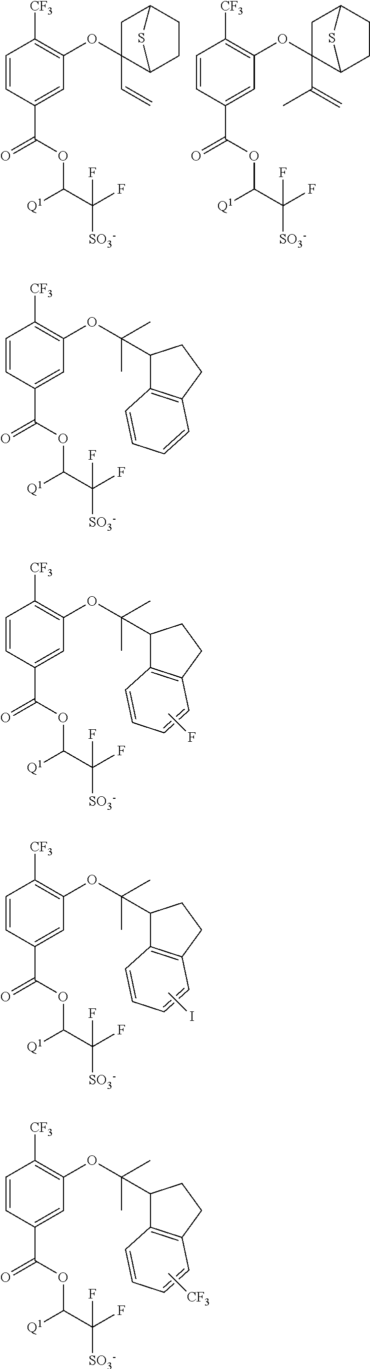

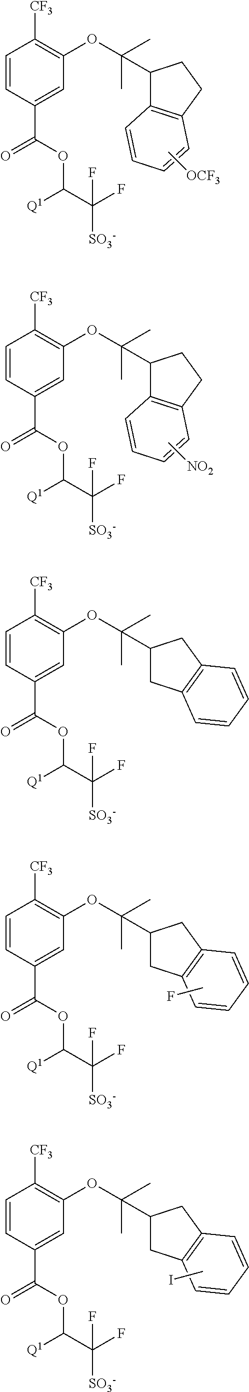

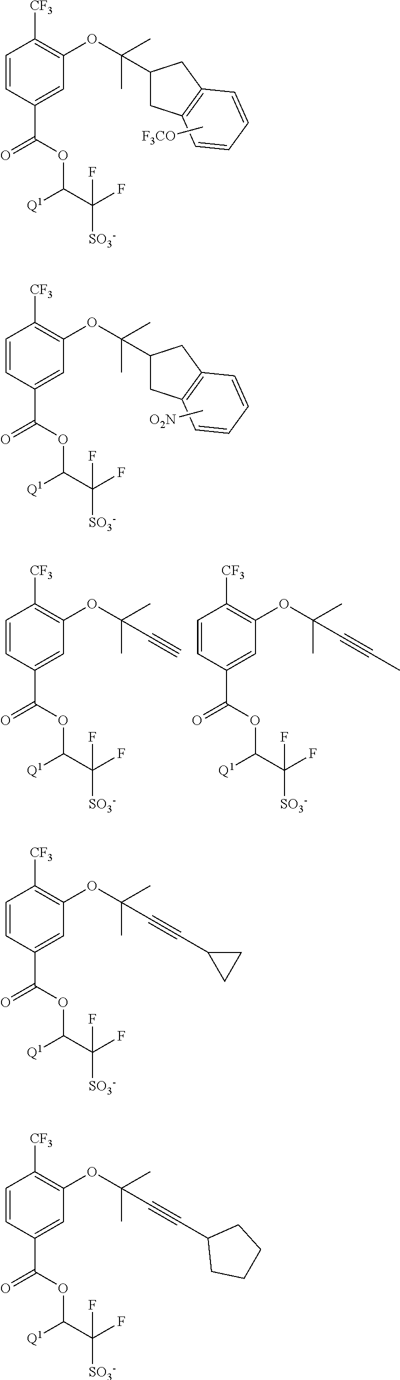

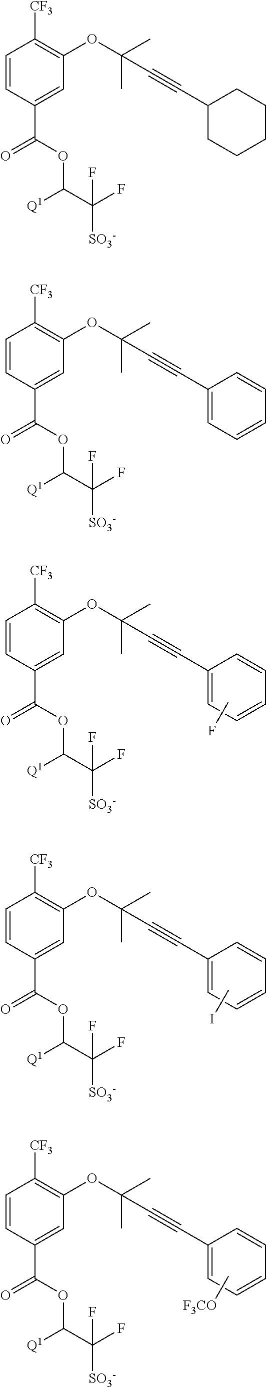

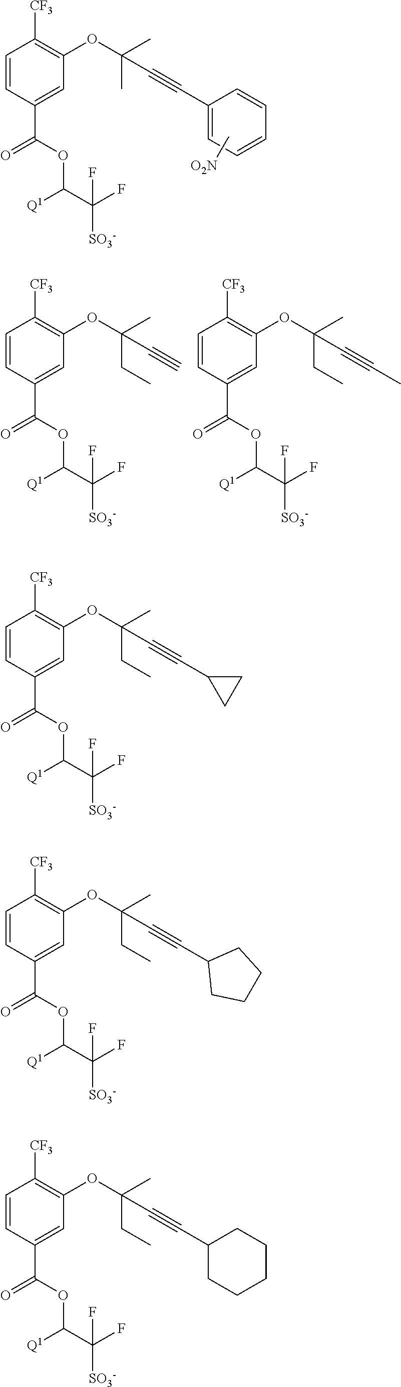

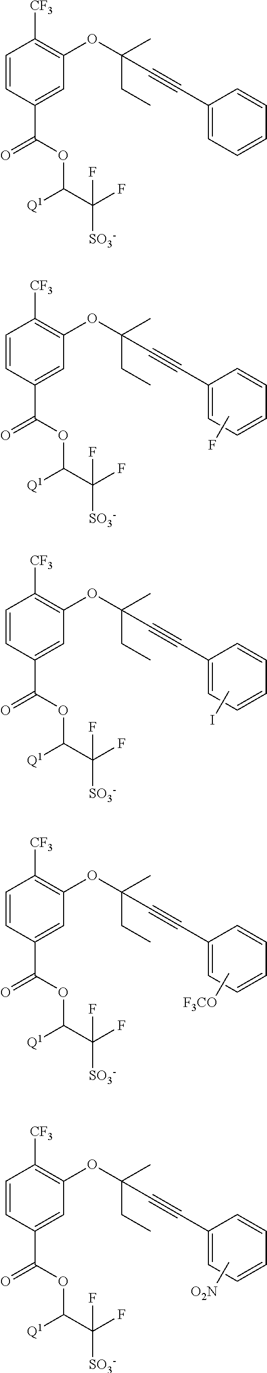

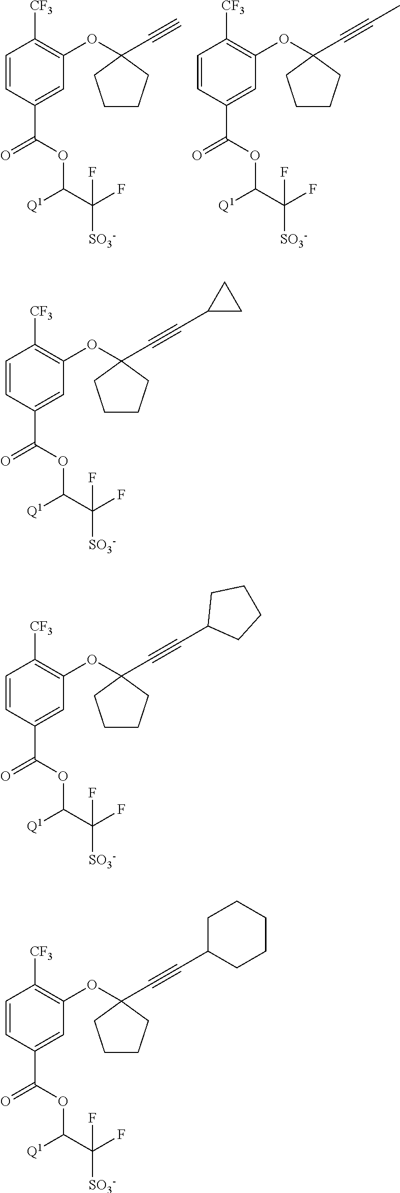

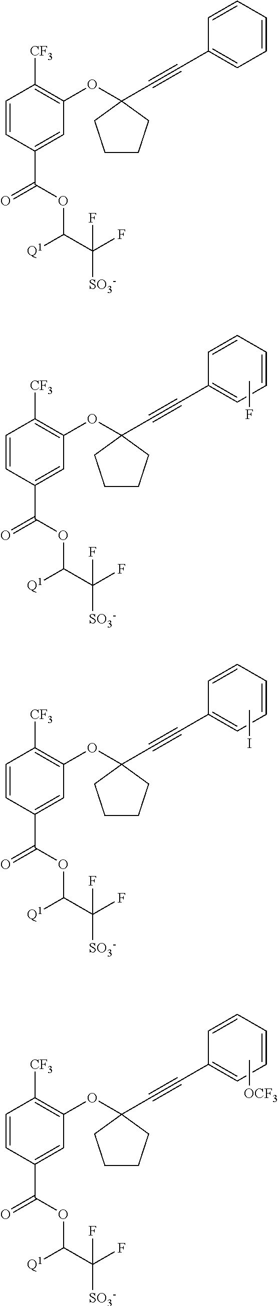

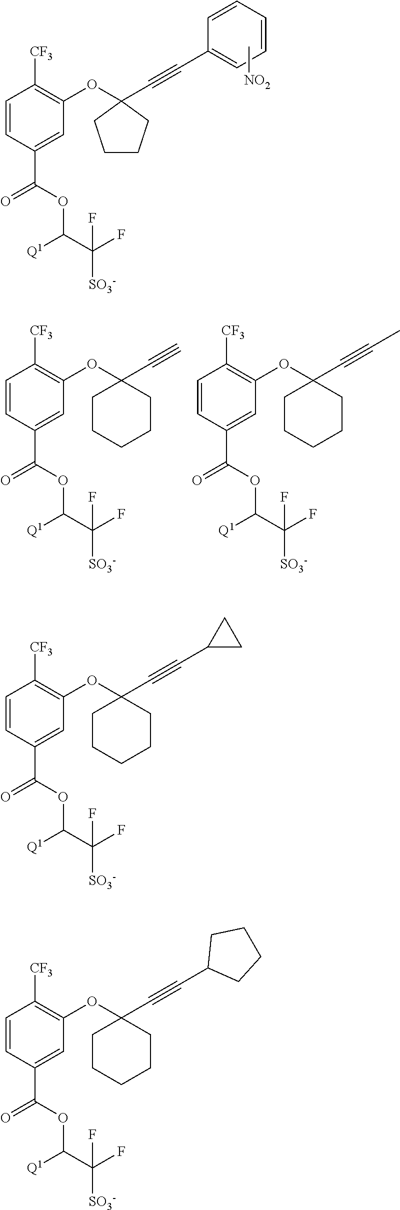

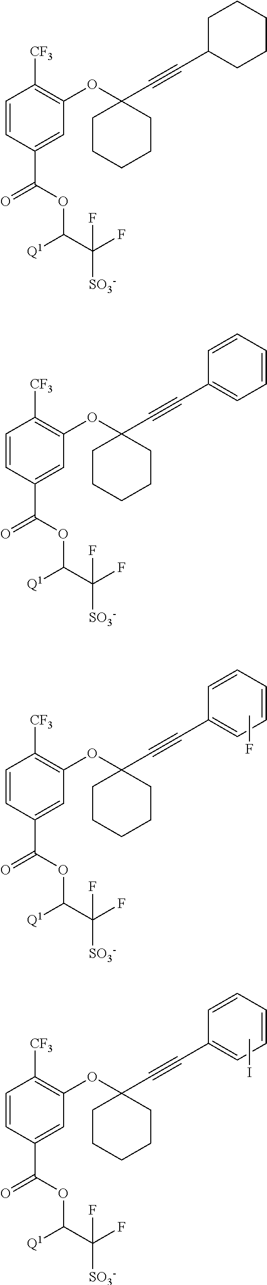

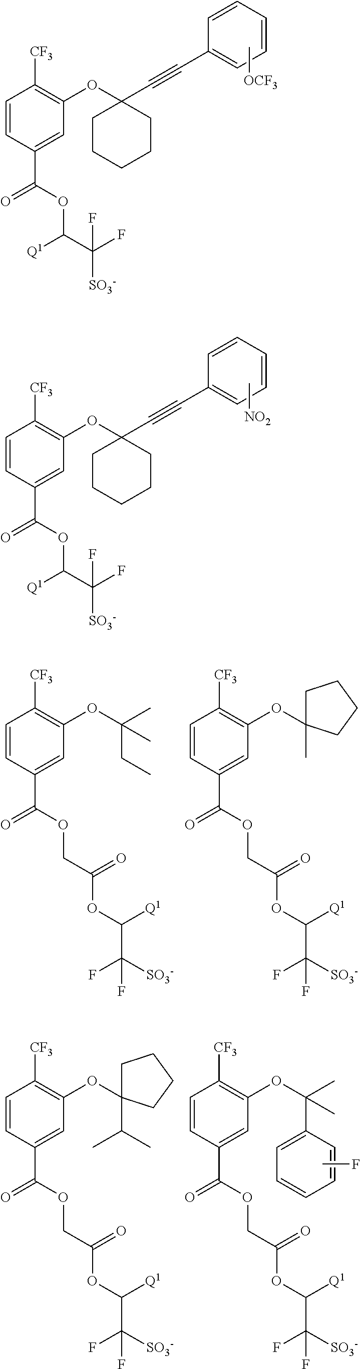

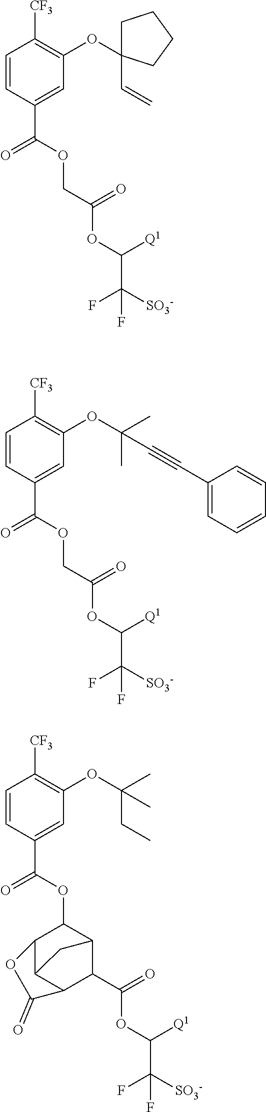

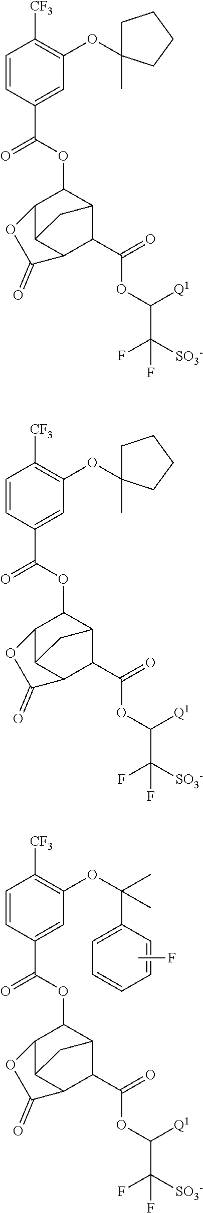

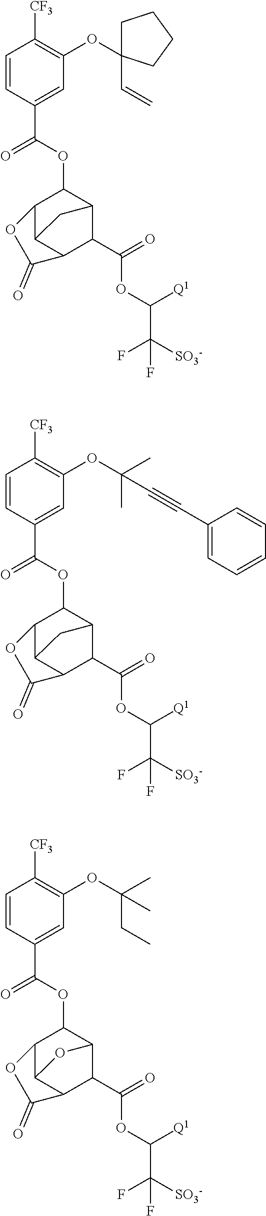

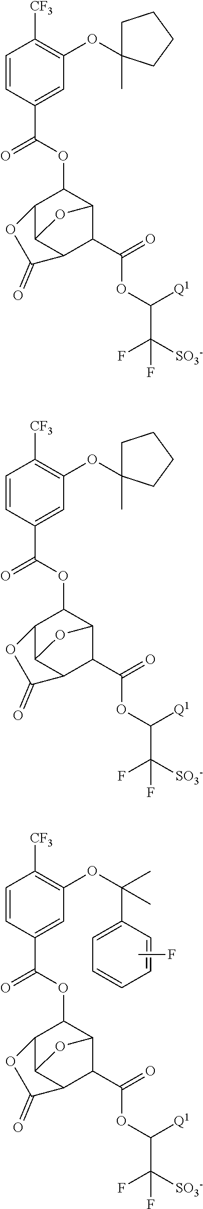

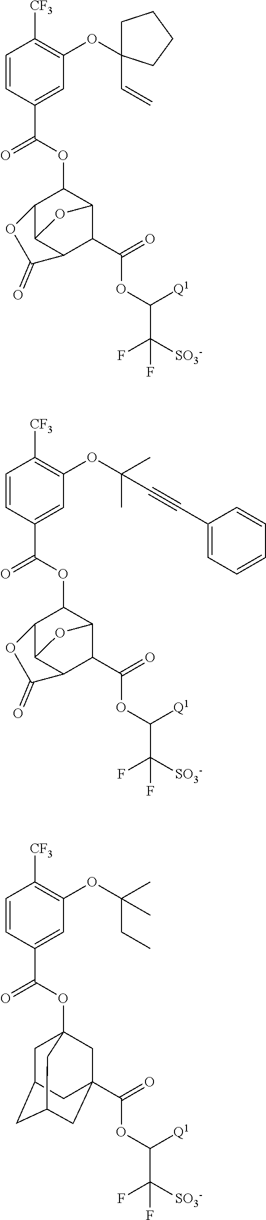

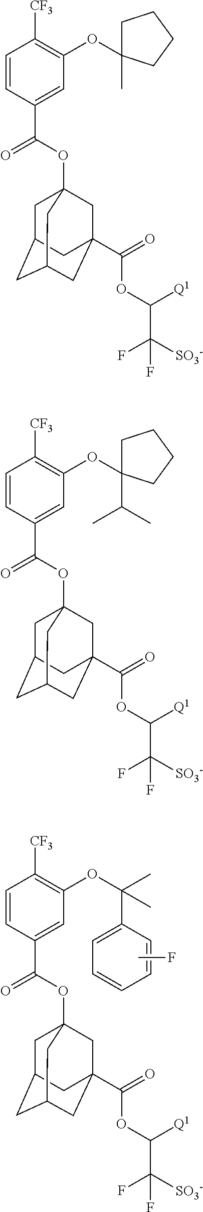

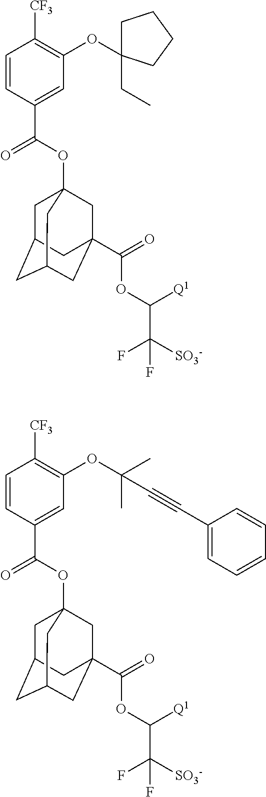

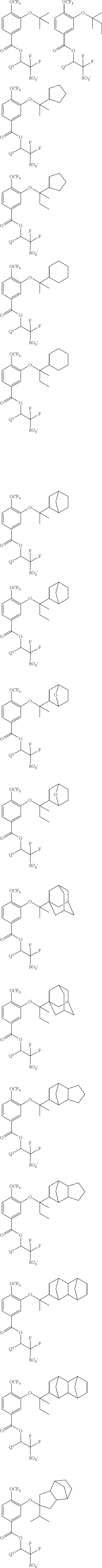

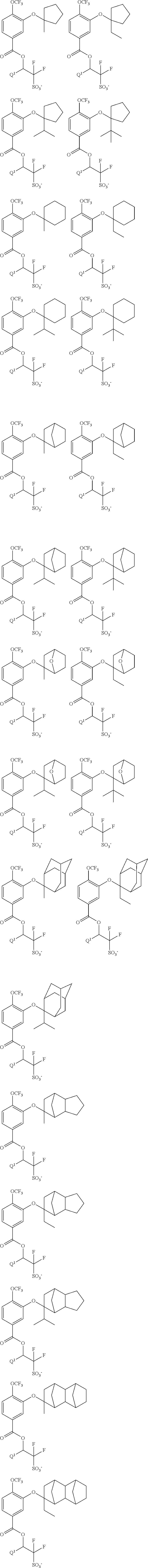

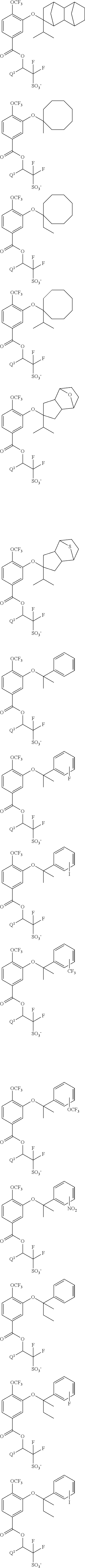

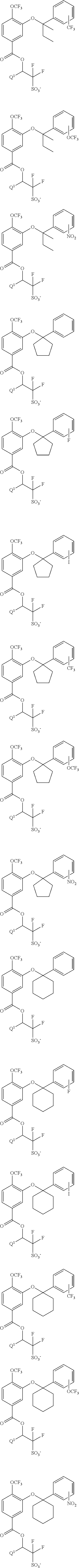

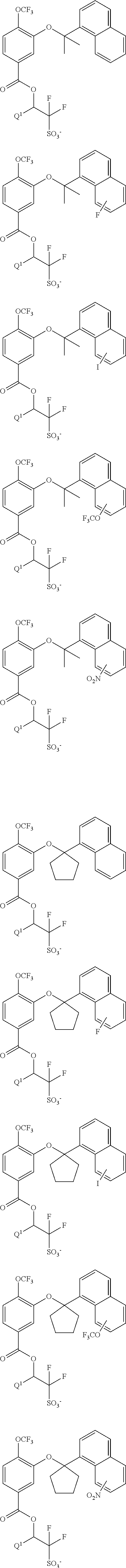

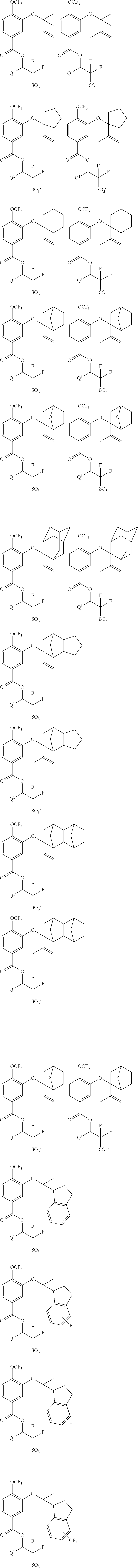

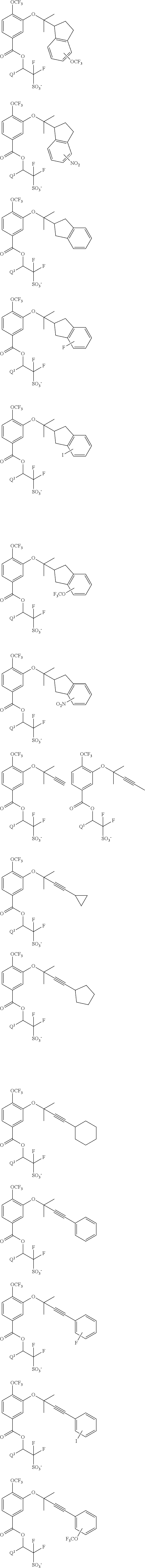

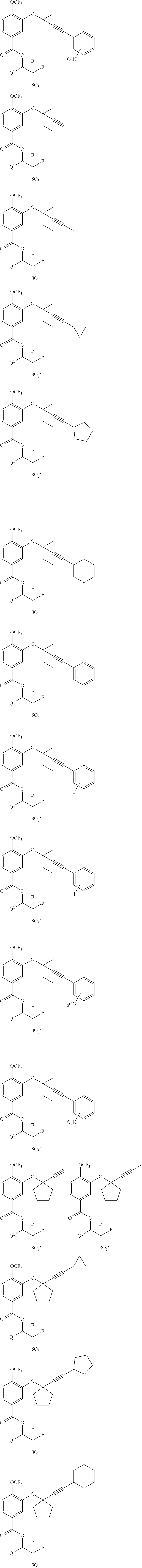

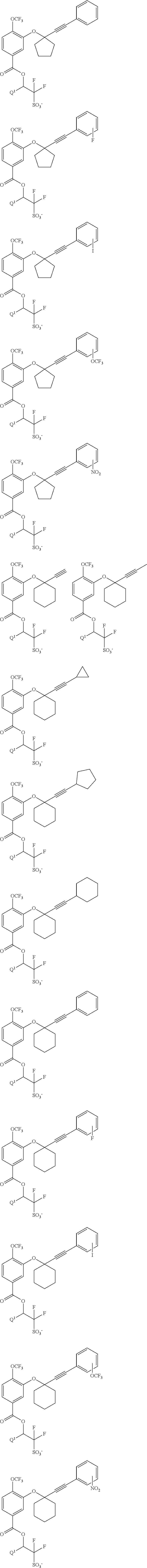

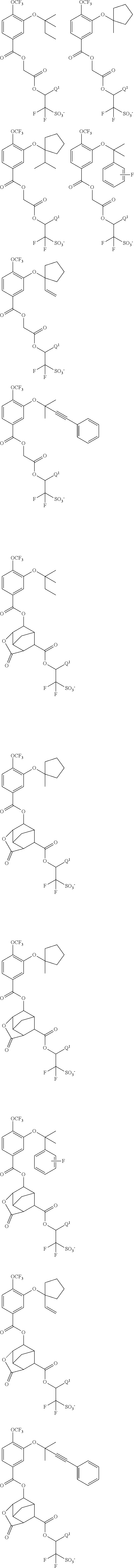

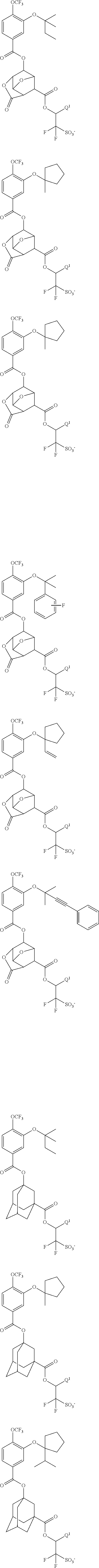

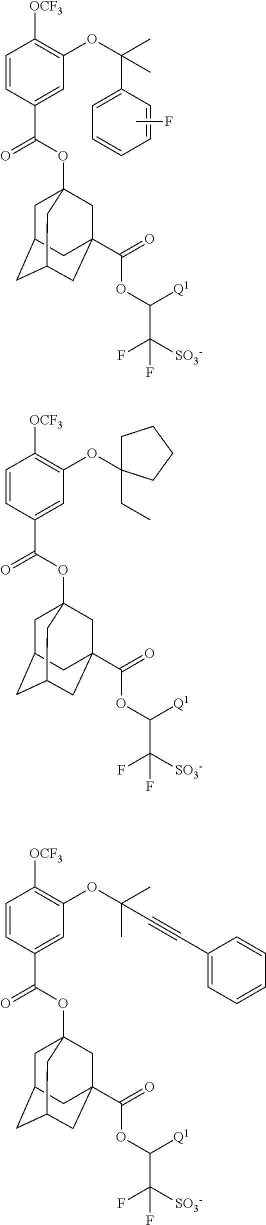

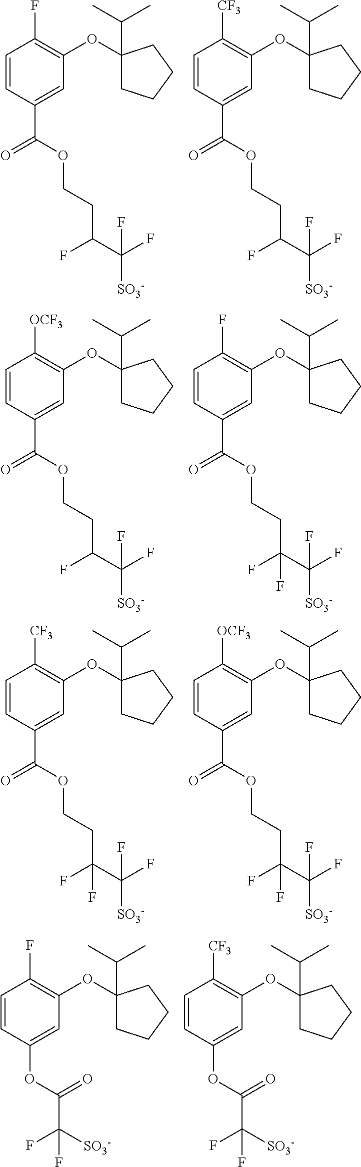

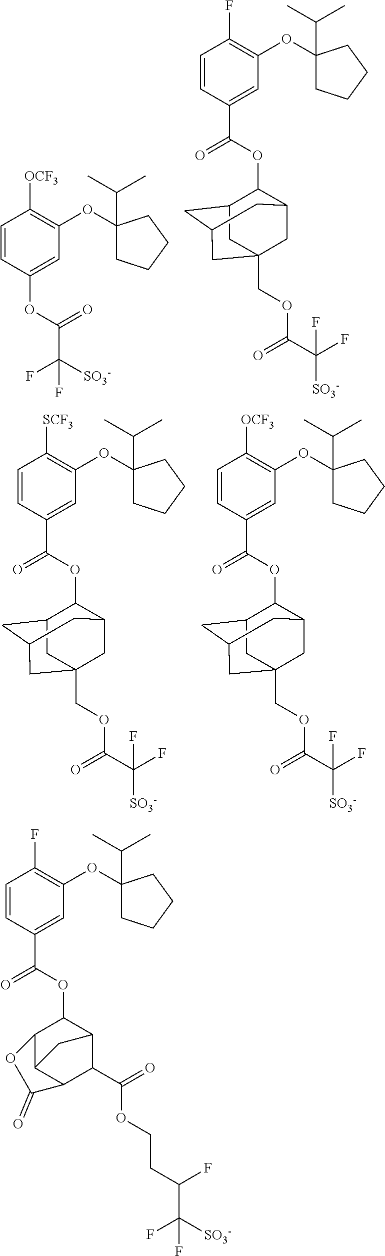

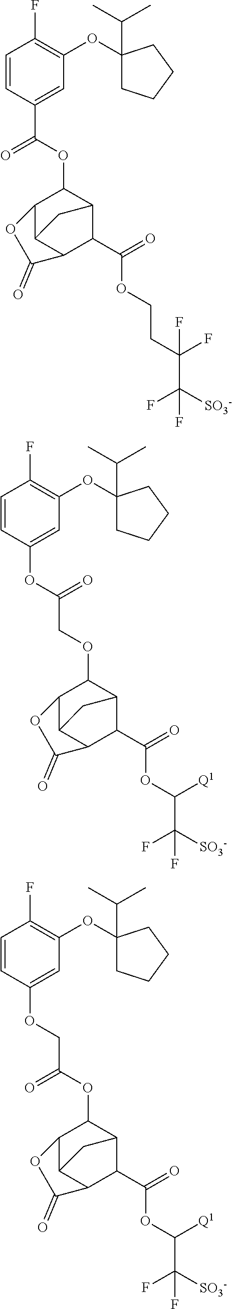

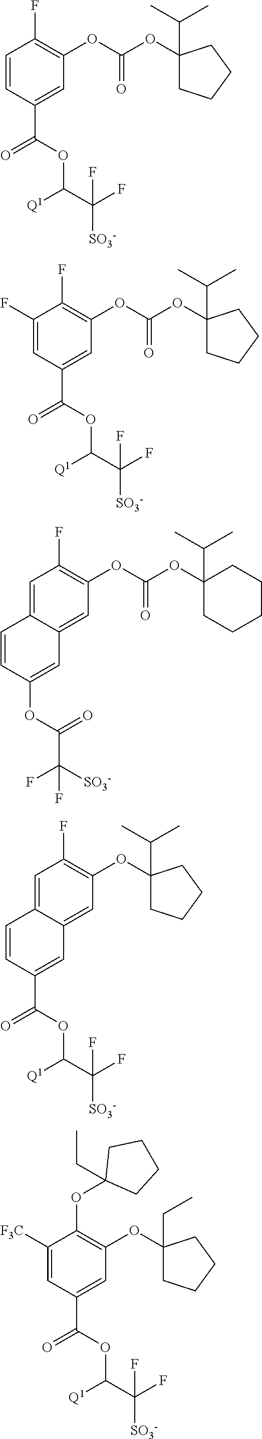

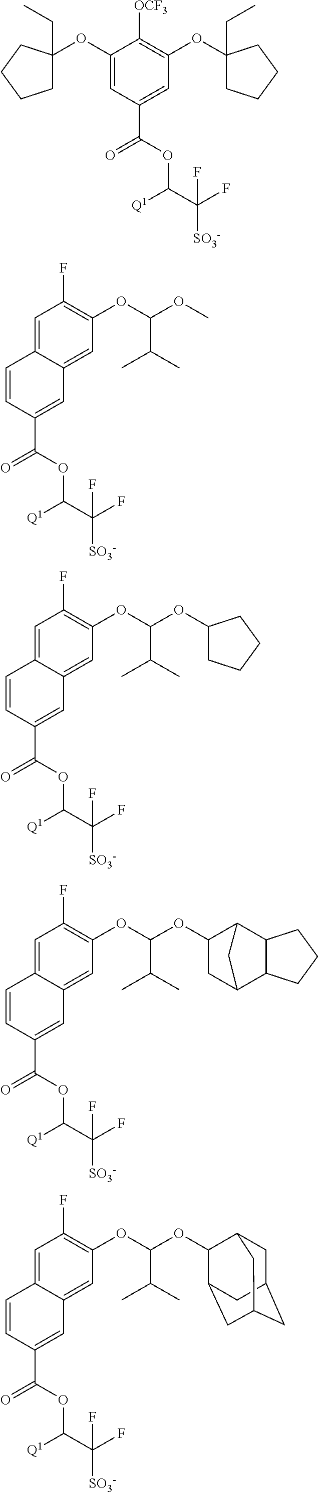

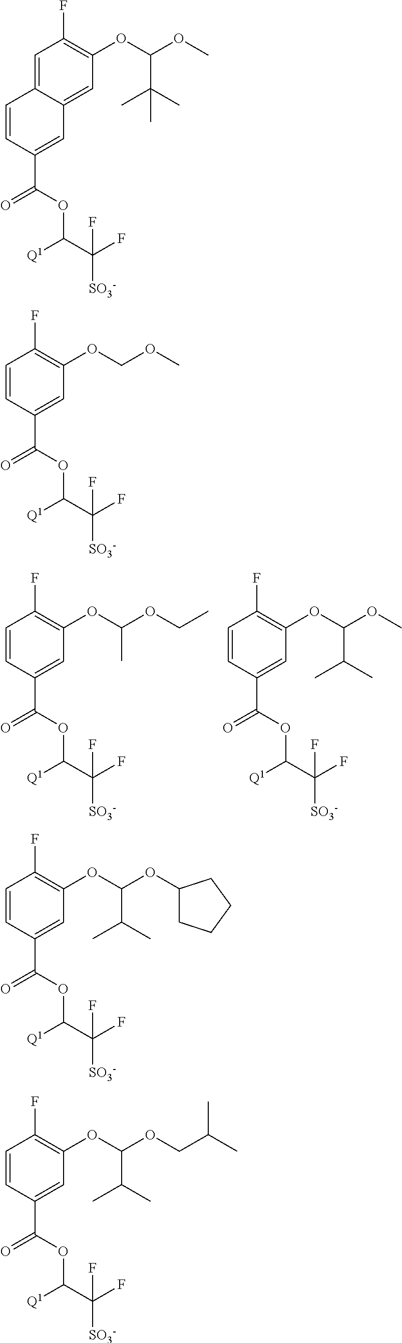

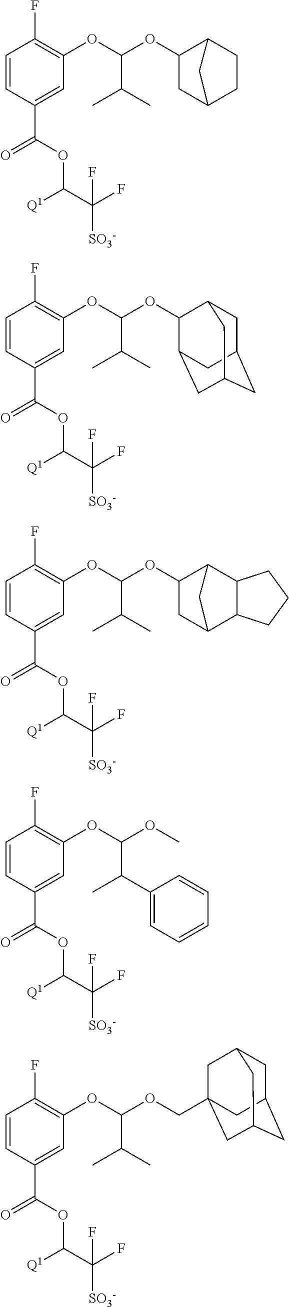

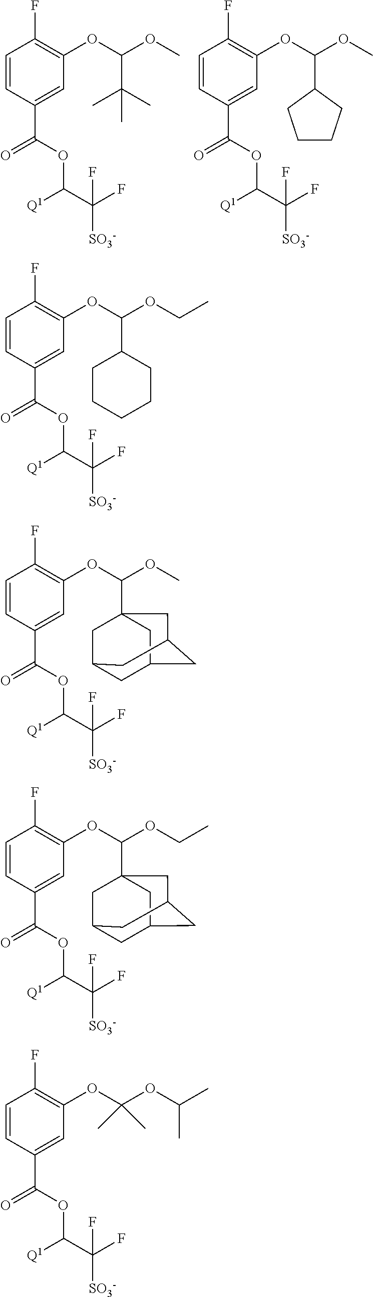

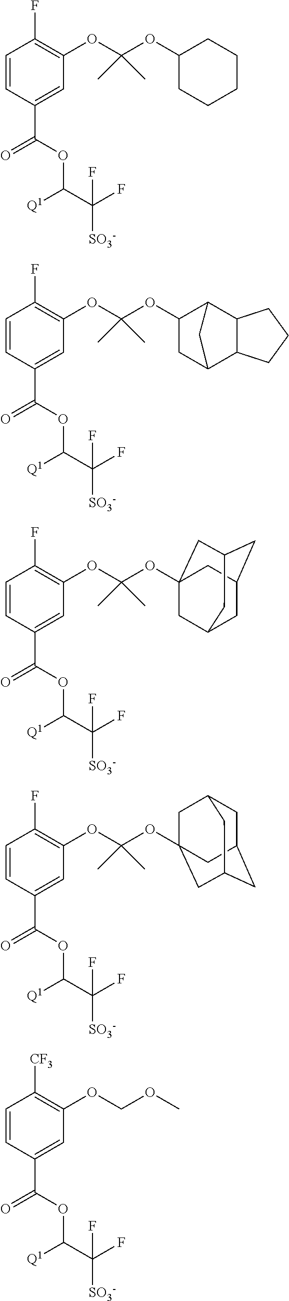

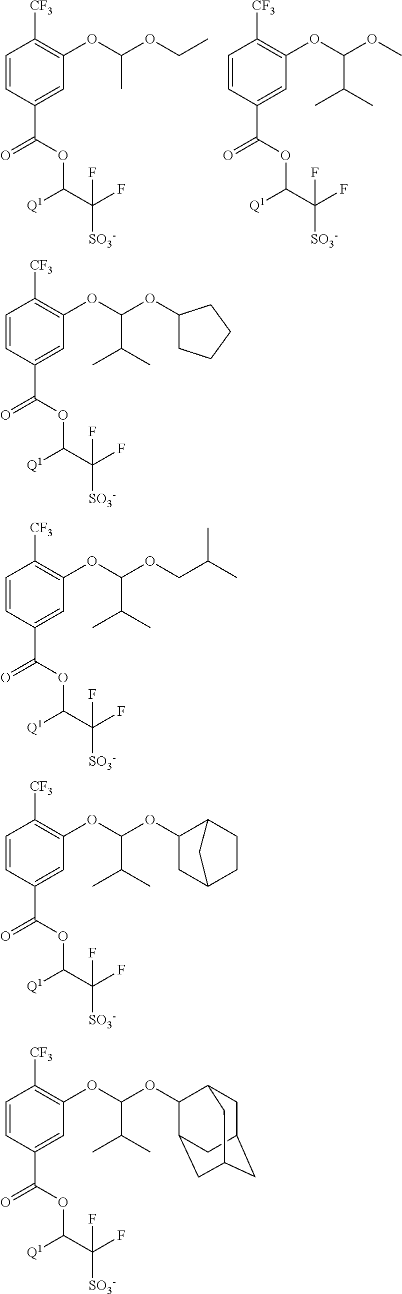

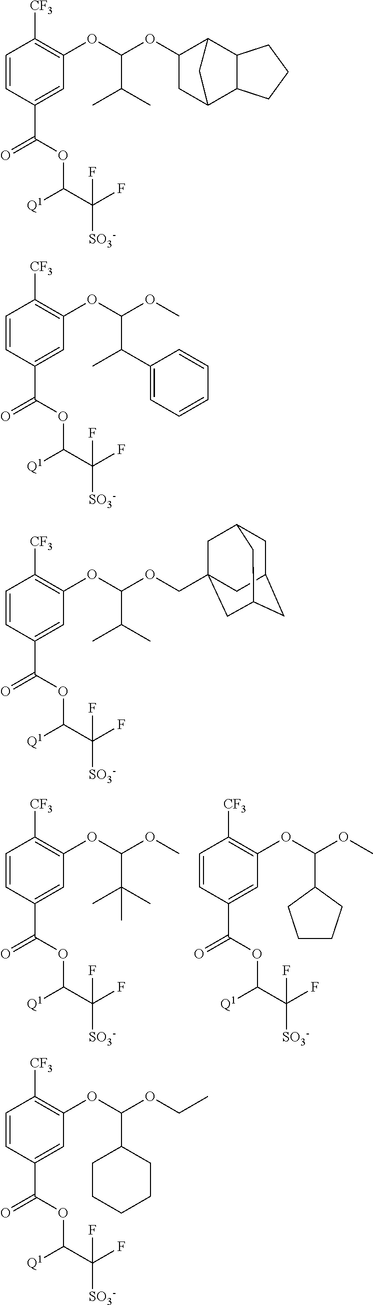

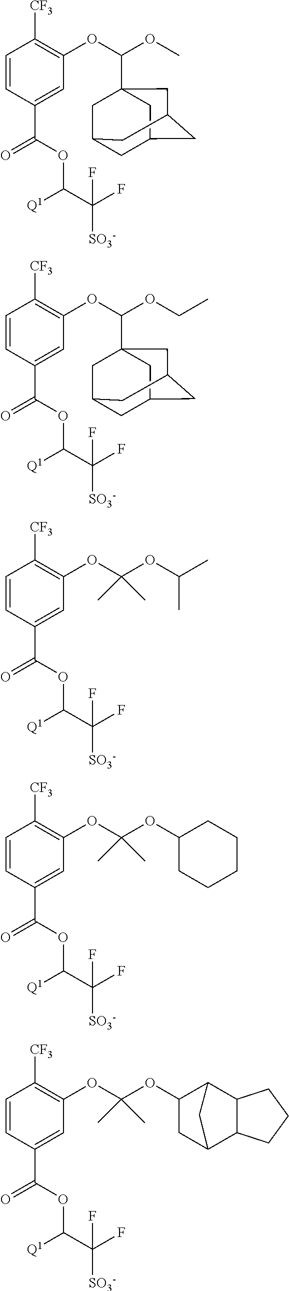

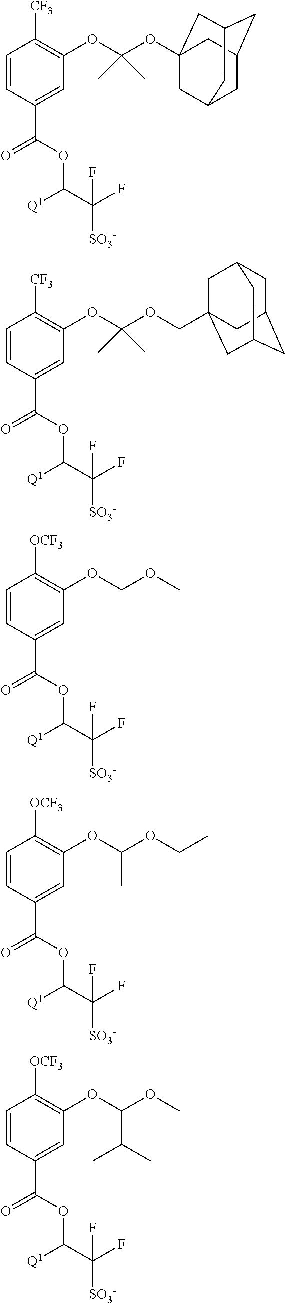

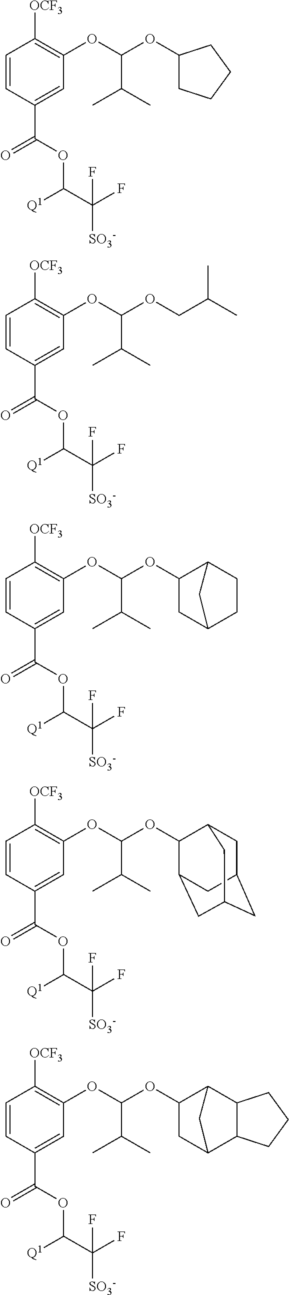

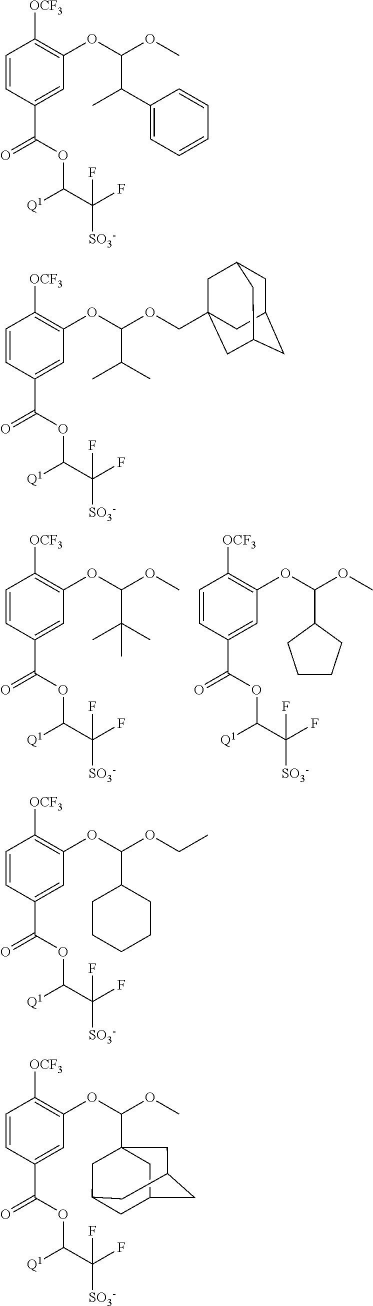

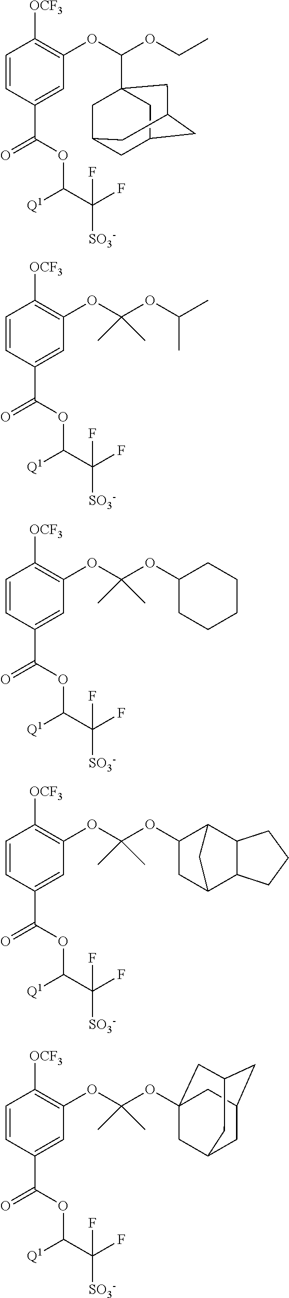

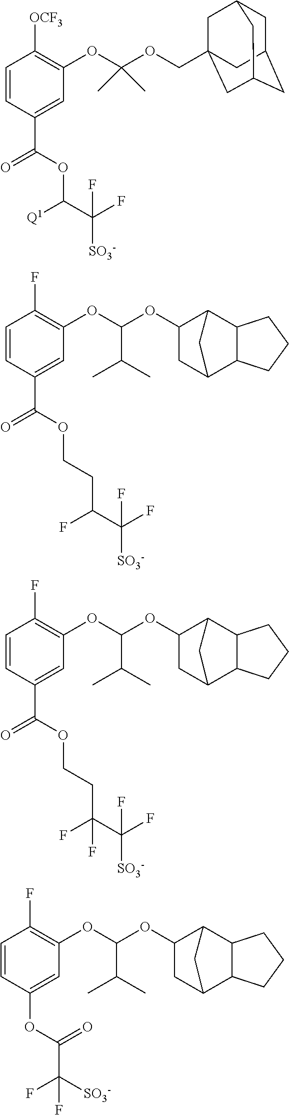

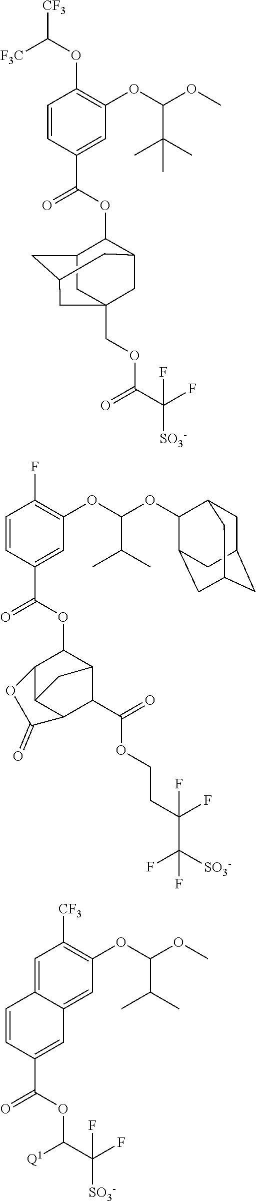

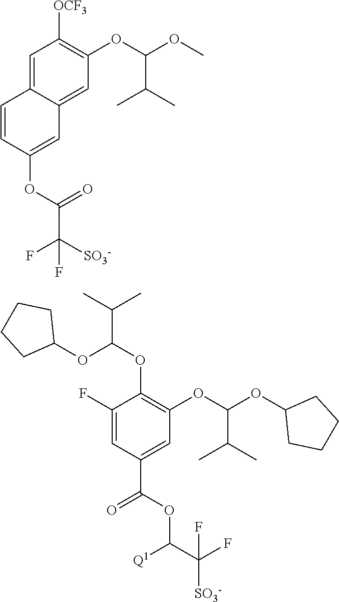

In formula (1) Z⁺ is an onium cation having the formula (cation-1) or (cation-2).

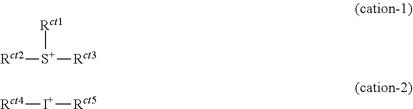

In formulae (cation-1) and (cation-2), R^{ct1} to R^{ct5} are each independently a C₁-C₃₀ hydrocarbyl group which may contain a heteroatom. The hydrocarbyl group may be saturated or unsaturated and straight, branched or cyclic. Examples thereof include alkyl groups such as methyl, ethyl, n-propyl, isopropyl, n-butyl, isobutyl, sec-butyl, tert-butyl; cyclic saturated hydrocarbyl groups such as cyclopropyl, cyclopentyl, cyclohexyl, cyclopropyhnethyl, 4-methylcyclohexyl, cyclohexyhnethyl, norboryl, adamantyl; alkenyl groups such as vinyl, allyl, propenyl, butenyl, hexenyl; cyclic unsaturated hydrocarbyl groups such as cyclohexenyl: aryl groups such as phenyl, naphthyl, thienyl; aralkyl groups such as benzyl, 1-phenylethyl, 2-phenylethyl, and combinations thereof. Inter alia, the aryl groups are preferred. In the hydrocarbyl groups, some hydrogen may be substituted by a moiety containing a heteroatom such as oxygen, sulfur, nitrogen or halogen, and a moiety containing a heteroatom such as oxygen, sulfur or nitrogen may intervene between carbon atoms, so that the group may contain a hydroxy, cyano, carbonyl, ether bond, ester bond, sulfonic ester bond, carbonate bond, lactone ring, sultone ring, carboxylic anhydride (—C(═O)—O—C(═O)—) or haloalkyl moiety.

Also, R^{ct1} and R^{ct2} may bond together to form a ring with the sulfur atom to which they are attached. Examples of the sulfonium cation having formula (cation-1) in this embodiment are shown below.

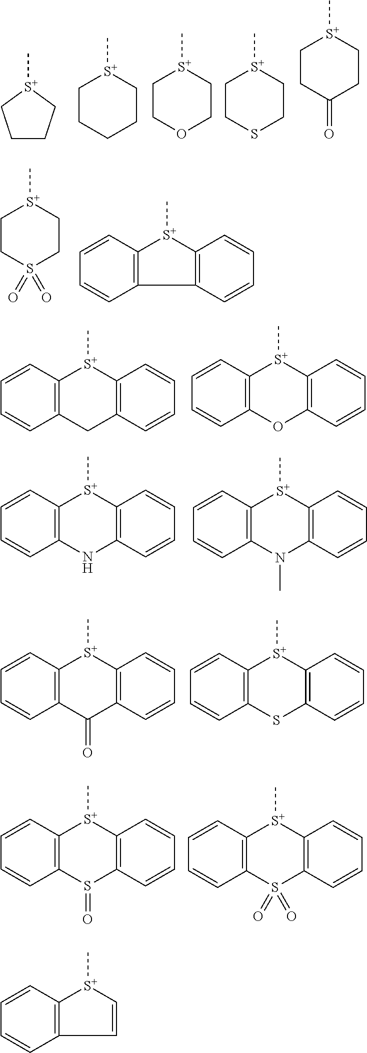

The broken line designates a point of attachment to R^{ct3}.

Examples of the sulfonium cation having formula (cation-1) are shown below, but not limited thereto.

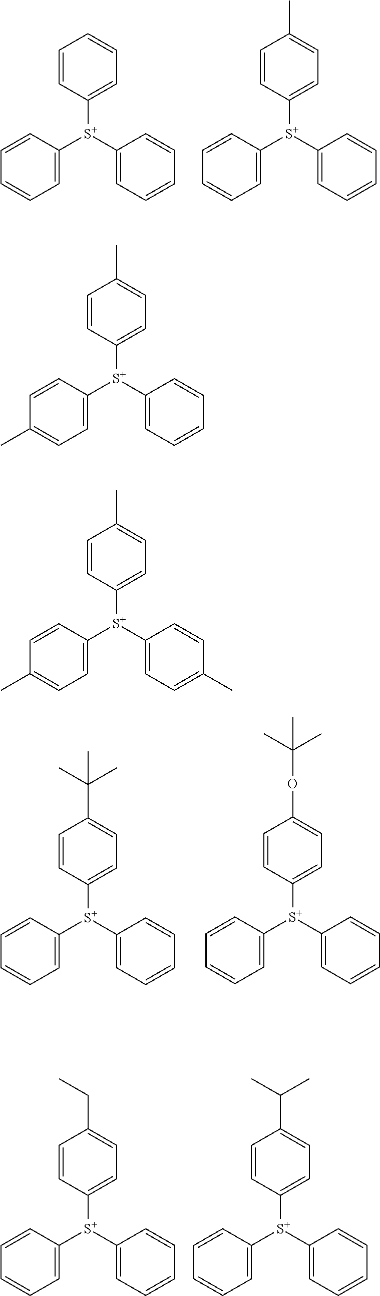

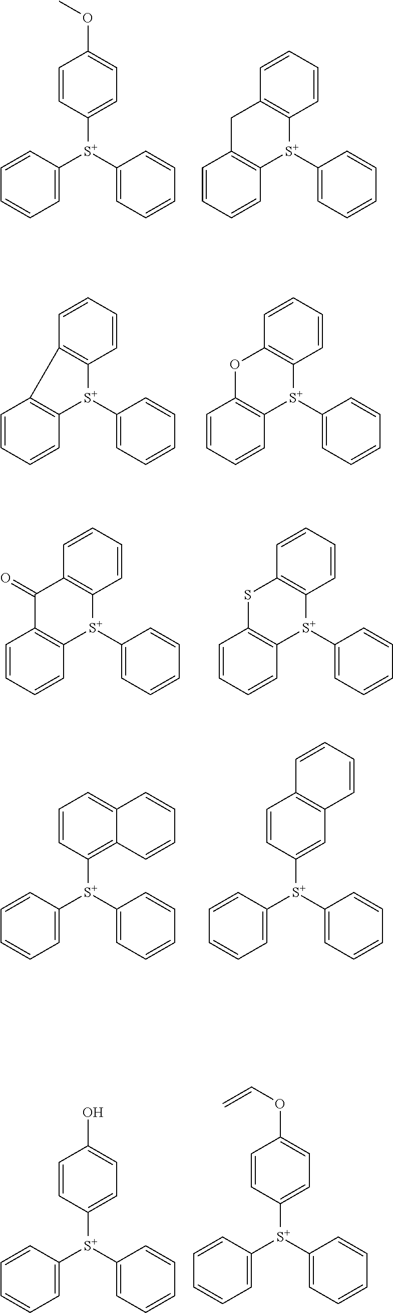

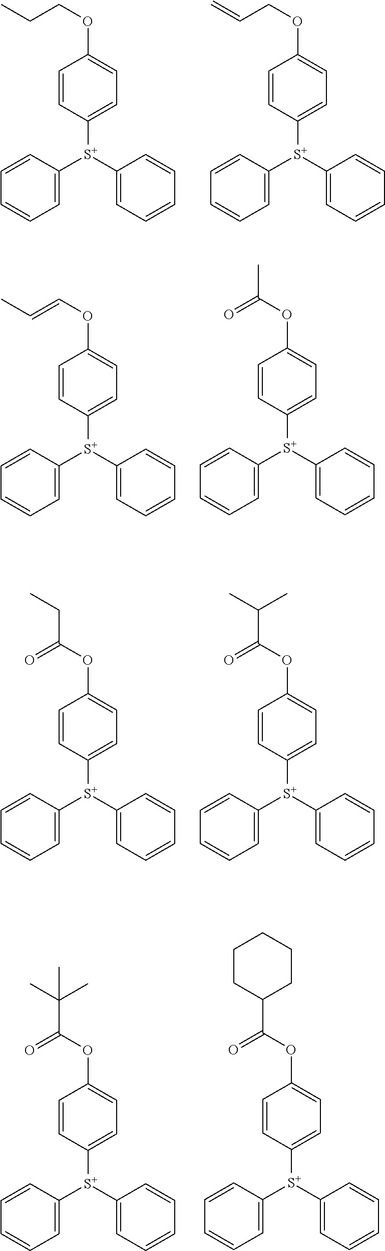

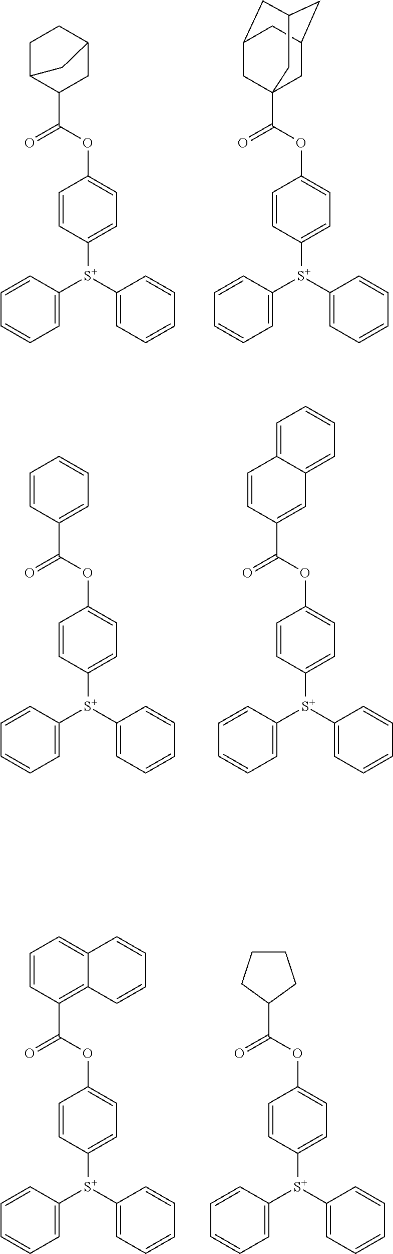 [SMILES: O=C(Oc1ccc([S+](c2ccccc2)c2ccccc2)cc1)C1CC2CCC1C2 ; O=C(Oc1ccc([S+](c2ccccc2)c2ccccc2)cc1)C12CC3CC(CC(C3)C1)C2 ; O=C(Oc1ccc([S+](c2ccccc2)c2ccccc2)cc1)c1ccccc1 ; O=C(Oc1ccc([S+](c2ccccc2)c2ccccc2)cc1)c1ccc2ccccc2c1 ; O=C(Oc1ccc([S+](c2ccccc2)c2ccccc2)cc1)c1cccc2ccccc12 ; O=C(Oc1ccc([S+](c2ccccc2)c2ccccc2)cc1)C1CCCC1]

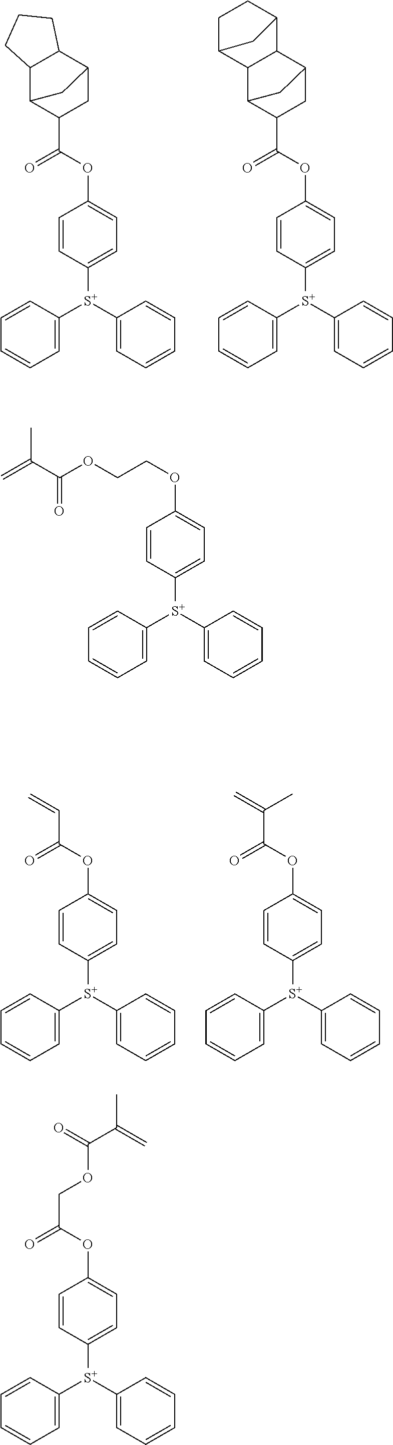 [SMILES: O=C(Oc1ccc([S+](c2ccccc2)c2ccccc2)cc1)C1CC2CC1C1CCCC21 ; O=C(Oc1ccc([S+](c2ccccc2)c2ccccc2)cc1)C1CC2CC1C1C3CCC(C3)C21 ; C=C(C)C(=O)OCCOc1ccc([S+](c2ccccc2)c2ccccc2)cc1 ; C=CC(=O)Oc1ccc([S+](c2ccccc2)c2ccccc2)cc1 ; C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1]

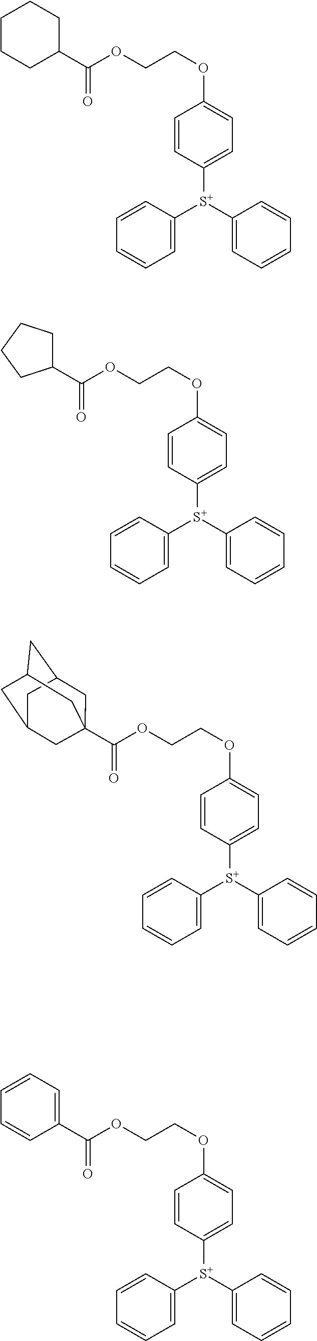 [SMILES: O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1]

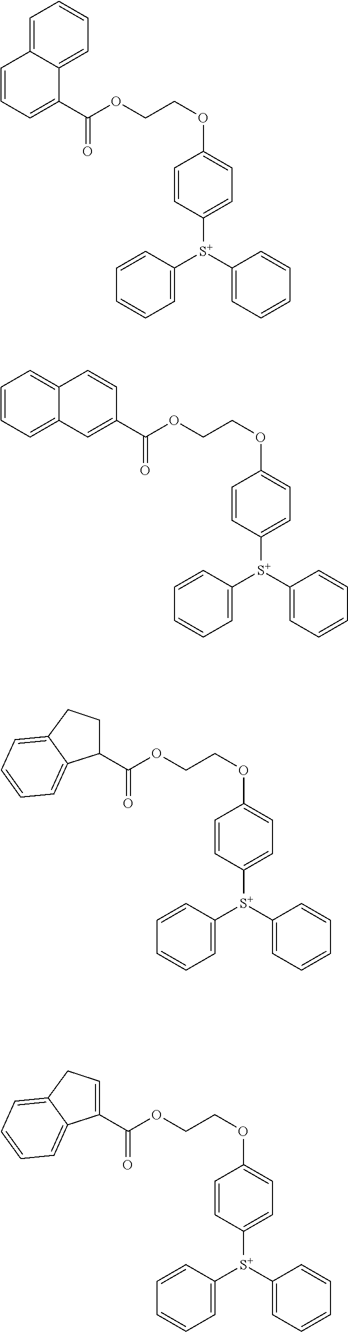 [SMILES: O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

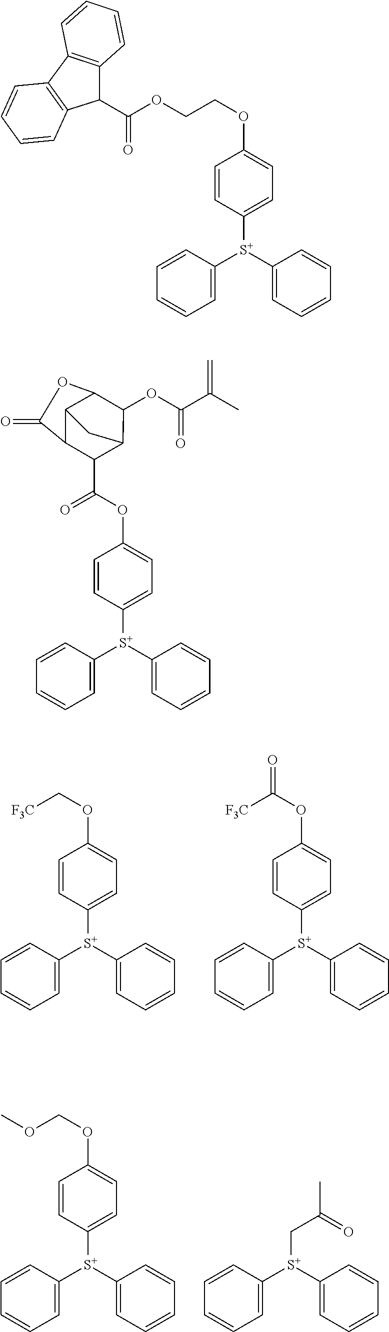 [SMILES: O=C(OCCOc1ccc([S+](c2ccccc2)c2ccccc2)cc1)C1c2ccccc2-c2ccccc21 ; C=C(C)C(=O)OC1C2CC3C1OC(=O)C3C2C(=O)Oc1ccc([S+](c2ccccc2)c2ccccc2)cc1 ; FC(F)(F)COc1ccc([S+](c2ccccc2)c2ccccc2)cc1 ; O=C(Oc1ccc([S+](c2ccccc2)c2ccccc2)cc1)C(F)(F)F ; COCOc1ccc([S+](c2ccccc2)c2ccccc2)cc1 ; CC(=O)C[S+](c1ccccc1)c1ccccc1]

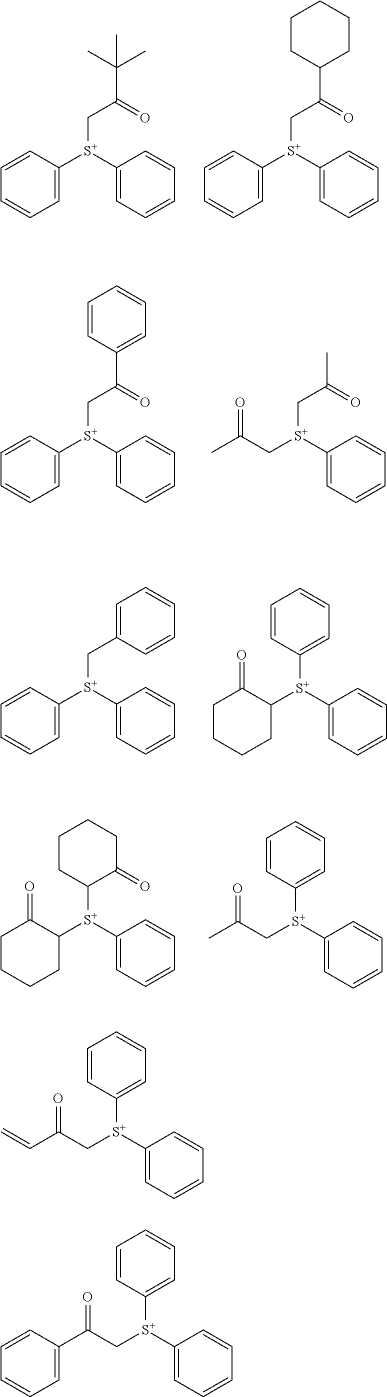

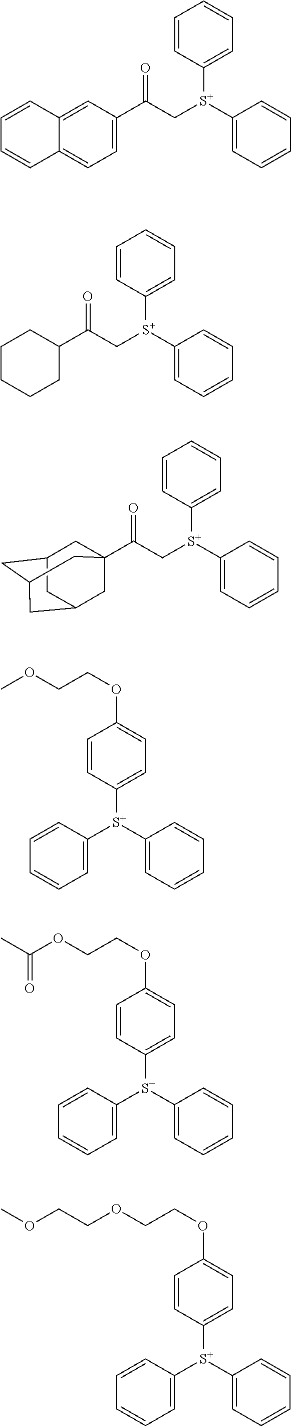 [SMILES: O=C(C[S+](c1ccccc1)c1ccccc1)c1ccc2ccccc2c1 ; O=C(C[S+](c1ccccc1)c1ccccc1)C1CCCCC1 ; O=C(C[S+](c1ccccc1)c1ccccc1)C12CC3CC(CC(C3)C1)C2 ; COCCOc1ccc([S+](c2ccccc2)c2ccccc2)cc1 ; CC(=O)OCCOc1ccc([S+](c2ccccc2)c2ccccc2)cc1 ; COCCOCCOc1ccc([S+](c2ccccc2)c2ccccc2)cc1]

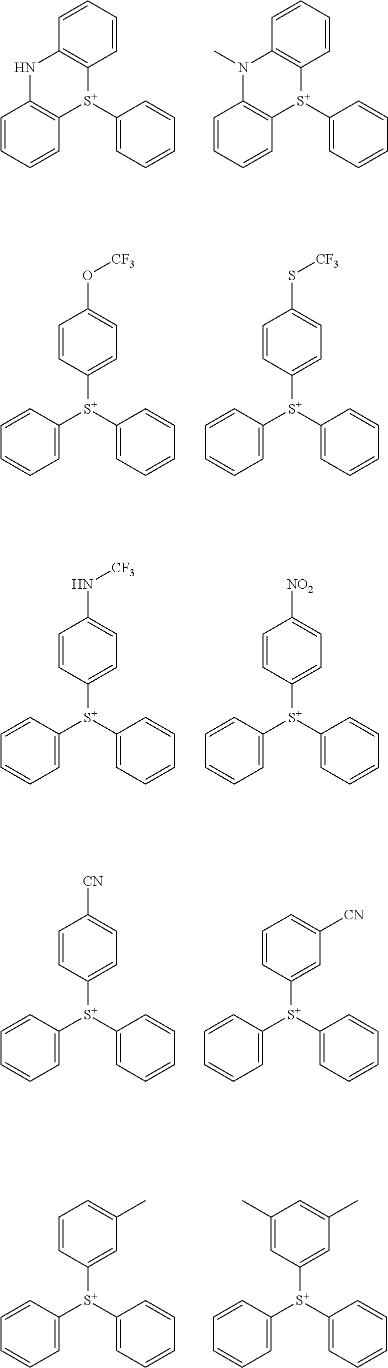

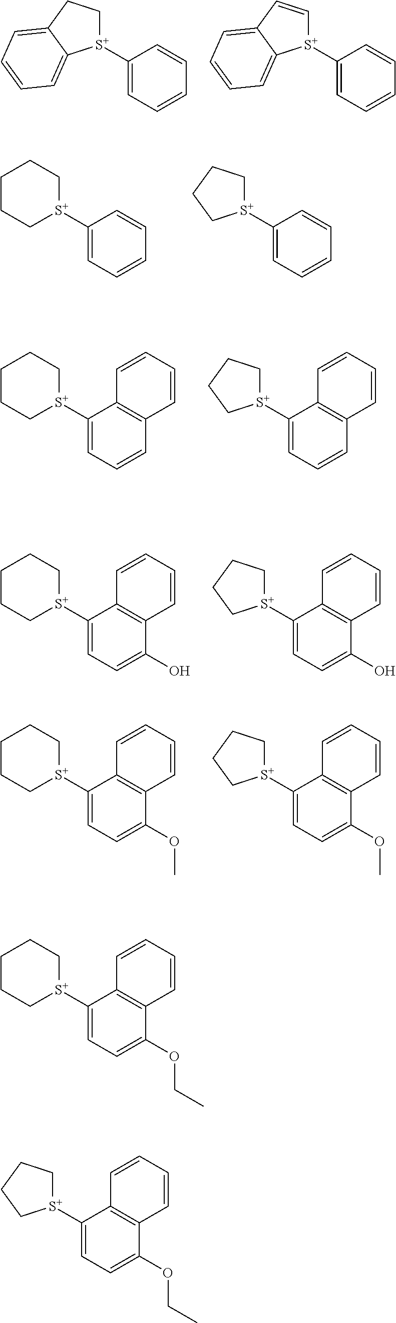

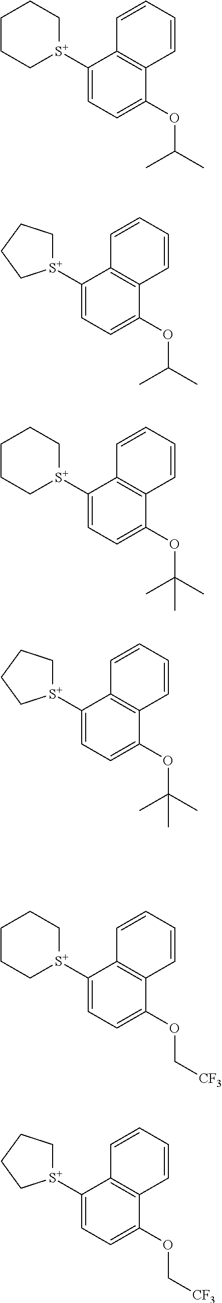 [SMILES: CC(C)Oc1ccc([S+]2CCCCC2)c2ccccc12 ; CC(C)Oc1ccc([S+]2CCCC2)c2ccccc12 ; CC(C)(C)Oc1ccc([S+]2CCCCC2)c2ccccc12 ; CC(C)(C)Oc1ccc([S+]2CCCC2)c2ccccc12 ; FC(F)(F)COc1ccc([S+]2CCCCC2)c2ccccc12 ; FC(F)(F)COc1ccc([S+]2CCCC2)c2ccccc12]

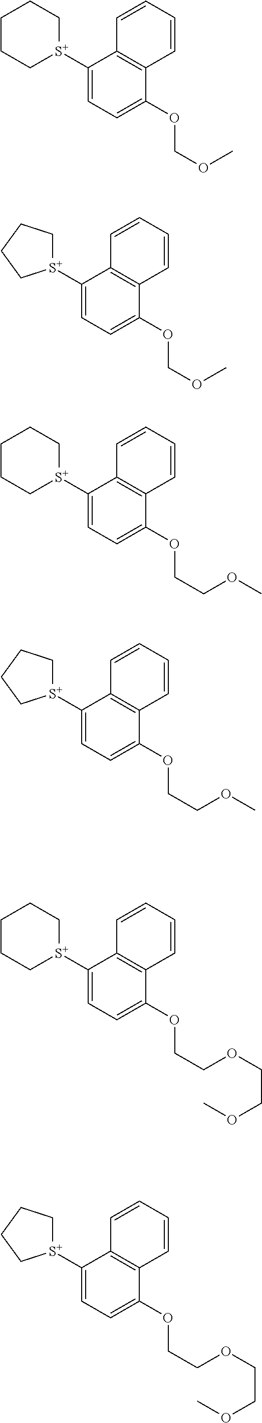 [SMILES: COCOc1ccc([S+]2CCCCC2)c2ccccc12 ; COCOc1ccc([S+]2CCCC2)c2ccccc12 ; COCCOc1ccc([S+]2CCCCC2)c2ccccc12 ; COCCOc1ccc([S+]2CCCC2)c2ccccc12 ; COCCOCCOc1ccc([S+]2CCCCC2)c2ccccc12 ; COCCOCCOc1ccc([S+]2CCCC2)c2ccccc12]

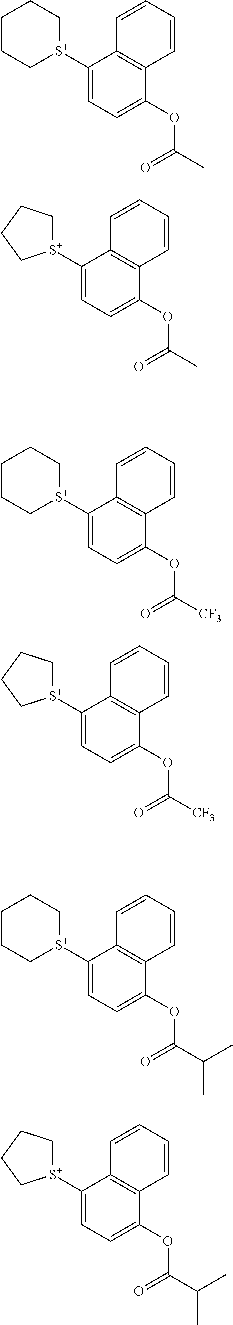 [SMILES: CC(=O)Oc1ccc([S+]2CCCCC2)c2ccccc12 ; CC(=O)Oc1ccc([S+]2CCCC2)c2ccccc12 ; O=C(Oc1ccc([S+]2CCCCC2)c2ccccc12)C(F)(F)F ; O=C(Oc1ccc([S+]2CCCC2)c2ccccc12)C(F)(F)F ; CC(C)C(=O)Oc1ccc([S+]2CCCCC2)c2ccccc12 ; CC(C)C(=O)Oc1ccc([S+]2CCCC2)c2ccccc12]

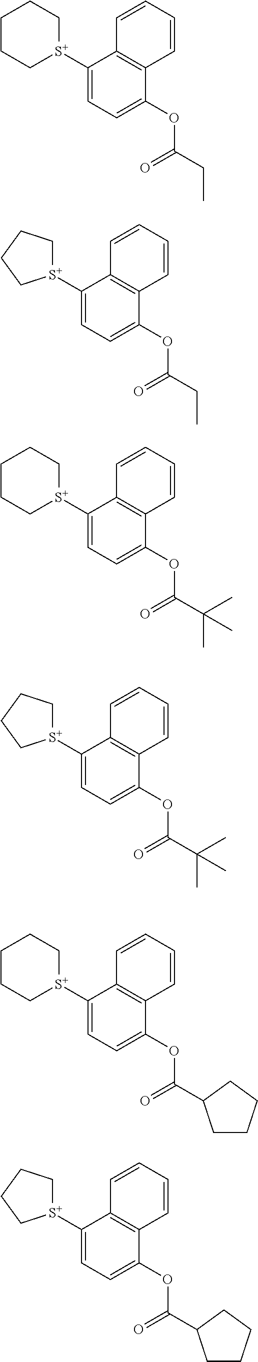 [SMILES: CCC(=O)Oc1ccc([S+]2CCCCC2)c2ccccc12 ; CCC(=O)Oc1ccc([S+]2CCCC2)c2ccccc12 ; CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1]

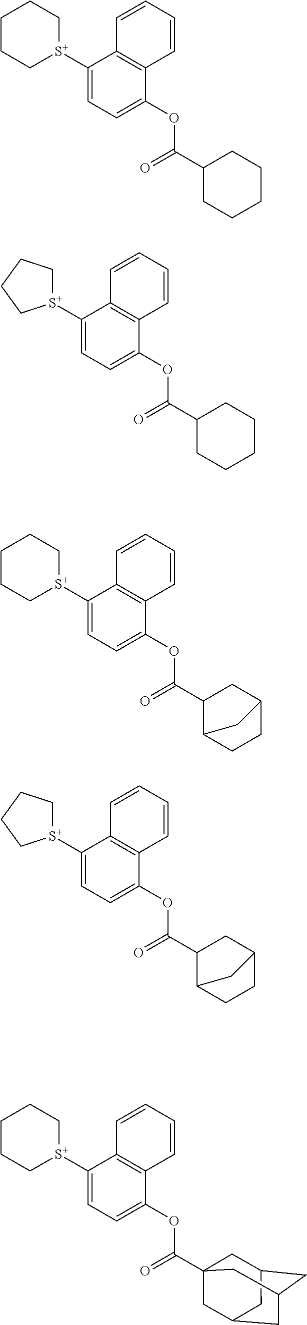 [SMILES: O=C(Oc1ccc([S+]2CCCCC2)c2ccccc12)C1CCCCC1 ; O=C(Oc1ccc([S+]2CCCC2)c2ccccc12)C1CCCCC1 ; O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2]

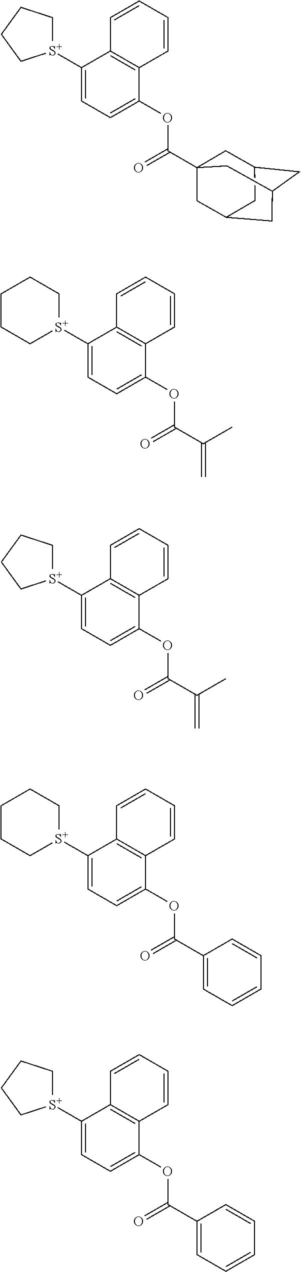 [SMILES: O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12 ; O=C(Oc1ccc([S+]2CCCCC2)c2ccccc12)c1ccccc1 ; O=C(Oc1ccc([S+]2CCCC2)c2ccccc12)c1ccccc1]

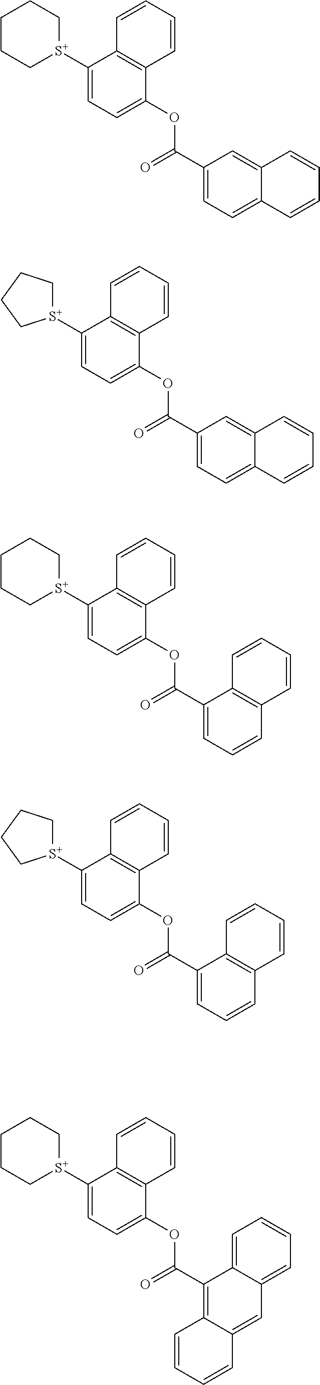 [SMILES: O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12 ; O=C(Oc1ccc([S+]2CCCCC2)c2ccccc12)c1c2ccccc2cc2ccccc12]

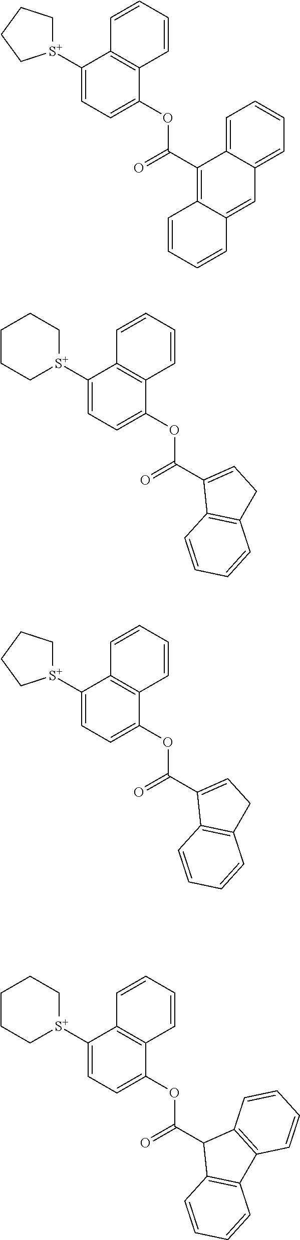 [SMILES: O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21 ; O=C(Oc1ccc([S+]2CCCCC2)c2ccccc12)C1c2ccccc2-c2ccccc21]

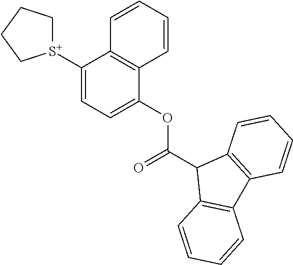 [SMILES: O=C(Oc1ccc([S+]2CCCC2)c2ccccc12)C1c2ccccc2-c2ccccc21]

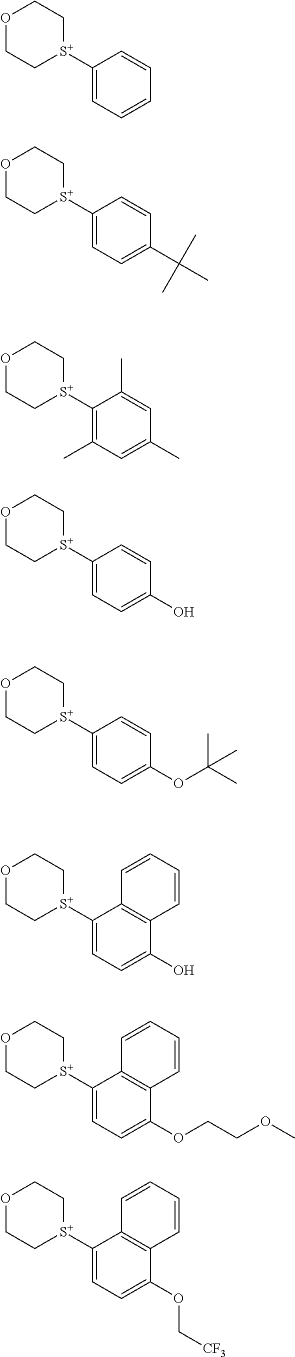

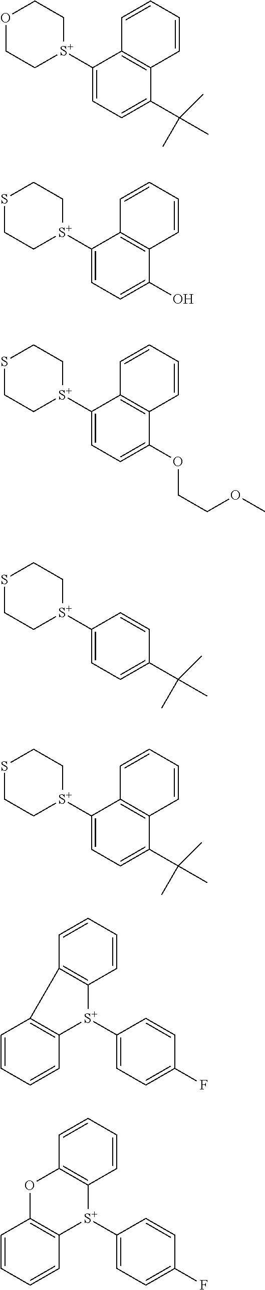

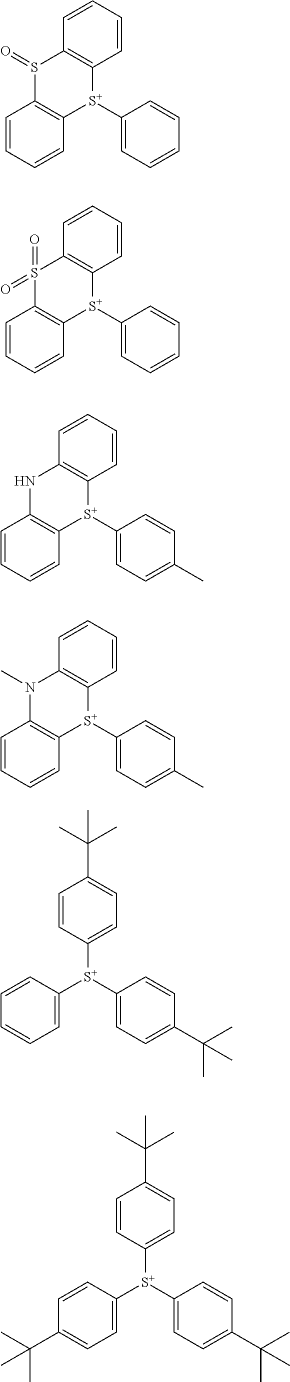

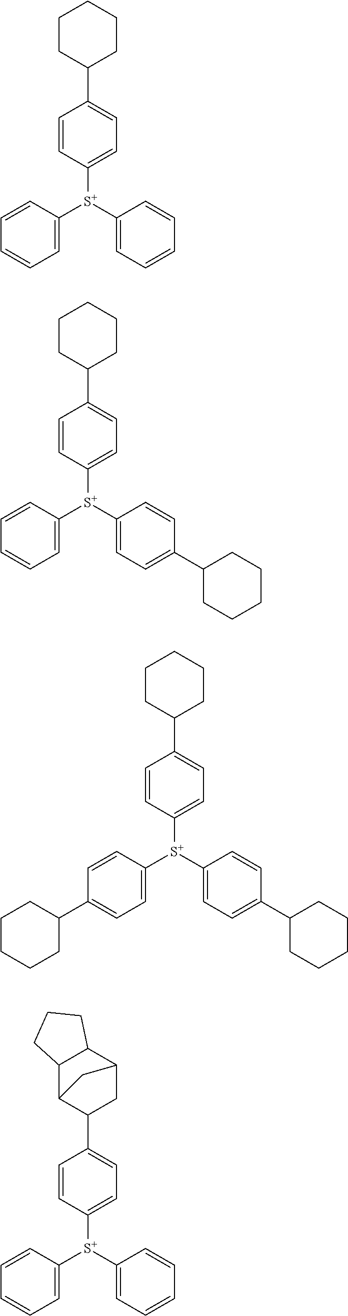 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1]

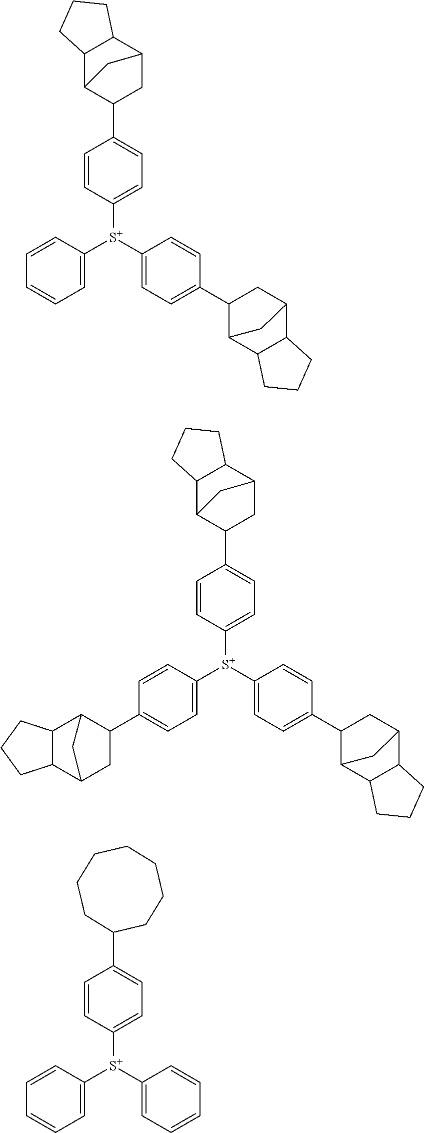 [SMILES: c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21 ; c1ccc([S+](c2ccccc2)c2ccc(C3CCCCCCC3)cc2)cc1]

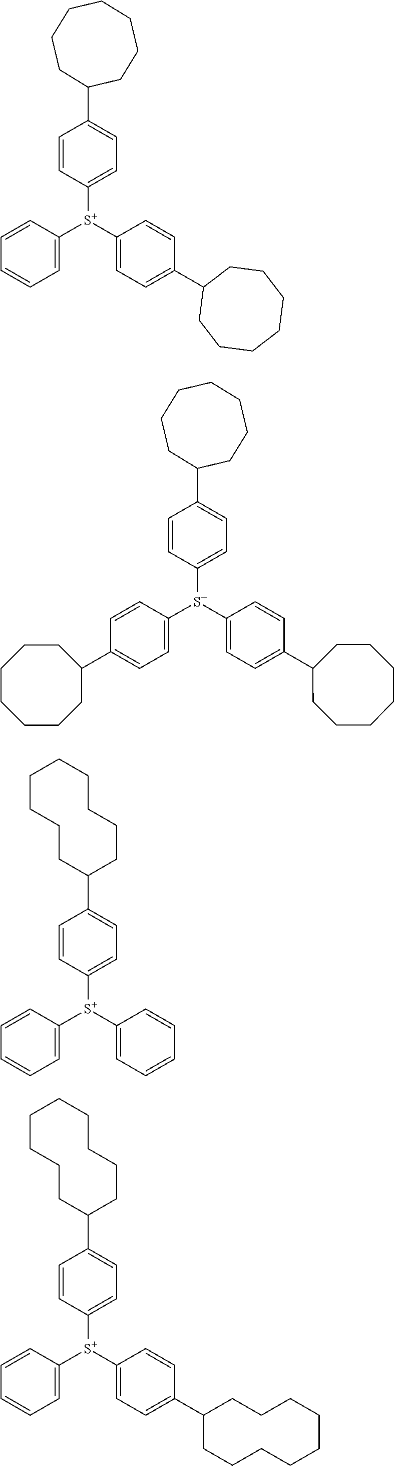 [SMILES: c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1]

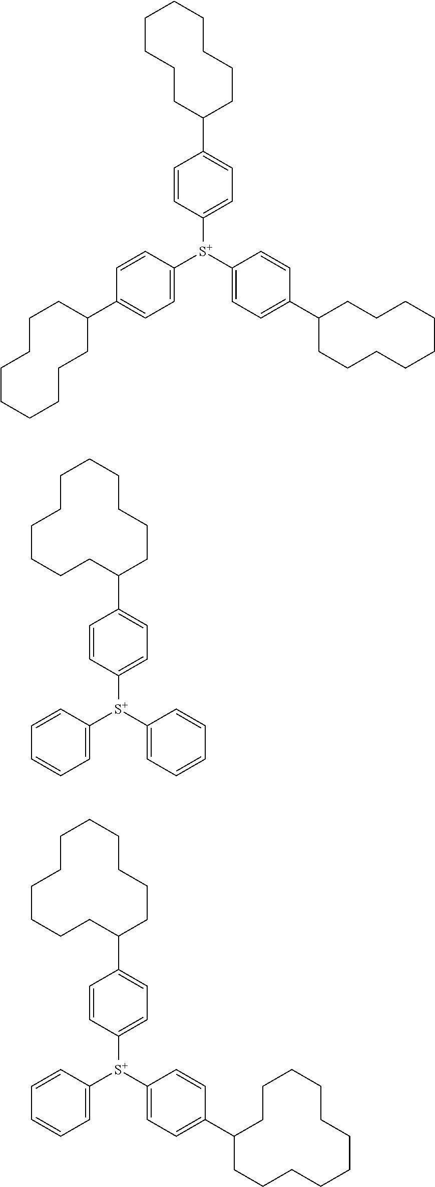 [SMILES: c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1]

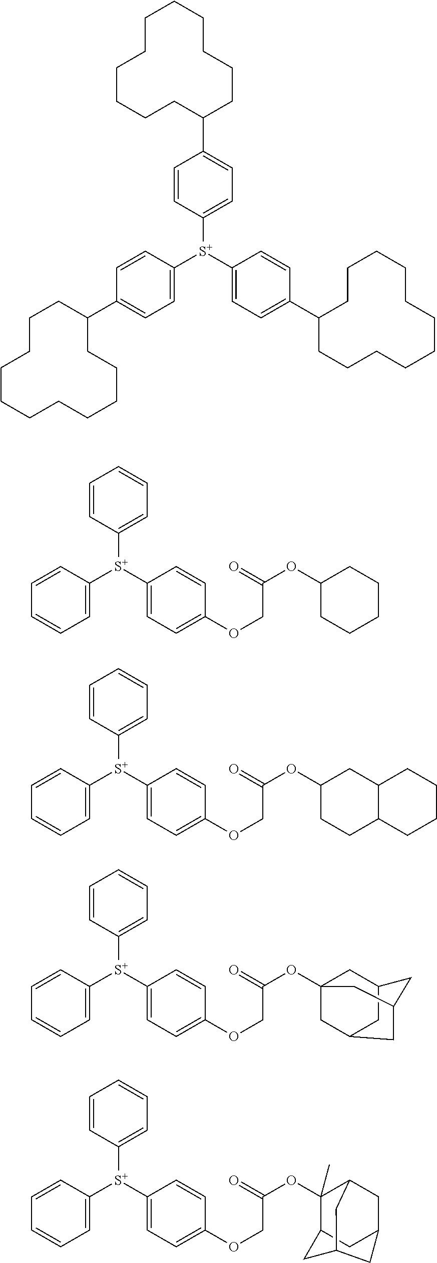 [SMILES: c1cc([S+](c2ccc(C3CCCCCCCCCCC3)cc2)c2ccc(C3CCCCCCCCCCC3)cc2)ccc1C1CCCCCCCCCCC1 ; O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1 ; O=C(COc1ccc([S+](c2ccccc2)c2ccccc2)cc1)OC12CC3CC(CC(C3)C1)C2 ; CC1(OC(=O)COc2ccc([S+](c3ccccc3)c3ccccc3)cc2)C2CC3CC(C2)CC1C3]

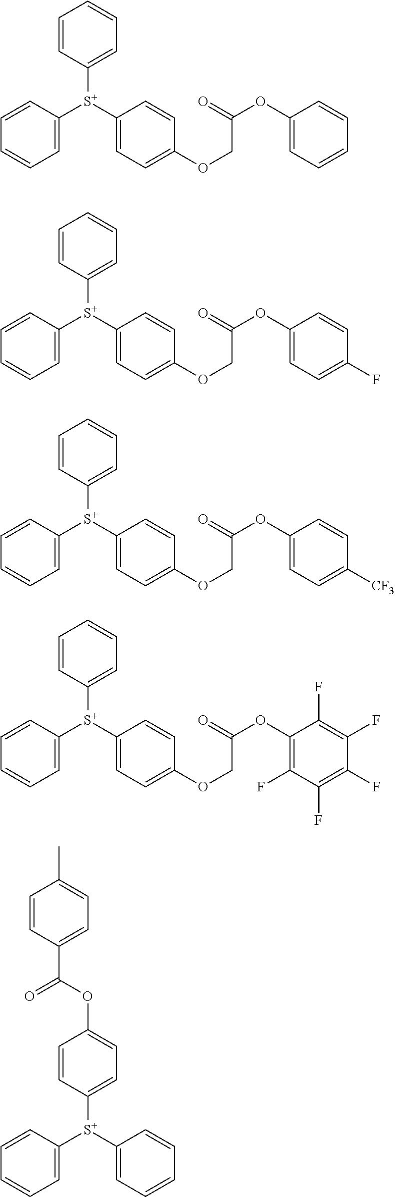 [SMILES: O=C(COc1ccc([S+](c2ccccc2)c2ccccc2)cc1)Oc1ccccc1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; Cc1ccc(C(=O)Oc2ccc([S+](c3ccccc3)c3ccccc3)cc2)cc1]

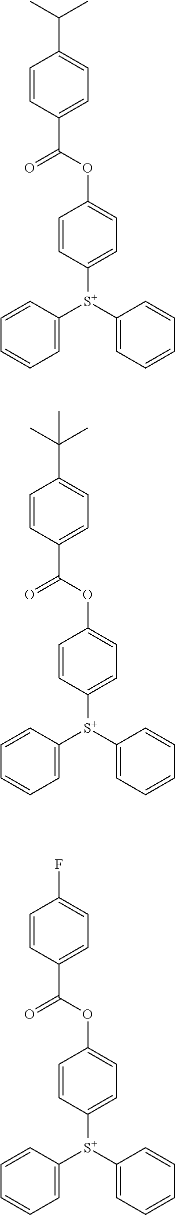 [SMILES: CC(C)c1ccc(C(=O)Oc2ccc([S+](c3ccccc3)c3ccccc3)cc2)cc1 ; CC(C)(C)c1ccc(C(=O)Oc2ccc([S+](c3ccccc3)c3ccccc3)cc2)cc1 ; O=C(Oc1ccc([S+](c2ccccc2)c2ccccc2)cc1)c1ccc(F)cc1]

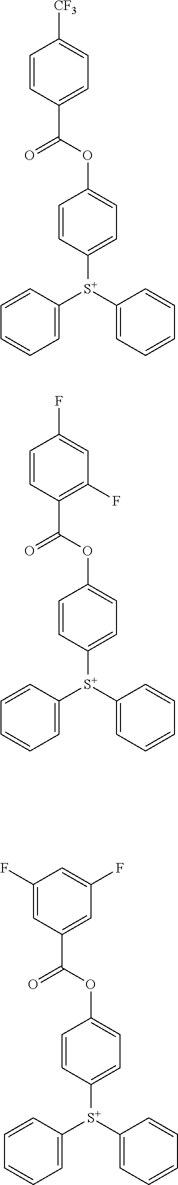 [SMILES: O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1]

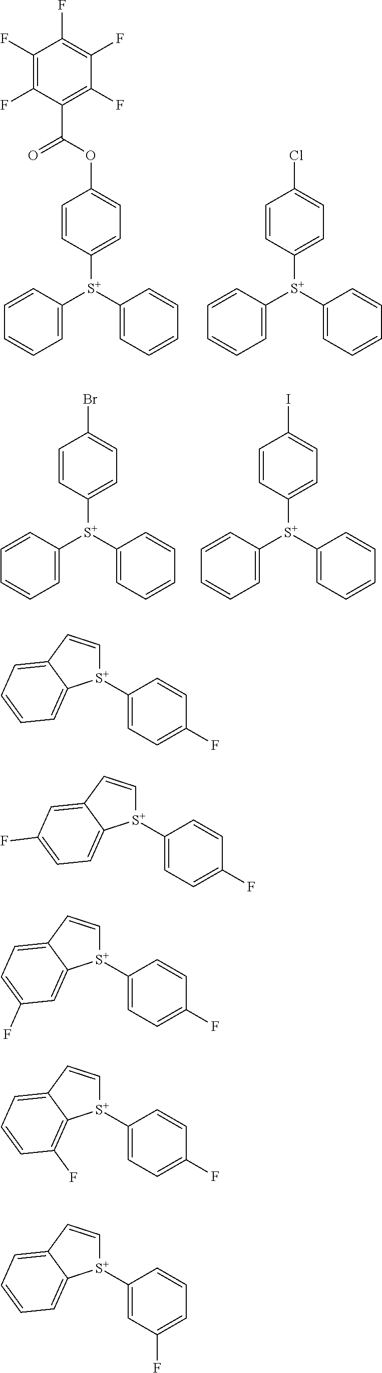

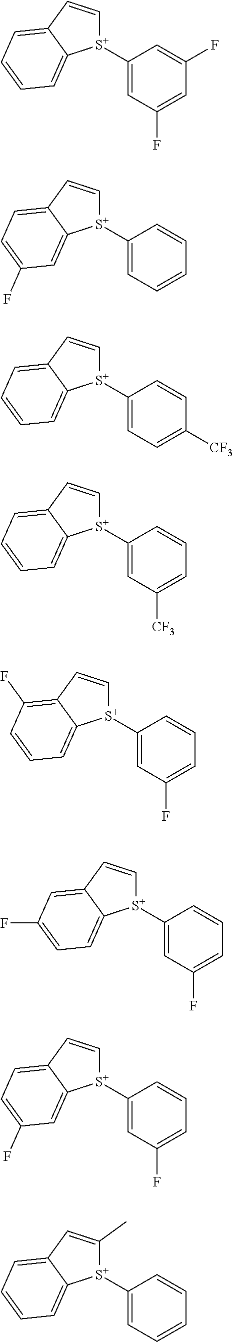

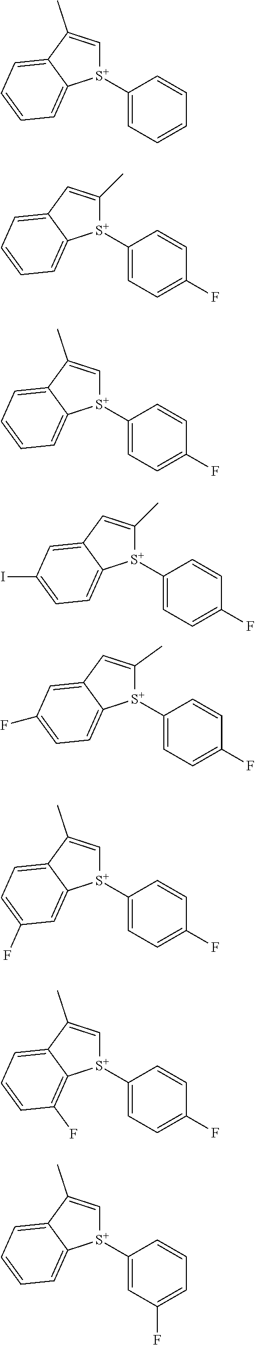

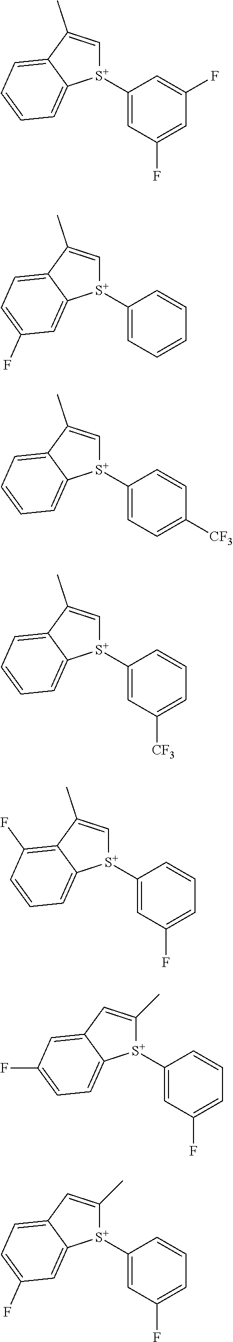

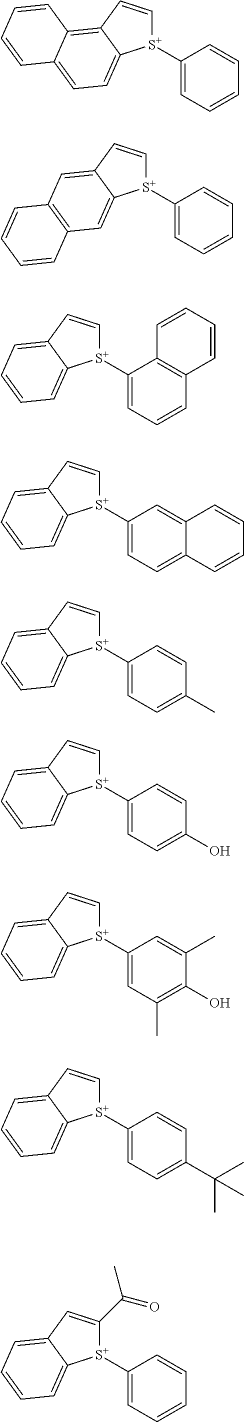

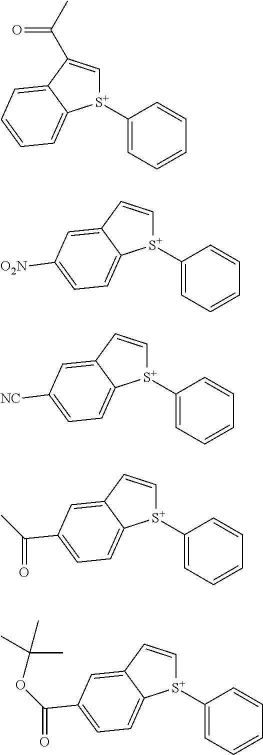 [SMILES: CC(=O)c1c[s+](-c2ccccc2)c2ccccc12 ; O=[N+]([O-])c1ccc2c(cc[s+]2-c2ccccc2)c1 ; N#Cc1ccc2c(cc[s+]2-c2ccccc2)c1 ; CC(=O)c1ccc2c(cc[s+]2-c2ccccc2)c1 ; CC(C)(C)OC(=O)c1ccc2c(cc[s+]2-c2ccccc2)c1]

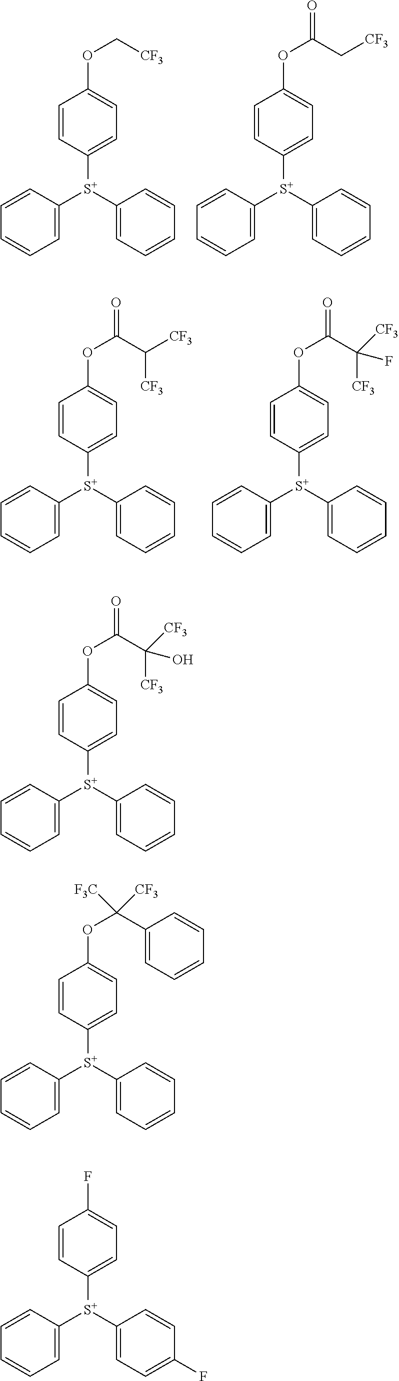

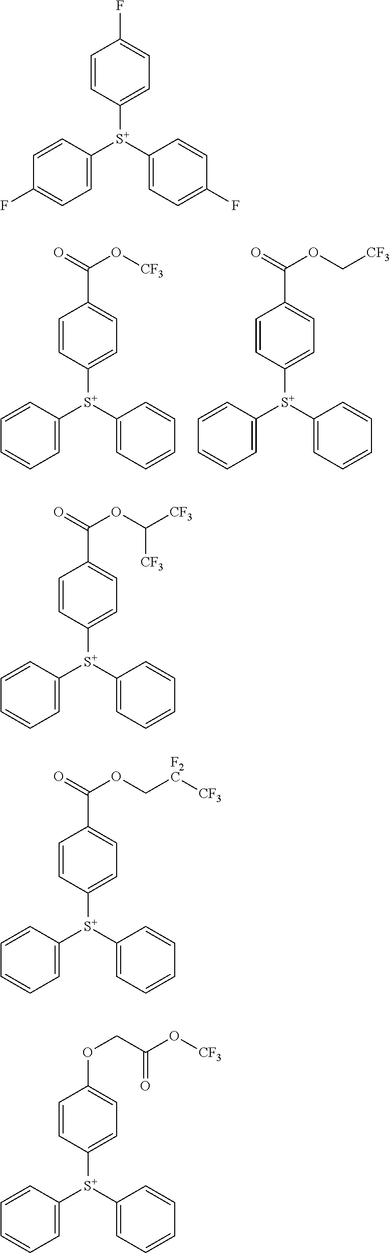 [SMILES: Fc1ccc([S+](c2ccc(F)cc2)c2ccc(F)cc2)cc1 ; O=C(OC(F)(F)F)c1ccc([S+](c2ccccc2)c2ccccc2)cc1 ; O=C(OCC(F)(F)F)c1ccc([S+](c2ccccc2)c2ccccc2)cc1 ; O=C(OC(C(F)(F)F)C(F)(F)F)c1ccc([S+](c2ccccc2)c2ccccc2)cc1 ; O=C(OCC(F)(F)C(F)(F)F)c1ccc([S+](c2ccccc2)c2ccccc2)cc1 ; O=C(COc1ccc([S+](c2ccccc2)c2ccccc2)cc1)OC(F)(F)F]

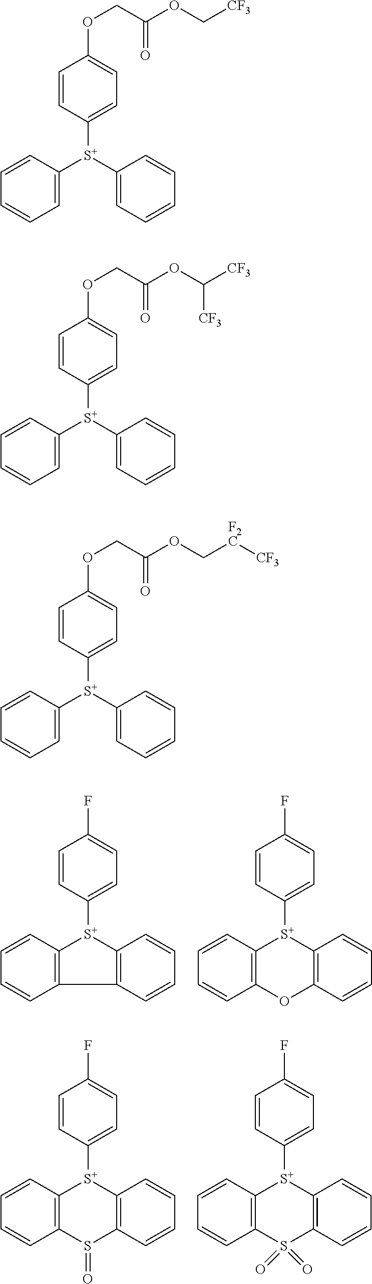

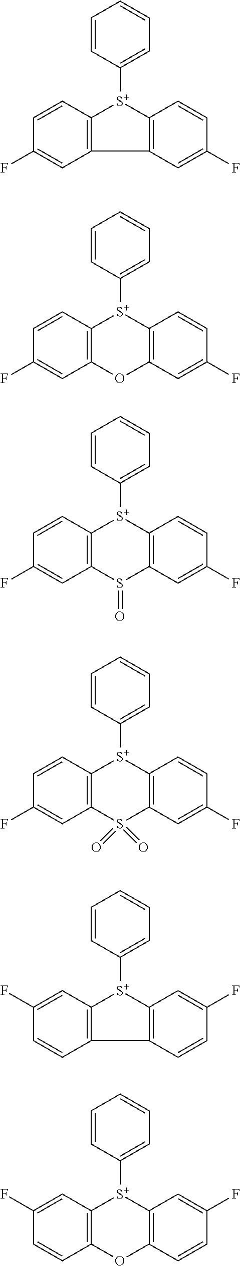 [SMILES: Fc1ccc2c(c1)c1cc(F)ccc1[s+]2-c1ccccc1 ; Fc1ccc2c(c1)Oc1cc(F)ccc1[S+]2c1ccccc1 ; O=S1c2cc(F)ccc2[S+](c2ccccc2)c2ccc(F)cc21 ; O=S1(=O)c2cc(F)ccc2[S+](c2ccccc2)c2ccc(F)cc21 ; Fc1ccc2c3ccc(F)cc3[s+](-c3ccccc3)c2c1 ; Fc1ccc2c(c1)[S+](c1ccccc1)c1cc(F)ccc1O2]

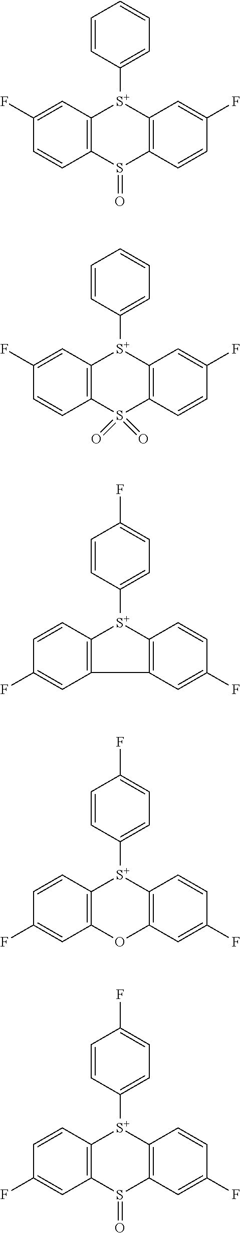 [SMILES: O=S1c2ccc(F)cc2[S+](c2ccccc2)c2cc(F)ccc21 ; O=S1(=O)c2ccc(F)cc2[S+](c2ccccc2)c2cc(F)ccc21 ; Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21]

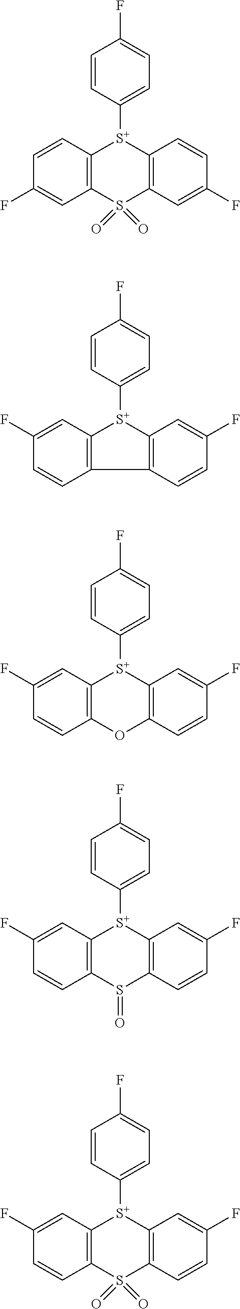 [SMILES: O=S1(=O)c2cc(F)ccc2[S+](c2ccc(F)cc2)c2ccc(F)cc21 ; Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21]

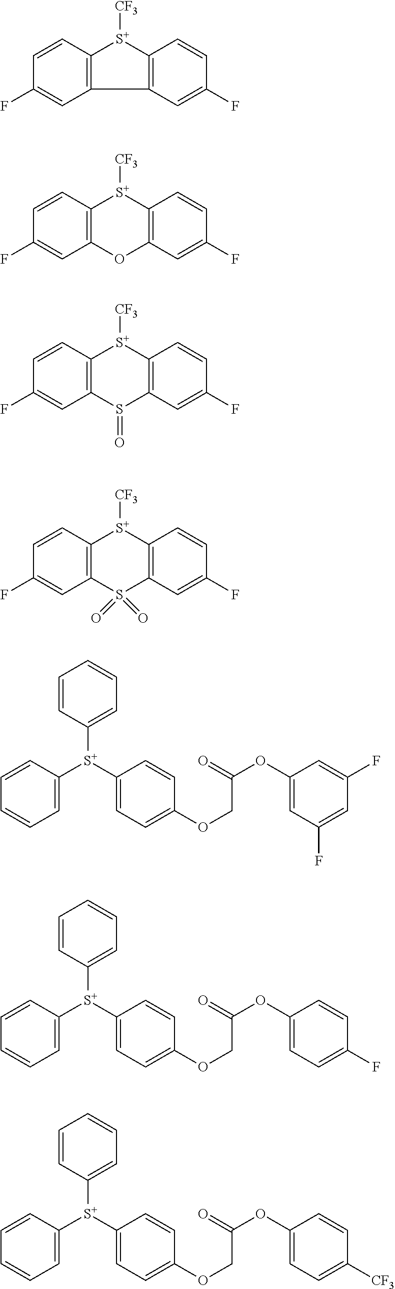

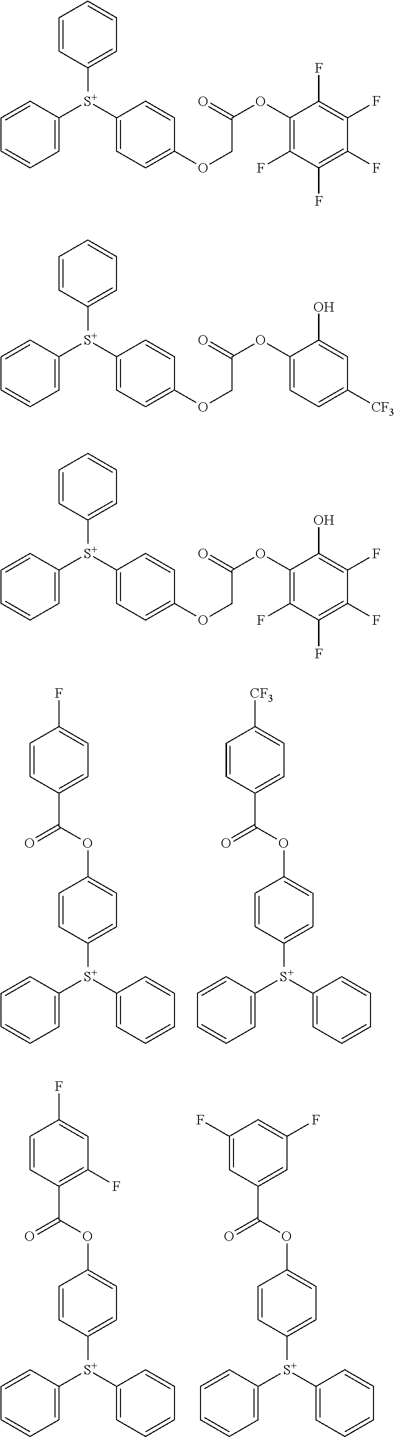

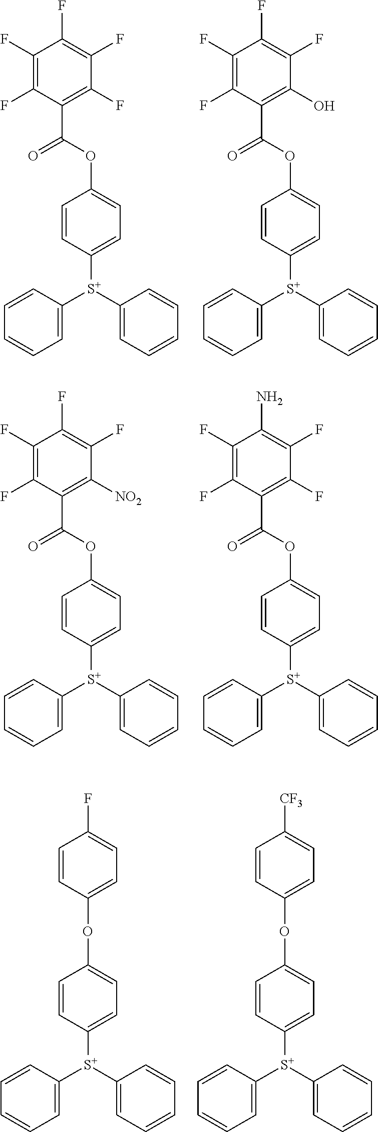 [SMILES: O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F ; O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1]

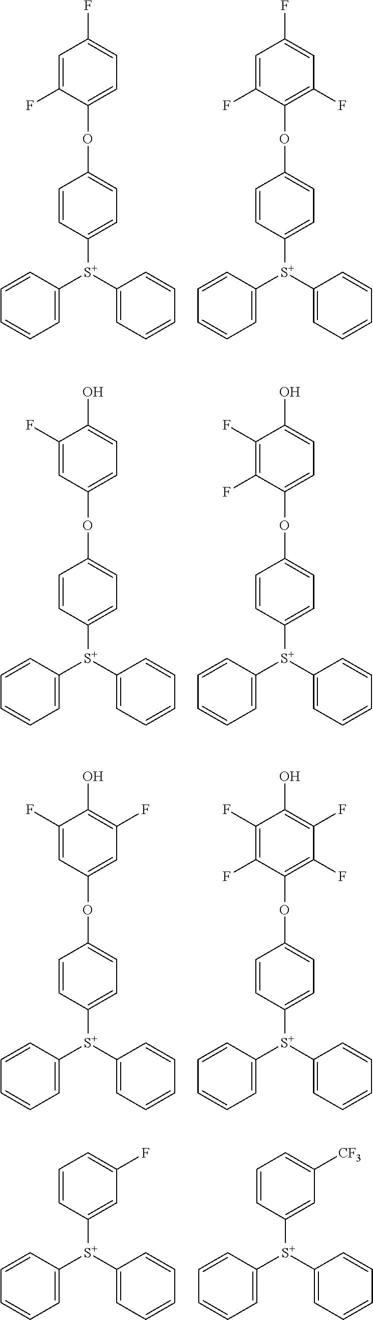

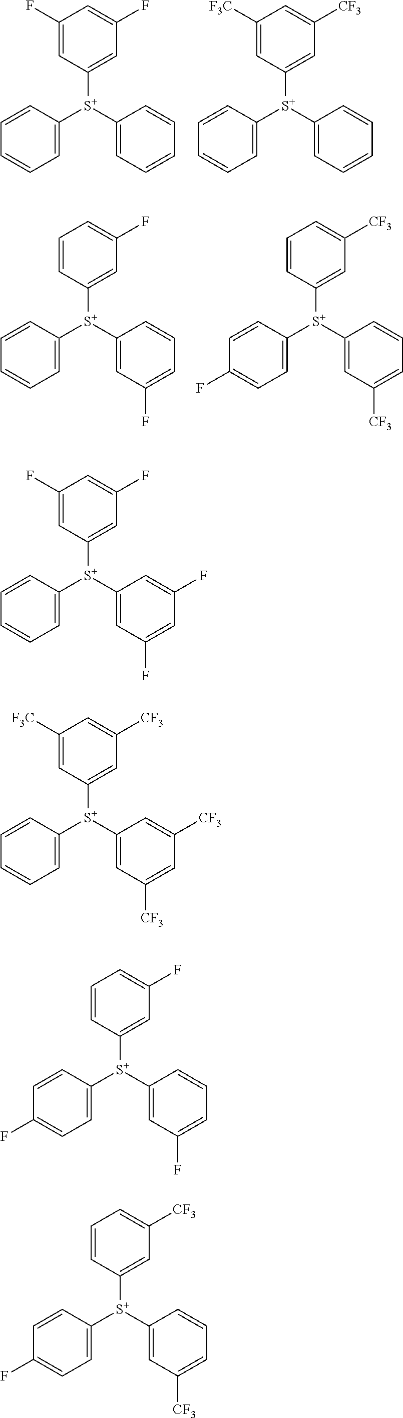

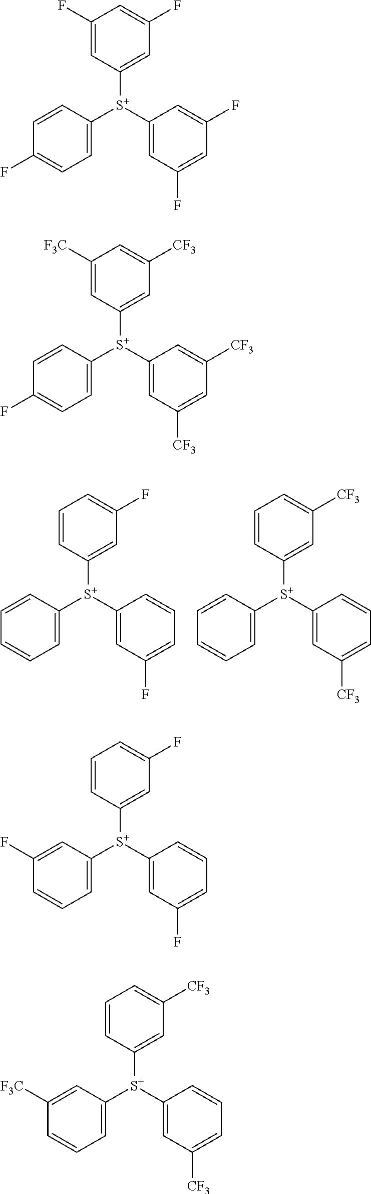 [SMILES: Fc1ccc([S+](c2cc(F)cc(F)c2)c2cc(F)cc(F)c2)cc1 ; Fc1ccc([S+](c2cc(C(F)(F)F)cc(C(F)(F)F)c2)c2cc(C(F)(F)F)cc(C(F)(F)F)c2)cc1 ; Fc1cccc([S+](c2ccccc2)c2cccc(F)c2)c1 ; FC(F)(F)c1cccc([S+](c2ccccc2)c2cccc(C(F)(F)F)c2)c1 ; Fc1cccc([S+](c2cccc(F)c2)c2cccc(F)c2)c1 ; FC(F)(F)c1cccc([S+](c2cccc(C(F)(F)F)c2)c2cccc(C(F)(F)F)c2)c1]

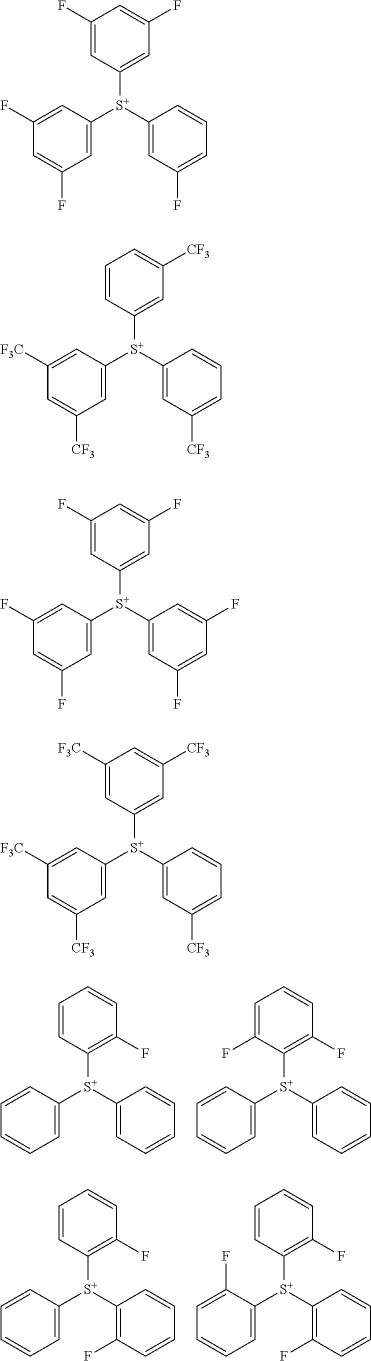

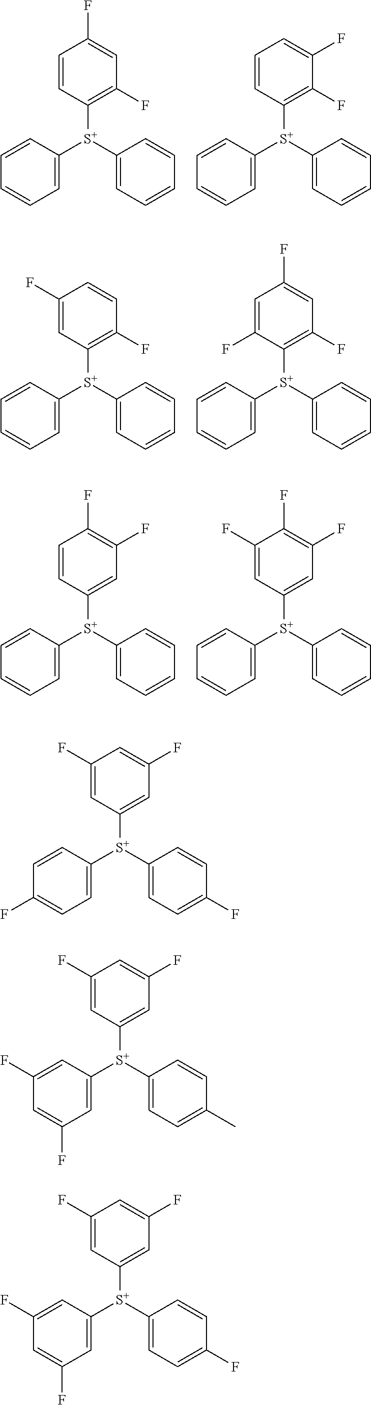

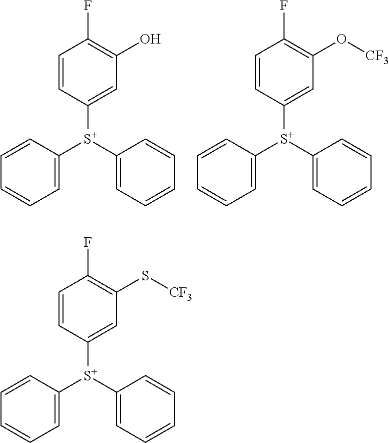 [SMILES: Oc1cc([S+](c2ccccc2)c2ccccc2)ccc1F ; Fc1ccc([S+](c2ccccc2)c2ccccc2)cc1OC(F)(F)F ; Fc1ccc([S+](c2ccccc2)c2ccccc2)cc1SC(F)(F)F]

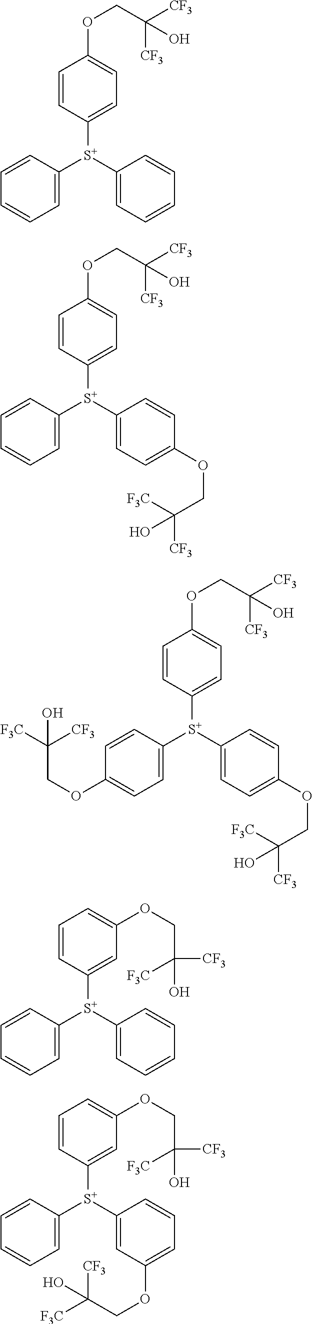 [SMILES: OC(COc1ccc([S+](c2ccccc2)c2ccccc2)cc1)(C(F)(F)F)C(F)(F)F ; OC(COc1ccc([S+](c2ccccc2)c2ccc(OCC(O)(C(F)(F)F)C(F)(F)F)cc2)cc1)(C(F)(F)F)C(F)(F)F ; OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F ; OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F]

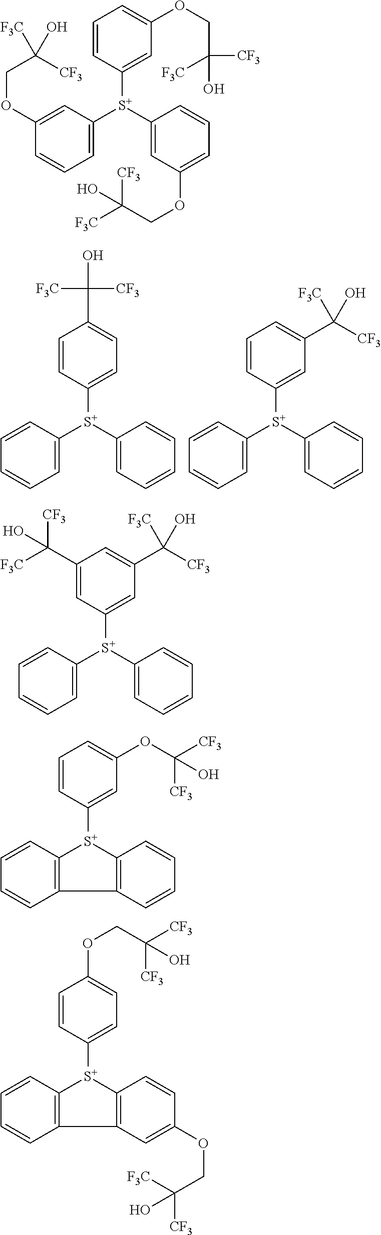 [SMILES: OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F ; OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F ; OC(Oc1cccc(-[s+]2c3ccccc3c3ccccc32)c1)(C(F)(F)F)C(F)(F)F ; OC(COc1ccc(-[s+]2c3ccccc3c3cc(OCC(O)(C(F)(F)F)C(F)(F)F)ccc32)cc1)(C(F)(F)F)C(F)(F)F]

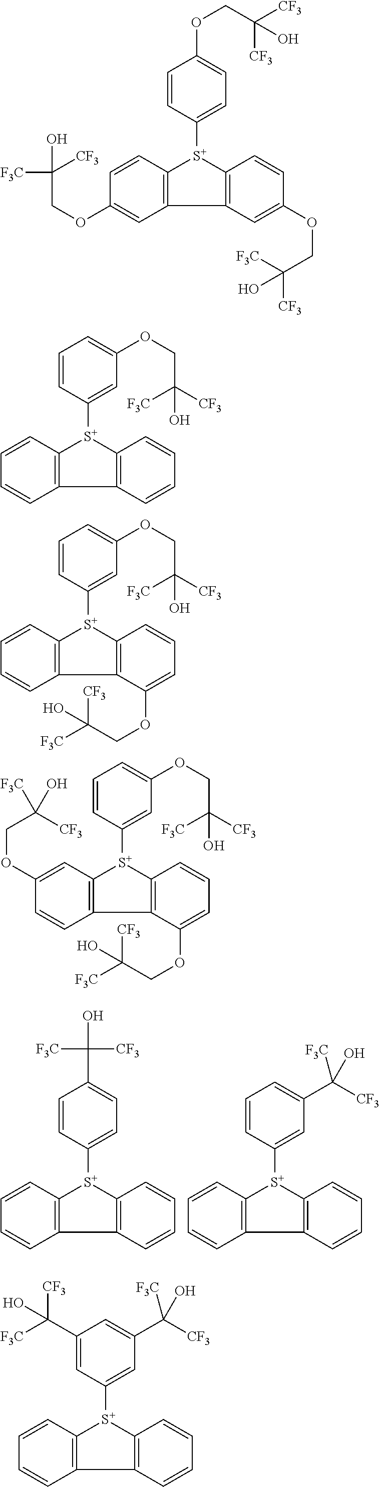

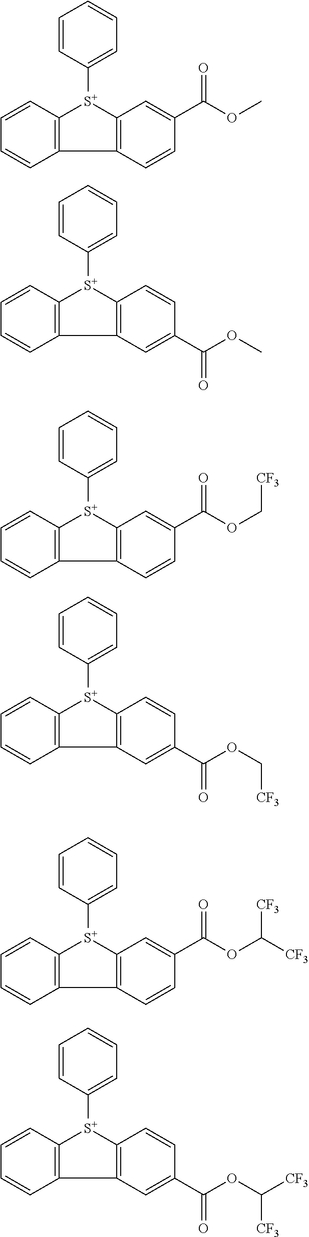 [SMILES: COC(=O)c1ccc2c3ccccc3[s+](-c3ccccc3)c2c1 ; COC(=O)c1ccc2c(c1)c1ccccc1[s+]2-c1ccccc1 ; O=C(OCC(F)(F)F)c1ccc2c3ccccc3[s+](-c3ccccc3)c2c1 ; O=C(OCC(F)(F)F)c1ccc2c(c1)c1ccccc1[s+]2-c1ccccc1 ; O=C(OC(C(F)(F)F)C(F)(F)F)c1ccc2c3ccccc3[s+](-c3ccccc3)c2c1 ; O=C(OC(C(F)(F)F)C(F)(F)F)c1ccc2c(c1)c1ccccc1[s+]2-c1ccccc1]

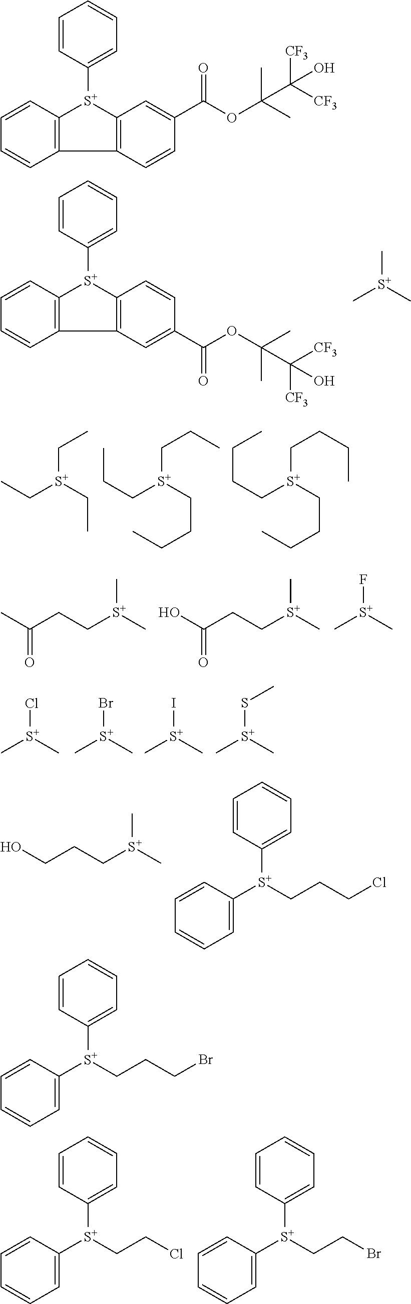

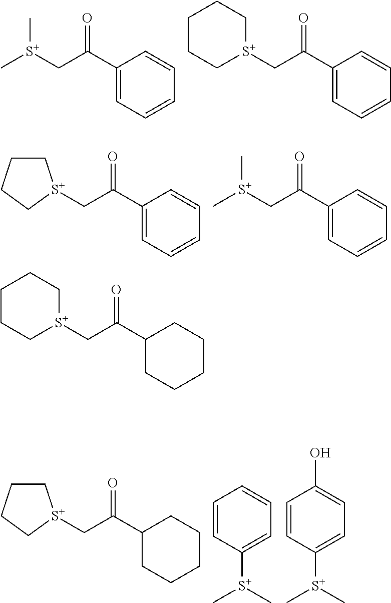

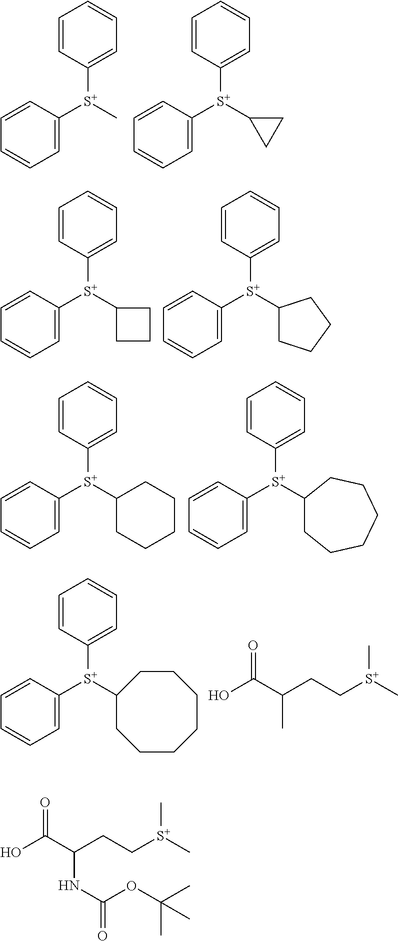

Examples of the iodonium cation having formula (cation-2) are shown below, but not limited thereto.

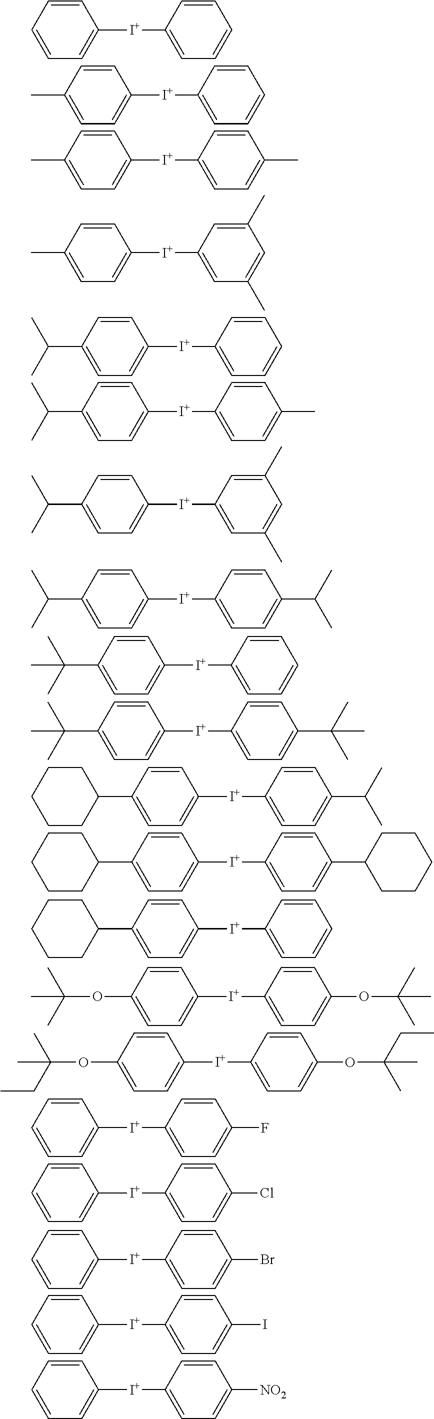

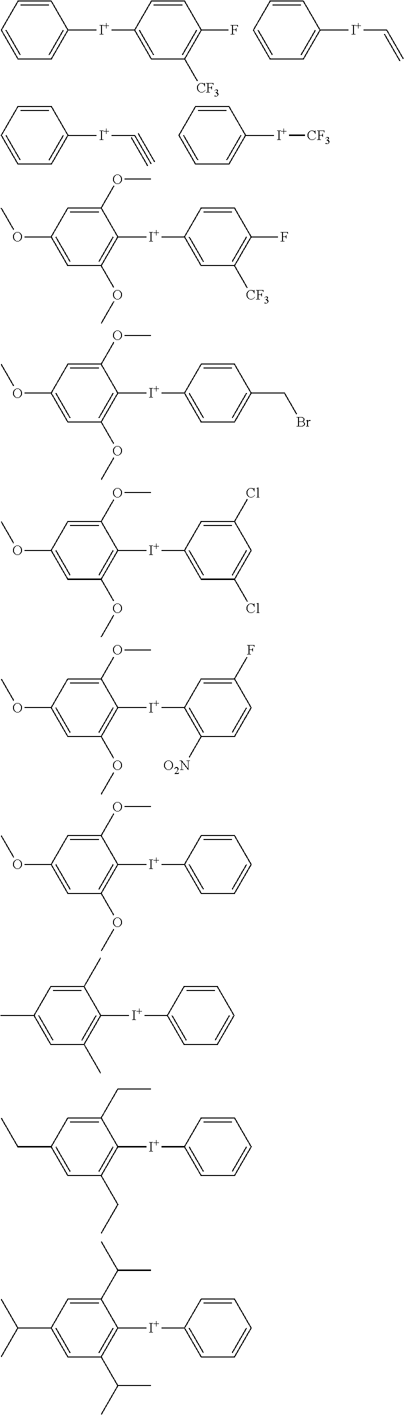

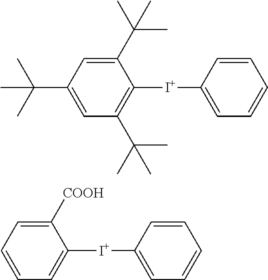 [SMILES: CC(C)(C)c1cc(C(C)(C)C)c([I+]c2ccccc2)c(C(C)(C)C)c1 ; O=C(O)c1ccccc1[I+]c1ccccc1]

Examples of the onium salt include arbitrary combinations of anions with cations, both as exemplified above.

The onium salt having formula (1) can be synthesized by well-known methods. For example, a method of preparing an onium salt having formula (PAG-1-ex) is described.

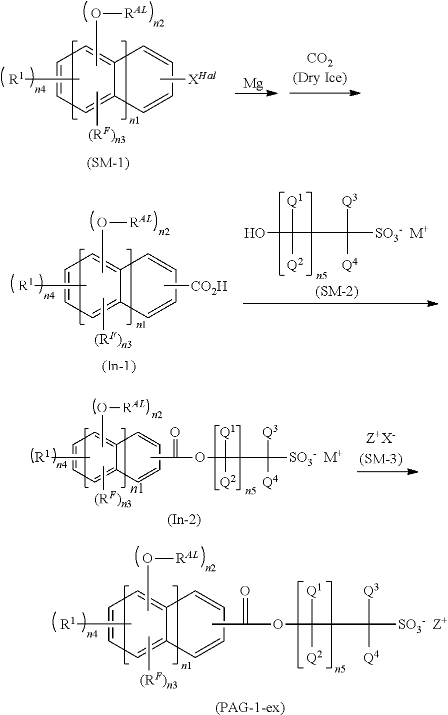

Herein R^{AL}, R^{F}, R¹, Q¹ to Q⁴, n1 to n5, and Z⁺ are as defined above, X^{Ha1} is chlorine, bromine or iodine, M⁺ is a counter cation, and X⁻ is a counter anion.

The first step is to produce Intermediate In-1 by preparing a Grignard reagent from reactant SM-1, which is commercially available or can be synthesized by any well-known synthesis method, and reacting it with carbon dioxide (or dry ice). The reaction may be performed by any well-known organic synthesis method. Specifically, the Grignard reagent is prepared by suspending metallic magnesium in an ether solvent such as diethyl ether or tetrahydrofiran (THF) and adding a dilute solution of reactant SM-1 in the solvent dropwise thereto. Where X^{Ha1} in reactant SM-1 is bromine or iodine, the activating agent for metallic magnesium is not always necessary. Where X^{Ha1} in reactant SM-1 is chlorine, the Grignard reagent can be smoothly prepared by using a small amount of 1,2-dibromoethane or iodine as the activating agent. The reaction temperature ranges from room temperature to near the boiling point of the solvent. Once the Grignard reagent is prepared, dry ice is suspended in the solvent used for preparation, and the Grignard reagent is added dropwise thereto. The reaction time is determined as appropriate by monitoring the reaction process by silica gel thin-layer chromatography (TLC) because it is desirable from the yield aspect to drive the reaction to completion. Usually the reaction time is about 5 to 30 minutes. Thereafter, the magnesium salt is dissolved in dilute hydrochloric acid or the like, and the desired compound is extracted from the reaction mixture. Through ordinary aqueous work-up, Intermediate In-1 is recovered. The resulting Intermediate In-1 may be purified by a standard technique such as chromatography or re-crystallization if necessary.

The second step is to react Intermediate In-1 with reactant SM-2 to form Intermediate In-2. Any condensing agents may be used when an ester bond is formed directly from the carboxy group in Intermediate In-1 and the hydroxy group in reactant SM-2. Suitable condensing agents include N,N′-dicyclohexylcarbodiimide. N,N′-diisopropylcarbodiimide, 1-[3-(dimetahylamino)propyl]-3-ethylcarbodiimide, and 1-ethyl-3-(3-dimethylaminopropyl)carbodiimide hydrochloride. From the aspect of easy removal of a urea compound formed as the by-product after the reaction, it is preferred to use 1-ethyl-3-(3-dimethylaminopropyl)carbodiimide hydrochloride. The reaction is performed by dissolving Intermediate In-1 and reactant SM-2 in a halide solvent such as methylene chloride, and adding a condensing agent thereto. The reaction rate may be accelerated by adding 4-dimethylaminopyridine (DMAP) as a catalyst. The reaction time is determined as appropriate by monitoring the reaction process by TLC because it is desirable from the yield aspect to drive the reaction to completion. Usually the reaction time is about 12 to 24 hours. After the reaction is stopped, the by-product, urea compound is removed by filtration or water washing if necessary. The reaction solution is subjected to ordinary aqueous work-up, obtaining Intermediate In-2. The resulting Intermediate In-2 may be purified by a standard technique such as chromatography or re-crystallization if necessary.

The third step is a salt exchange between the resulting Intermediate In-2 and an onium salt: Z⁺X⁻ to form the desired onium salt (PAG-1-ex). It is preferred that X be a chloride ion, bromide ion, iodide ion or methylsulfate anion, which allows the exchange reaction to run in a quantitative manner. It is preferred from the yield aspect to monitor the progress of reaction by TLC. By ordinary aqueous work-up, the onium salt (PAG-1-ex) is recovered from the reaction mixture. The onium salt (PAG-1-ex) may be purified by a standard technique such as chromatography or re-crystallization if necessary.

In the above reaction scheme, the ion exchange in the third step may be readily performed by any well-known technique, for example, with reference to JP-A 2007-145797.

The above-mentioned preparation method is merely exemplary and the method of preparing the inventive onium salt is not limited thereto.

The inventive onium salt has a characteristic structure that the anion possesses an acid labile group attached to a hydroxy group on the aromatic ring and a fluorinated substituent group, which are attached to the adjoining carbon atoms. In the exposed region, the acid labile group undergoes deprotection reaction under the catalysis of the generated acid to generate an aromatic hydroxy group. This acts to enhance the contrast between exposed and unexposed regions. The adjoining fluorinated substituent group serves to improve the resist solvent solubility of the sulfonium salt itself, and its electron attractiveness serves to enhance the acidity of the aromatic hydroxy group formed in the exposed region. When the resist film after exposure is developed in an alkaline developer, the exposed region is effectively removed by the alkaline developer due to the increased affinity of the aromatic hydroxy group to the alkaline developer. It is also contemplated that the aromatic hydroxy group adjoining the fluorinated substituent group does not draw the alkaline developer deeply into the unexposed region as compared with the carboxy group because of the water repellency of fluorine, which is effective for reducing swelling by the alkaline developer. This prevents the resist pattern in the unexposed region from collapsing. By virtue of the synergy of these effects, the positive resist composition comprising the inventive onium salt can form a resist pattern having a high dissolution contrast and improved LWR of line patterns or improved CDU of hole patterns. The pattern is fully resistant to collapse.

The onium salt is advantageously used as a PAG.

Chemically Amplified Resist Composition

(A) Photoacid Generator

Another embodiment of the invention is a chemically amplified resist composition essentially comprising (A) the onium salt having formula (1) as a photoacid generator.

In the chemically amplified resist composition, the amount of the PAG in the form of the onium salt having formula (1) as component (A) is preferably 0.1 to 40 parts by weight, more preferably 0.5 to 30 parts by weight per 80 parts by weight of a base polymer to be described just below. As long as the amount of component (A) is in the range, good sensitivity and resolution are achievable and the risk of foreign particles being formed after development or during stripping of resist film is avoided. The PAG may be used alone or in admixture as component (A).

(B) Base Polymer

The chemically amplified resist composition may comprise a base polymer as component (B). The base polymer (B) preferably contains repeat units having the formula (a1), which are also referred to as repeat units (a1).

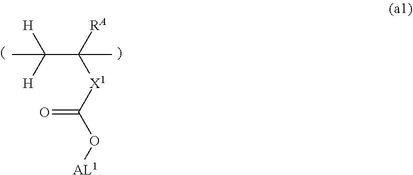

In formula (a1), R^{A} is hydrogen, fluorine, methyl or trifluoromethyl.

In formula (a1), X¹ is a single bond, phenylene group, naphthylene group or *—C(═O)—O—X¹¹—. The phenylene or naphthylene group may be substituted with an optionally fluorinated C₁-C₁₀ alkoxy moiety or halogen. X¹¹ is a C₁-C₁₀ saturated hydrocarbylene group, phenylene or naphthylene group, and the saturated hydrocarbylene group may contain a hydroxy moiety, ether bond, ester bond or lactone ring.

In formula (a1), AL¹ is an acid labile group. Examples of the acid labile group include those described in U.S. Pat. No. 8,574,817 (JP-A 2013-080033) and U.S. Pat. No. 8,846,303 (JP-A 2013-083821).

Typical of the acid labile group are groups of the following formulae (AL-3) to (AL-5).

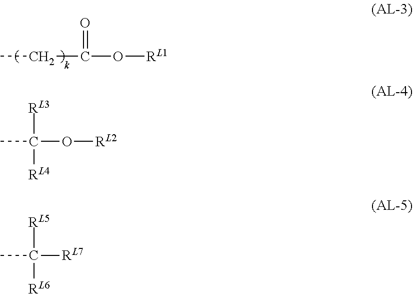

In formulae (AL-3) and (AL4), R^{L1} and R^{L2} are each independently a C₁-C₄₀ saturated hydrocarbyl group which may contain a heteroatom such as oxygen, sulfur, nitrogen or fluorine. The hydrocarbyl group may be straight, branched or cyclic. Inter alia, C₁-C₂₀ saturated hydrocarbyl groups are preferred.

In formula (AL-3), k is an integer of 0 to 10, preferably 1 to 5.

In formula (AL-4). R^{L3} and R^{L4} are each independently hydrogen or a C₁-C₂₀ saturated hydrocarbyl group which may contain a heteroatom such as oxygen, sulfur, nitrogen or fluorine. The hydrocarbyl group may be straight, branched or cyclic. Any two of R^{F}, R^{L} and R^{L4} may bond together to form a C₃-C₂₀ ring with the carbon atom or carbon and oxygen atoms to which they are attached. The ring preferably contains 4 to 16 carbon atoms and is typically alicyclic.

In formula (AL-5), R^{L5}, R^{L6} and R^{L7} are each independently a C₁-C₂₀ saturated hydrocarbyl group which may contain a heteroatom such as oxygen, sulfur, nitrogen or fluorine. The hydrocarbyl group may be straight, branched or cyclic. Any two of R^{L5}, R^{L6} and R^{L7} may bond together to form a C₃-C₂₀ ring with the carbon atom to which they are attached. The ring preferably contains 4 to 16 carbon atoms and is typically alicyclic.

Examples of repeat unit (a1) are shown below, but not limited thereto. Herein R^{A} and AL are as defined above.

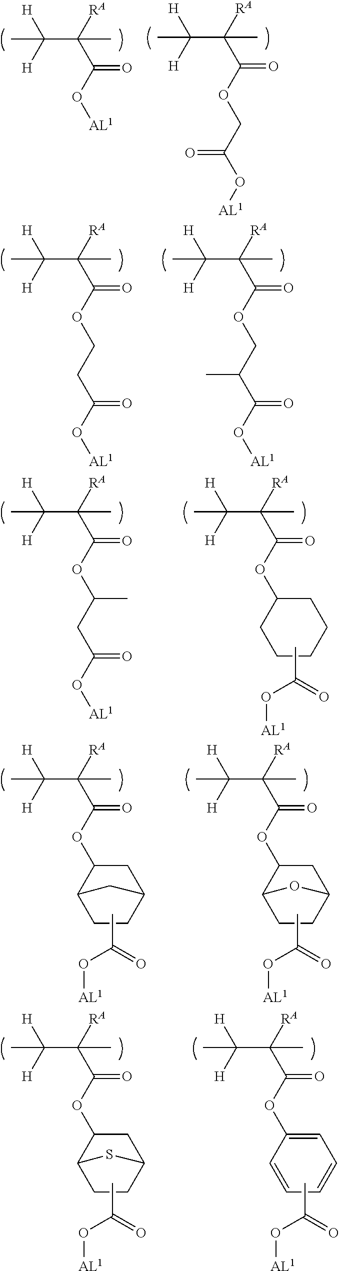

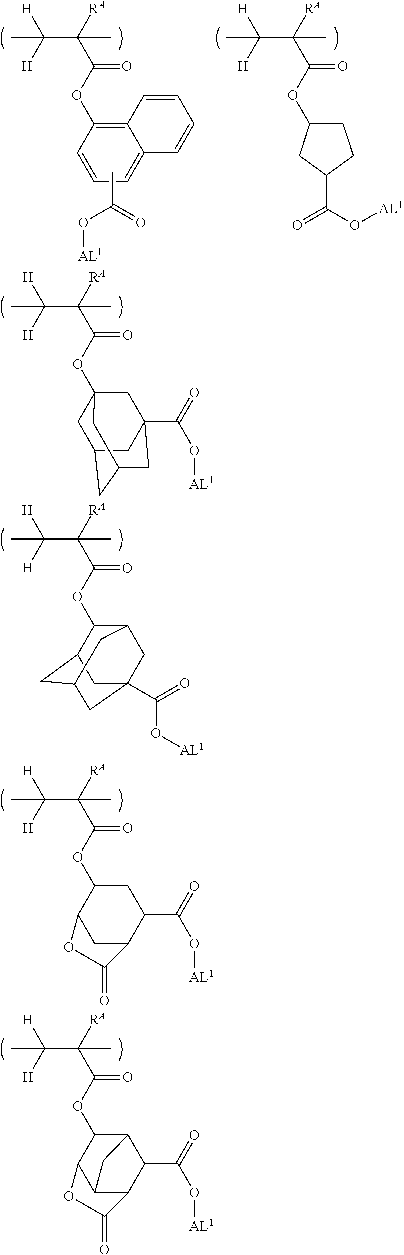

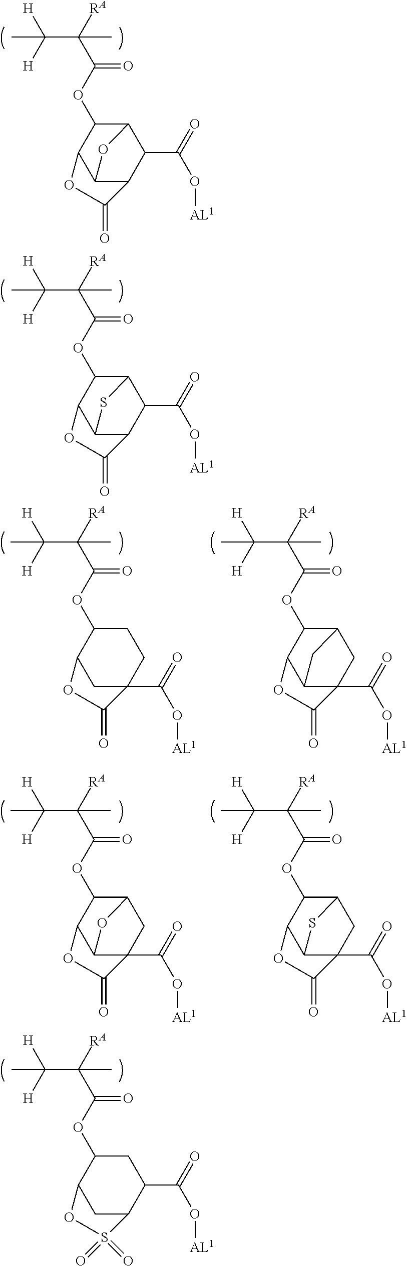

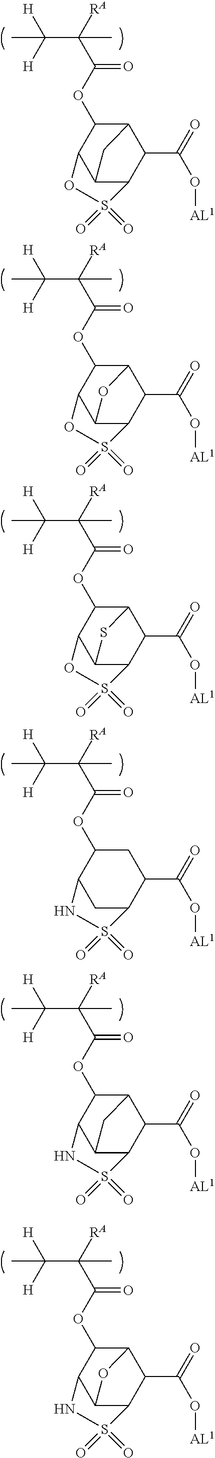

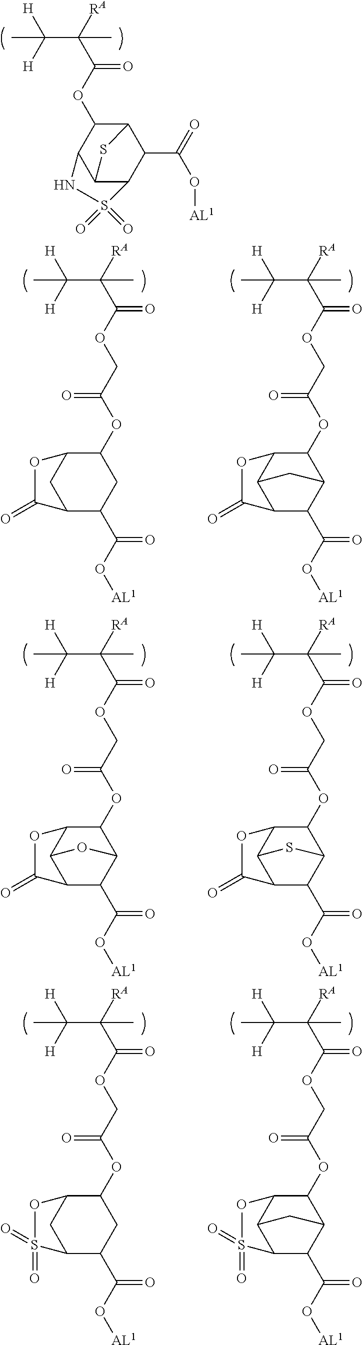

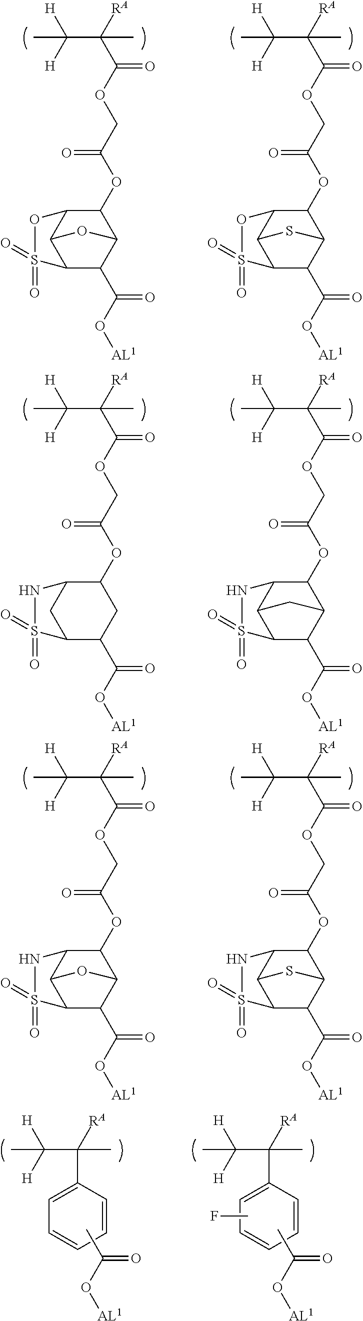

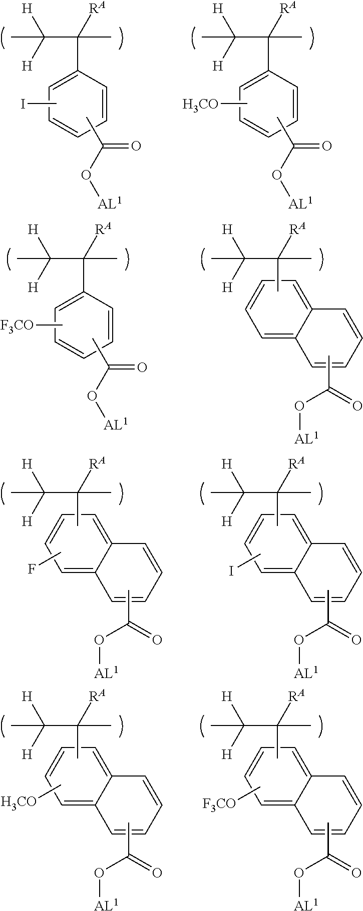

The base polymer (B) may further contain repeat units having the formula (a2), which are also referred to as repeat units (a2).

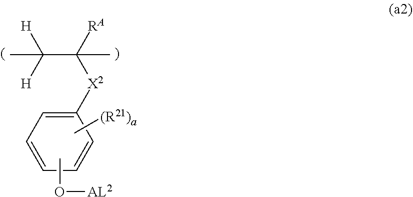

In formula (a2), R^{A} is hydrogen, fluorine, methyl or trifluoromethyl. X² is a single bond or *—C(═O)—O—. R²¹ is halogen, cyano, a C₁-C₂₀ hydrocarbyl group which may contain a heteroatom, a C₁-C₂ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. The subscript “a” is an integer of 0 to 4, preferably 0 or 1. AL² is an acid labile group, examples of which are as exemplified for the acid labile group AL¹.

Examples of the repeat unit (a2) are shown below, but not limited thereto. Herein R^{A} and AL are as defined above.

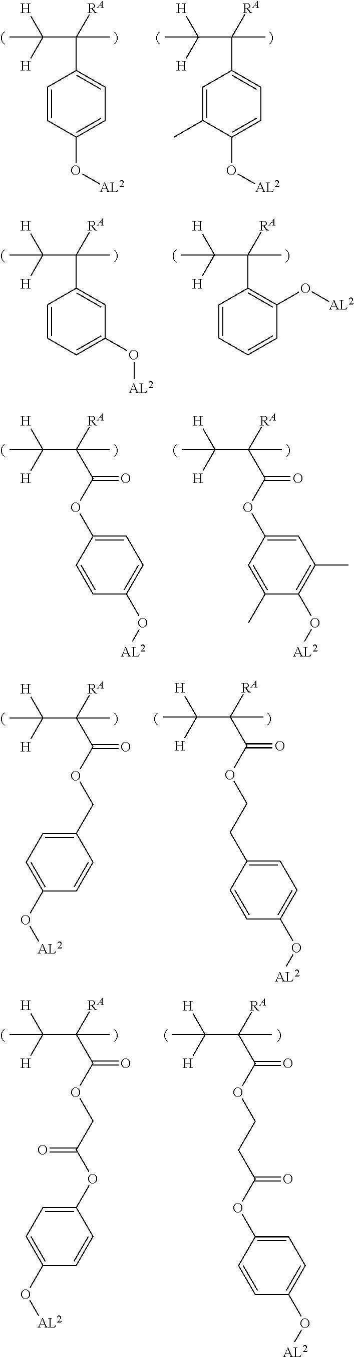

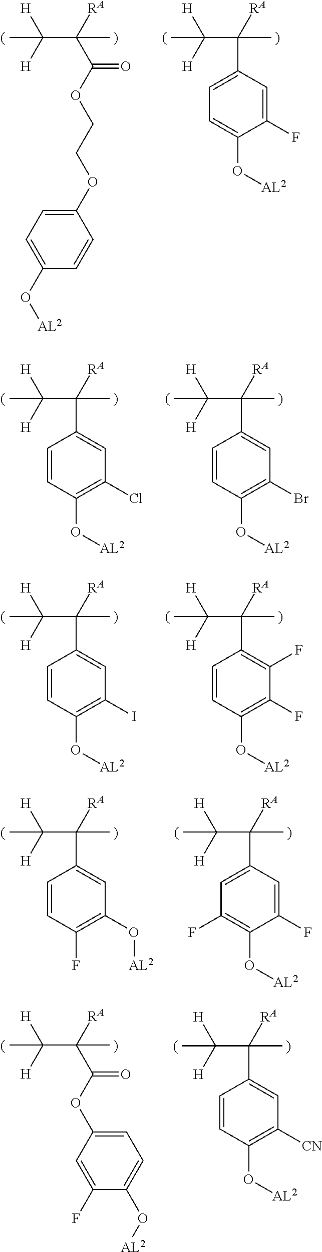

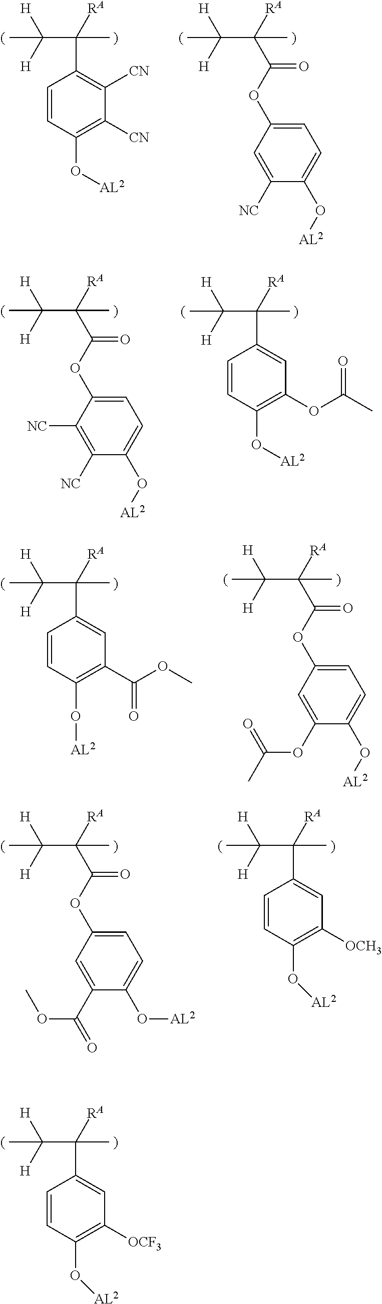

In a preferred embodiment, the base polymer further comprises repeat units having the formula (b1) or repeat units having the formula (b2), which are simply referred to as repeat units (b1) or (b2).

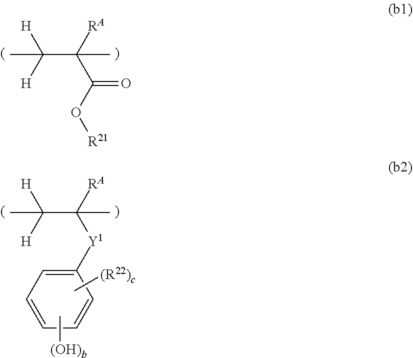

In formulae (b1) and (b2), R^{A} is each independently hydrogen, fluorine, methyl or trifluoromethyl. Y¹ is a single bond or *—C(═O)—O—. RP is hydrogen or a C₁-C₂₀ group containing at least one structure selected from among hydroxy other than phenolic hydroxy, cyano, carbonyl, carboxy, ether bond, ester bond, sulfonic ester bond, carbonate bond, lactone ring, sultone ring and carboxylic anhydride (—C(═O)—O—C(═O)—). R² is halogen, hydroxy, nitro, a C₁-C₂₀ hydrocarbyl group which may contain a heteroatom, C₁-C₂₀ hydrocarbyloxy group which may contain a heteroatom, C₂-C₂O hydrocarbylcarbonyl group which may contain a heteroatom, C₂-C₂₀ hydrocarbylcarbonyloxy group which may contain a heteroatom or C₂-C₂₀ hydrocarbyloxycarbonyl group which may contain a heteroatom. The subscript b is an integer of 1 to 4, c is an integer of 0 to 4, and b+c is from 1 to 5.

Examples of the repeat unit (b1) are shown below, but not limited thereto. Herein, R^{A} is as defined above.

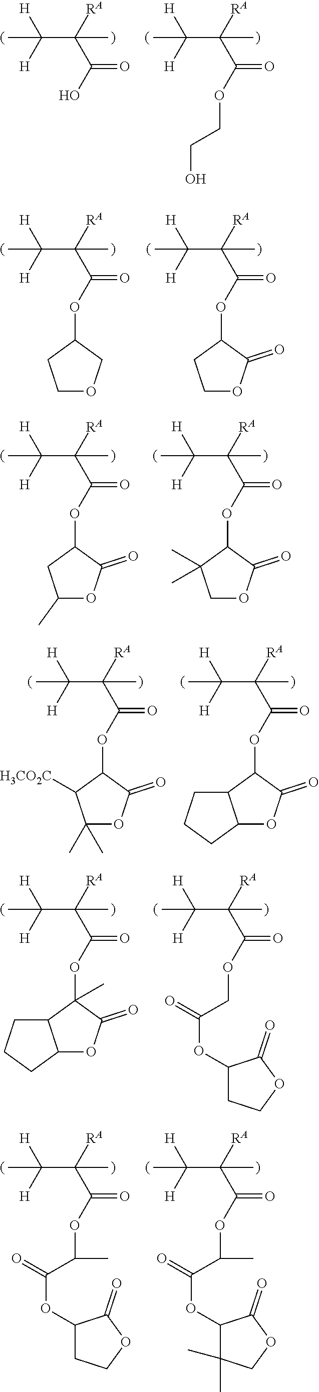

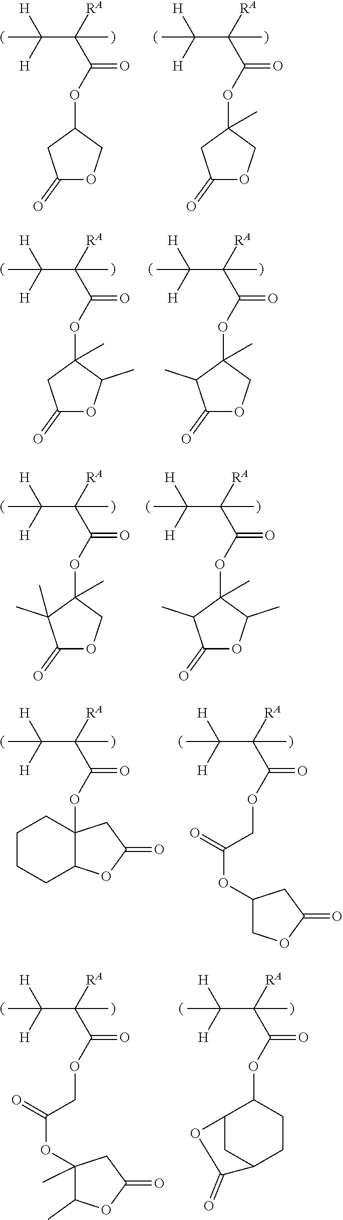

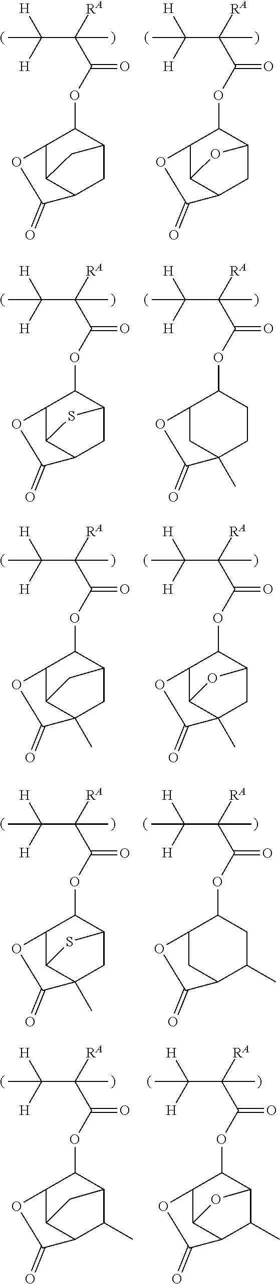

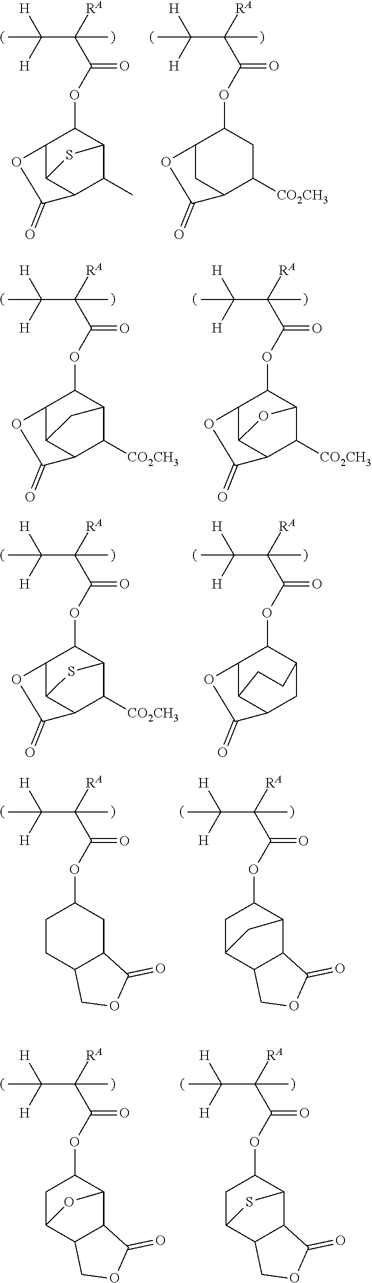

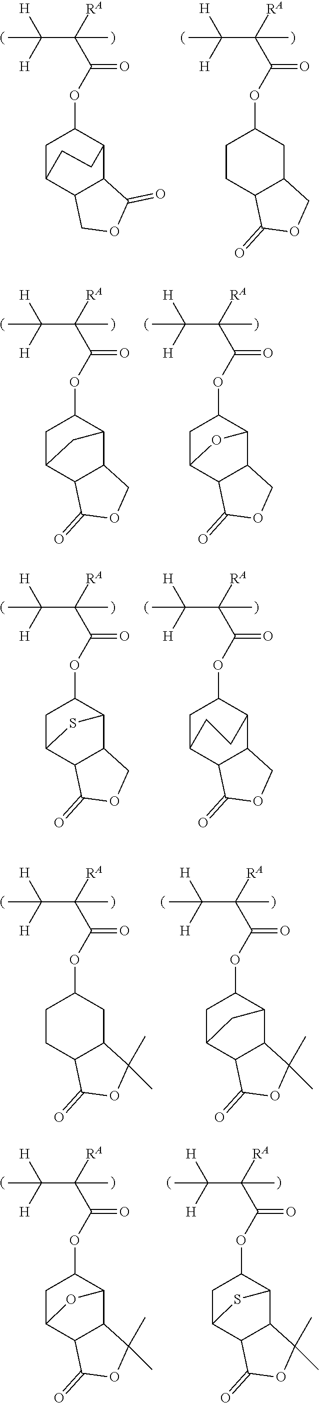

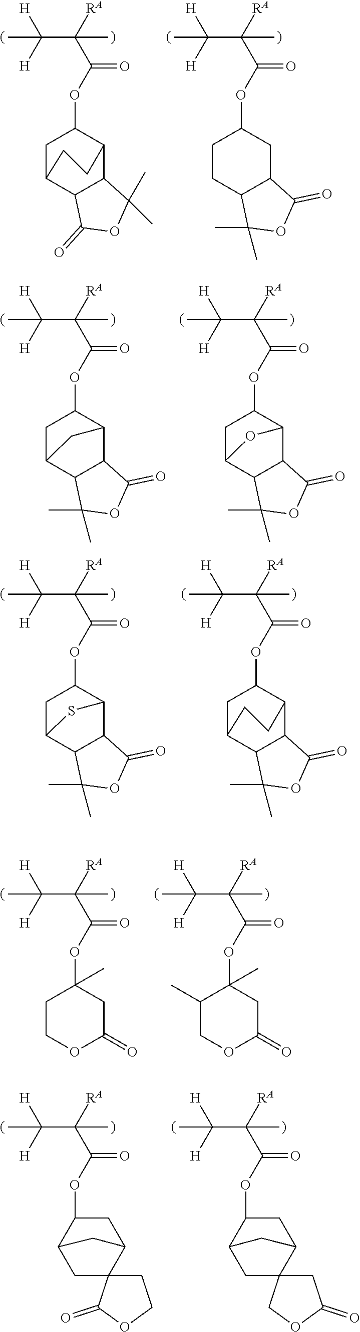

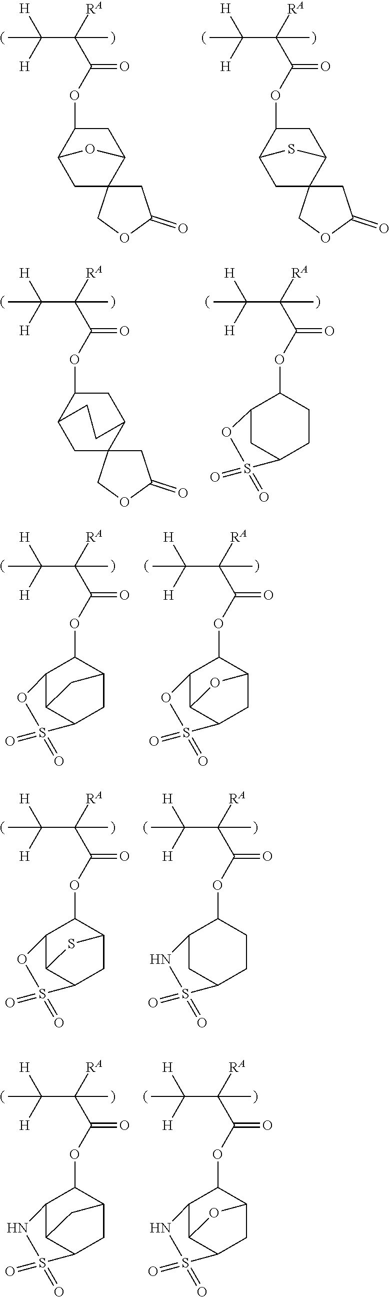

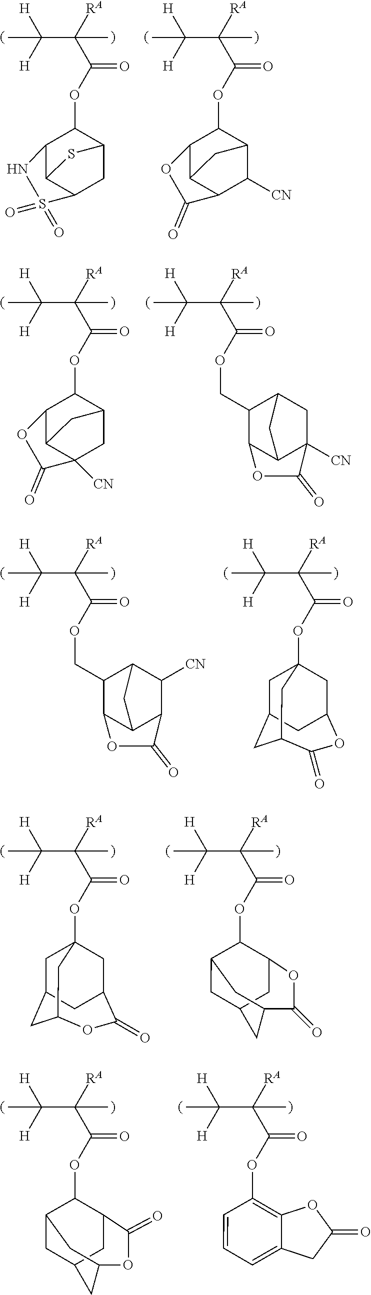

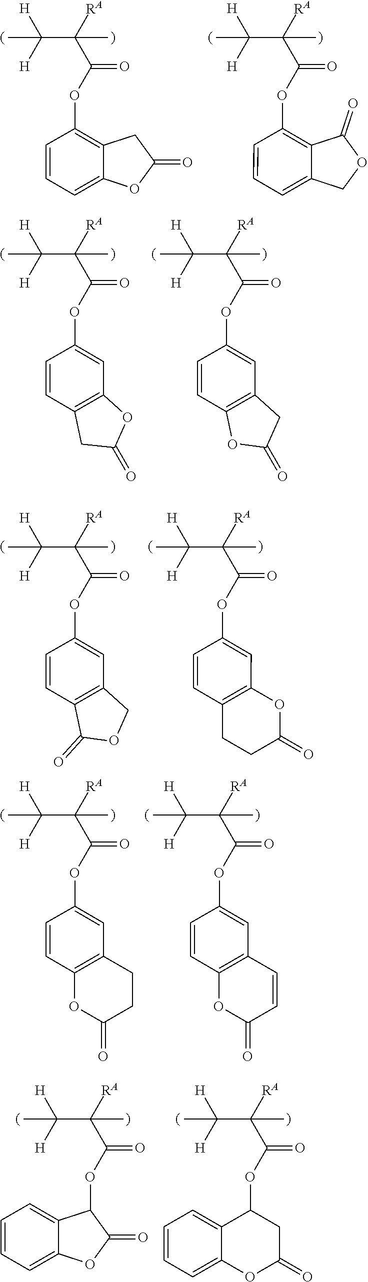

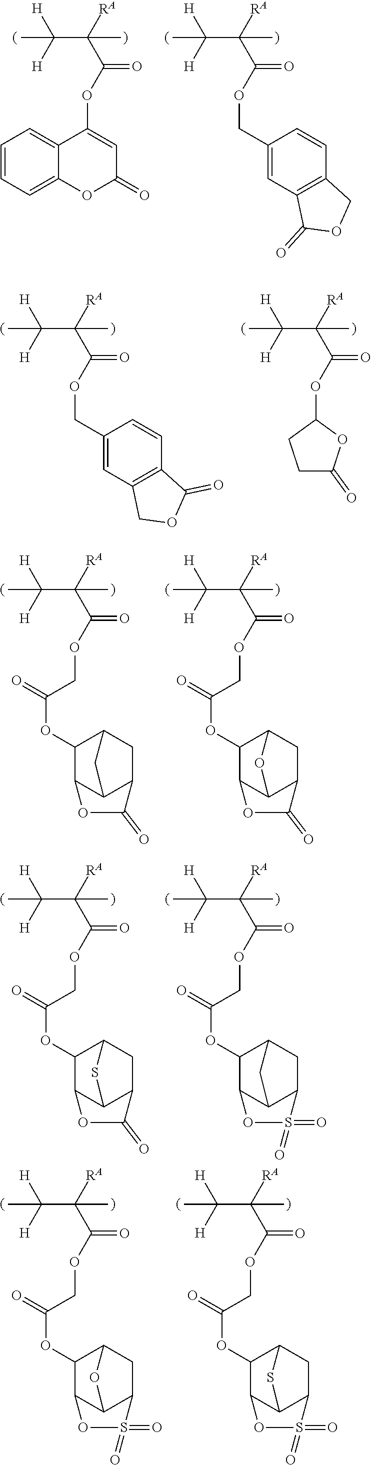

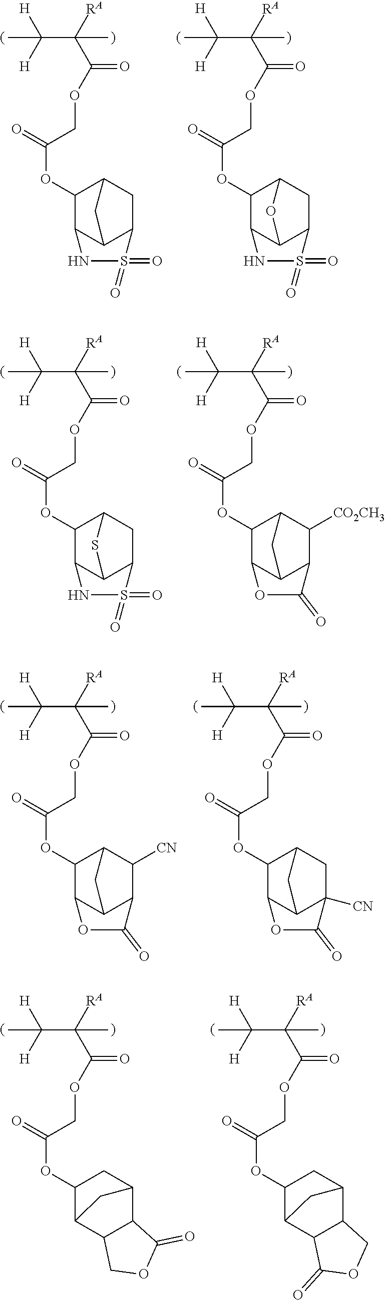

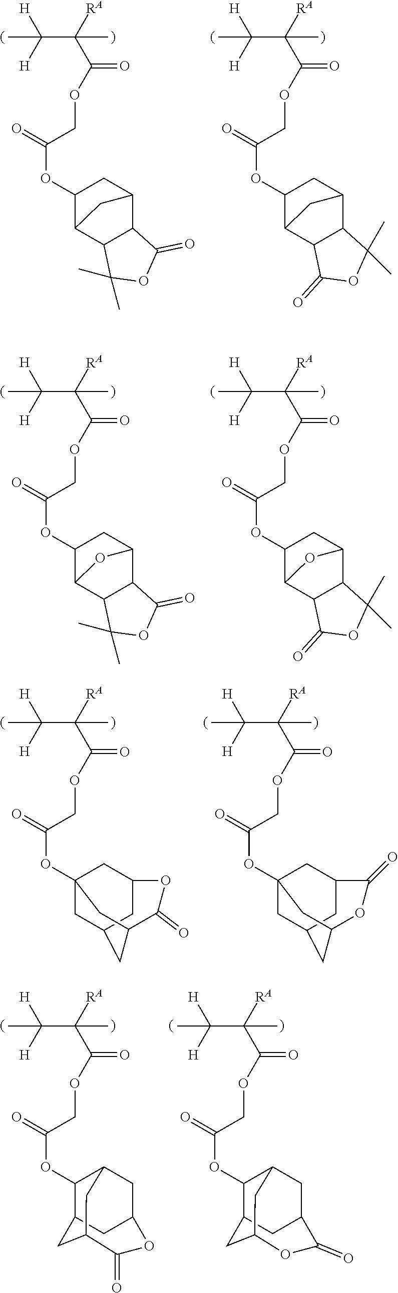

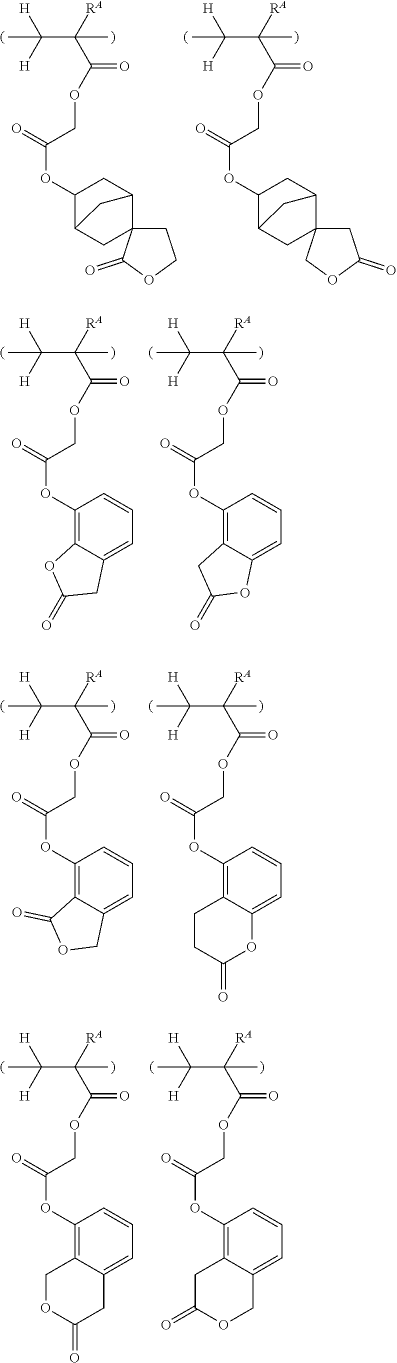

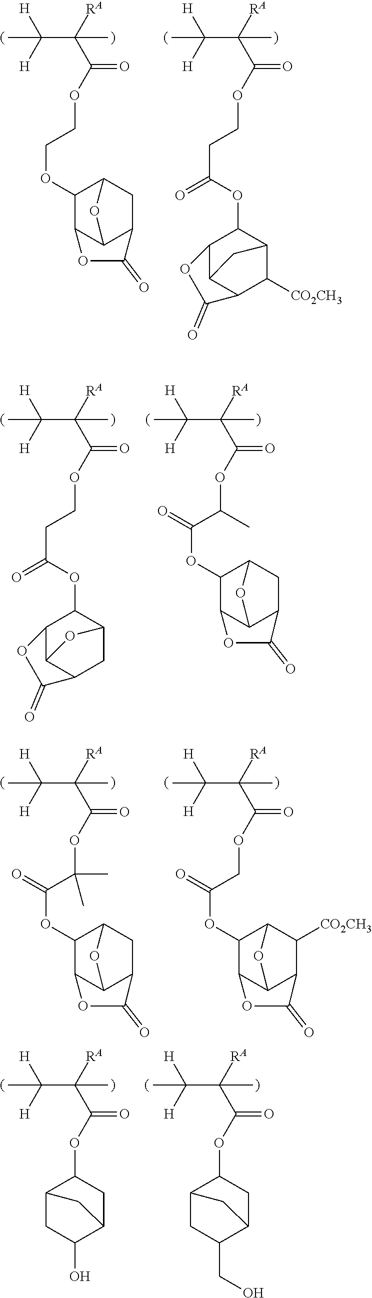

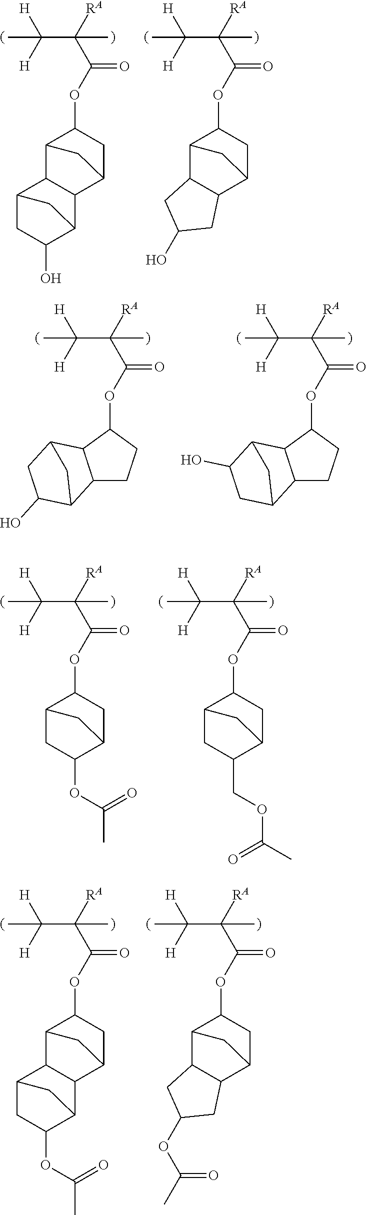

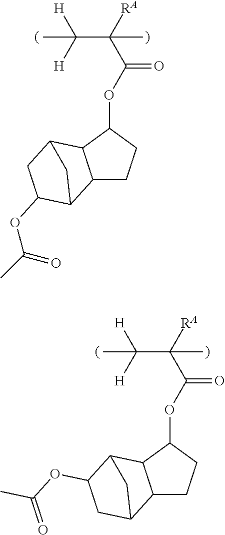

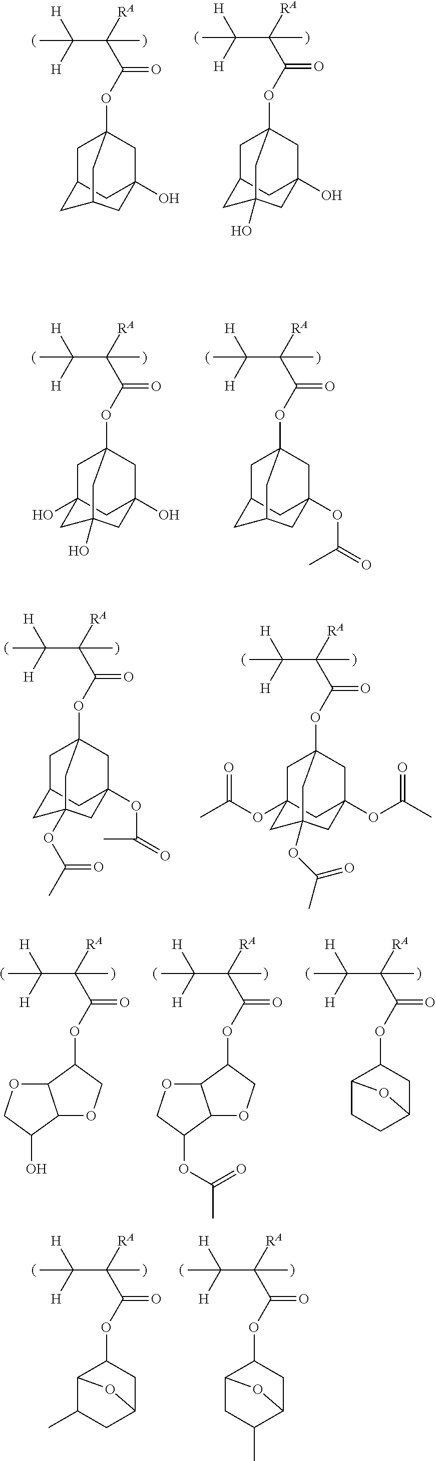

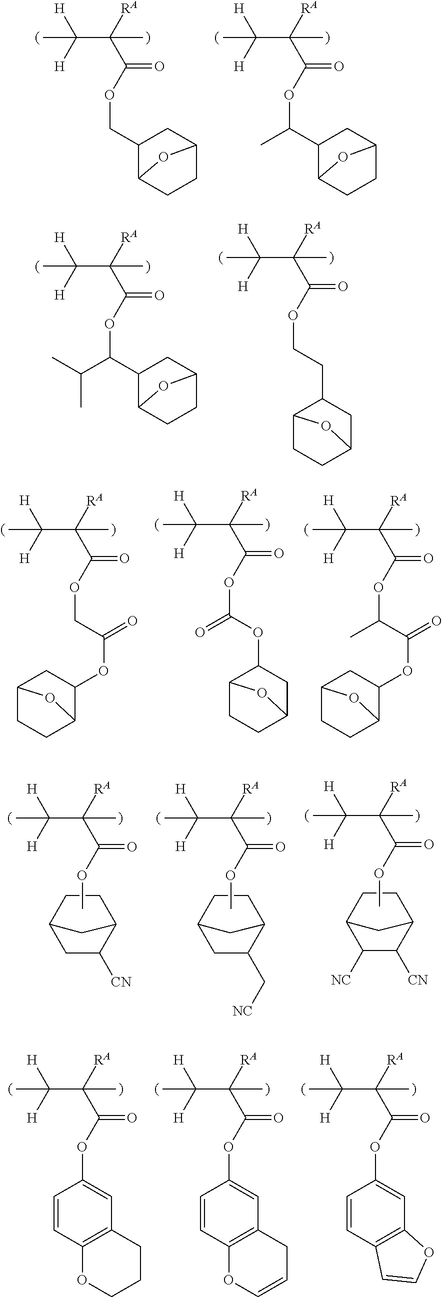

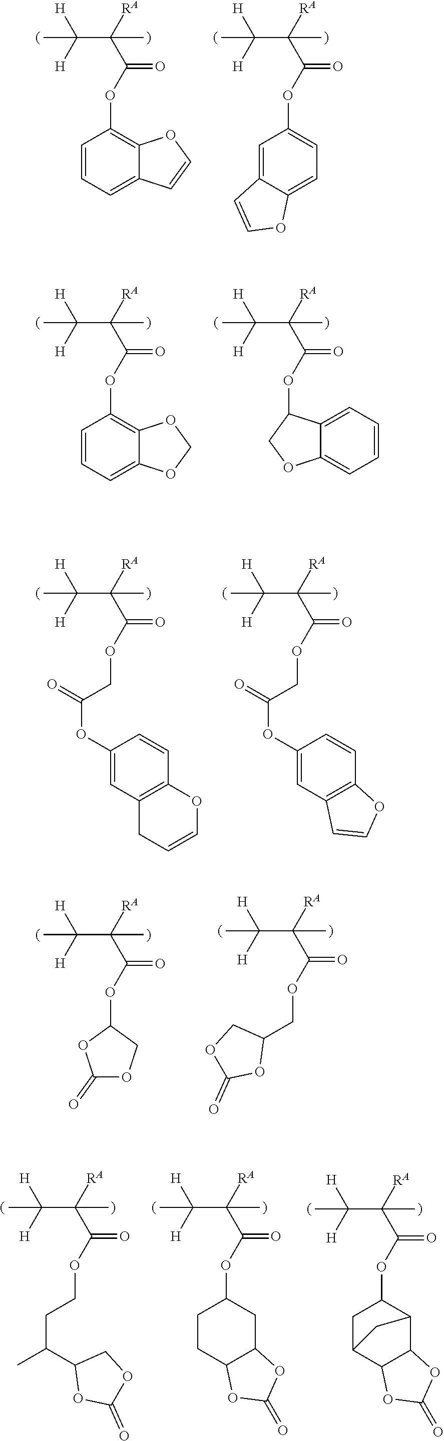

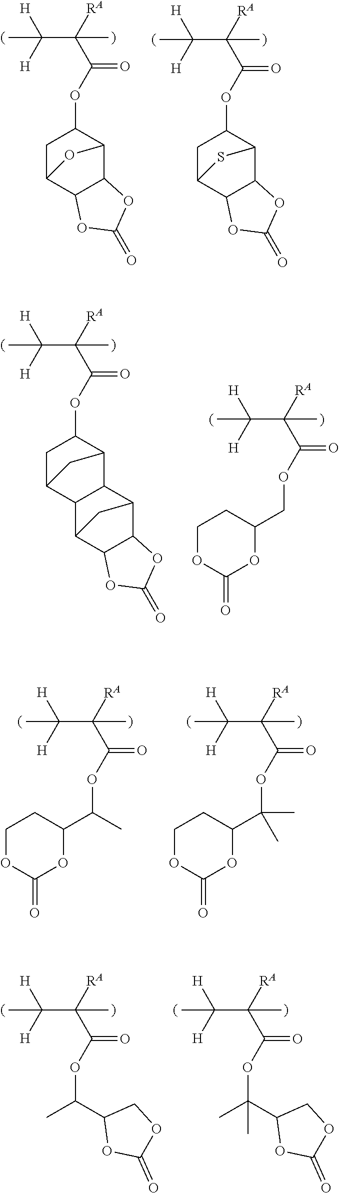

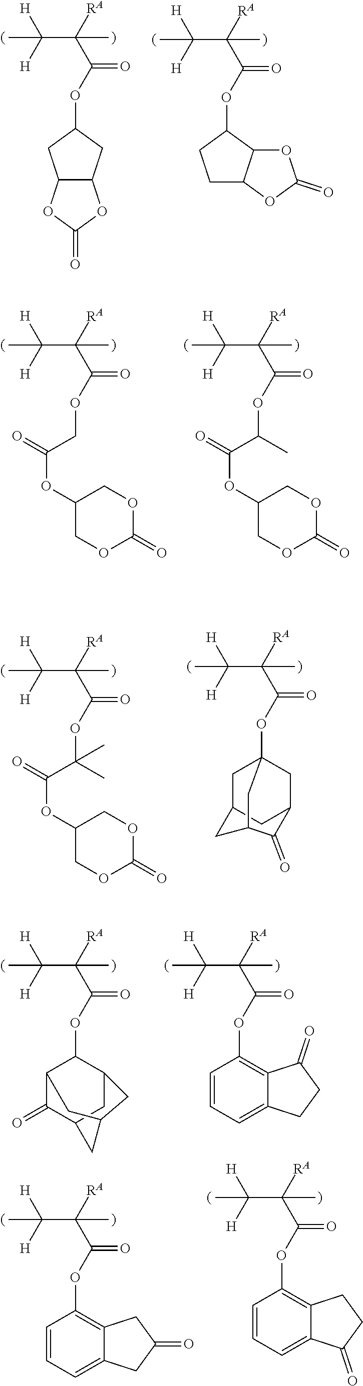

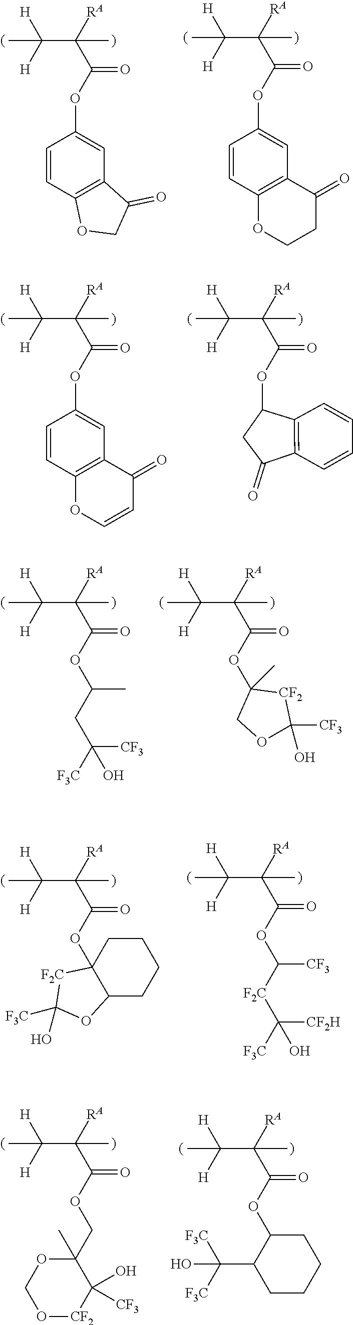

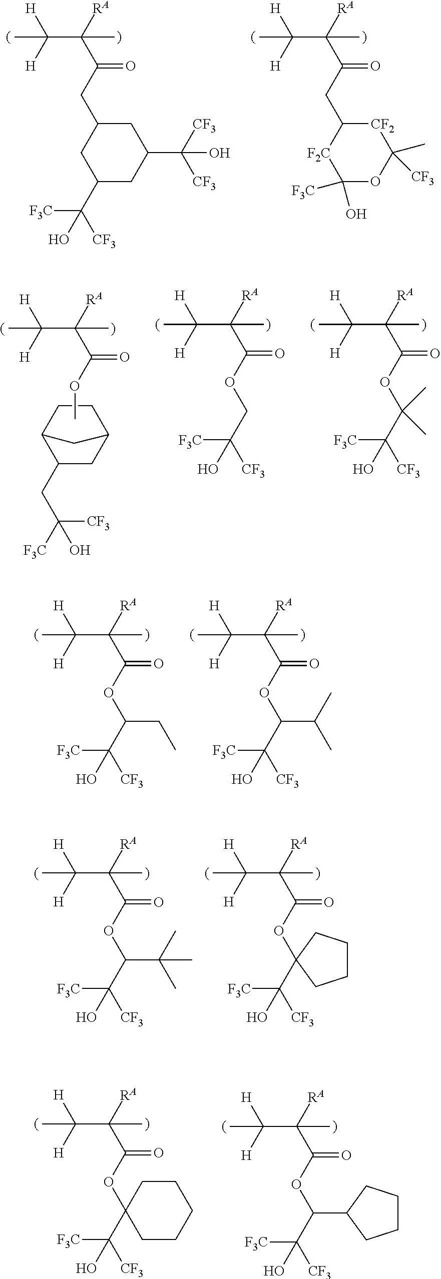

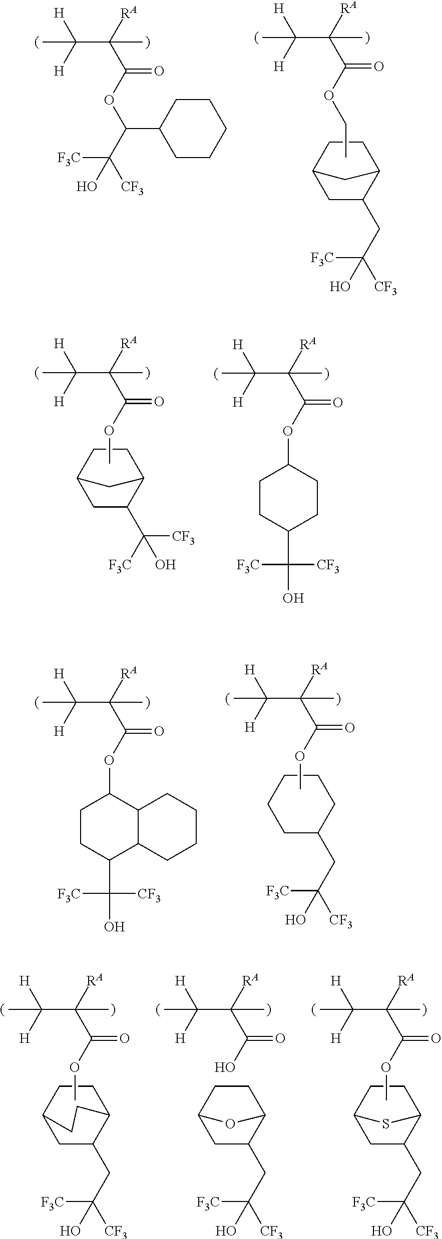

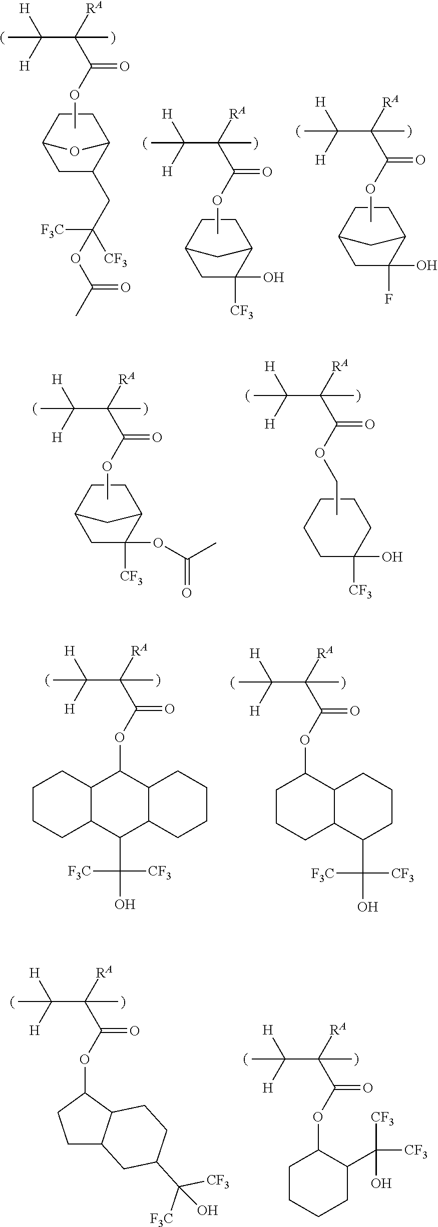

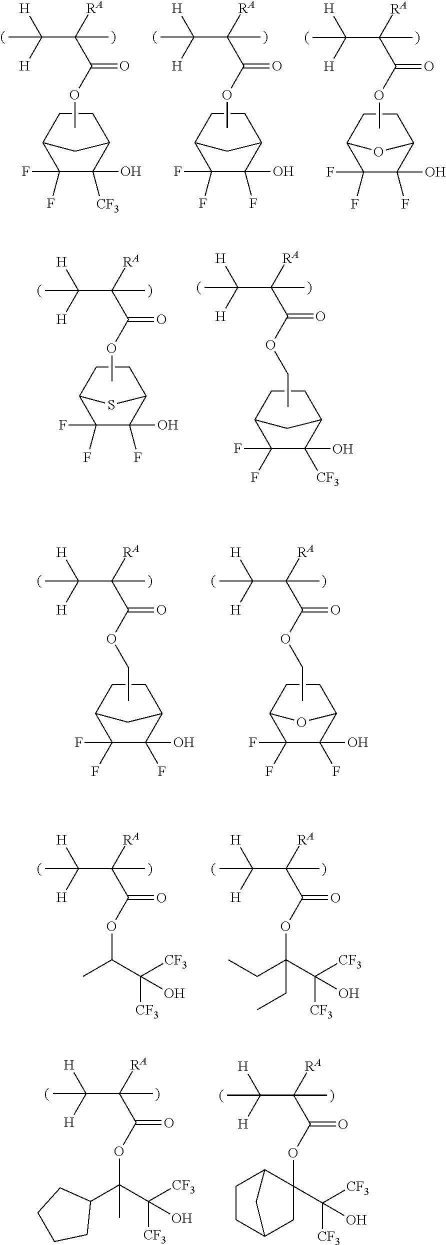

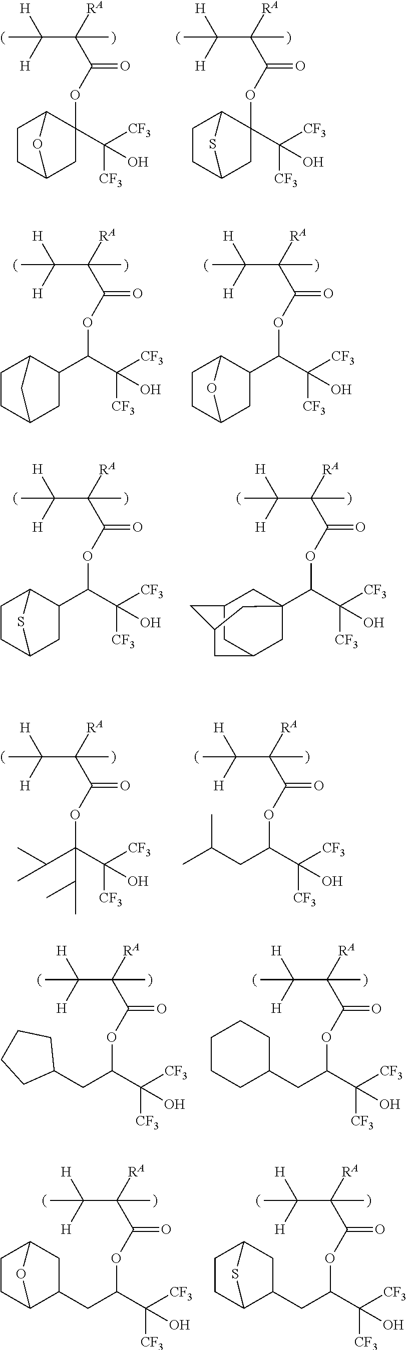

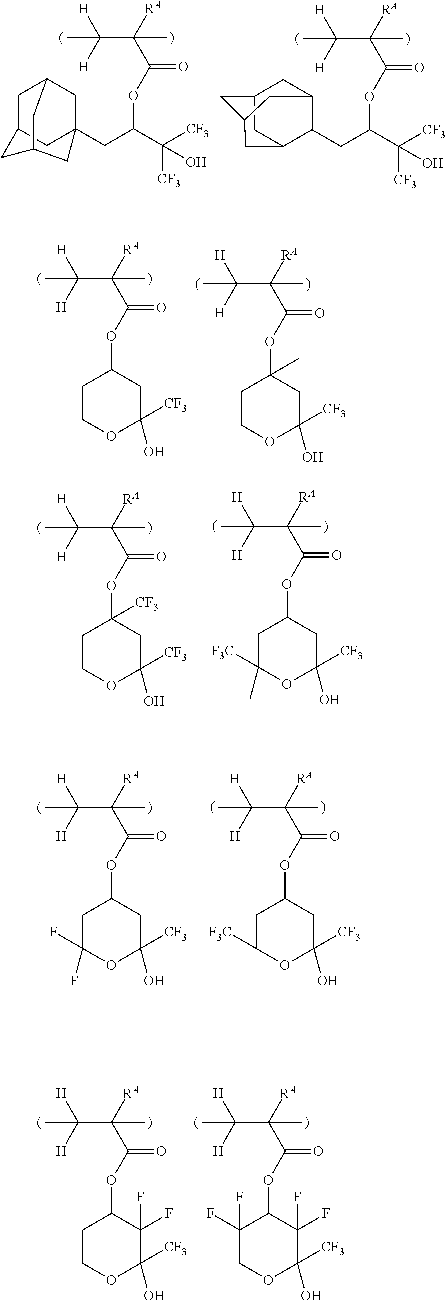

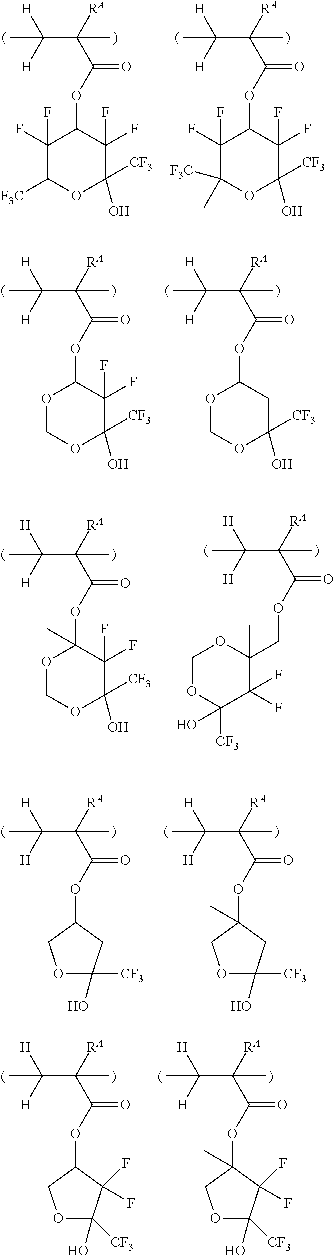

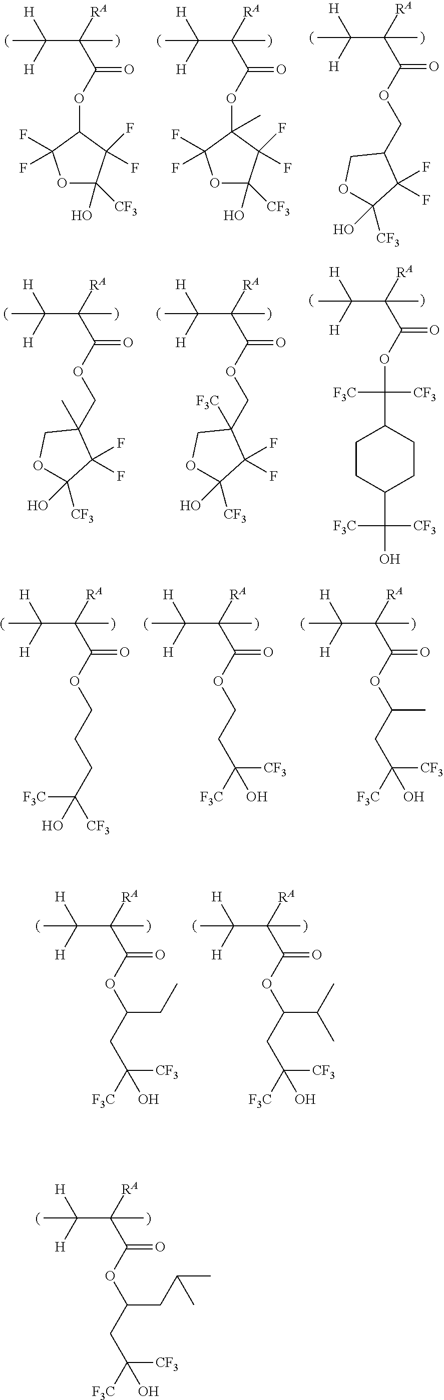

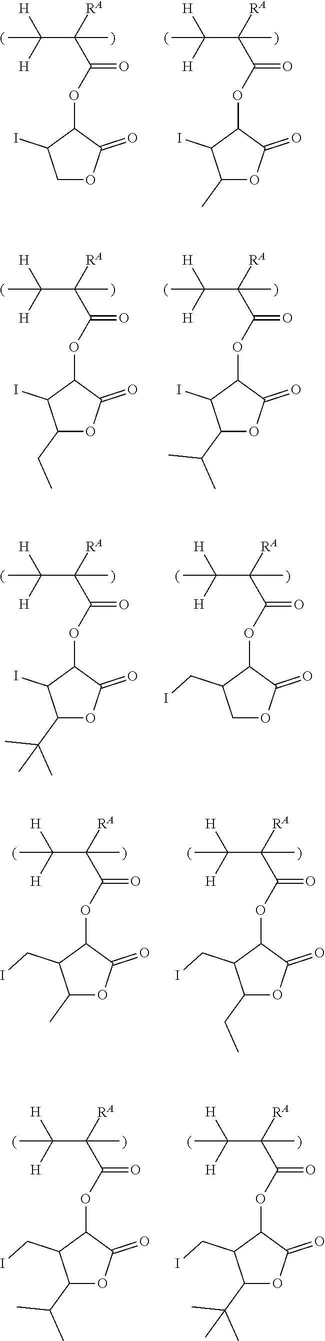

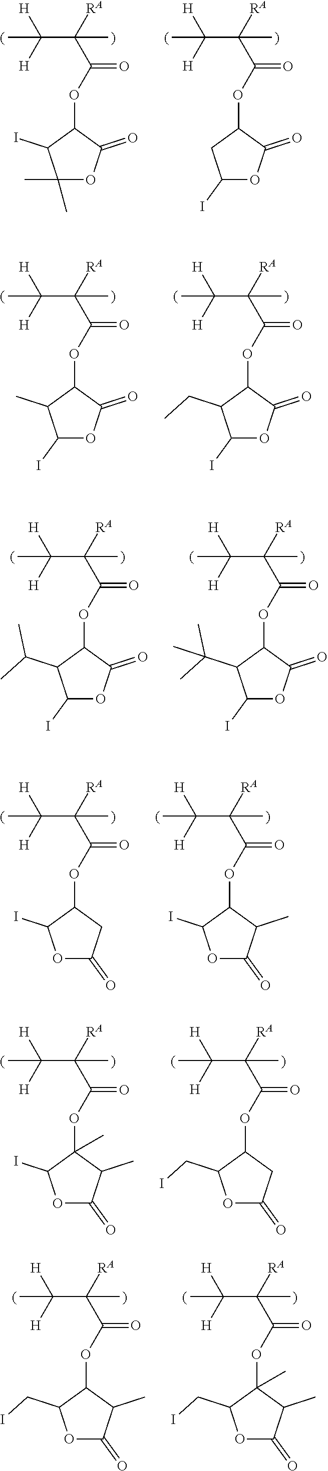

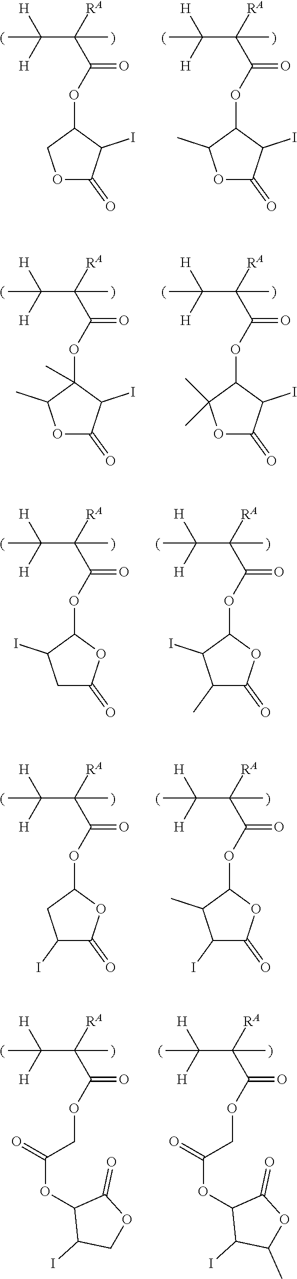

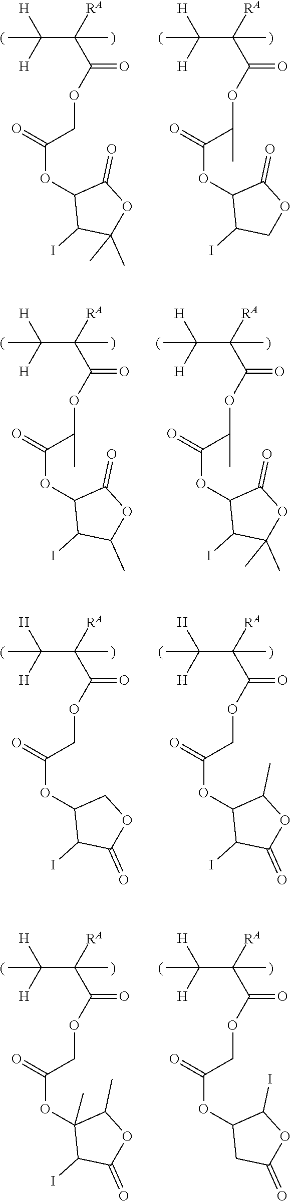

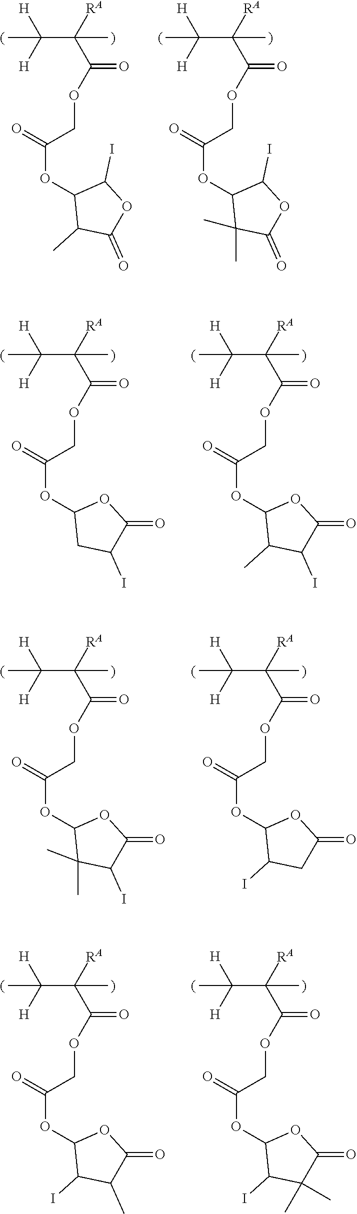

Examples of the repeat unit (b2) are shown below, but not limited thereto. Herein, R^{A} is as defined above.

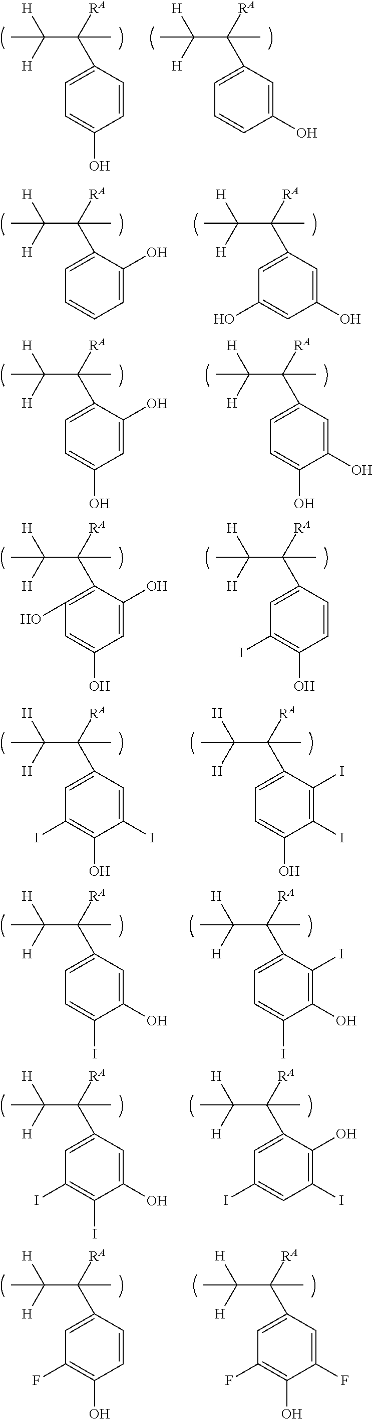

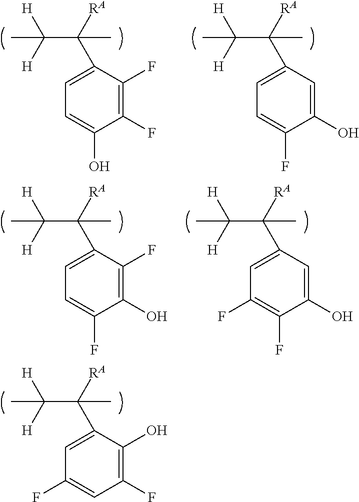

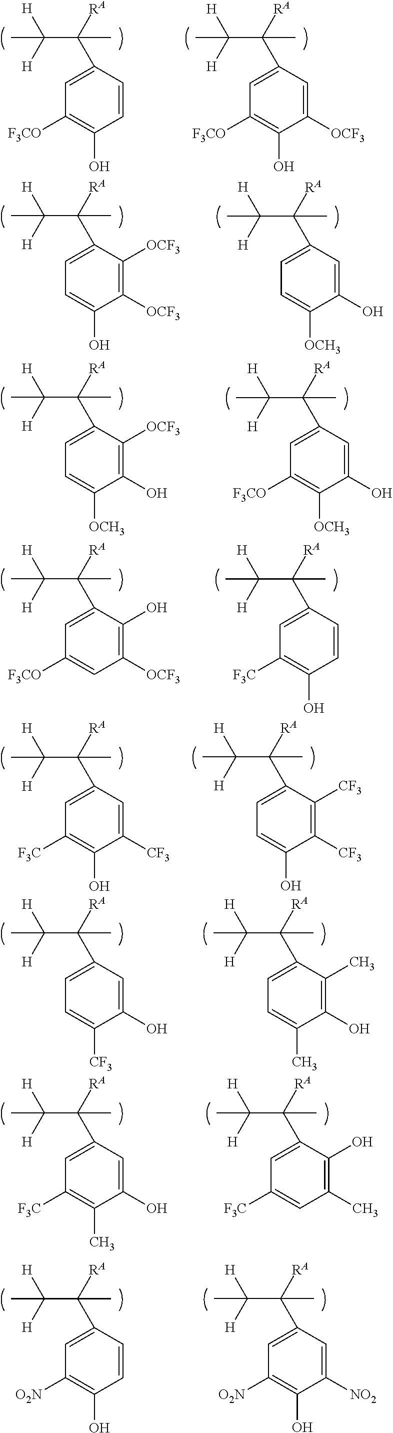

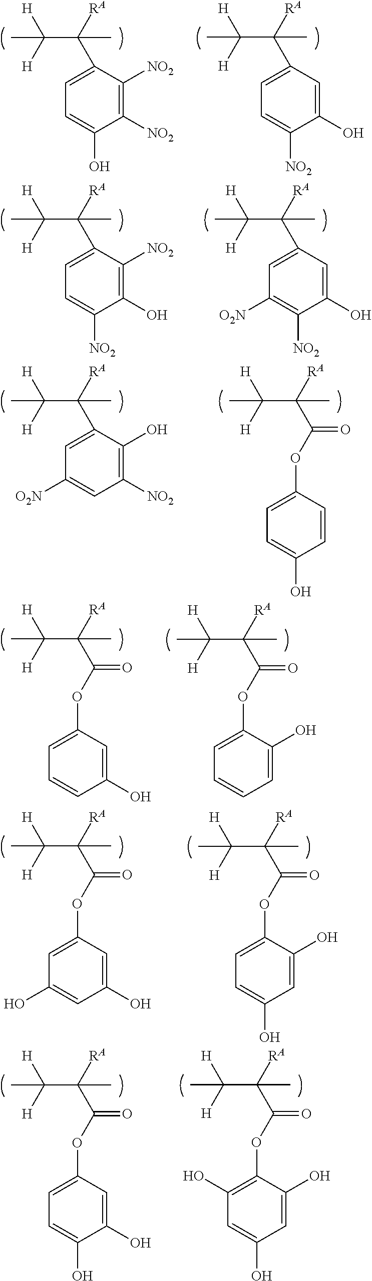

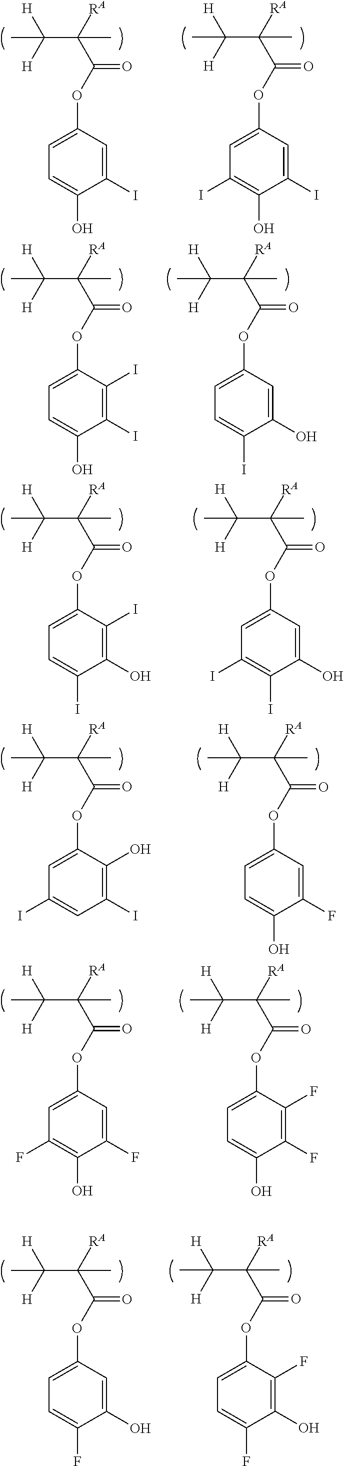

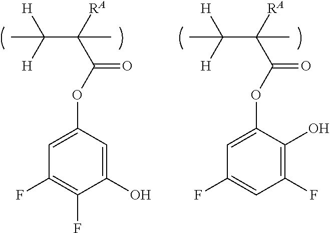

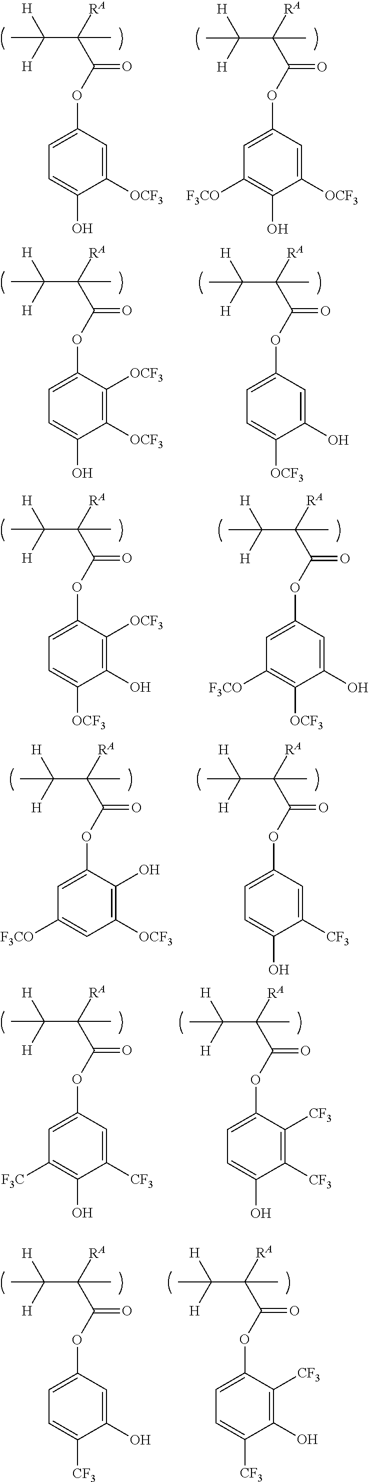

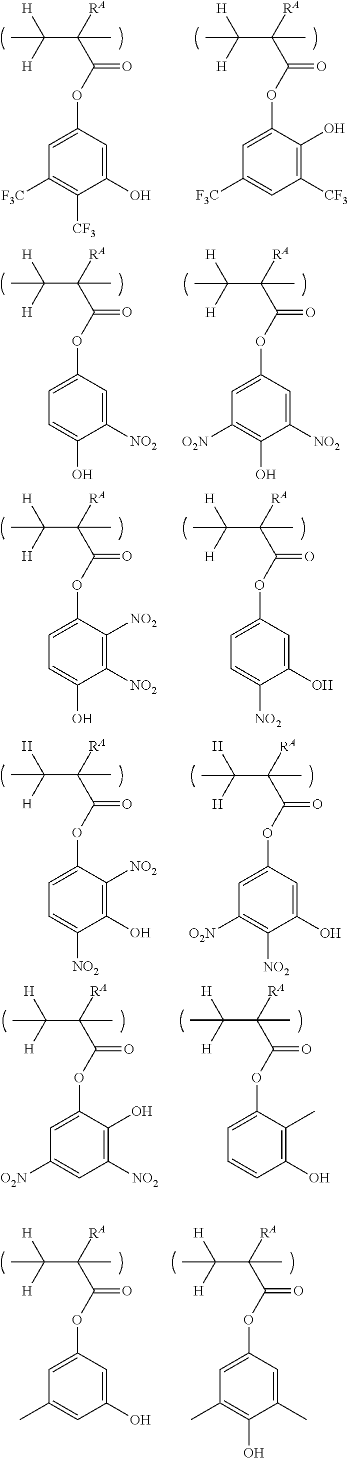

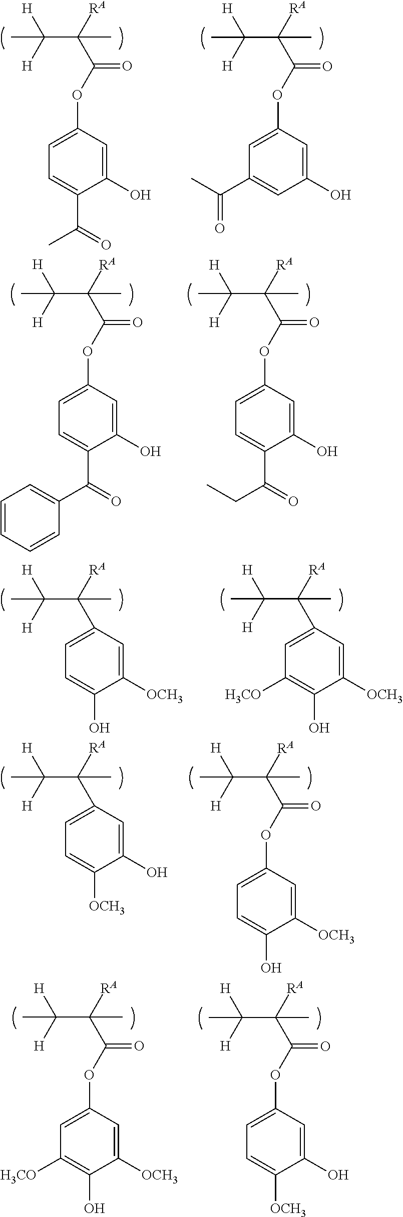

Of the repeat units (b1) and (b2), those units having a lactone ring as the polar group are preferred in the ArF lithography and those units having a phenolic site are preferred in the KrF, EB and EUV lithography.

The base polymer may further comprise repeat units of at least one type selected from repeat units having the formulae (c1) to (c4), which are simply referred to as repeat units (c1) to (c4).

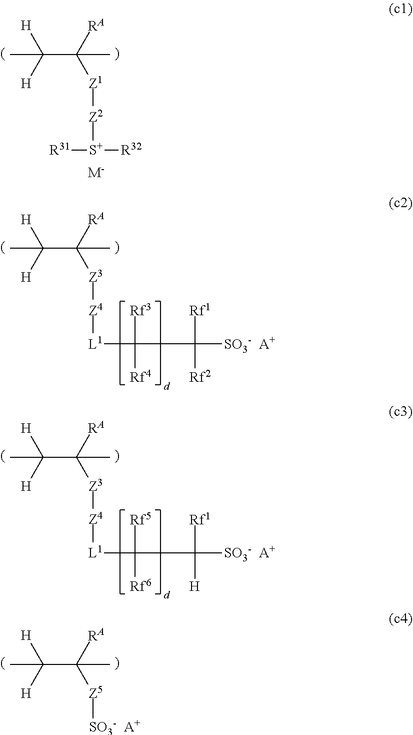

In formulae (c1) to (c4). R^{A} is each independently hydrogen, fluorine, methyl or trifluoromethyl. Z¹ is a single bond or phenylene group. Z² is *—C(═O)—O—Z²¹—, *—C(═O)—N(H)—Z²¹— or *—O—Z²¹—. Z²¹ is a C₁-C₆ aliphatic hydrocarbylene group, a phenylene group or a divalent group obtained by combining the foregoing, which may contain a carbonyl moiety, ester bond, ether bond or hydroxy moiety. Z³ is a single bond, phenylene group, naphthylene group or *—C(═O)—O—Z³¹—. Z³¹ is a C₁-C₁₀ aliphatic hydrocarbylene group which may contain a hydroxy moiety, ether bond, ester bond or lactone ring, or phenylene or naphthylene group. Z⁴ is a single bond or *—Z⁴¹—C(═O)—O—. Z⁴¹ is a C₁-C₂₀ hydrocarbylene group which may contain a heteroatom. Z⁵ is a single bond, methylene, ethylene, phenylene, fluorinated phenylene, trifluoromethyl-substituted phenylene group, *—C(═O)—O—Z⁵¹—, *—C(═O)—N(H)—Z⁵¹—, or *—O—Z⁵¹—. Z⁵¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety.

The aliphatic hydrocarbylene group represented by Z²¹, Z³¹ and Z³² may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-diinethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl and cyclohexanediyl, and combinations thereof.

The hydrocarbylene group Z⁴¹ may be saturated or unsaturated and straight, branched or cyclic. Examples thereof are shown below, but not limited thereto.

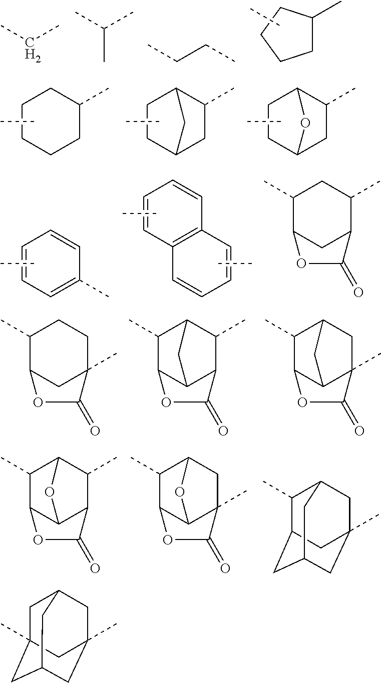

In formula (c1), R³¹ and R³² are each independently a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cyclopropyhnethyl, 4-methylcyclohexyl, cyclohexyhnethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl, naphthyl and thienyl; C₇-C₂₀ aralkyl groups such as benzyl, 1-phenylethyl, and 2-phenylethyl, and combinations thereof. Of these, aryl groups are preferred. In the hydrocarbyl group, some hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety.

R³¹ and R³² may bond together to form a ring with the sulfur atom to which they are attached. Examples of the ring are as exemplified above for the ring that R^{ct1} and R^{ct2} in formula (cation-1) form with the sulfur atom to which they are attached.

Examples of the cation in repeat unit (c1) are given below, but not limited thereto. Herein R^{A} is as defined above.

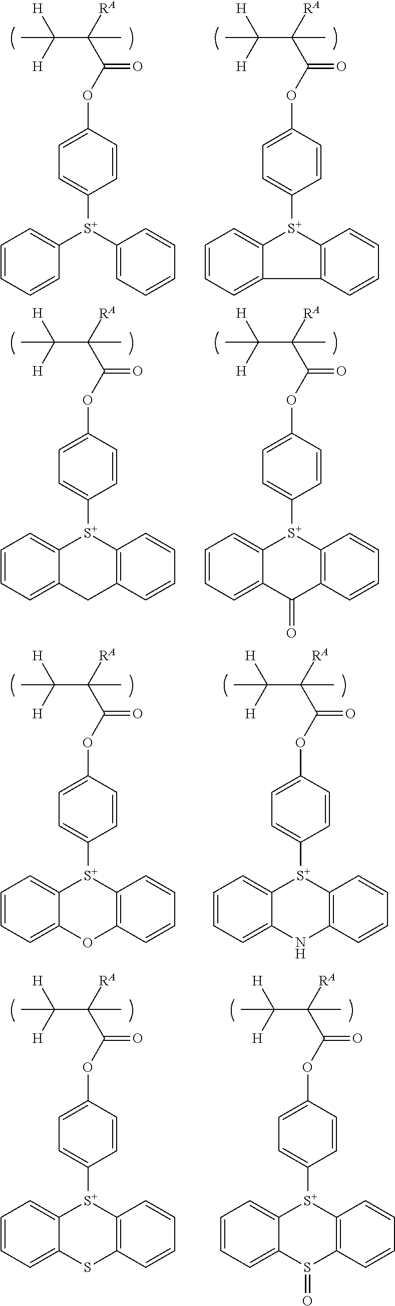

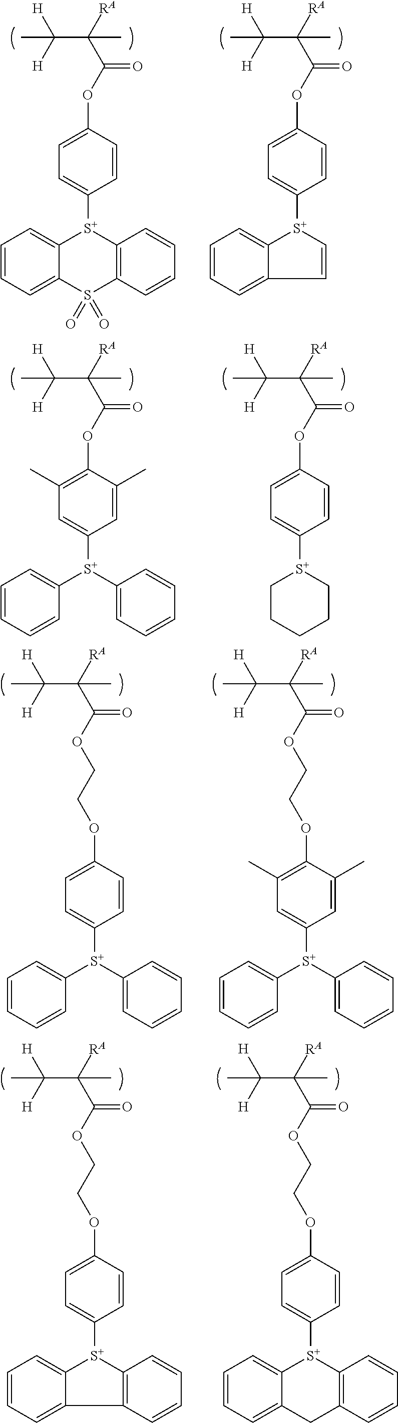

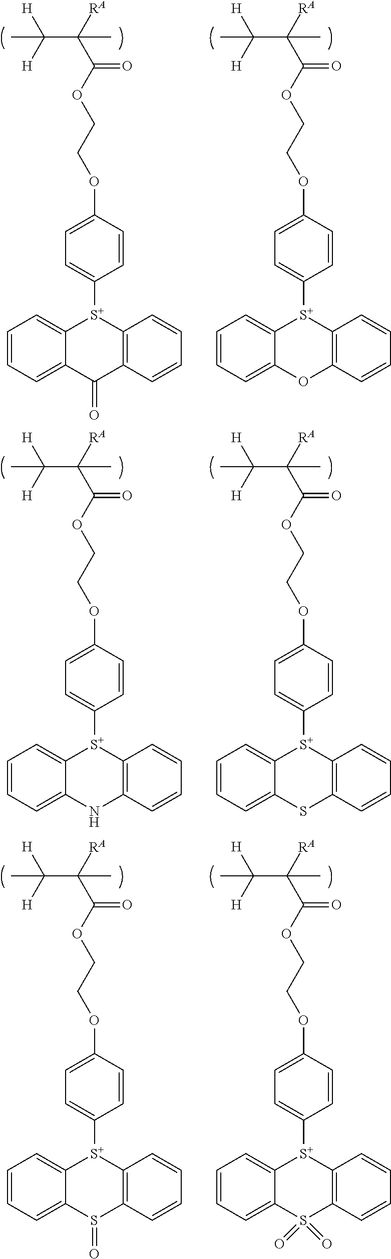

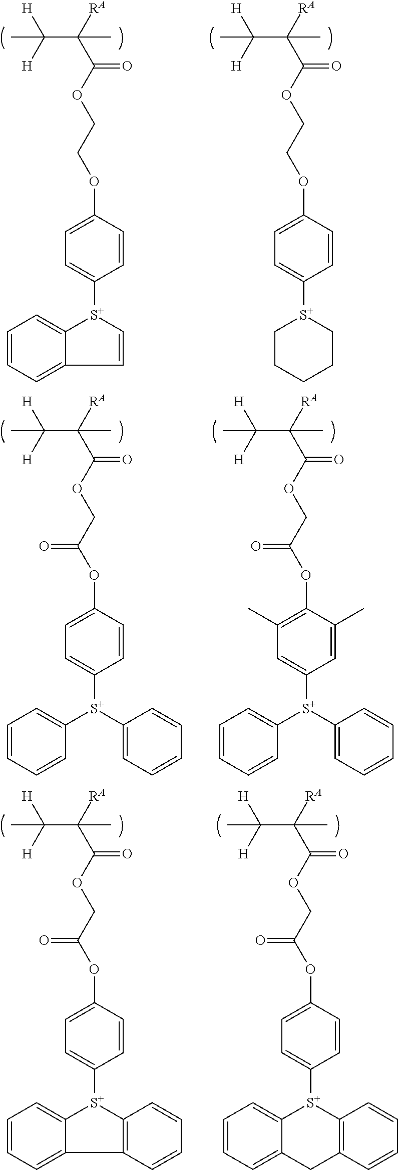

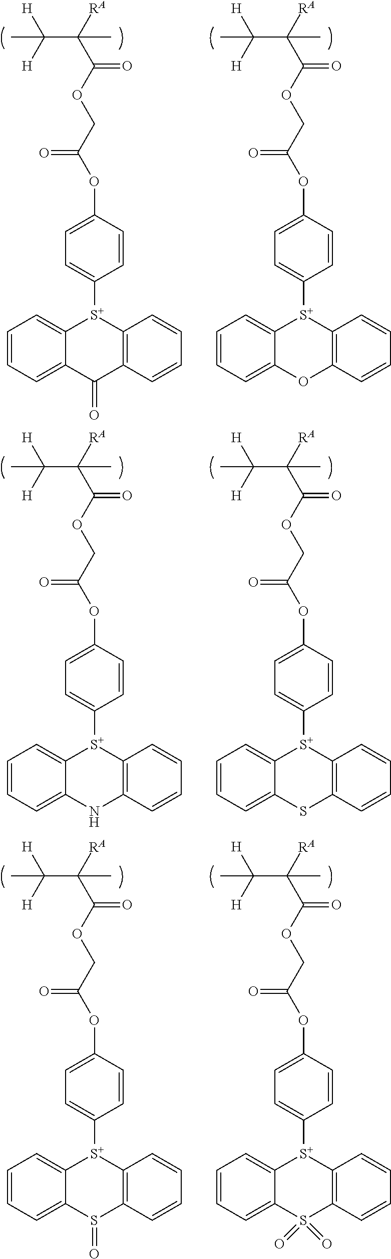

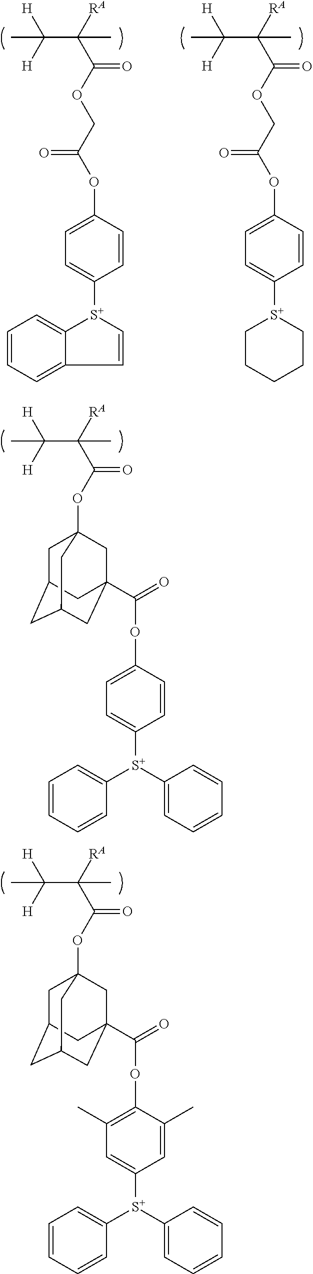

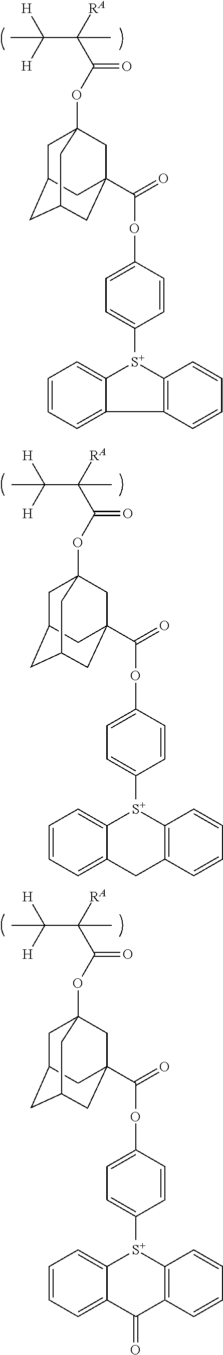

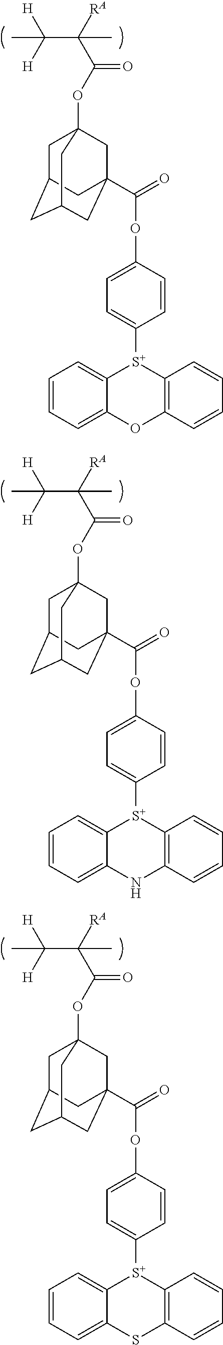

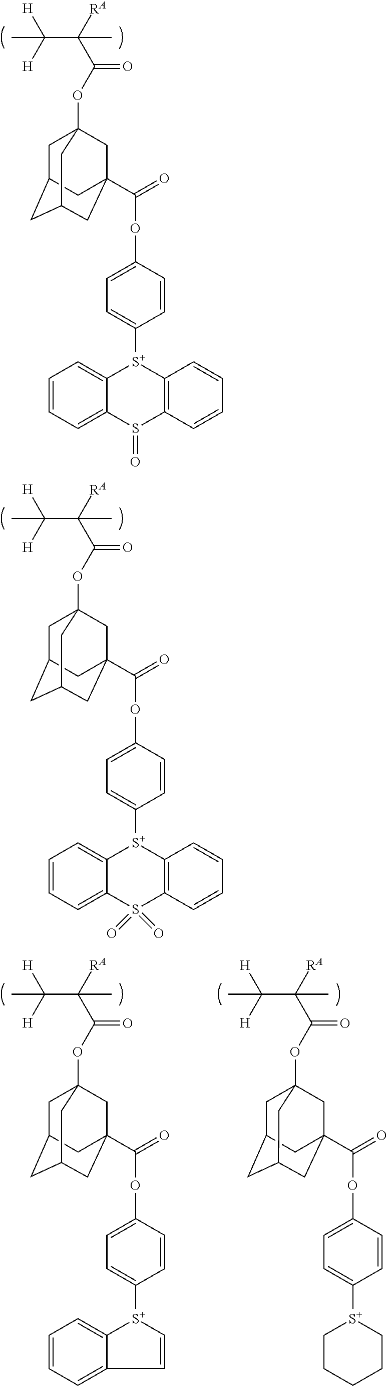

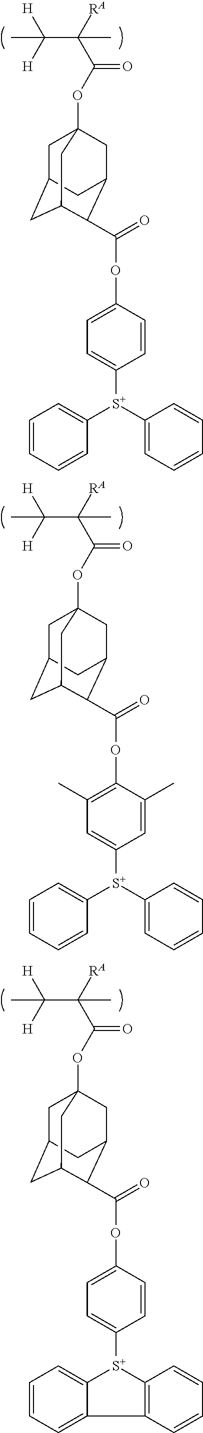

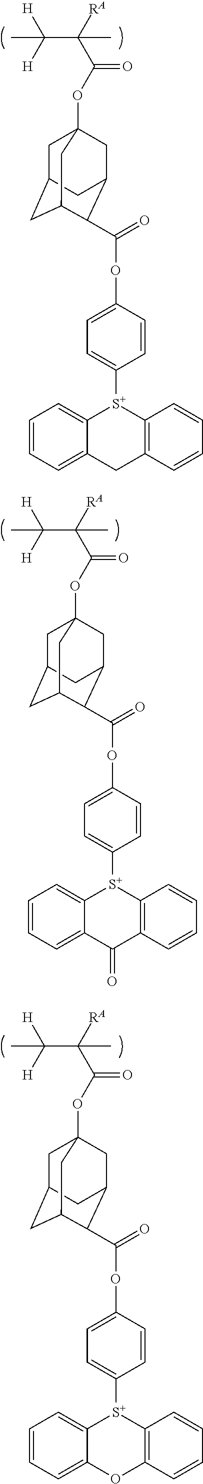

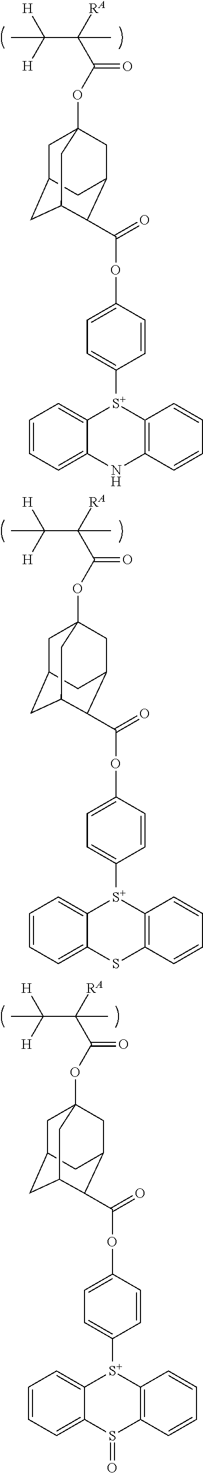

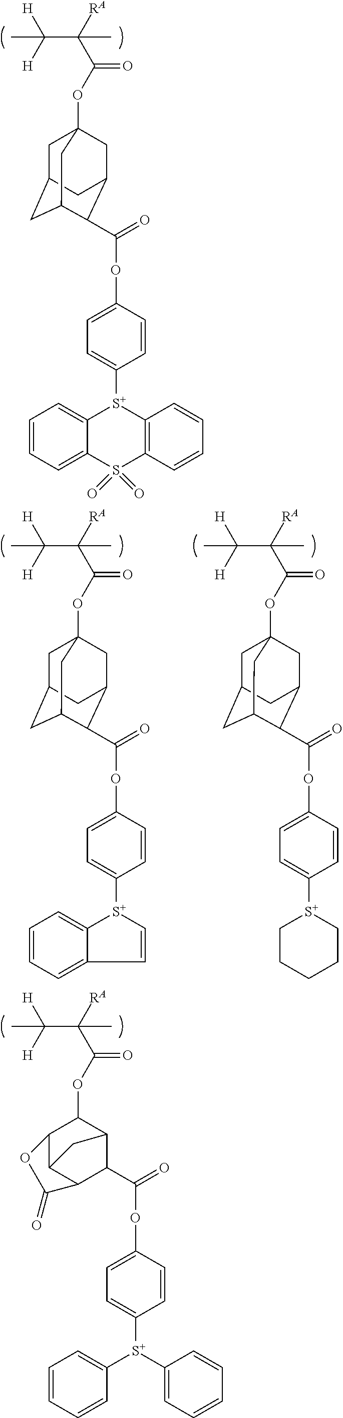

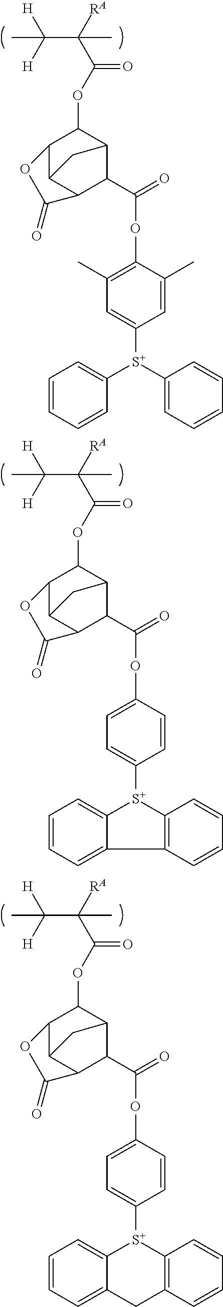

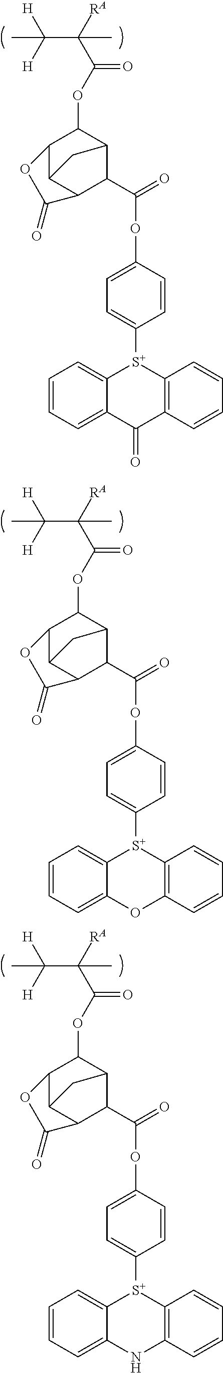

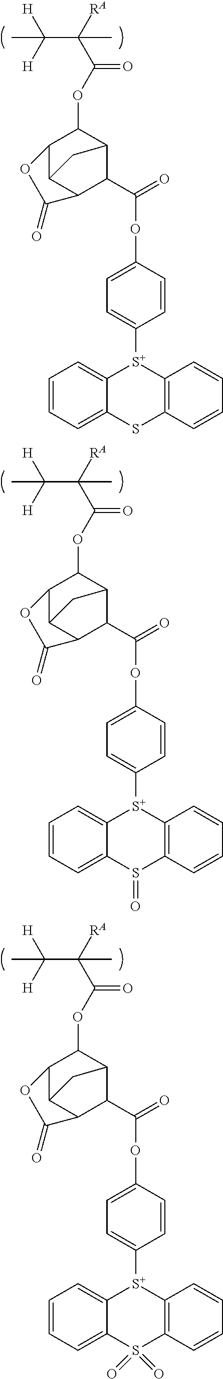

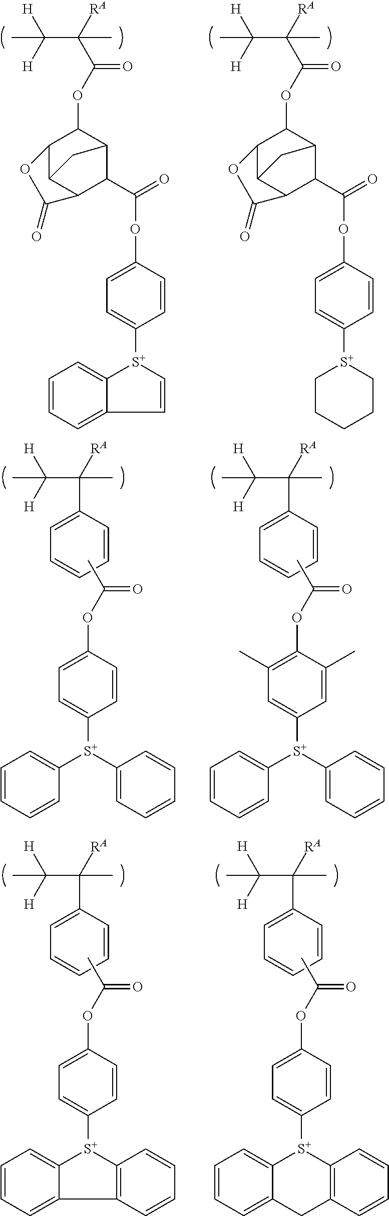

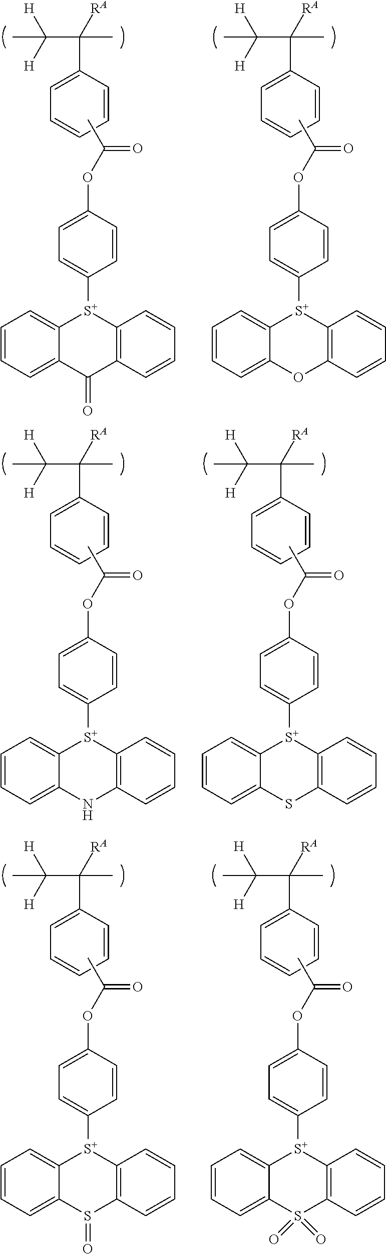

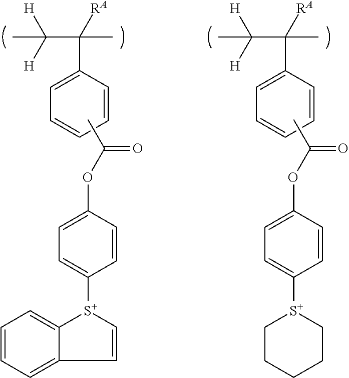

In formula (c1), M⁻ is a non-nucleophilic counter ion. Sulfonate anions, imide anions and methide anions are preferred. Examples of the non-nucleophilic counter ion include halide ions such as chloride and bromide ions; sulfonate anions, specifically fluoroalkylsulfonate ions such as triflate, 1,1,1-trifluoroethanesulfonate, and nonafluorobutanesulfonate, arylsulfonate ions such as tosylate, benzenesulfonate, 4-fluorobenzenesulfonate, and 1,2,3,4,5-pentafluorobenzenesulfonate, alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl)methide and tris(perfluoroethylsulfonyl)methide.

Anions having the following formulae (c1-1) to (c1-4) are also useful as the non-nucleophilic counter ion.

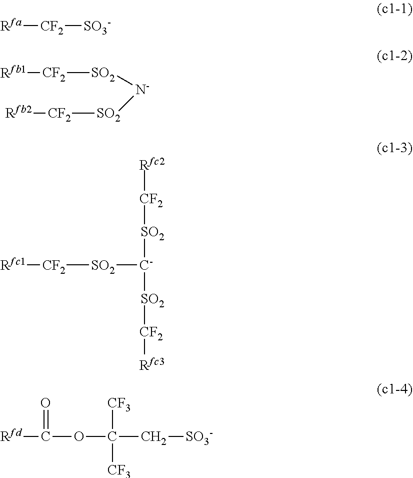

In formula (c1-1). R⁵ is fluorine or a C₁-C₄₀ hydrocarbyl group which may contain a heteroatom. The hydrocarbyl group may be saturated or unsaturated and straight, branched or cyclic. Examples thereof are as will be exemplified below for the hydrocarbyl group R^{fa1} in formula (c1-1-1).

Of the anions of formula c1-1 an anion having the formula (c1-1-1) is preferred.

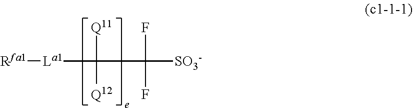

In formula (c1-1-1), Q¹¹ and Q¹² are each independently hydrogen, fluorine or a C₁-C₆ fluorinated saturated hydrocarbyl group. It is preferred for solvent solubility that at least one of Q¹¹ and Q¹² be trifluoromethyl. The subscript e is an integer of 0 to 4, preferably 1.

R^{fa1} is a C₁-C₃₅ hydrocarbyl group which may contain a heteroatom. As the heteroatom, oxygen, nitrogen, sulfur and halogen atoms are preferred, with oxygen being most preferred. Of the hydrocarbyl groups, those groups of 6 to 30 carbon atoms are preferred from the aspect of achieving a high resolution in forming patterns of small feature size. The hydrocarbyl group may be saturated or unsaturated and straight, branched or cyclic. Examples thereof include C₁-C₃₅ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl: C₃-C₃₅ cyclic saturated hydrocarbyl groups such as cyclopentyl, cyclohexyl, 1-adamantyl, 2-adamantyl, 1-adamantylmethyl, norbomyl, norbornylmethyl, tricyclodecyl, tetracyclododecyl, tetracyclododecylmethyl, and dicyclohexyhmethyl; C₂-C₃₅ unsaturated aliphatic hydrocarbyl groups such as allyl and 3-cyclohexenyl; C₆-C₃₅ aryl groups such as phenyl, 1-naphthyl, 2-naphthyl and 9-fluorenyl; and C₇-C₃₅ aralkyl groups such as benzyl and diphenylmethyl, and combinations thereof.

In the foregoing hydrocarbyl groups, some or all hydrogen may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

In formula (c1-1-1), L^{a1} is a single bond, ether bond, ester bond, sulfonic ester bond, carbonate bond or carbamate bond. From the aspect of synthesis, an ether bond or ester bond is preferred, with the ester bond being more preferred.

Examples of the anion having formula (c1-1) are shown below, but not limited thereto. Herein Q¹¹ is as defined above.

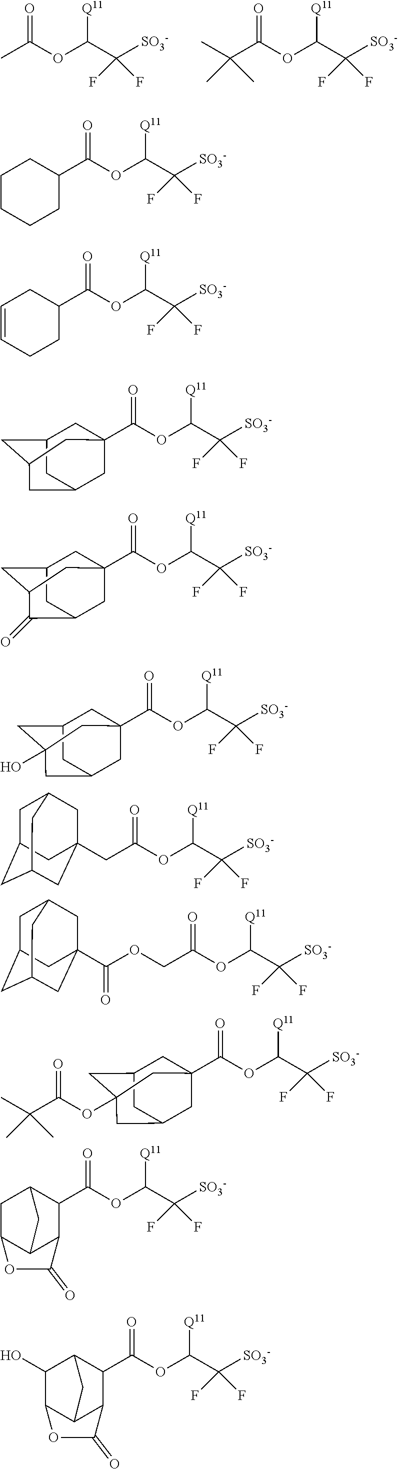

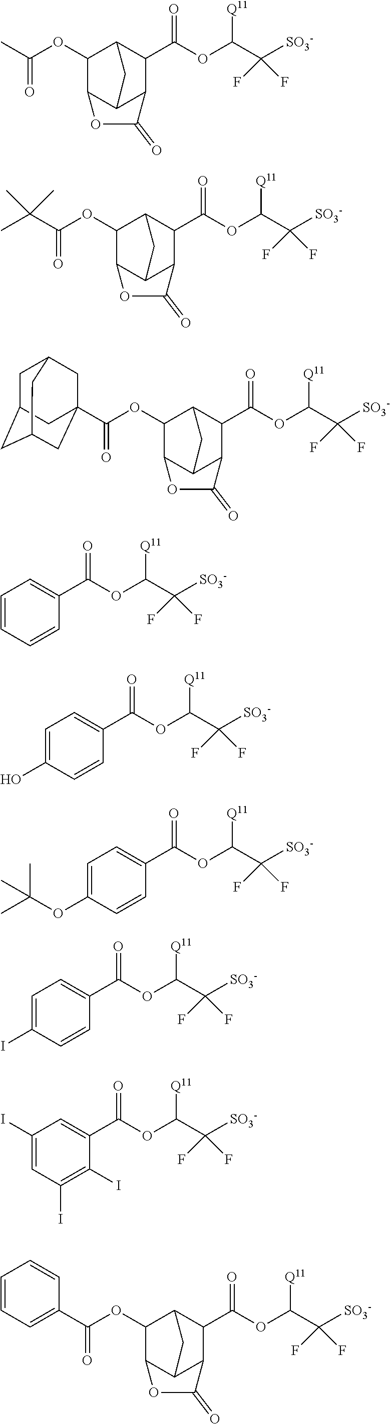

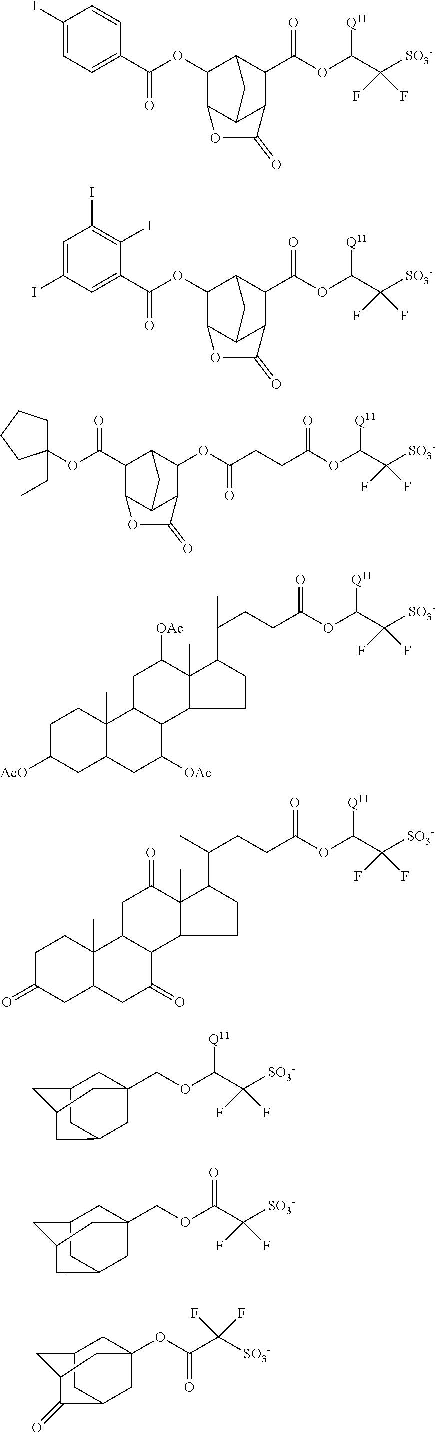

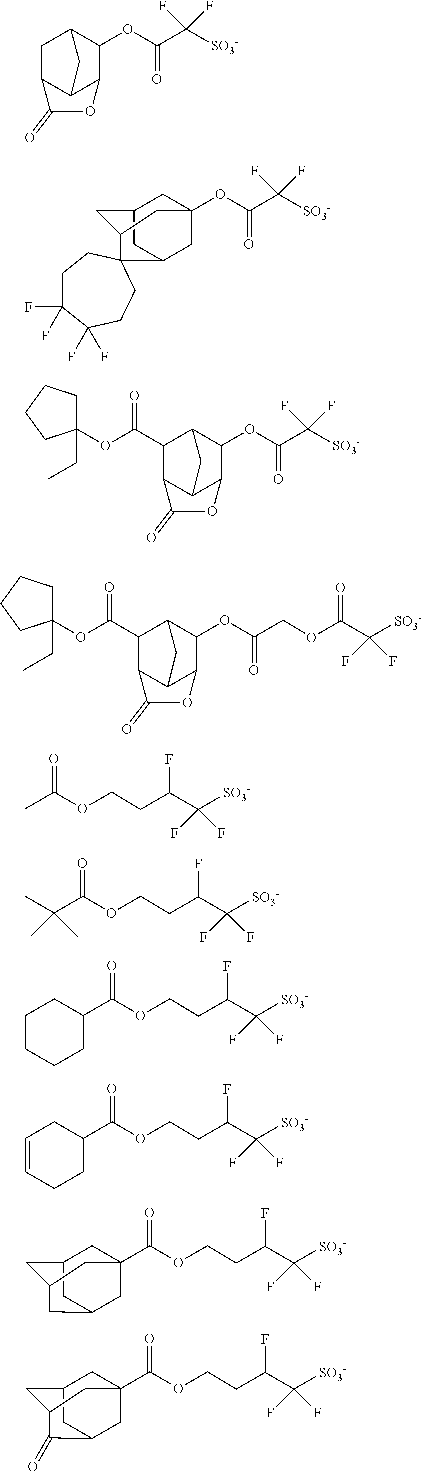

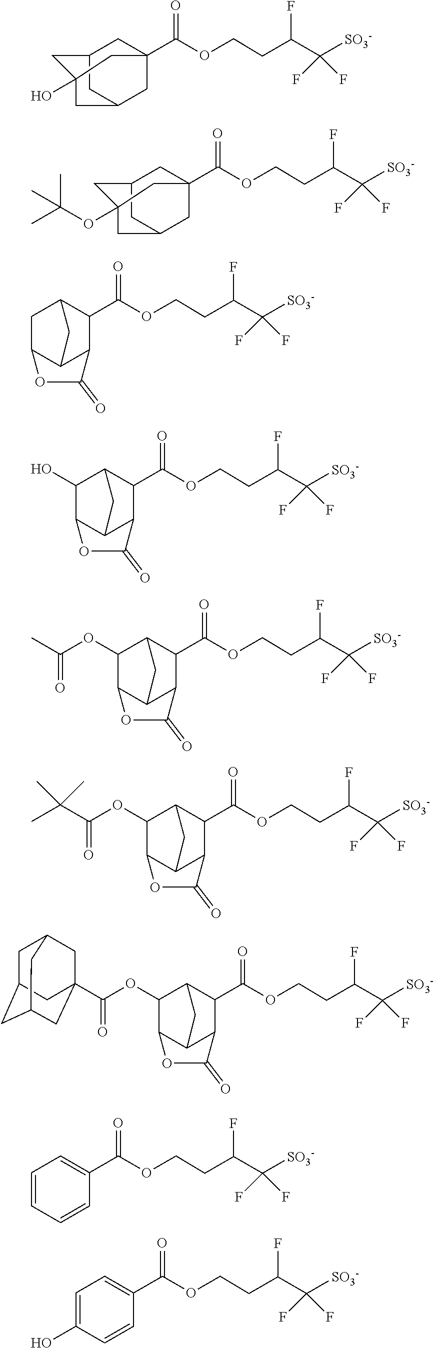

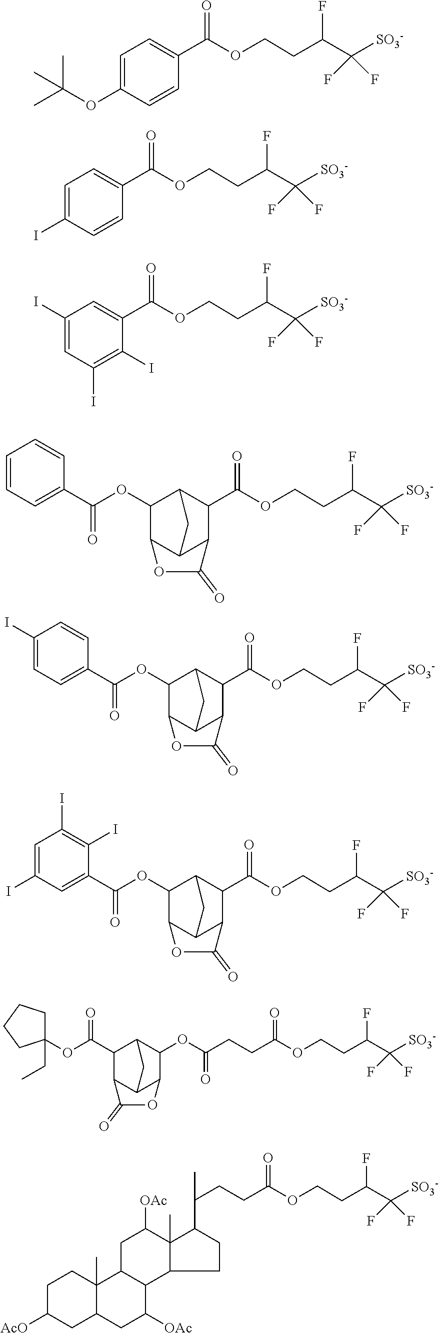

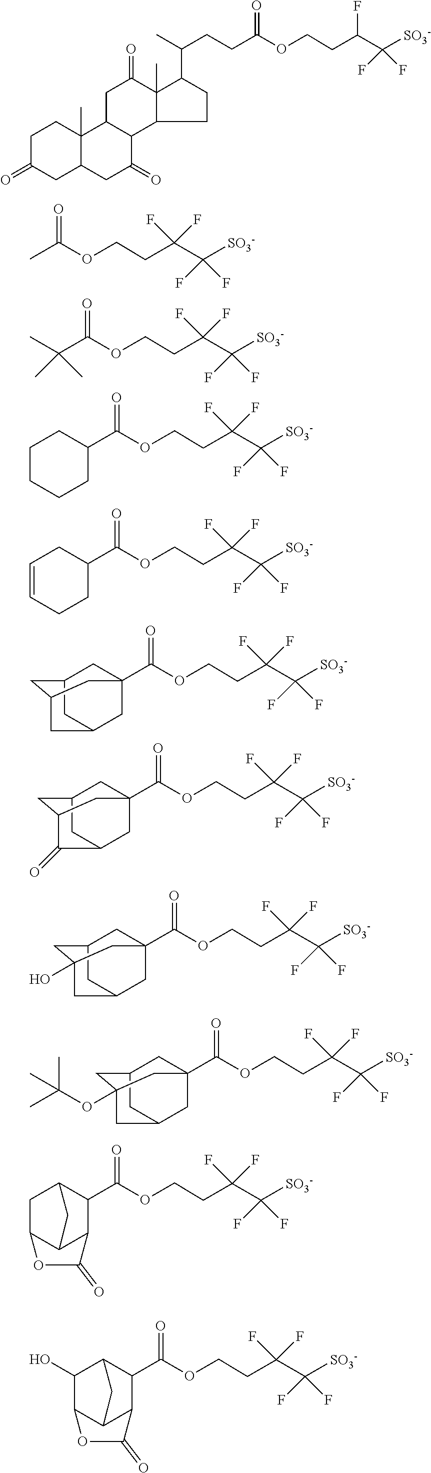

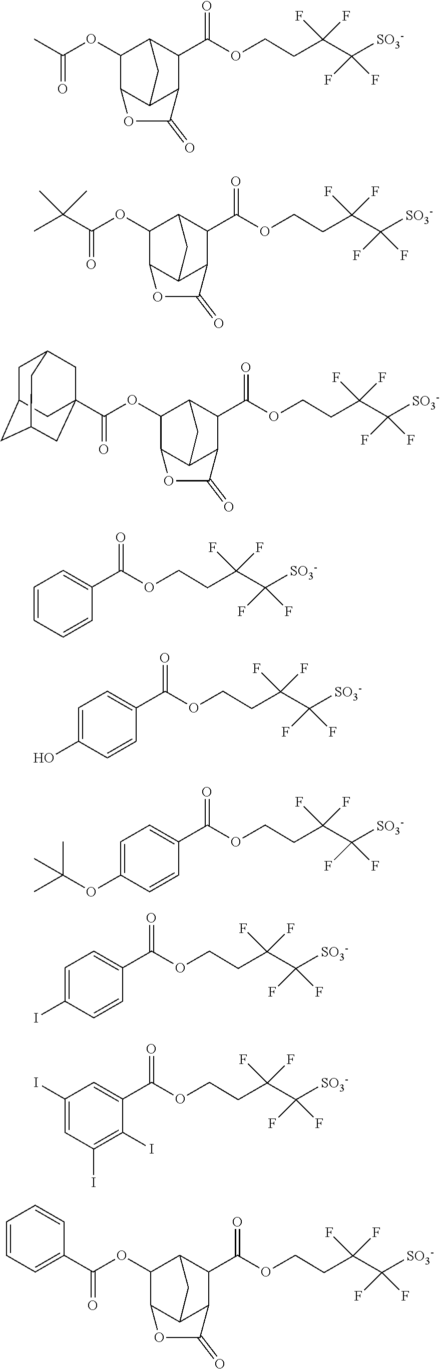

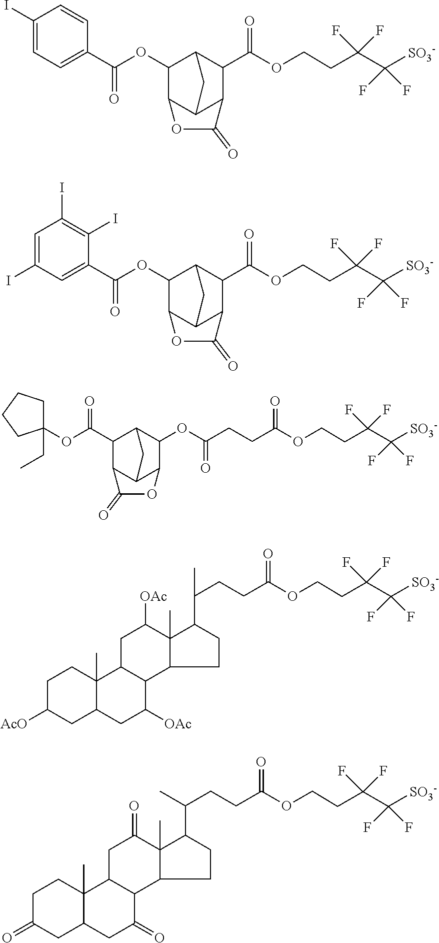

In formula (c1-2), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Examples thereof are as exemplified above for R^{fa1} in formula (c1-1-1). Preferably R^{fb1} and R^{fb2} are fluorine or C₁-C₄ straight fluorinated alkyl groups. Also, R^{fb1} and R^{fb2} may bond together to form a ring with the linkage: —CF₂—SO₂—N⁻—SO₂—CF₂— to which they are attached. It is preferred that a combination of R^{fb1} and R^{fb2} be a fluorinated ethylene or fluorinated propylene group.

In formula (c1-3), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Examples thereof are as exemplified for R^{fa1} in formula (c1-1-1). Preferably R^{fc1}, R^{fc2} and R^{fc3} are fluorine or C₁-C₄ straight fluorinated alkyl groups. Also, R^{fc1} and R^{fc2} may bond together to form a ring with the linkage: —CF₂—SO₂—C—SO₂—CF₂— to which they are attached. It is preferred that a combination of R^{fc1} and R^{fc2} be a fluorinated ethylene or fluorinated propylene group.

In formula (c1-4). R^{fd} is a C₁-C₄₀ hydrocarbyl group which may contain a heteroatom. The hydrocarbyl group may be saturated or unsaturated and straight, branched or cyclic. Examples thereof are as exemplified above for R^{fa1}.

Examples of the anion having formula (c1-4) are shown below, but not limited thereto.

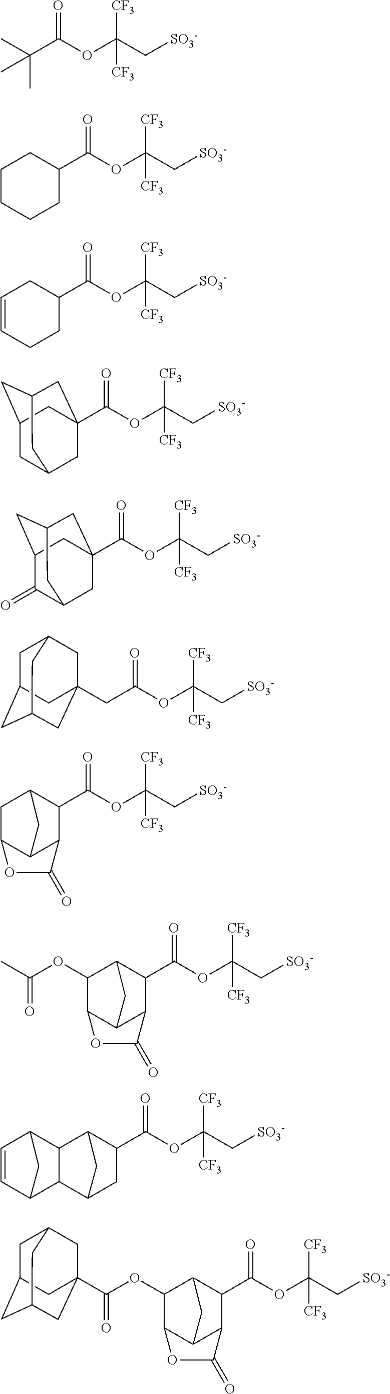

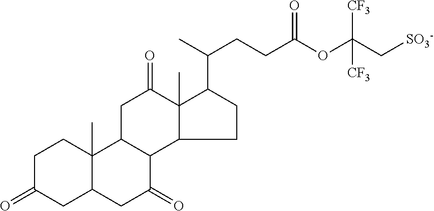

Anions having an iodized or brominated aromatic ring are also useful as the non-nucleophilic counter ion. These anions have the formula (c1-5).

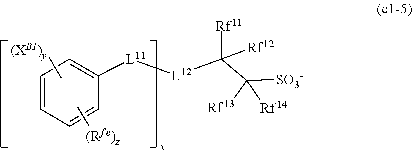

In formula (c1-5), x is an integer of 1 to 3, y is an integer of 1 to 5, z is an integer of 0 to 3, and y+z is from 1 to 5; preferably, y is an integer of 1 to 3, more preferably 2 or 3, and z is an integer of 0 to 2.

X^{BI} is iodine or bromine. A plurality of X^{BI} may be identical or different when x and/or y is 2 or more.

L¹¹ is a single bond, ether bond, ester bond, or a C₁-C₆ saturated hydrocarbylene group which may contain an ether bond or ester bond. The saturated hydrocarbylene group may be straight, branched or cyclic.

L¹² is a single bond or a C₁-C₂₀ divalent linking group when x=1, or a C₁-C₂₀ (x+1)-valent linking group when x=2 or 3. The linking group may contain an oxygen, sulfur or nitrogen atom.

R^{fe} is hydroxy, carboxy, fluorine, chlorine, bromine, amino group, or a C₁-C₂₀ hydrocarbyl, C₁-C₂₀ hydrocarbyloxy, C₂-C₂₀ hydrocarbylcarbonyl, C₂-C₂₀ hydrocarbyloxycarbonyl. C₂-C₂₀ hydrocarbylcarbonyloxy, or C₁-C₂₀ hydrocarbylsulfonyloxy group, which may contain fluorine, chlorine, bromine, hydroxy, amino or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen, or a C₁-C₆ saturated hydrocarbyl group which may contain halogen, hydroxy. C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R¹ may be identical or different when x and/or z is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

Rf¹¹ to Rf¹⁴ are each independently hydrogen, fluorine or trifluoromethyl, at least one of Rf¹¹ to Rf¹⁴ is fluorine or trifluoromethyl. Rf¹¹ and Rf¹², taken together, may form a carbonyl group. More preferably, both Rf¹³ and Rf¹⁴ are fluorine.

Examples of the anion having formula (c1-5) are shown below, but not limited thereto. X^{BI} is as defined above.

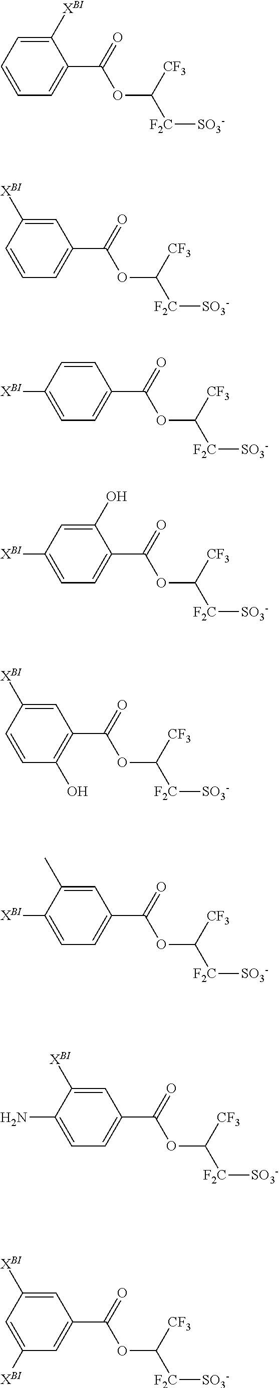

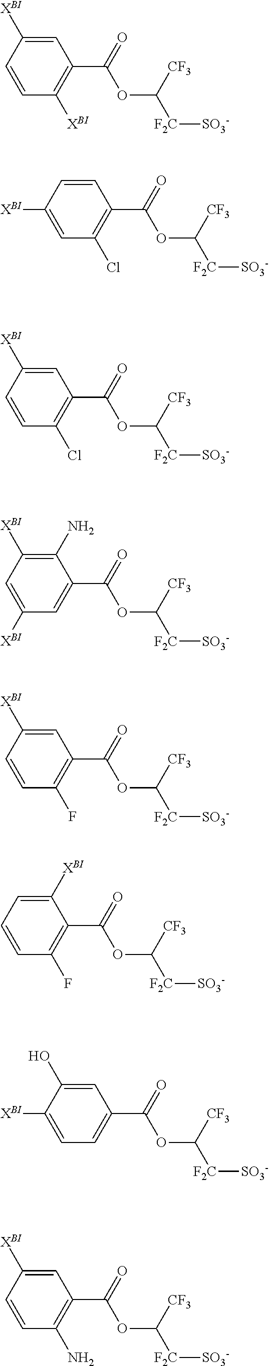

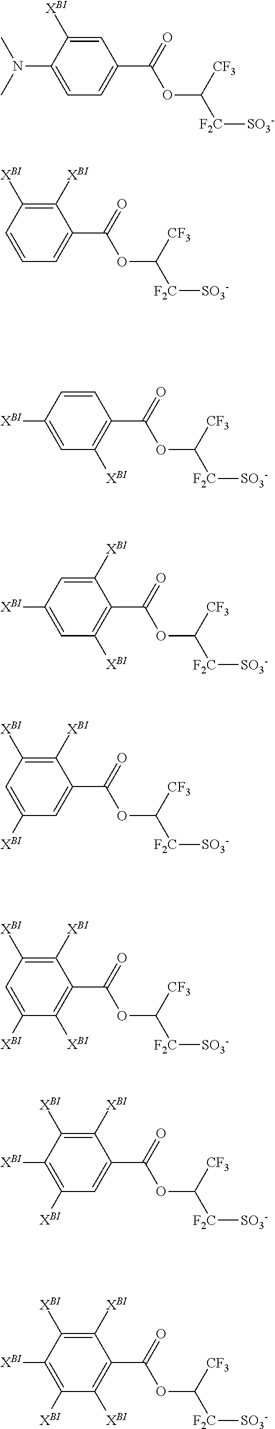

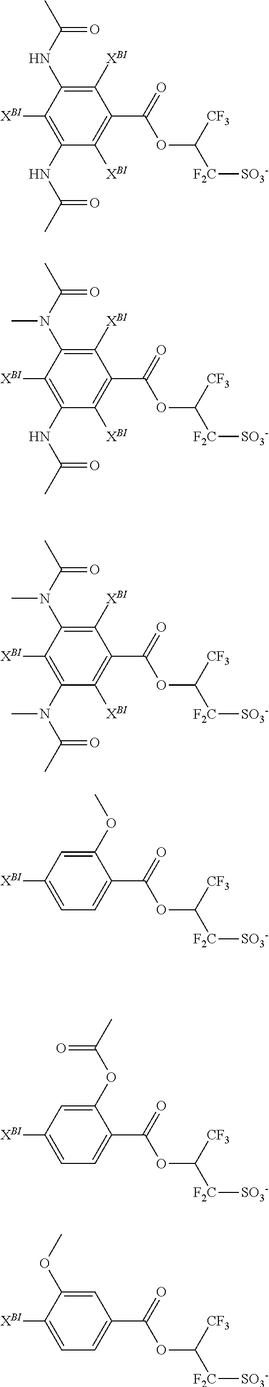

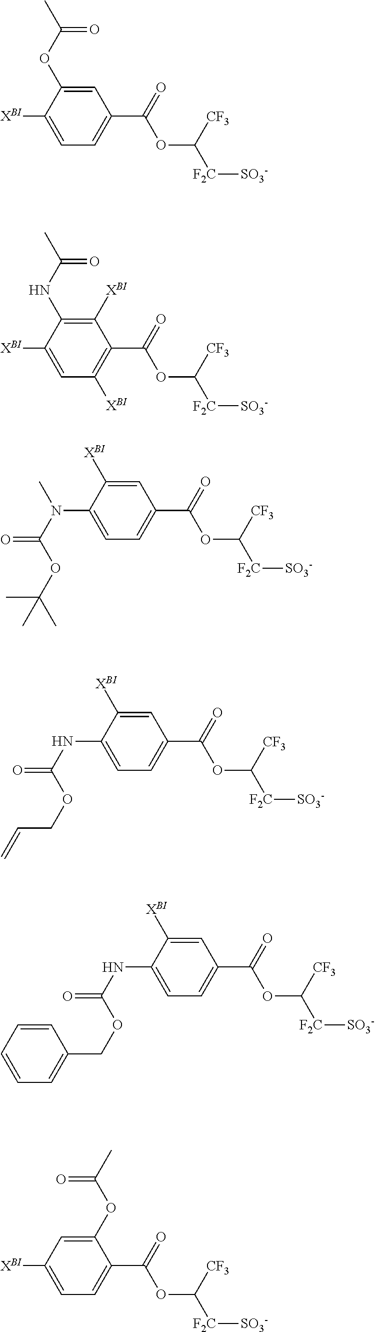

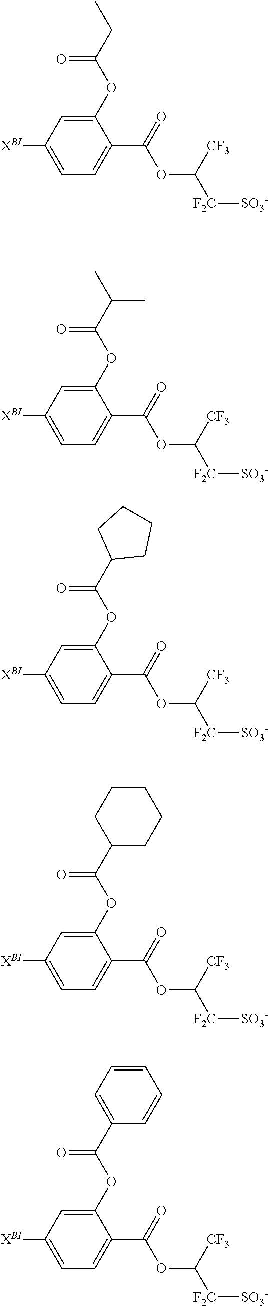

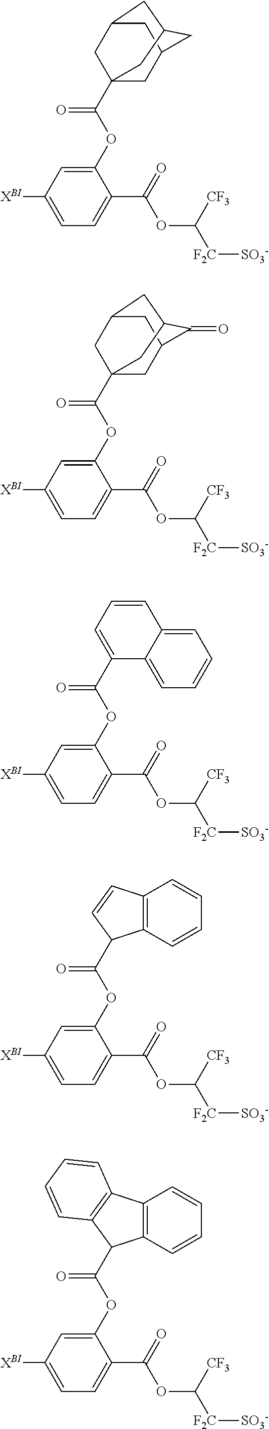

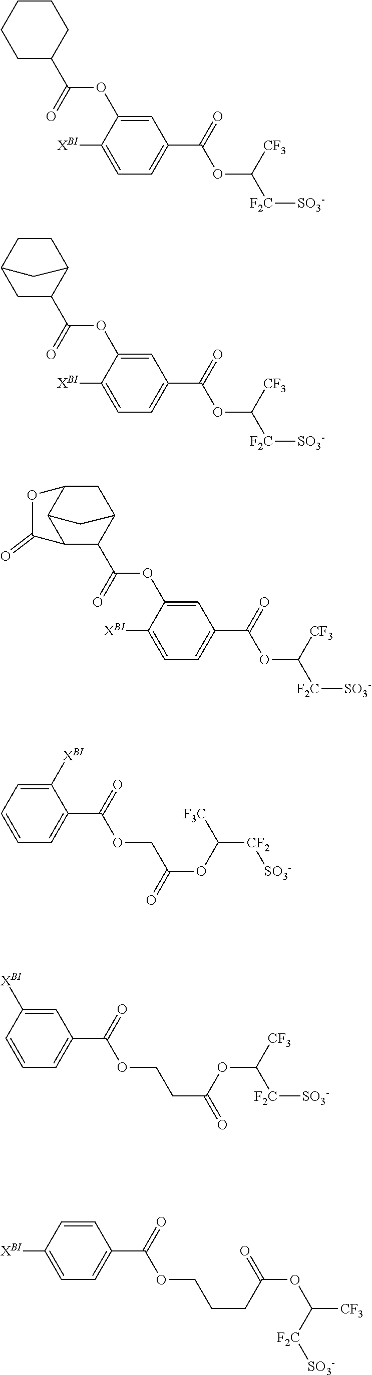

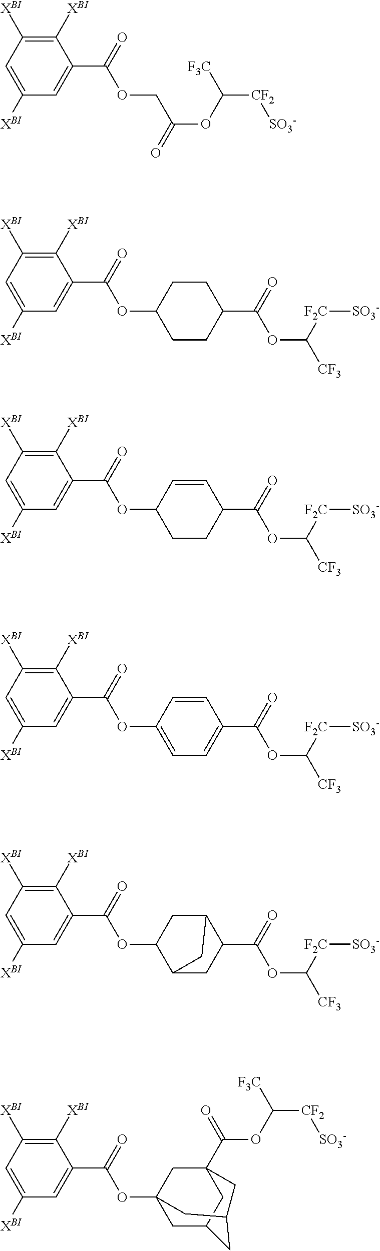

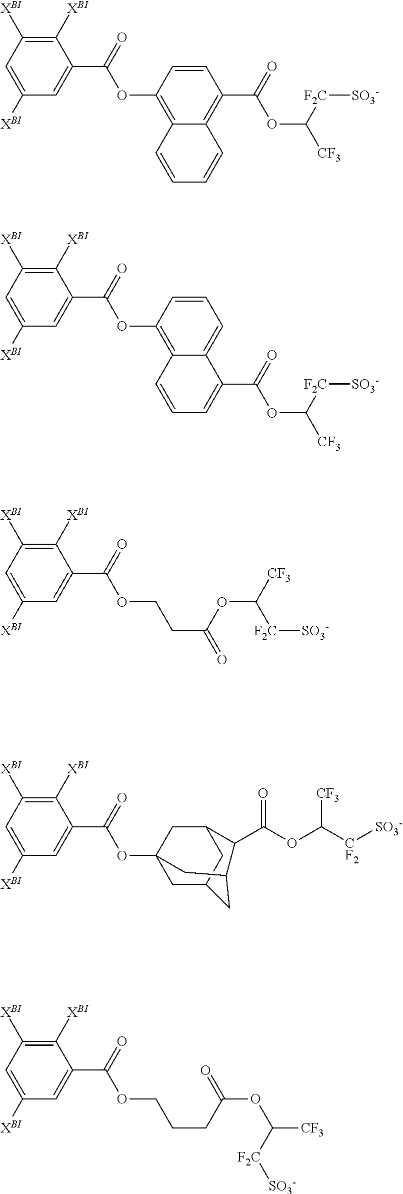

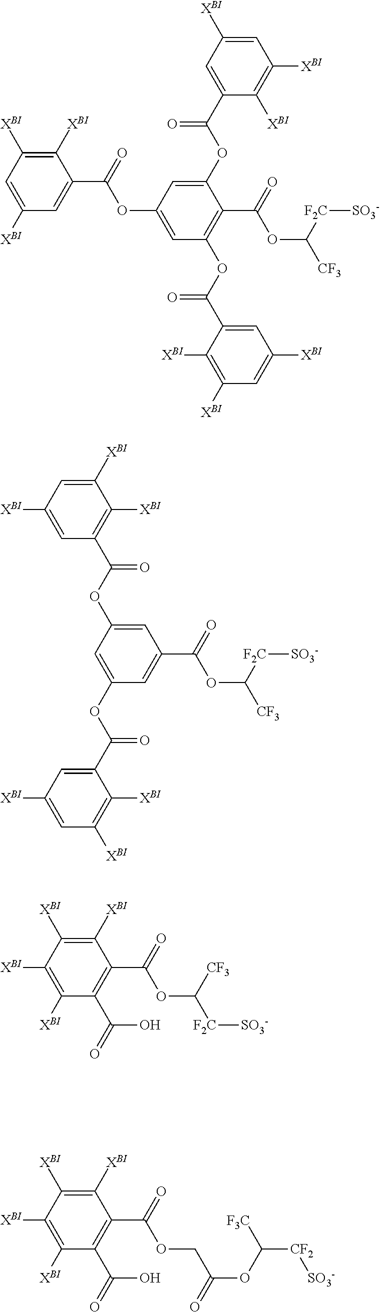

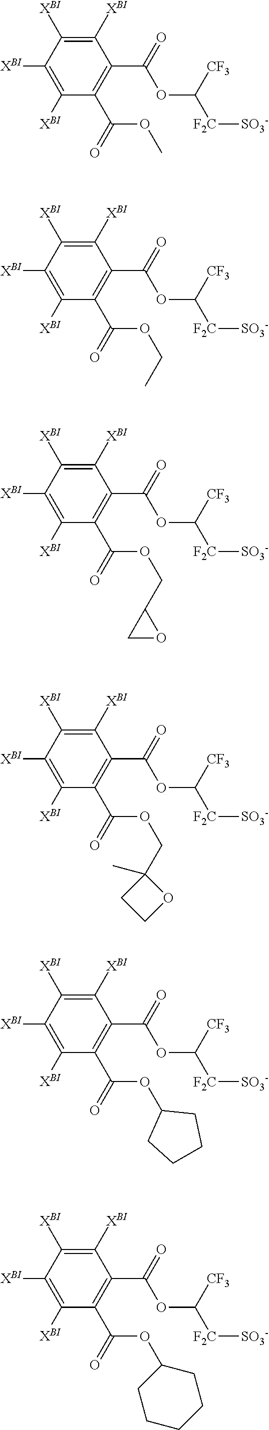

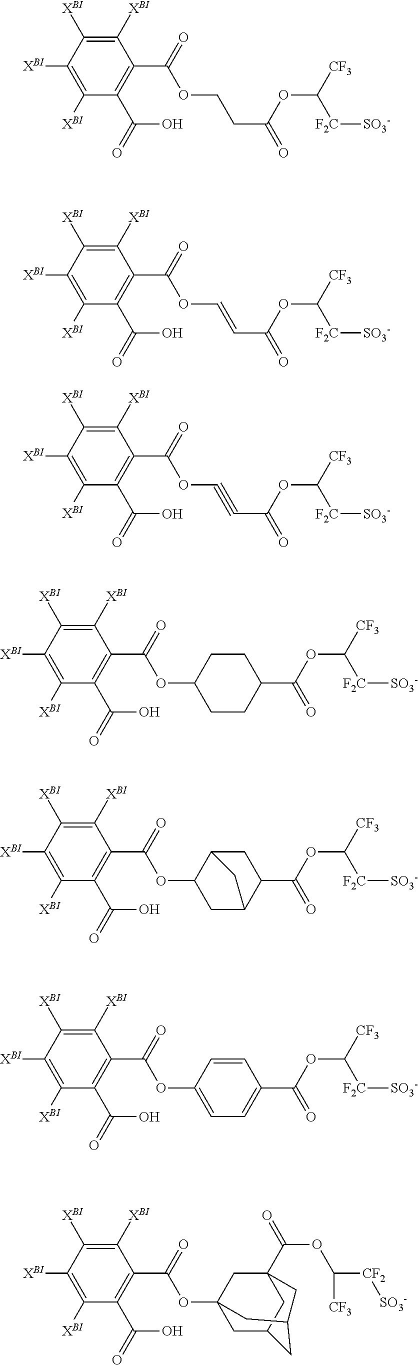

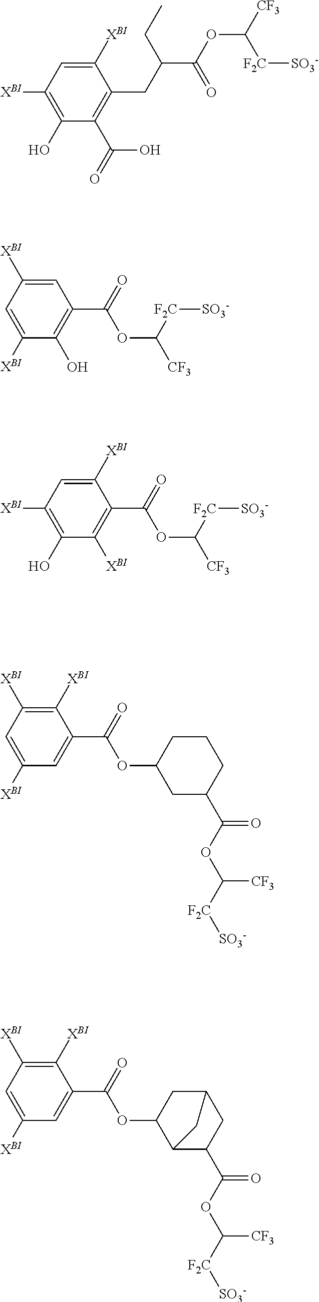

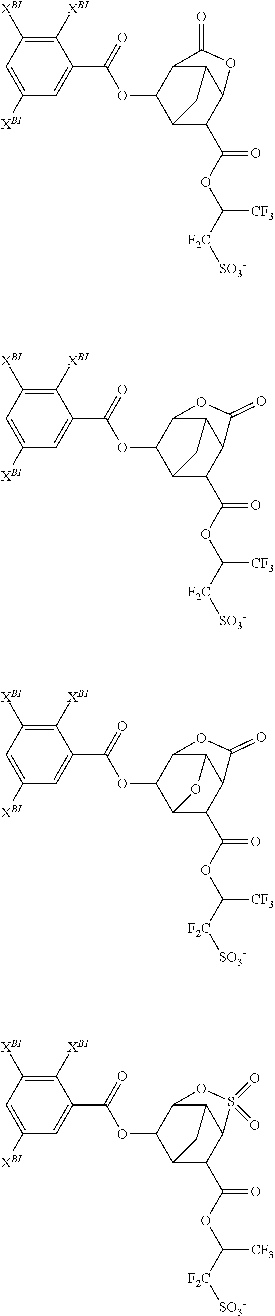

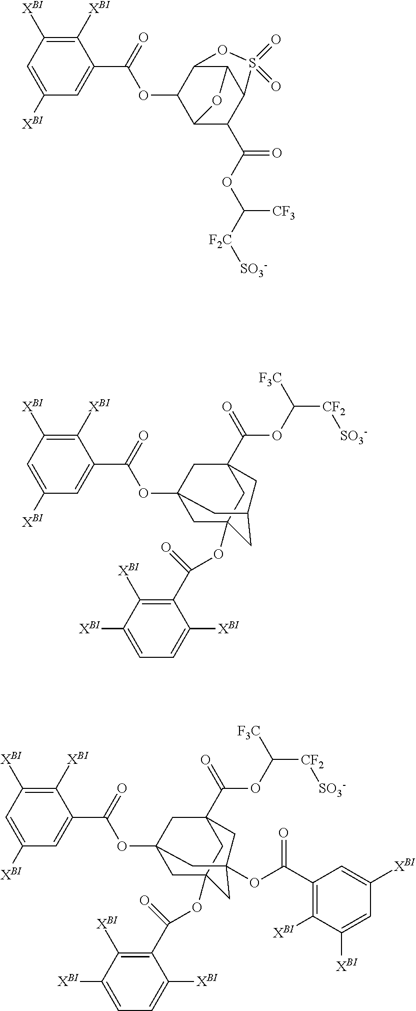

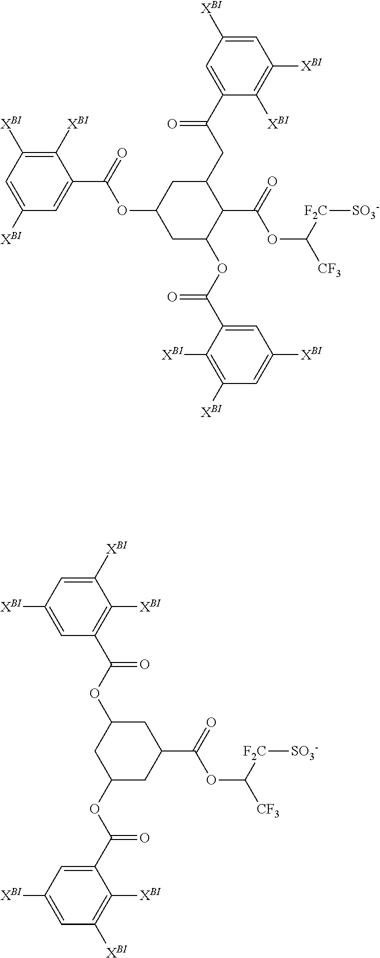

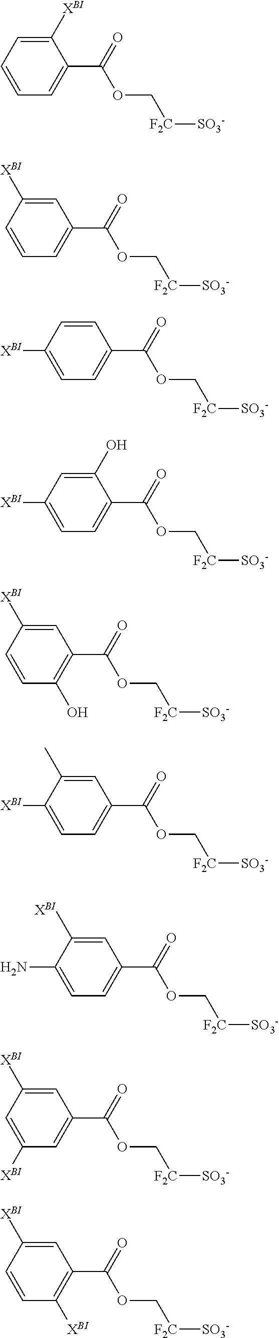

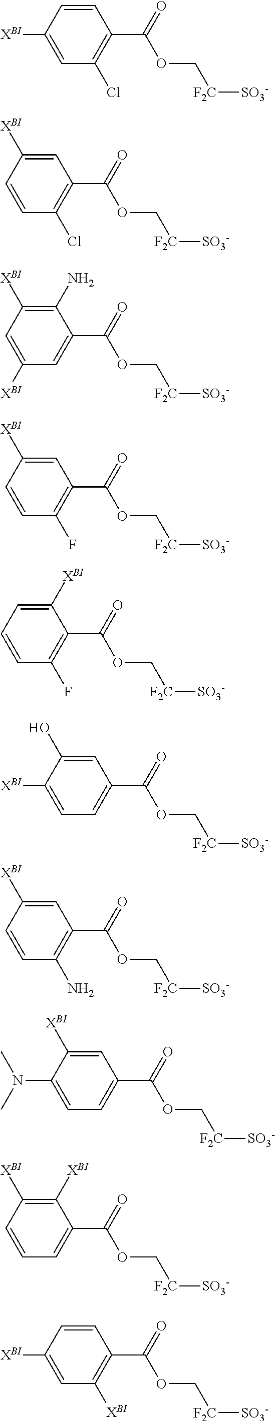

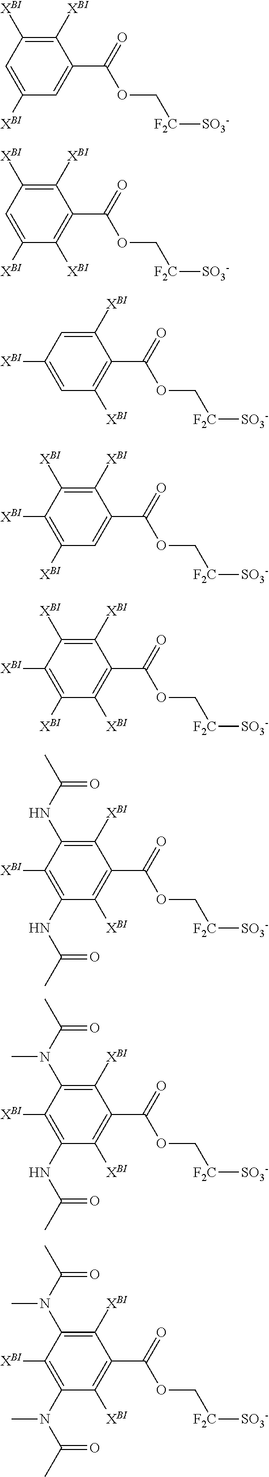

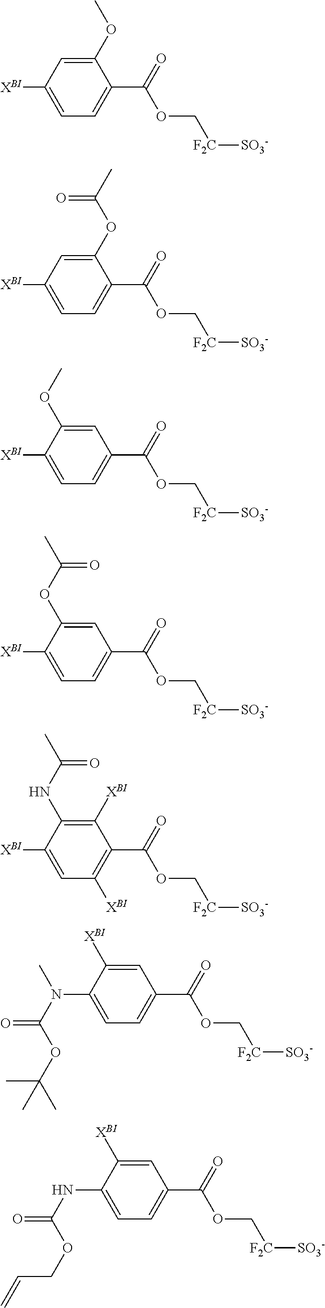

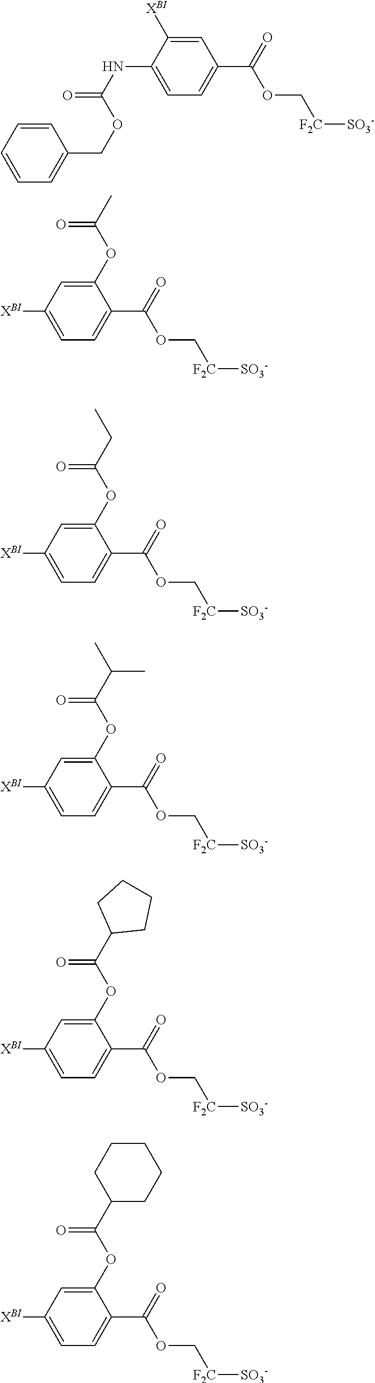

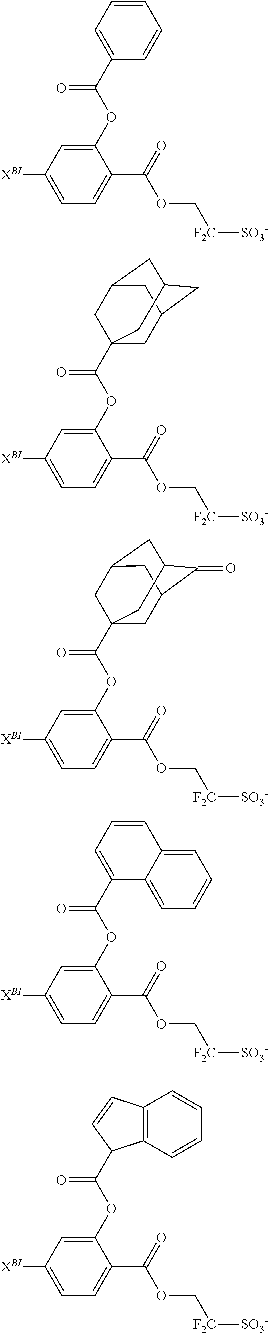

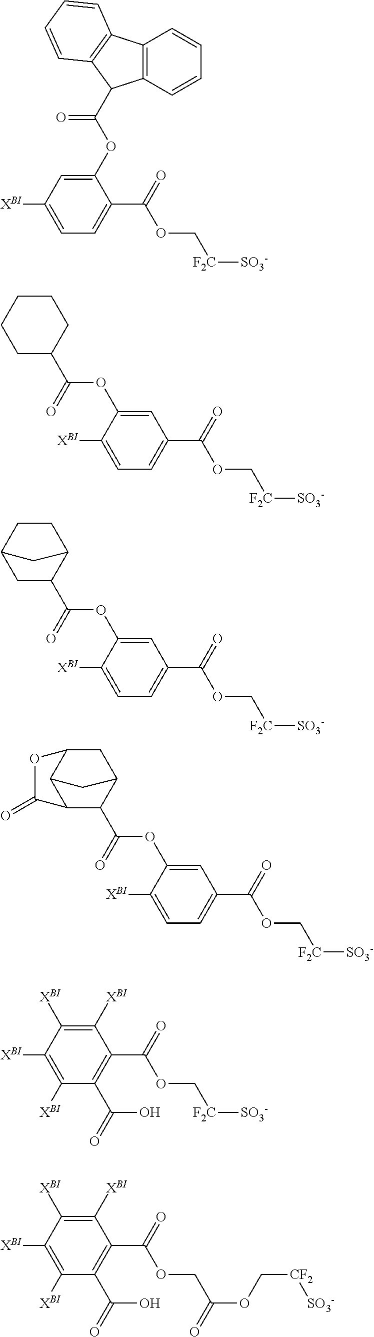

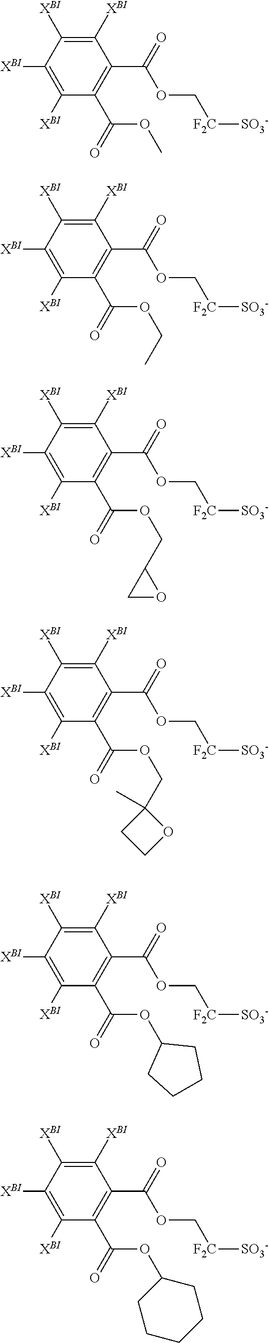

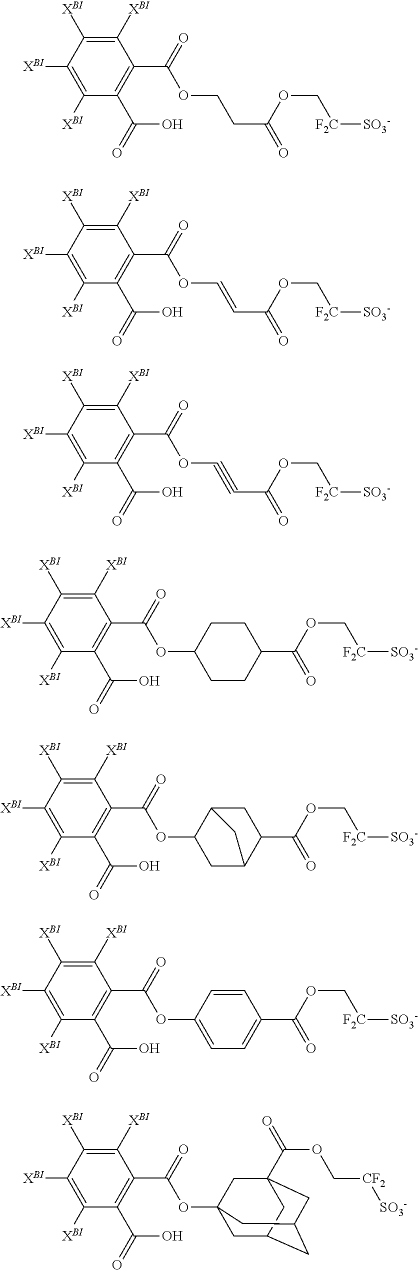

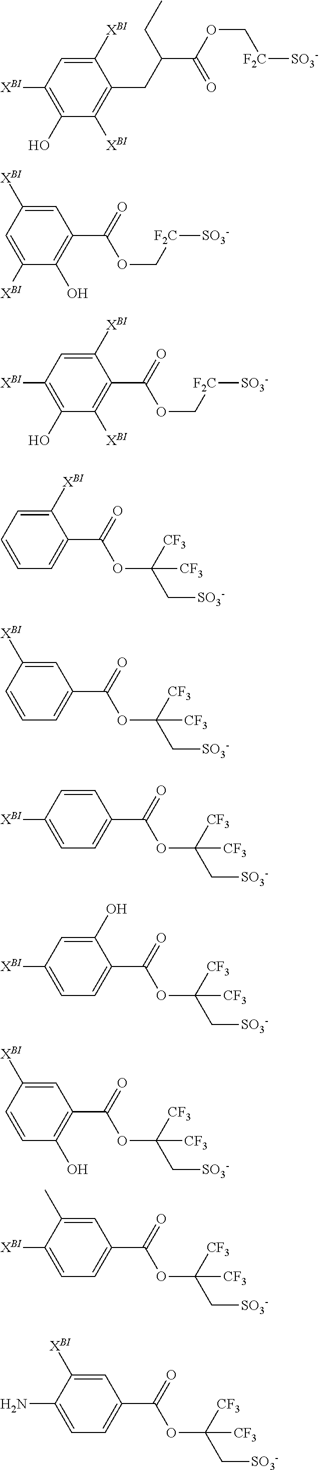

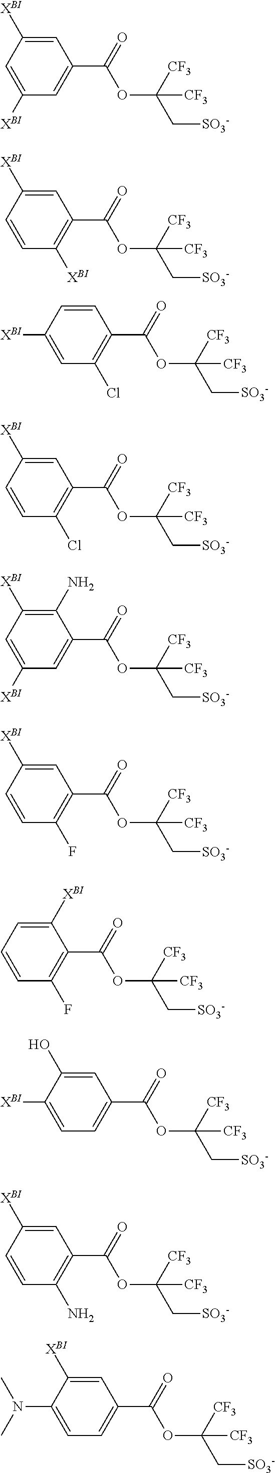

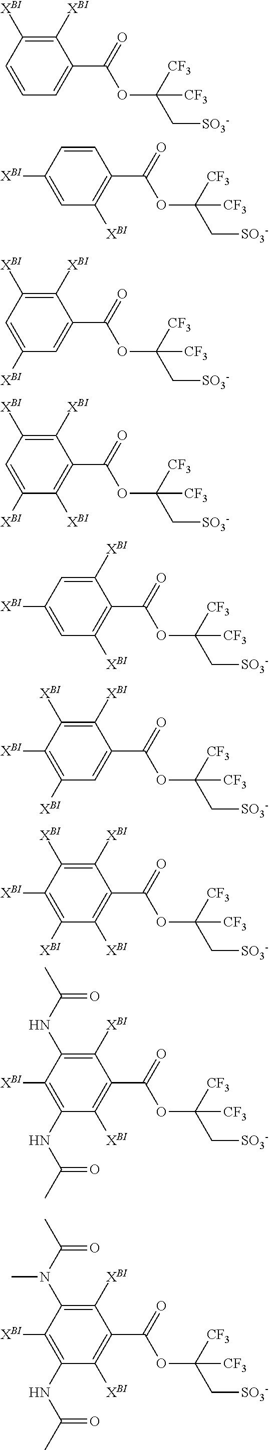

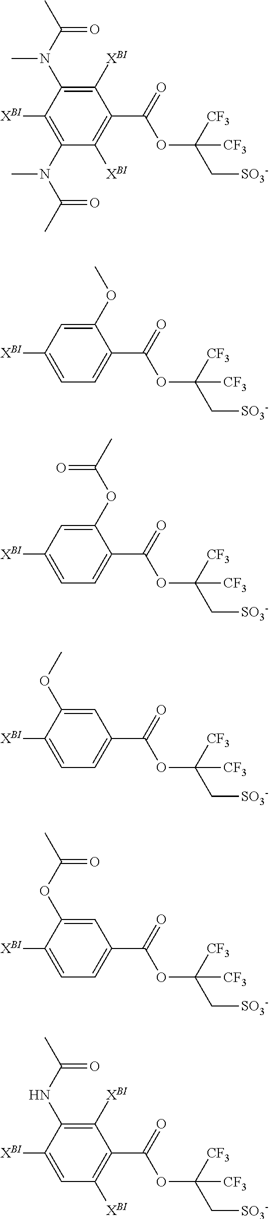

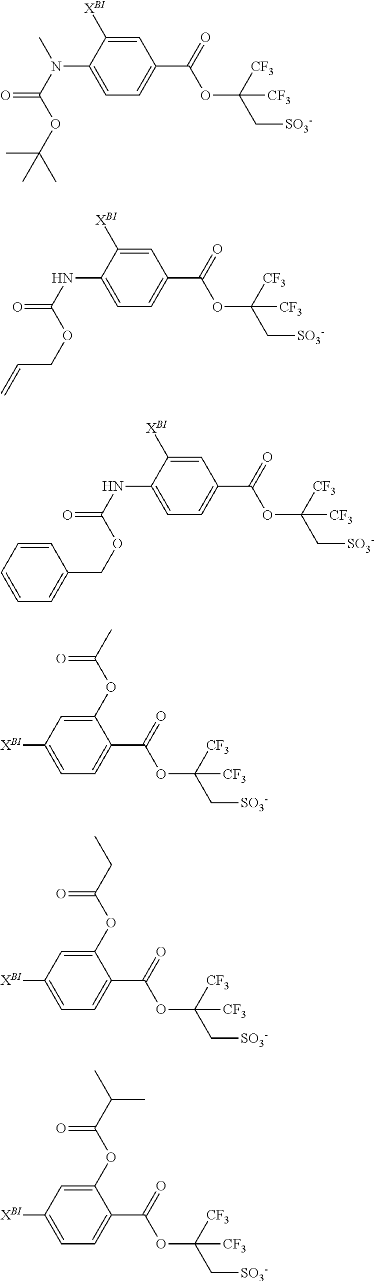

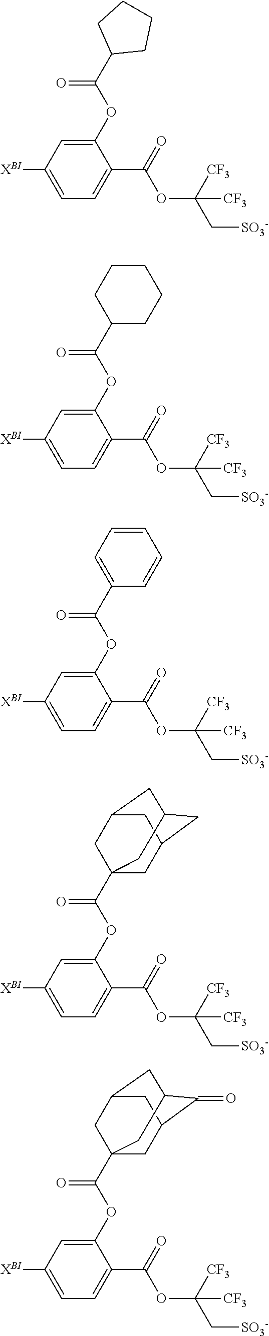

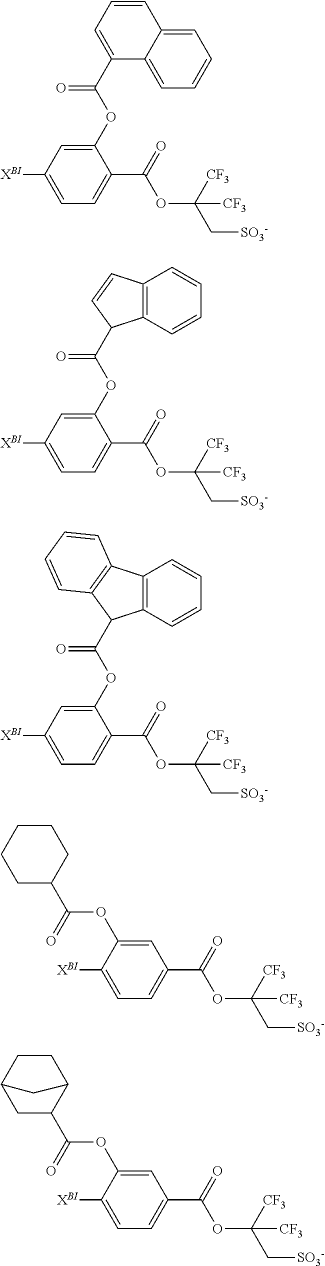

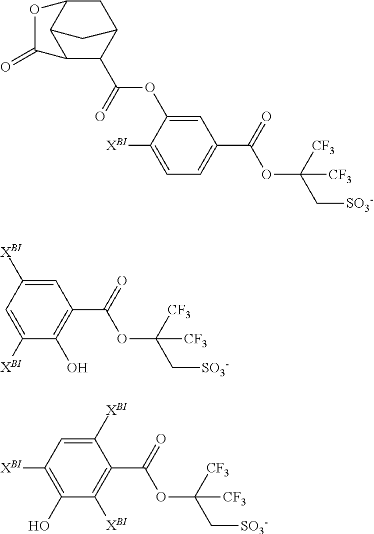

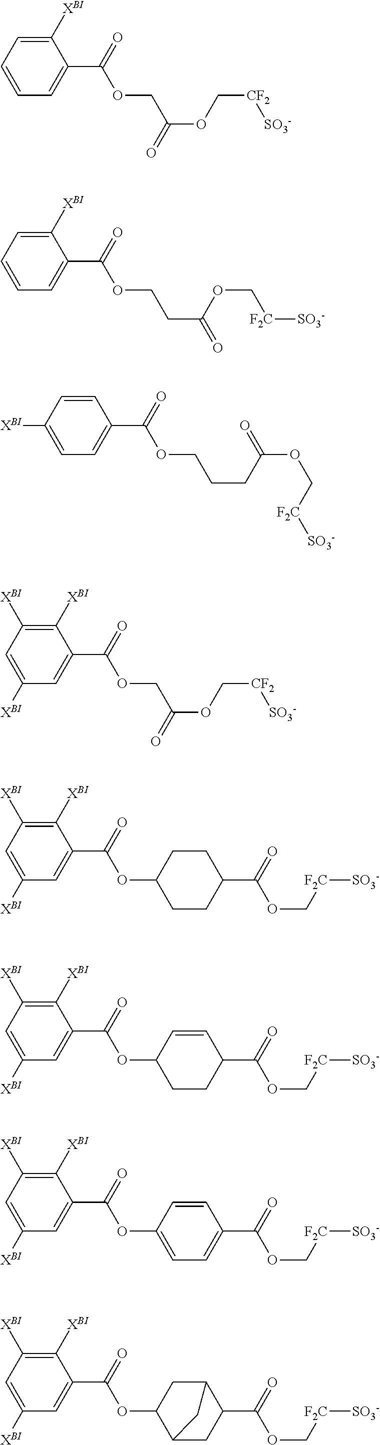

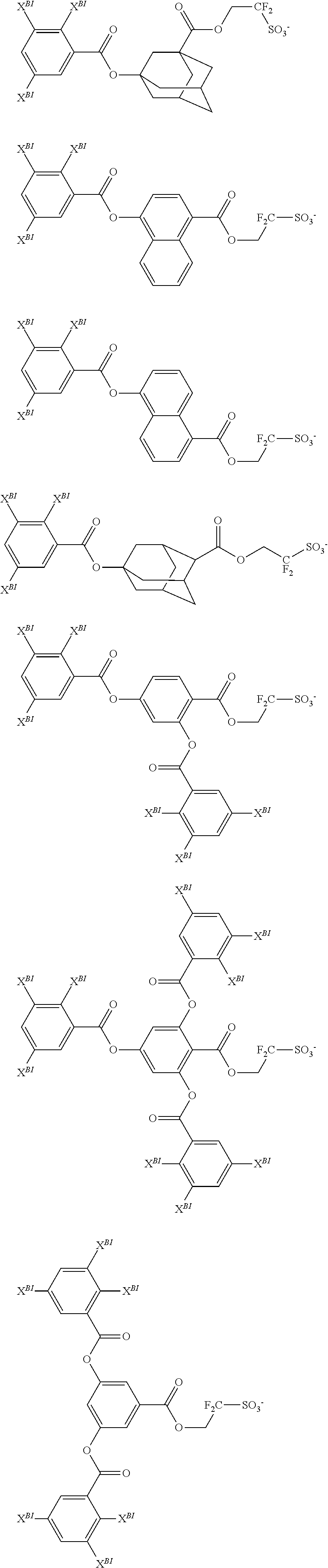

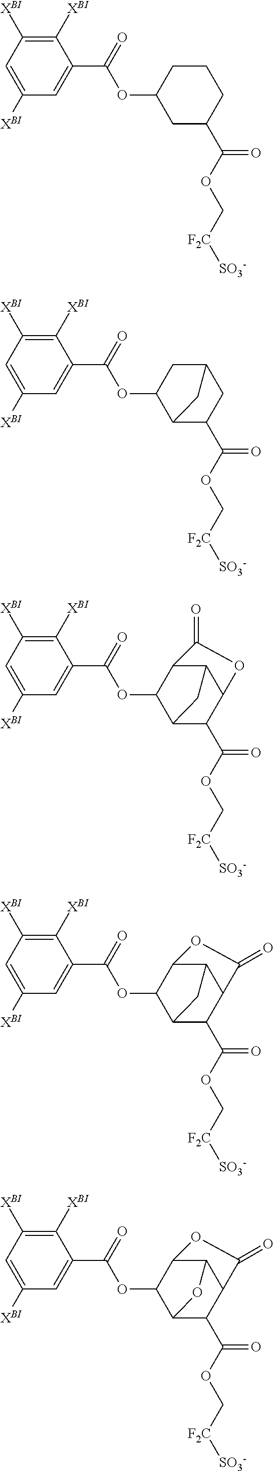

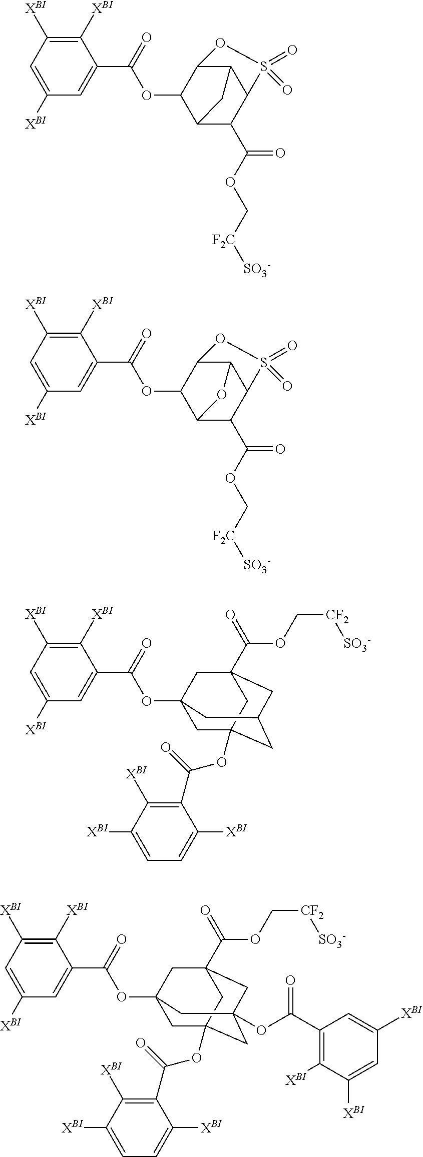

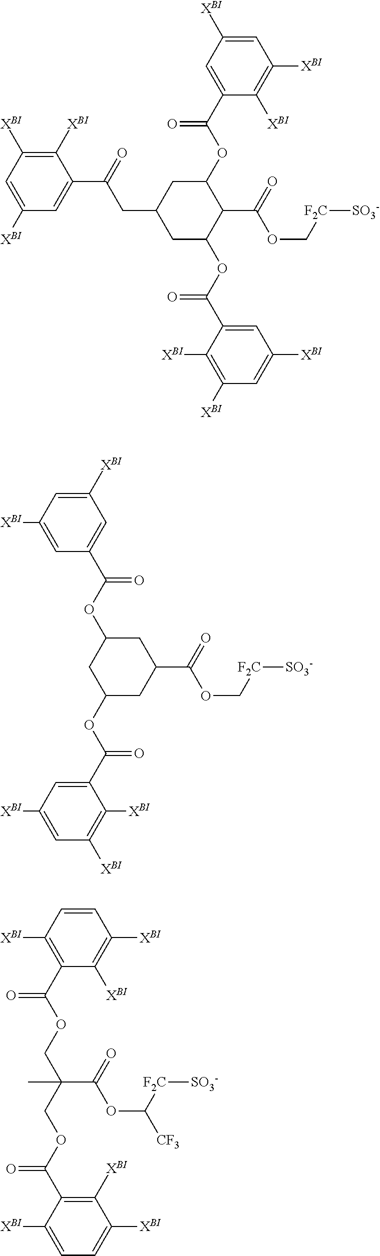

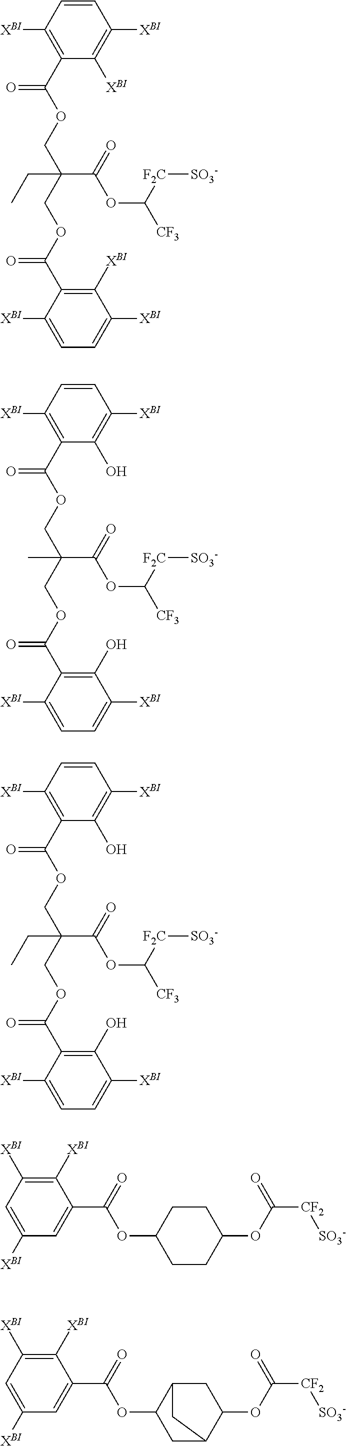

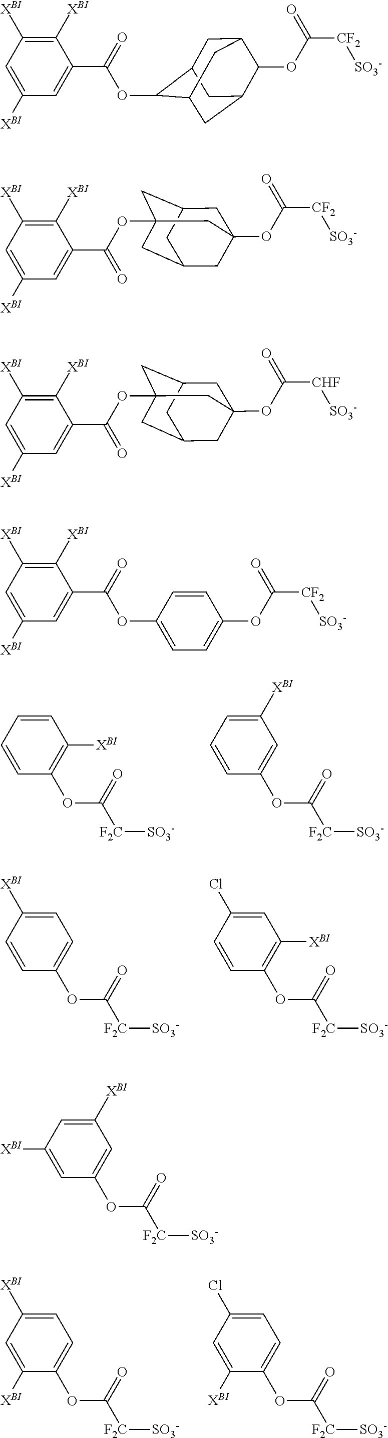

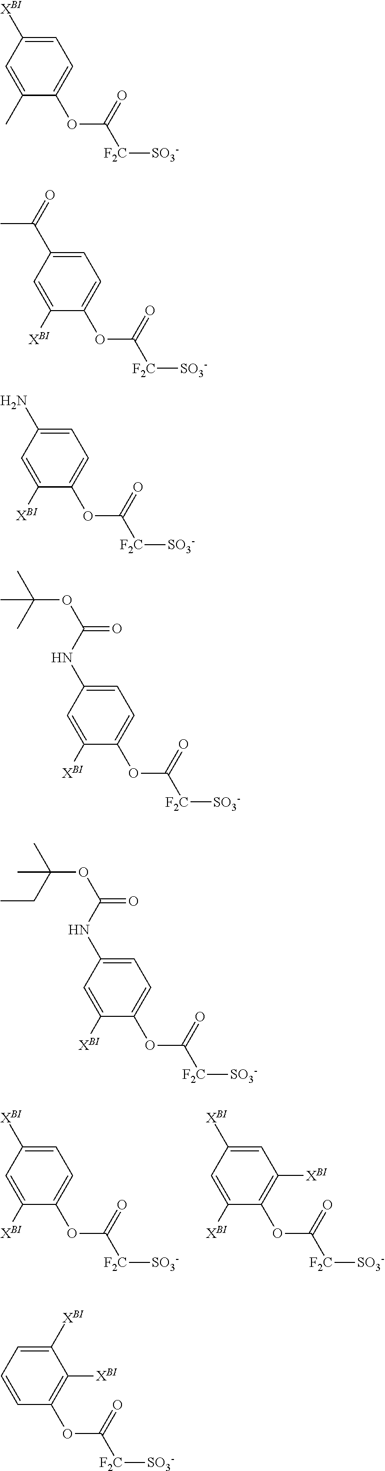

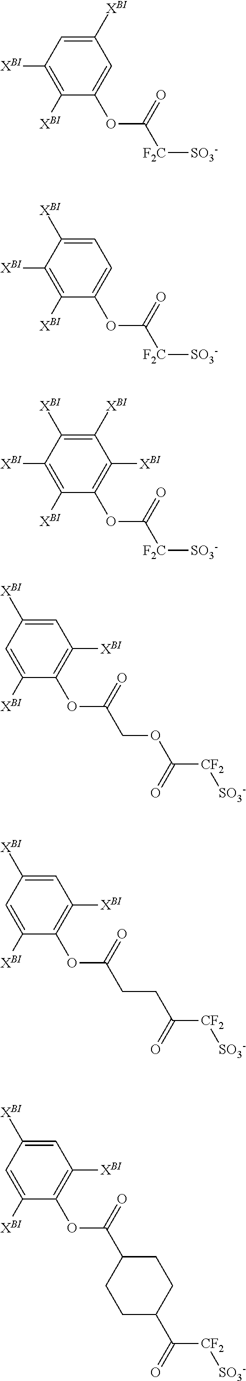

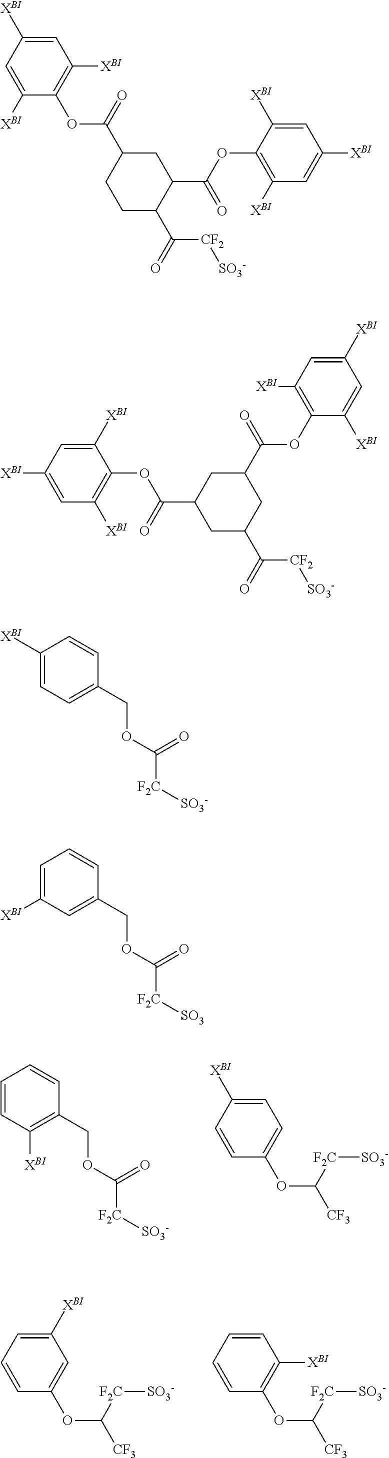

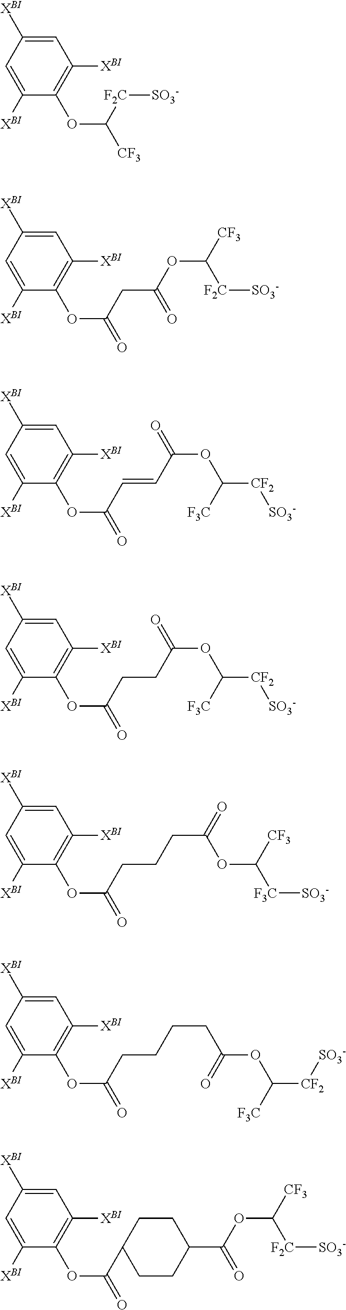

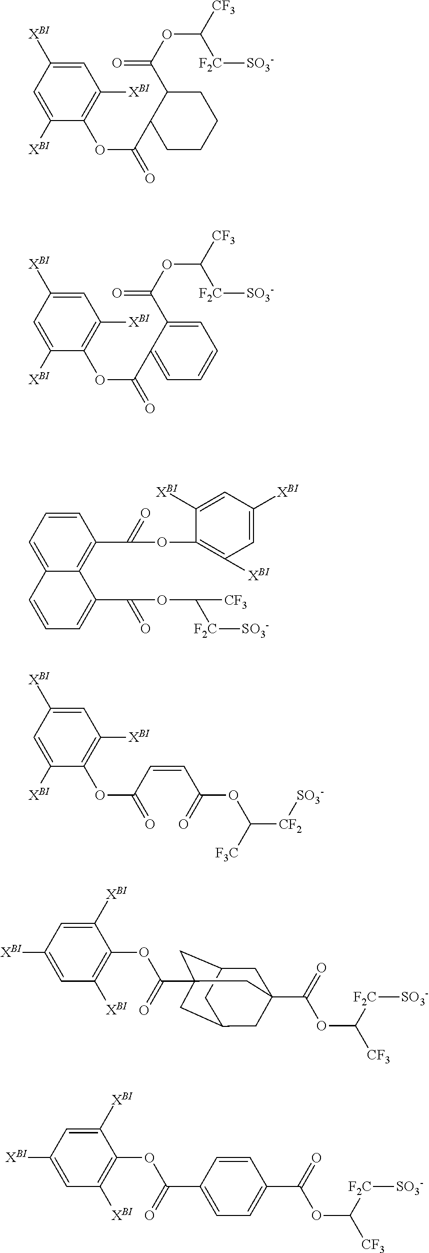

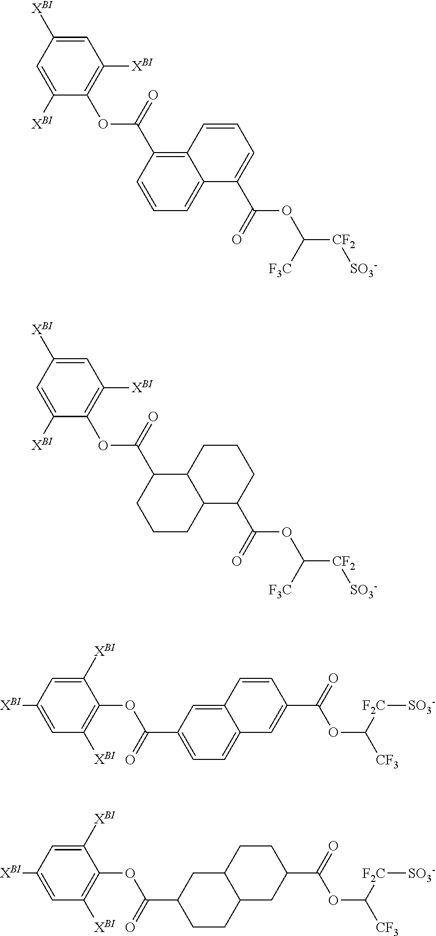

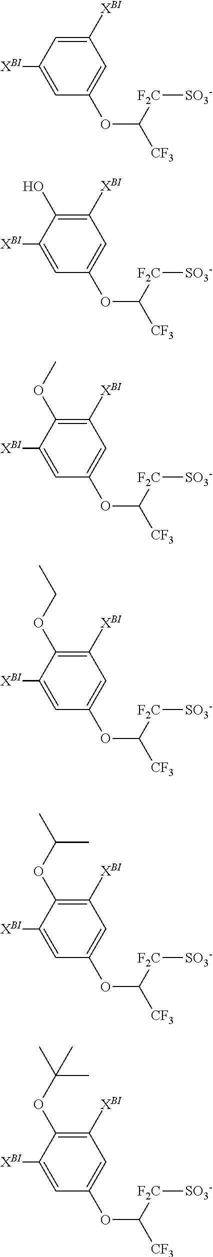

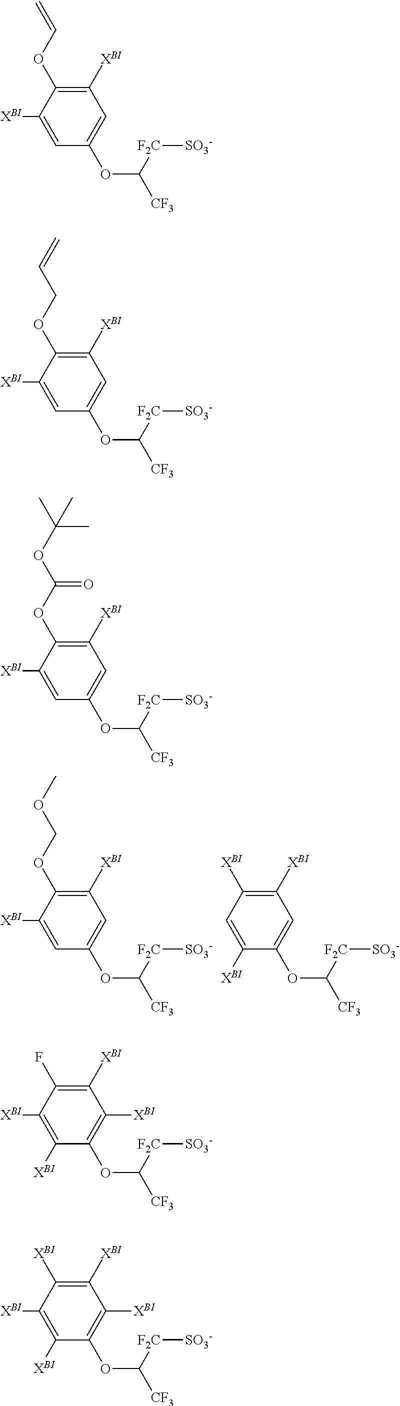

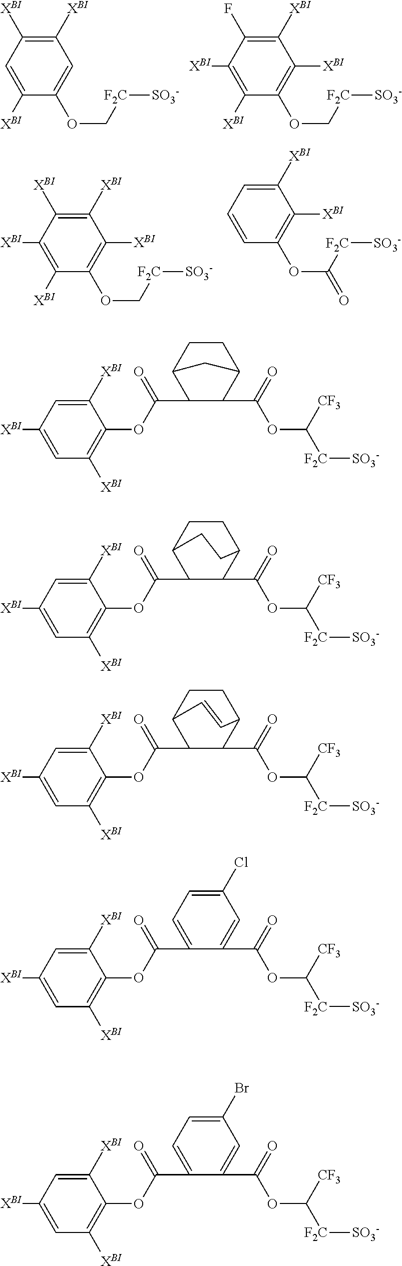

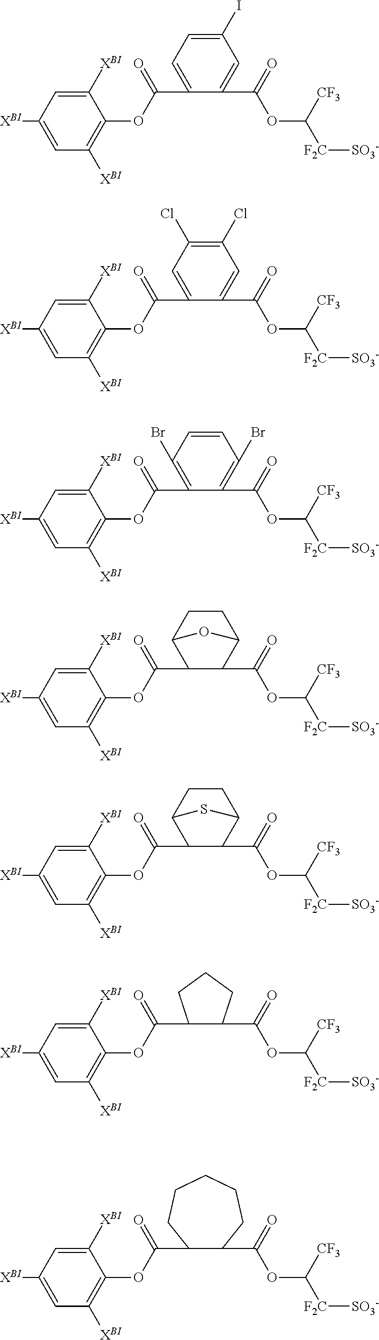

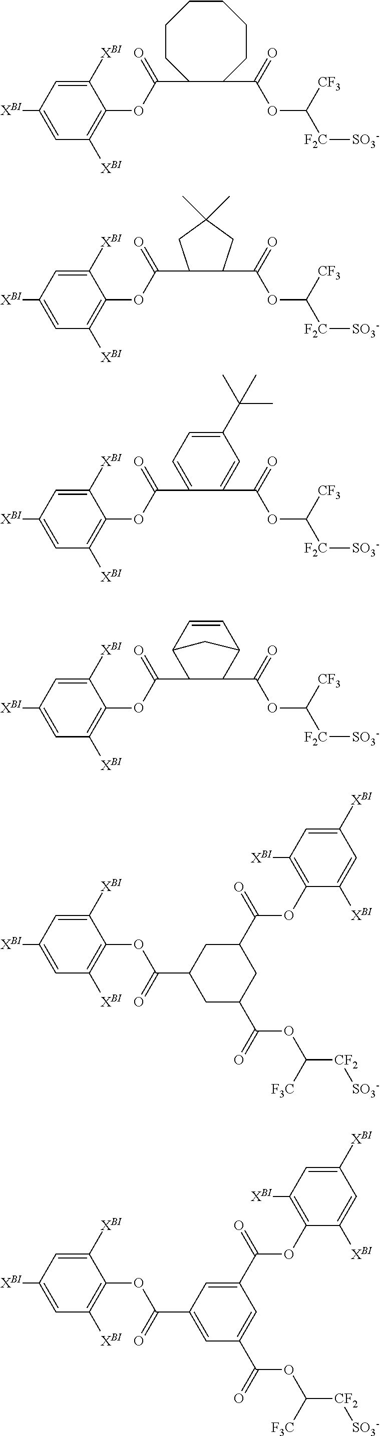

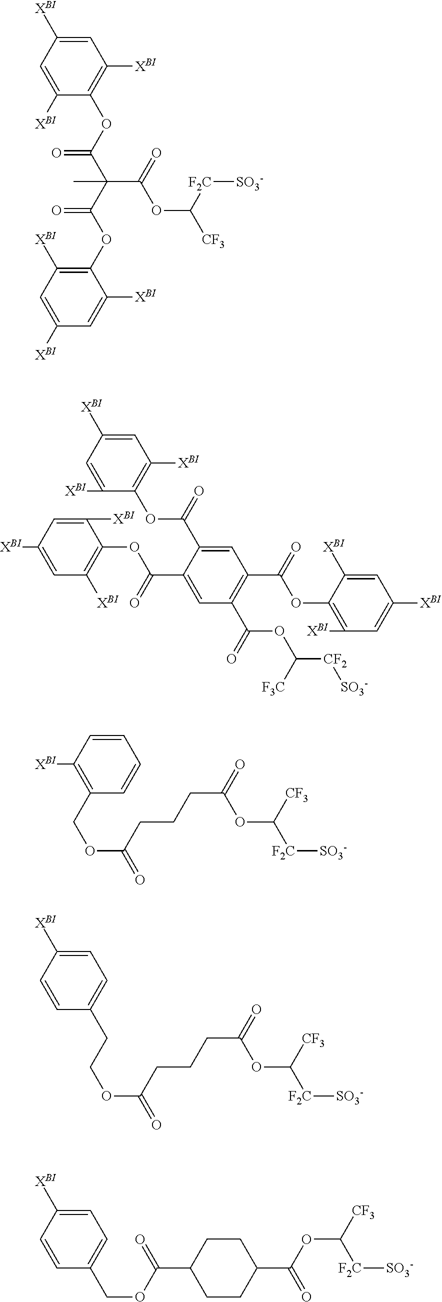

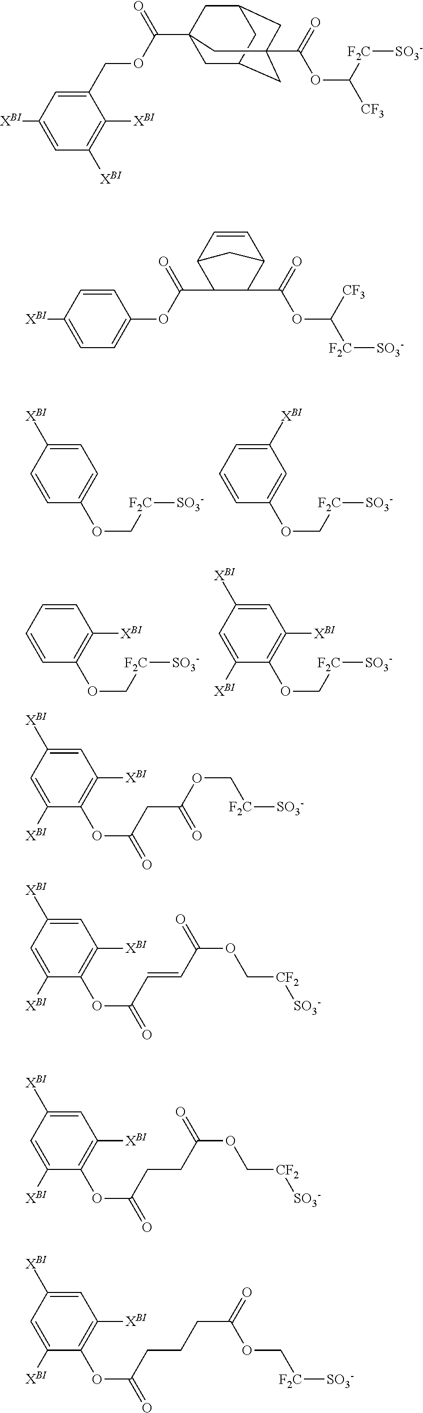

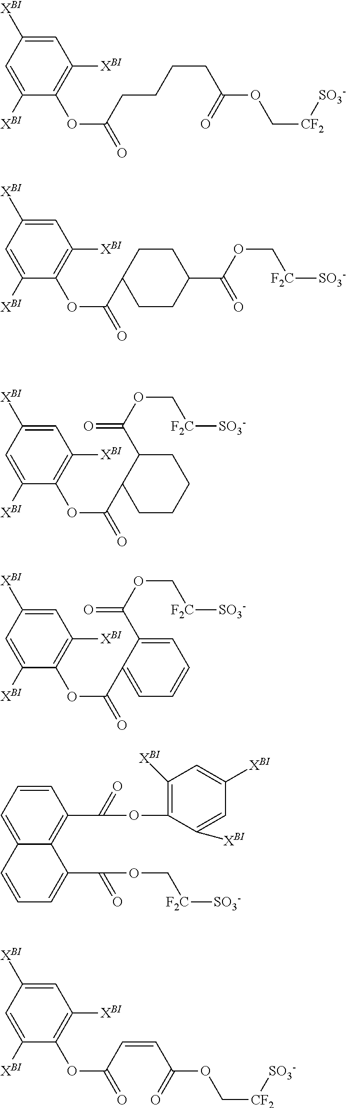

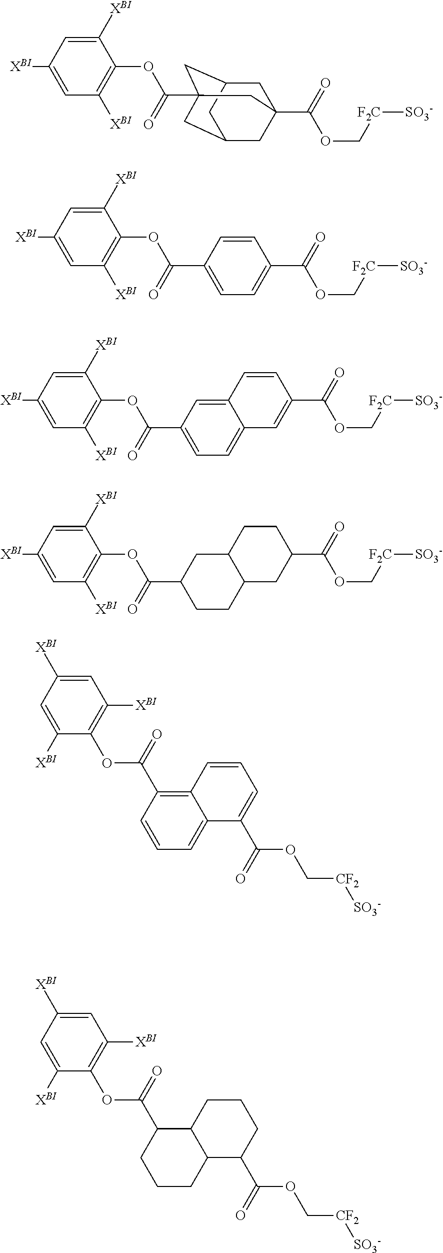

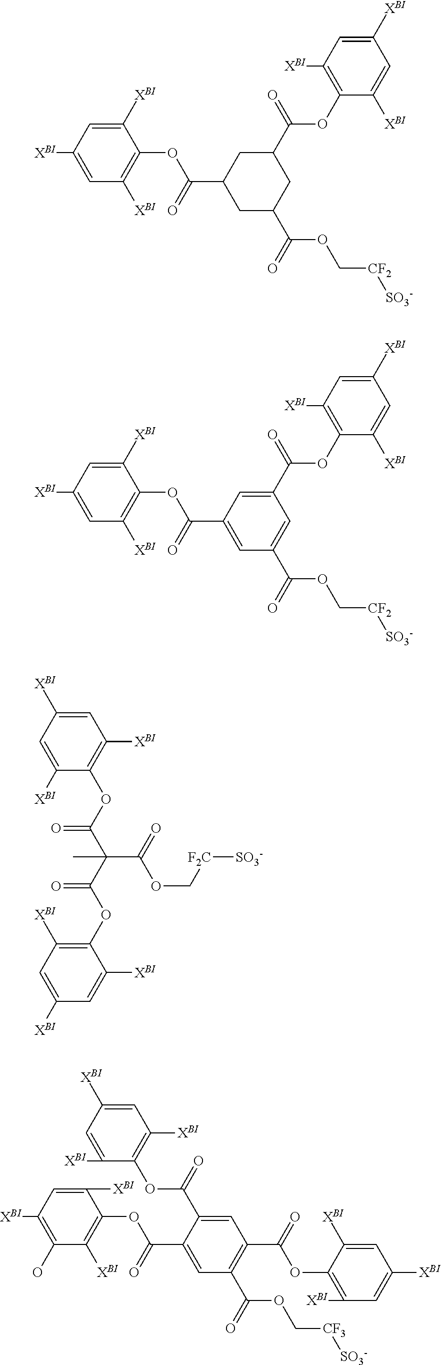

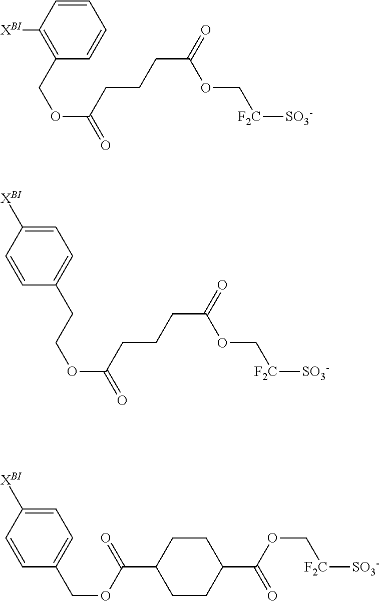

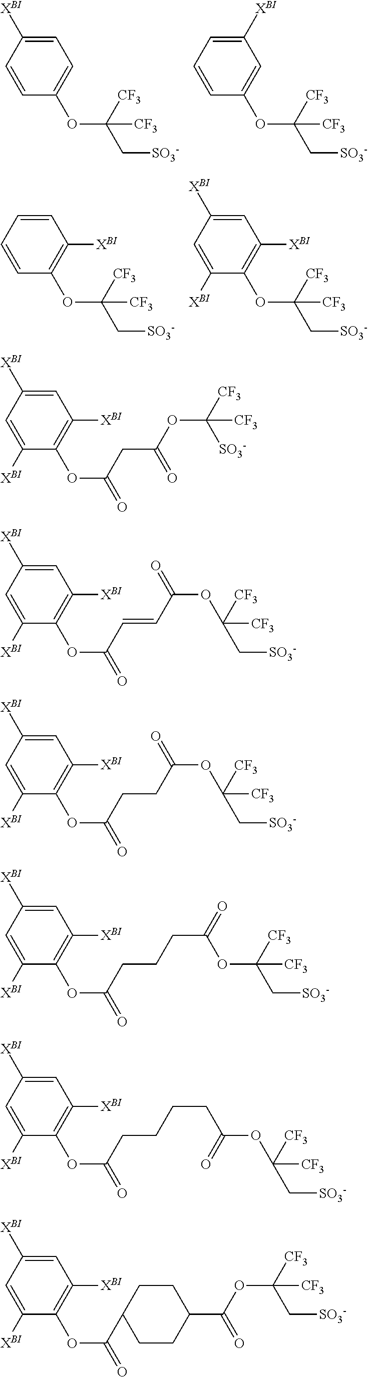

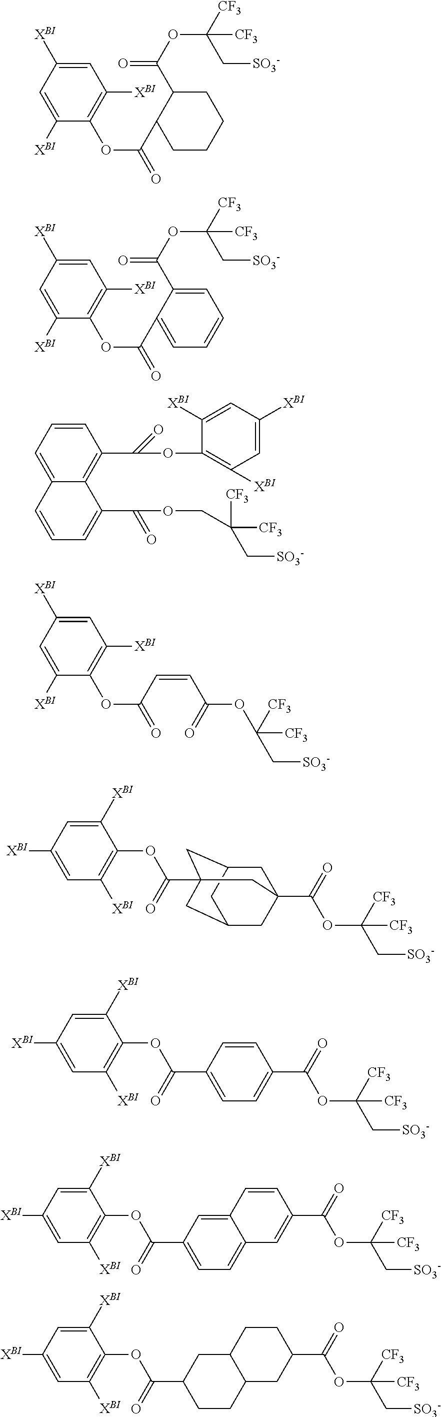

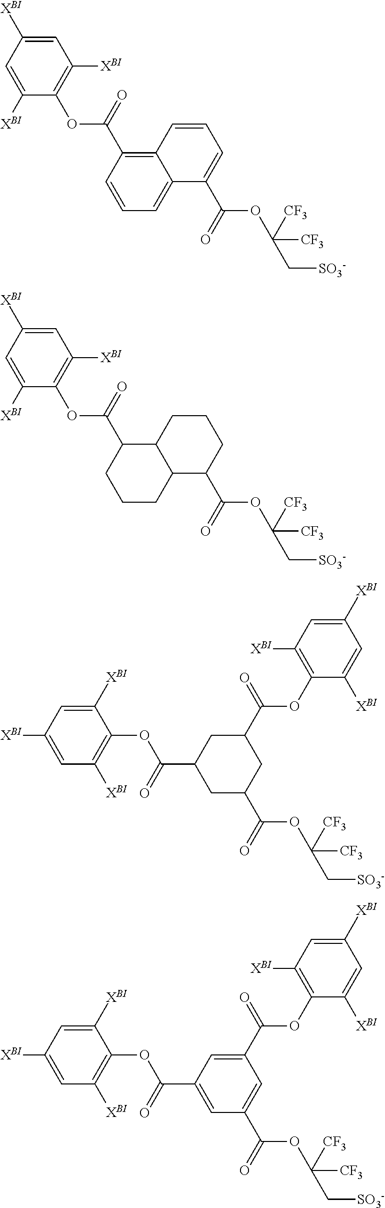

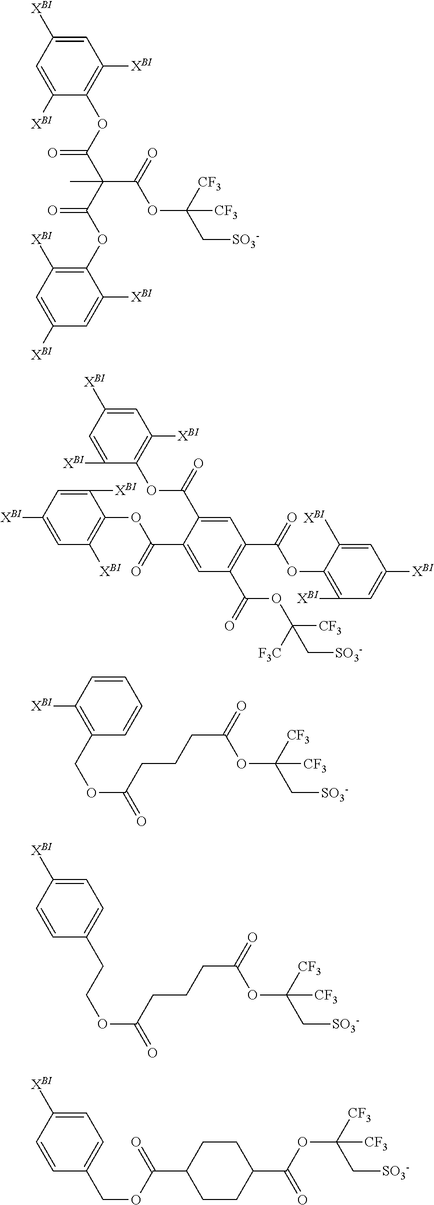

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159.

Further useful examples of the non-nucleophilic counter ion include bulky fluorine-free benzenesulfonic acid anions as described in JP-A 2006-276759, JP-A 2015-117200, JP-A 2016-065016, and JP-A 2019-202974; fluorine-free benzenesulfonic acid or alkylsulfonic acid anions having an iodized aromatic group bonded thereto as described in JP 6645464.

Also useful are bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989.

In formulae (c2) and (c3), L¹ is a single bond, ether bond, ester bond, carbonyl, sulfonic ester bond, carbonate bond or carbamate bond. From the aspect of synthesis, an ether bond, ester bond or carbonyl is preferred, with the ester bond or carbonyl being more preferred.

In formula (c2), Rf¹ and Rf² are each independently fluorine or a C₁-C₆ fluorinated saturated hydrocarbyl group. It is preferred that both Rf¹ and Rf² be fluorine because the generated acid has a higher acid strength. Rf³ and Rf⁴ are each independently hydrogen, fluorine or a C₁-C₆ fluorinated saturated hydrocarbyl group. It is preferred for solvent solubility that at least one of Rf⁵ and Rf⁶ be trifluoromethyl.

In formula (c3), Rf⁵ and Rf⁶ are each independently hydrogen, fluorine or a C₁-C₆ fluorinated saturated hydrocarbyl group. It is excluded that all Rf⁵ and Rf⁶ are hydrogen at the same time. It is preferred for solvent solubility that at least one of Rf⁵ and Rf⁶ be trifluoromethyl.

In formulae (c2) and (c3), d is an integer of 0 to 3, preferably 1.

Examples of the repeat unit (c2) are shown below, but not limited thereto. R^{A} is as defined above.

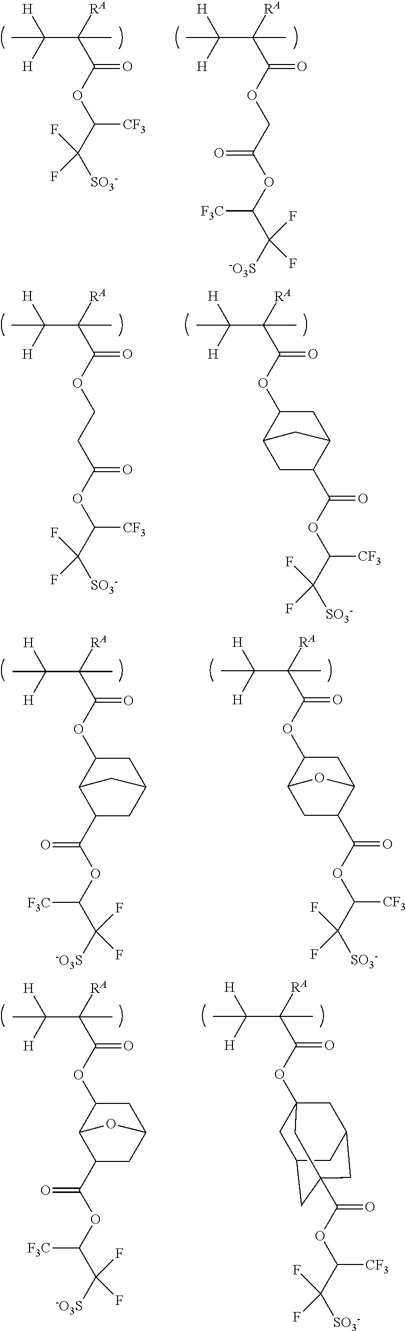

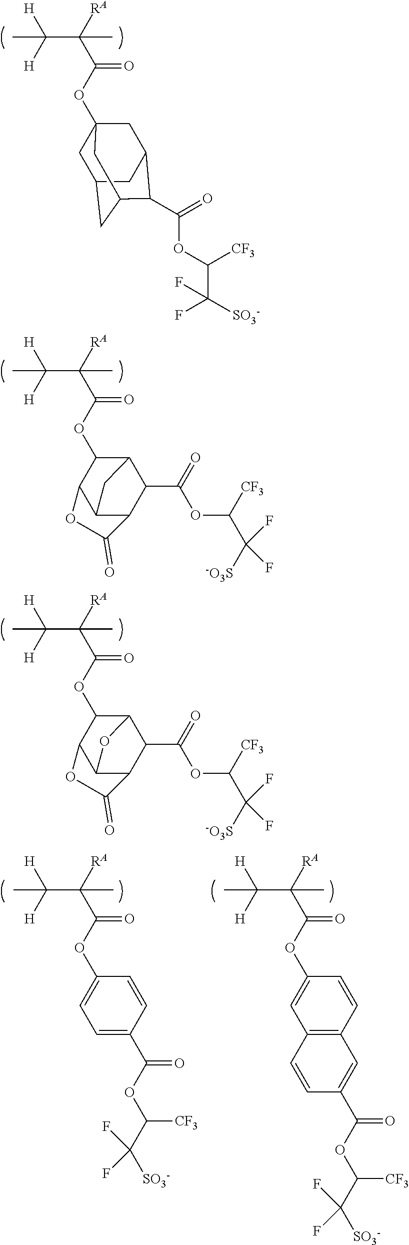

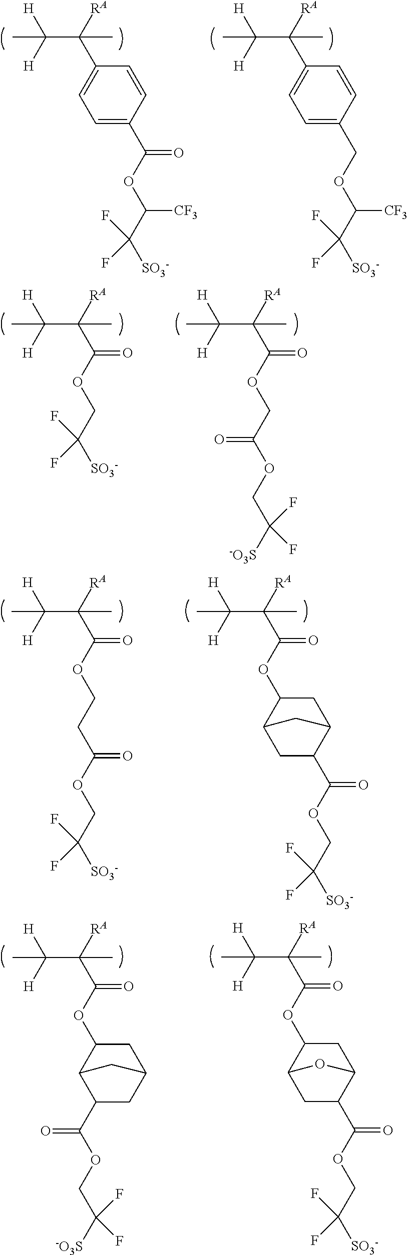

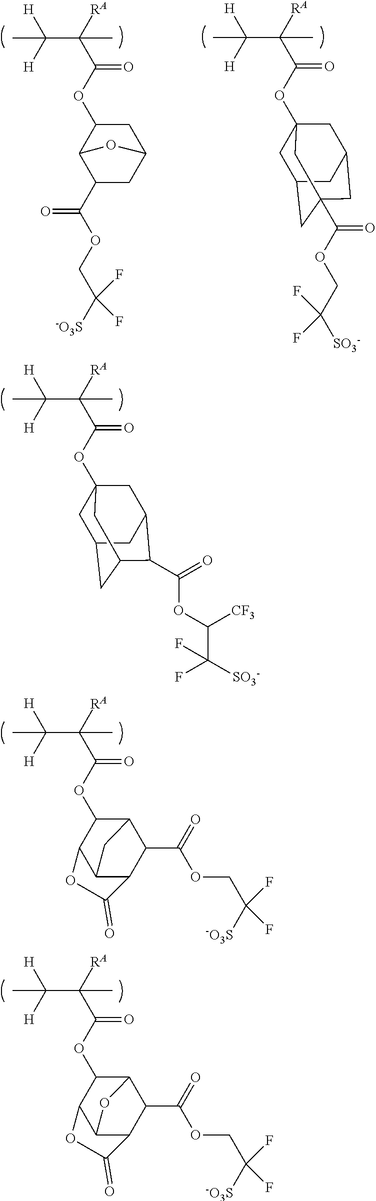

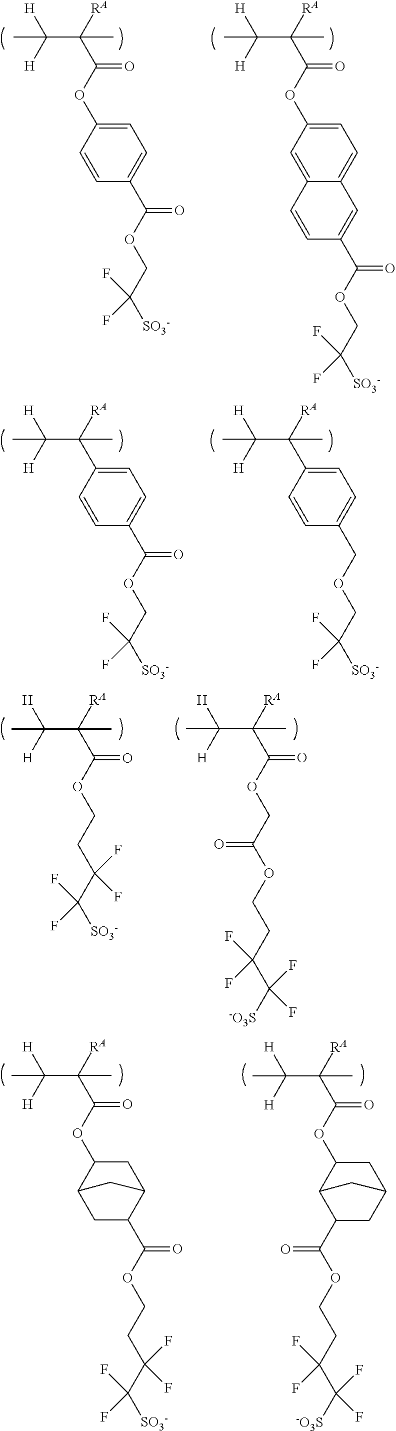

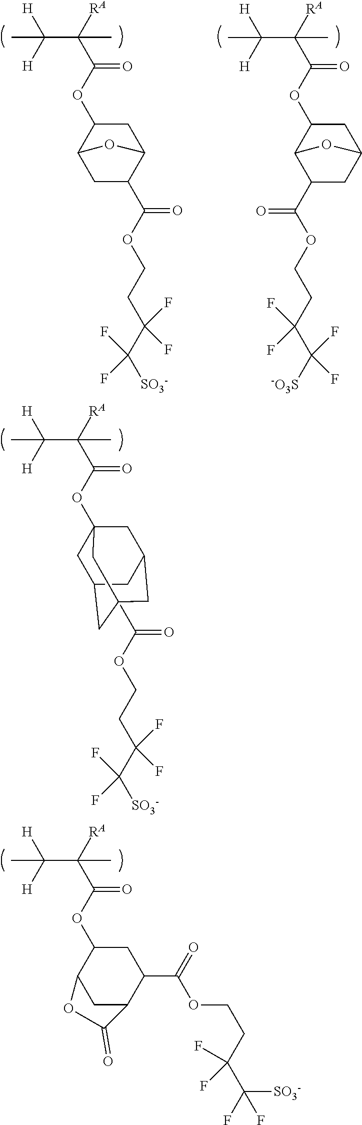

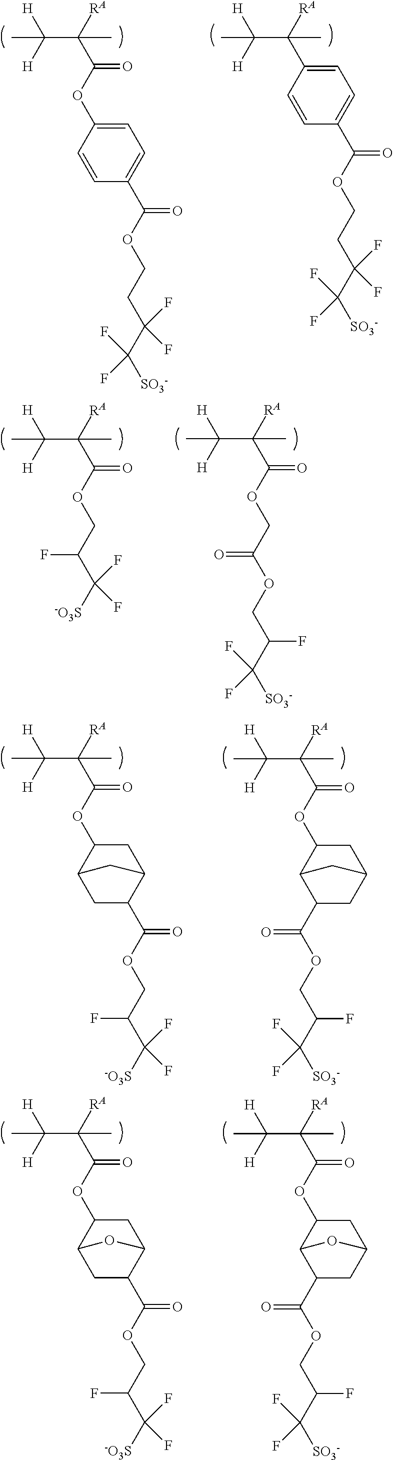

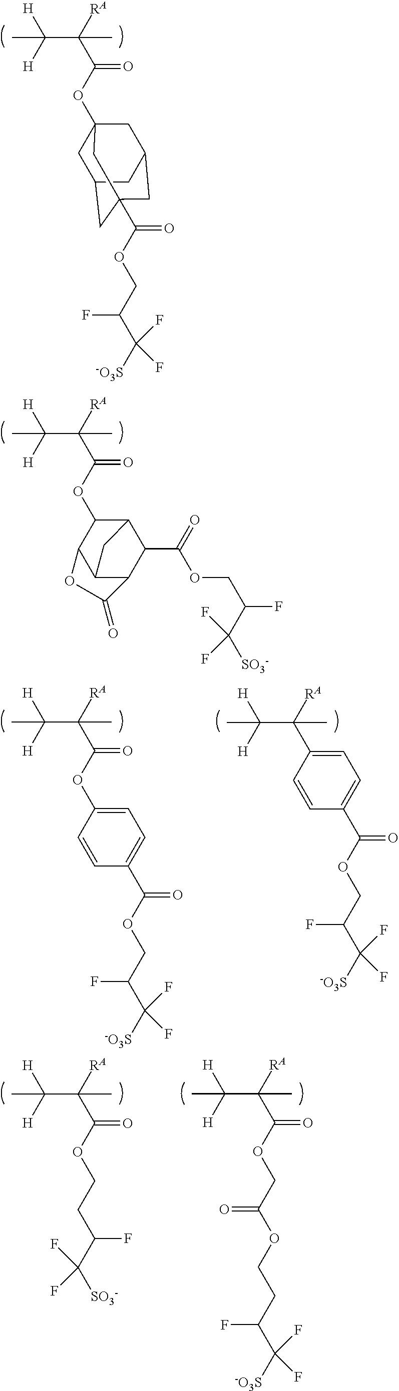

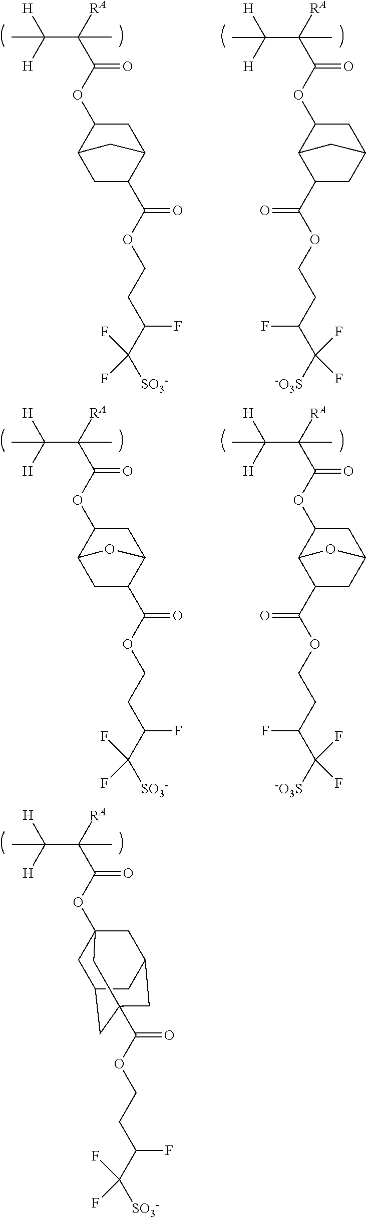

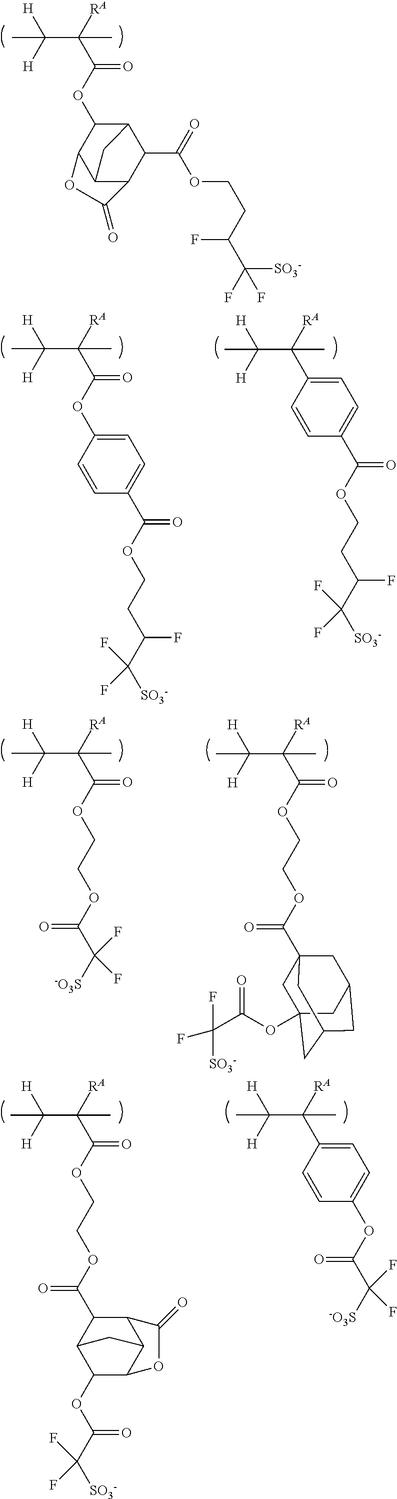

Examples of the anion in repeat unit (c3) are shown below, but not limited thereto. R^{A} is as defined above.

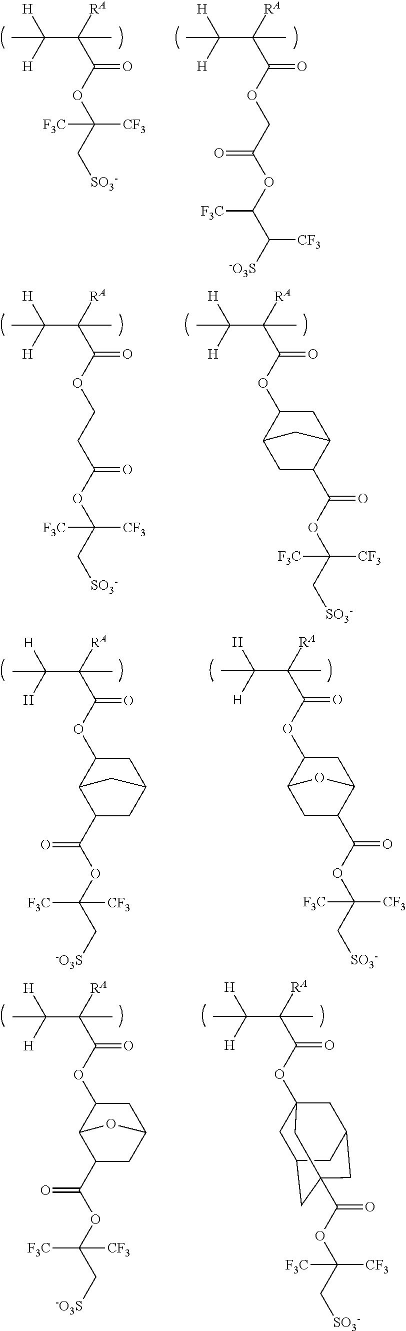

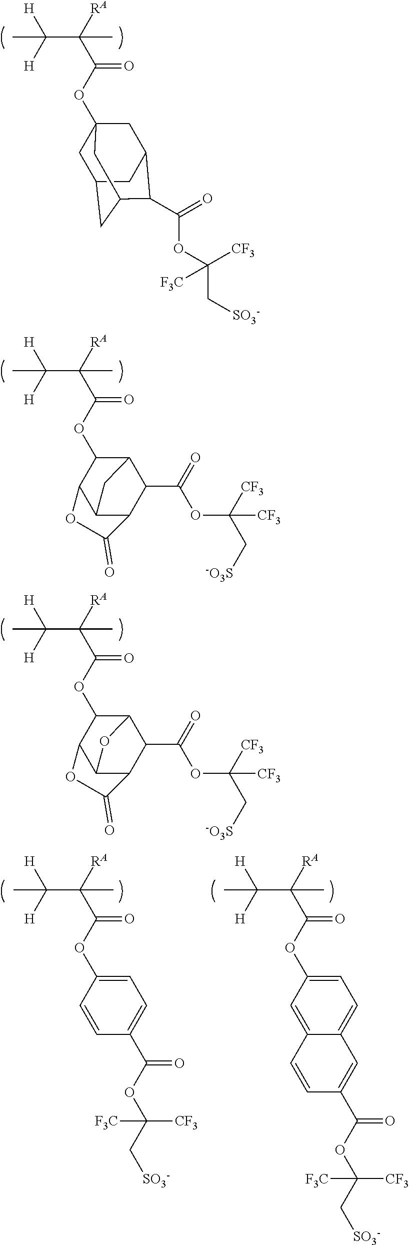

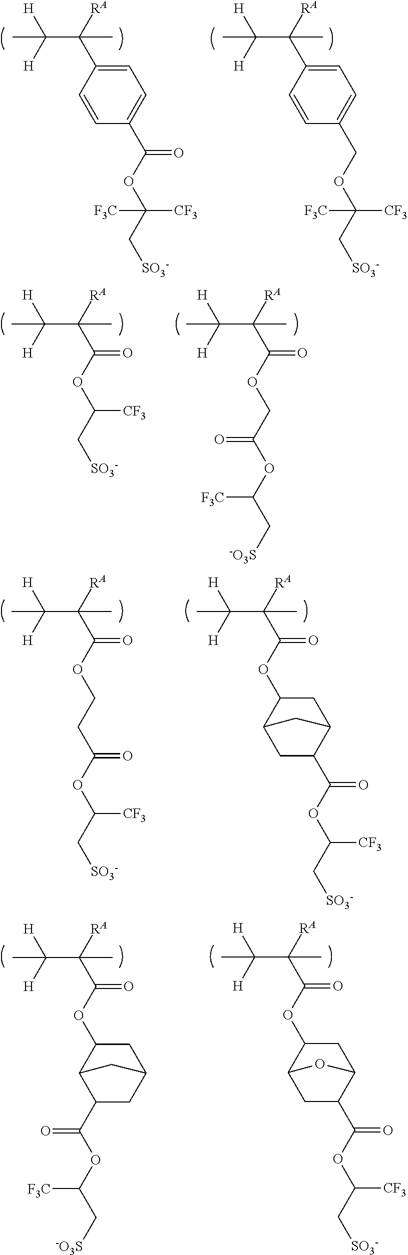

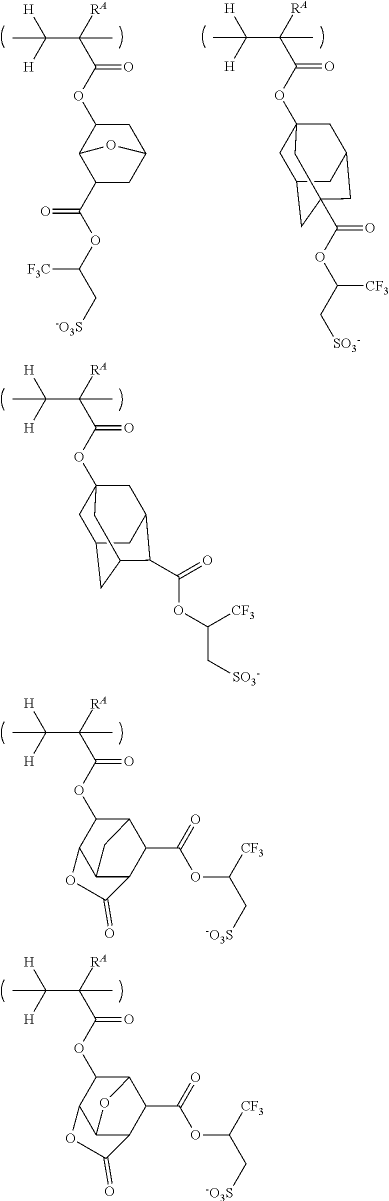

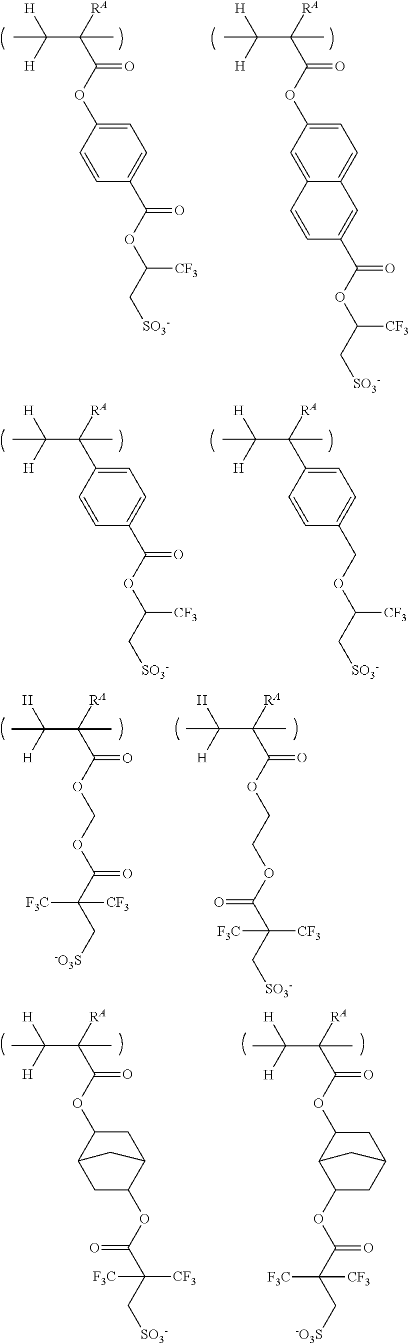

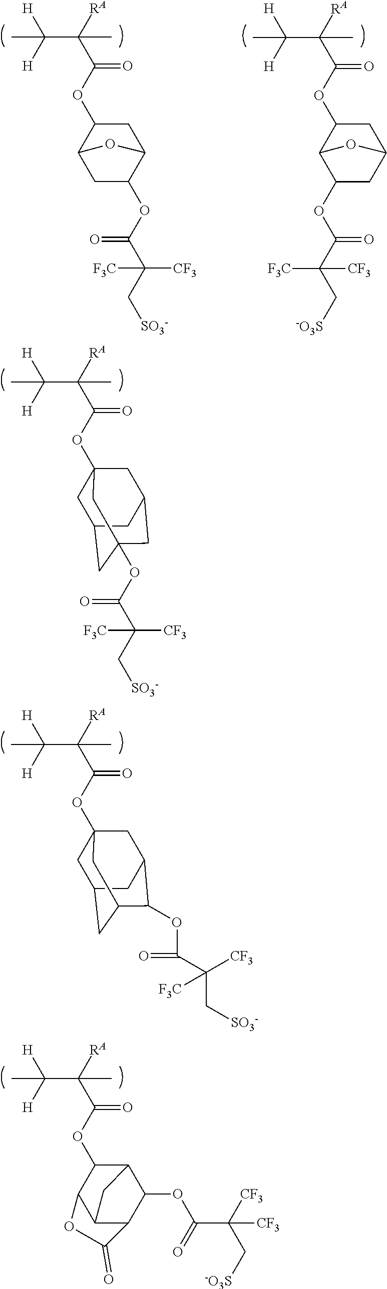

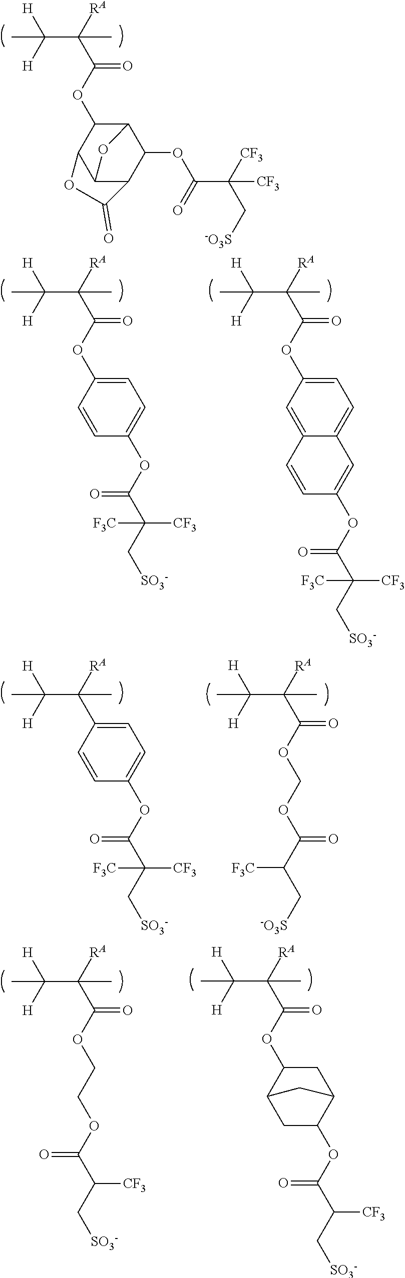

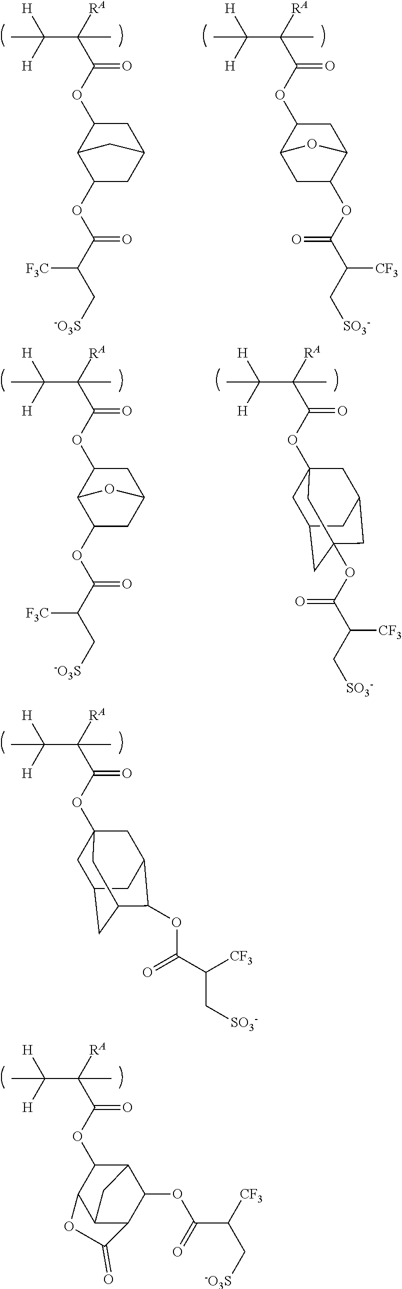

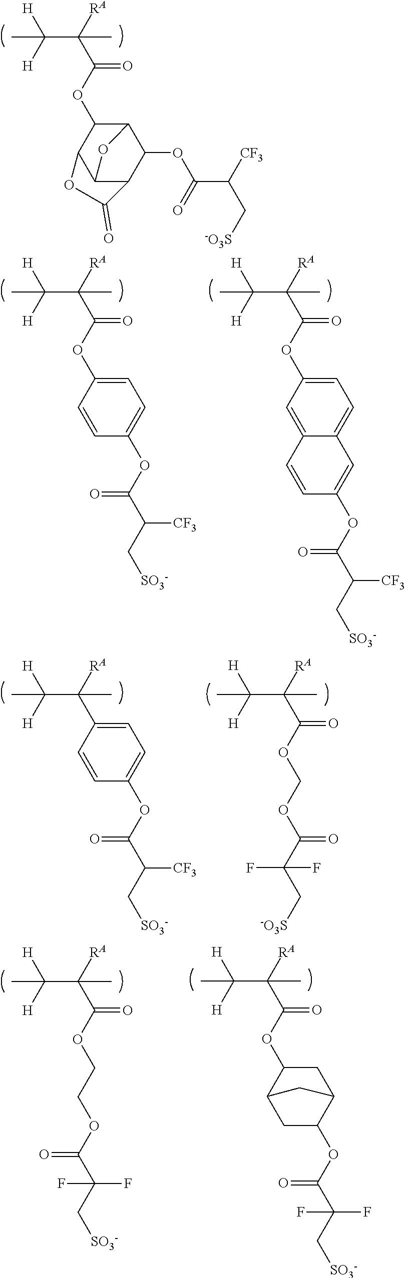

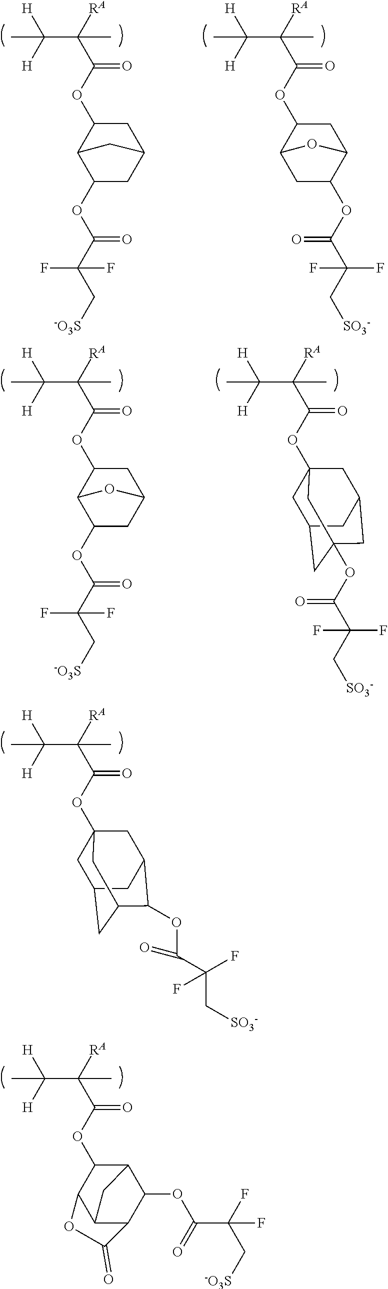

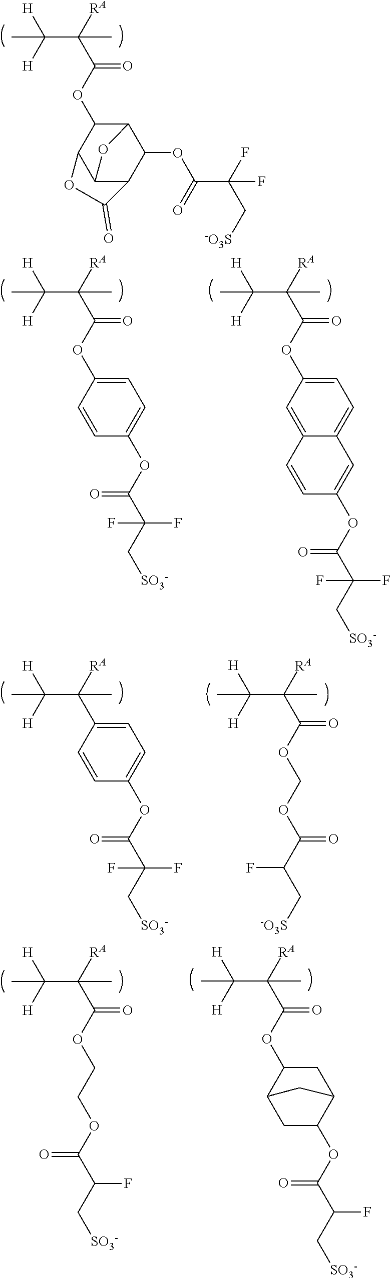

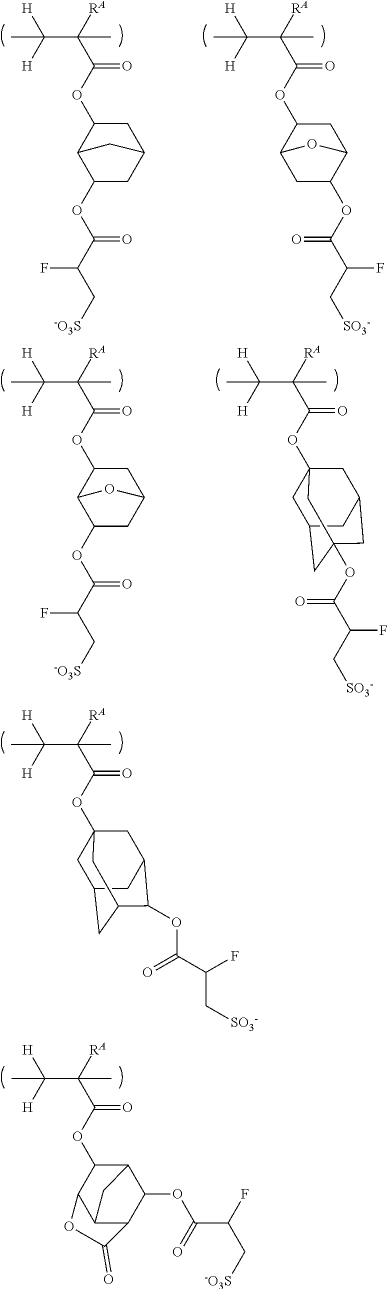

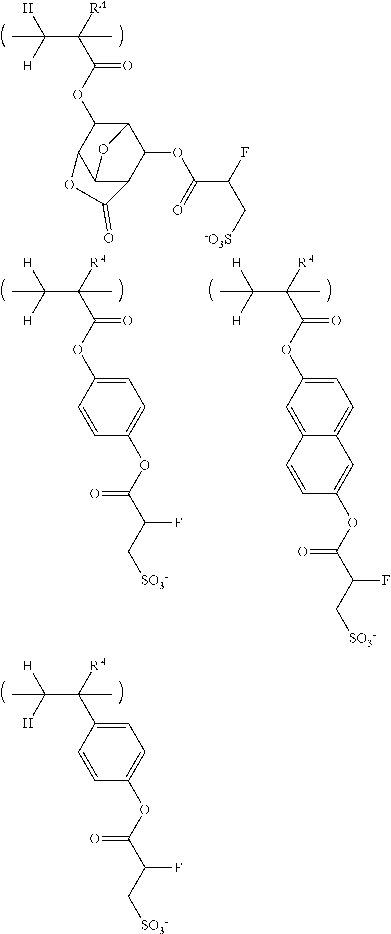

Examples of the anion in repeat unit (c4) are shown below, but not limited thereto. R^{A} is as defined above.

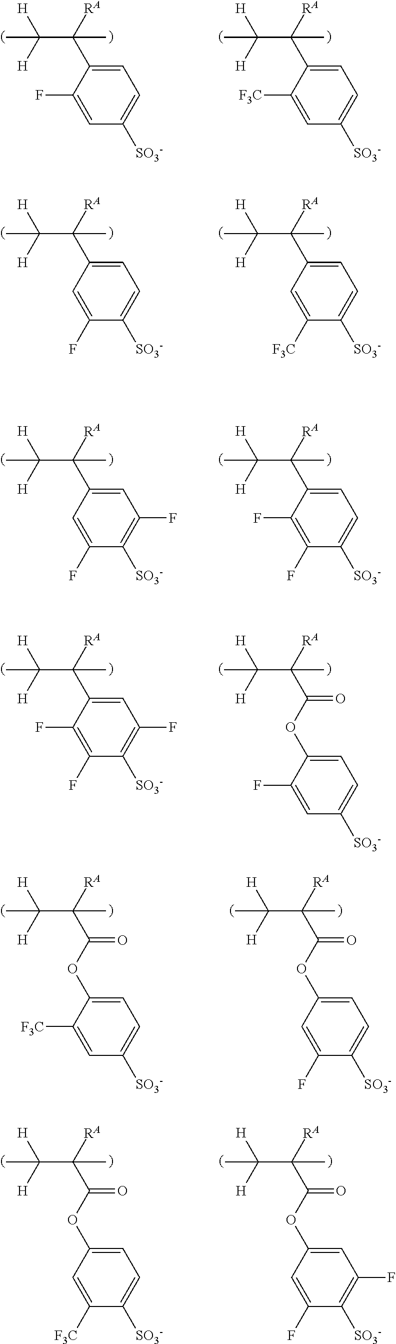

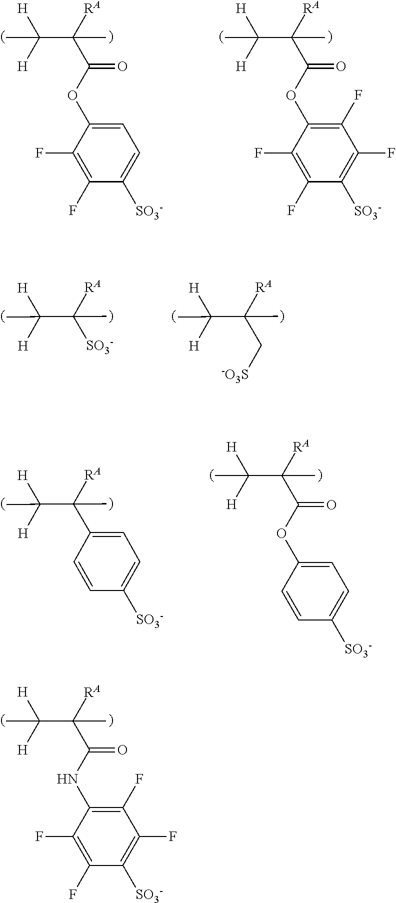

In formulae (c2) to (c4). A⁺ is an onium cation. Suitable onium cations include ammonium, sulfonium and iodonium cations, with the sulfonium and iodonium cations being preferred. Examples thereof are as exemplified above for the cation having formula (cation-1) and the cation having formula (cation-2) and as will be exemplified later for the cation having formula (cation-3).

Exemplary structures of the repeat units (c1) to (c4) include arbitrary combinations of anions with cations, both as exemplified above.

Of the repeat units (c1) to (c4), repeat units (c2), (c3) and (c4) are preferred from the aspect of controlling acid diffusion, repeat units (c2) and (c4) are more preferred from the aspect of the acid strength of generated acid, and repeat units (c2) are most preferred from the aspect of solvent solubility.

The base polymer may further comprise repeat units (d) of a structure having a hydroxy group protected with an acid labile group. The repeat unit (d) is not particularly limited as long as the unit includes one or more structures having a hydroxy group protected with a protective group such that the protective group is decomposed to generate the hydroxy group under the action of acid. Repeat units having the formula (d1) are preferred.

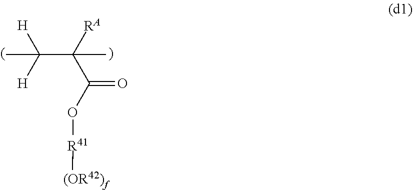

In formula (d1), R^{A} is as defined above. R⁴¹ is a C₁-C₃₀ (f+1)-valent hydrocarbon group which may contain a heteroatom. R⁴² is an acid labile group, and f is an integer of 1 to 4.

In formula (d1), the acid labile group R⁴² is deprotected under the action of acid so that a hydroxy group is generated. The structure of R⁴² is not particularly limited, an acetal structure, ketal structure, alkoxycarbonyl group and alkoxymethyl group having the following formula (d2) are preferred, with the alkoxymethyl group having formula (d2) being more preferred.

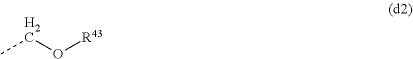

Herein R⁴³ is a C₁-C₁₅ hydrocarbyl group.

Illustrative examples of the acid labile group R⁴², the alkoxymethyl group having formula (d2), and the repeat units (d) are as exemplified for the repeat units (d) in JP-A 2020-111564 (US 20200223796).

In addition to the foregoing units, the base polymer may further comprise repeat units (e) derived from indene, benzofiuran, benzothiophene, acenaphthylene, chromone, coumarin, norbornadiene and derivatives thereof. Examples of the monomer from which repeat units (e) are derived are shown below, but not limited thereto.

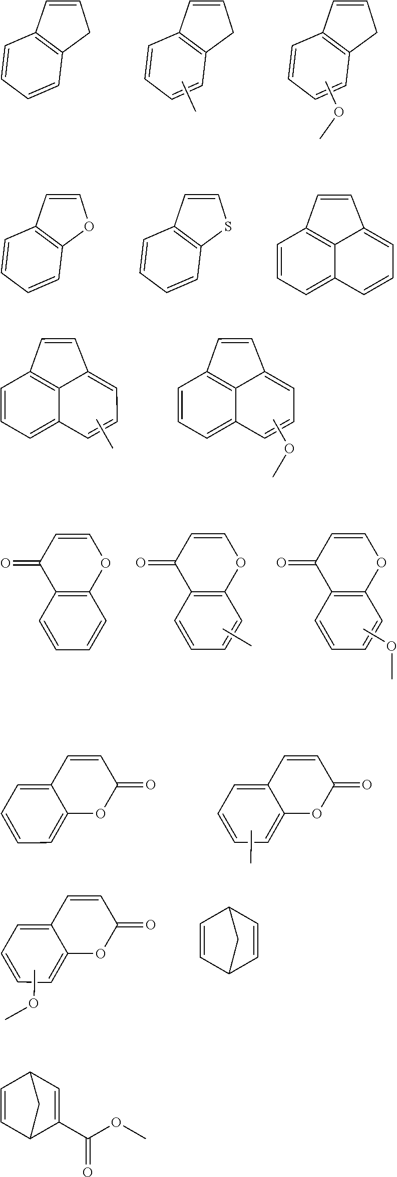

The base polymer may further comprise repeat units (f) derived from indane, vinylpyridine or vinylcarbazole.

In the polymer, repeat units (a1), (a2), (b1). (b2), (c1) to (c4), (d), (e), and (f) are incorporated in a ratio of preferably 0<a1≤0.8, 0≤a2≤0.8, 0≤b1≤0.6, 0≤b2≤0.6, 0≤c1≤0.4, 0≤c2≤0.4, 0≤c3≤0.4, 0≤c4≤0.4, 0d≤0.5, 0≤e≤0.3, and 0≤f≤0.3; more preferably 0<a1≤0.7, 0≤a2≤0.7, 0≤b1≤0.5, 0≤b2≤0.5, 0≤c1≤50.3, 0≤c2≤0.3, 0≤c3≤0.3, 0≤c4≤0.3, 0≤d≤0.3, 0≤e≤0.3, and 0≤f≤0.3.

The polymer should preferably have a weight average molecular weight (Mw) in the range of 1,000 to 500,000, and more preferably 3,000 to 100,000, as measured by GPC versus polystyrene standards using tetrahydrofiuran (THF) or N,N-dimethylformamide (DMF) solvent. A Mw in the range ensures that the resist film has etch resistance and eliminates the risk of resolution decline by a failure to provide a difference in dissolution rate before and after exposure.

The influence of Mw/Mn becomes stronger as the pattern rule becomes finer. Therefore, the polymer should preferably have a narrow dispersity (Mw/Mn) of 1.0 to 2.0 in order to provide a resist composition suitable for micropatterning to a small feature size. A Mw/Mn in the range ensures that the contents of lower and higher molecular weight polymer fractions are low and eliminates a possibility that foreign matter is left on the pattern or the pattern profile is degraded.

The polymer may be synthesized, for example, by dissolving a monomer or monomers corresponding to the above-mentioned repeat units in an organic solvent, adding a radical polymerization initiator, and heating for polymerization.

Examples of the organic solvent which can be used for polymerization include toluene, benzene, THF, diethyl ether, dioxane, cyclohexane, cyclopentane, methyl ethyl ketone (MEK), propylene glycol monomethyl ether acetate (PGMEA), and y-butyrolactone (GBL). Examples of the polymerization initiator used herein include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours in view of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form a ultra-high-molecular-weight polymer, it is preferred from the standpoint of quality control to prepare the monomer solution and the initiator solution separately and add them dropwise. The acid labile group that has been incorporated in the monomer may be kept as such, or polymerization may be followed by protection or partial protection. During the polymer synthesis, any known chain transfer agent such as dodecyl mercaptan or 2-mercaptoethanol may be added for molecular weight control purpose. The amount of chain transfer agent added is preferably 0.01 to 20 mol % based on the total of monomers.

When a hydroxy-containing monomer is copolymerized, the hydroxy group is substituted with an acetal group which is susceptible to deprotection with acid, typically ethoxyethoxy, prior to polymerization, and the polymerization is followed by deprotection with weak acid and water. Alternatively, the hydroxy group is substituted with an acetyl, formyl or pivaloyl group prior to polymerization, and the polymerization is followed by alkaline hydrolysis.

When hydroxystyrene or hydroxyvinylnaphthalene is copolymerized, one method is dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or polyhydroxyvinylnaphthalene.

For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The amounts of monomers in the monomer solution may be determined appropriate so as to provide the preferred fractions of repeat units.

It is now described how to use the polymer obtained by the above preparation method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145] (U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol: keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as y-butyrolactone (GBL); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflonl, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The base polymer (B) may be used alone or as a blend of two or more polymers which differ in compositional ratio, Mw and/or Mw/Mn. Component (B) may also be a blend of the base polymer defined above and a hydrogenated product of ROMP. For the ROMP, reference is made to JP-A 2003-066612.

(C) Organic Solvent

The resist composition may comprise an organic solvent as component (C). The organic solvent (C) is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA): ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as y-butyrolactone (GBL), and mixtures thereof.

Of the foregoing organic solvents, it is recommended to use 1-ethoxy-2-propanol, PGMEA, cyclohexanone, GBL, DAA and mixtures thereof because the base polymer (B) is most soluble therein.

The organic solvent (C) is preferably added in an amount of 200 to 5,000 parts by weight, and more preferably 400 to 3,500 parts by weight per 80 parts by weight of the base polymer (B). The organic solvent may be used alone or in admixture.

(D) Quencher

The resist composition may further comprise a quencher as component (D). As used herein, the quencher refers to a compound capable of trapping the acid generated by the PAG in the resist composition to prevent the acid from diffusing to the unexposed region, for thereby forming the desired pattern.

Onium salts having the formulae (2) and (3) are useful as the quencher (D).

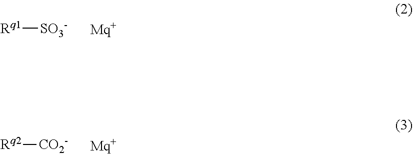

In formula (2), R^{q1} is hydrogen or a C₁-C₄₀ hydrocarbyl group which may contain a heteroatom, exclusive of the hydrocarbyl group in which the hydrogen atom bonded to the carbon atom at α-position of the sulfo group is substituted by fluorine or fluoroalkyl.

The hydrocarbyl group R^{q1} may be saturated or unsaturated and straight, branched or cyclic. Examples thereof include C₁-C₄ alkyl groups such as methyl, ethyl, n-propyl, isopropyl, n-butyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentyhmethyl, cyclopentylethyl, cyclopentylbutyl, cyclohexylmethyl, cyclohexylethyl, cyclohexylbutyl, norbomyl, tricyclo[5.2.1.0^{2,6}]decyl, and adamantyl: C₆-C₄₀ aryl groups such as phenyl, naphthyl and anthracenyl, and combinations thereof. In these hydrocarbyl groups, some or all hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety.

In formula (3), R^{V} is hydrogen or a C₁-C₄₀ hydrocarbyl group which may contain a heteroatom. Examples of the hydrocarbyl group R^{V} include those exemplified above for R^{q1} and fluorinated saturated hydrocarbyl groups such as trifluoromethyl and trifluoroethyl, and fluorinated aryl groups such as pentafluorophenyl and 4-trifluoromethylphenyl.

Examples of the anion in the onium salt having formula (2) are shown below, but not limited thereto.

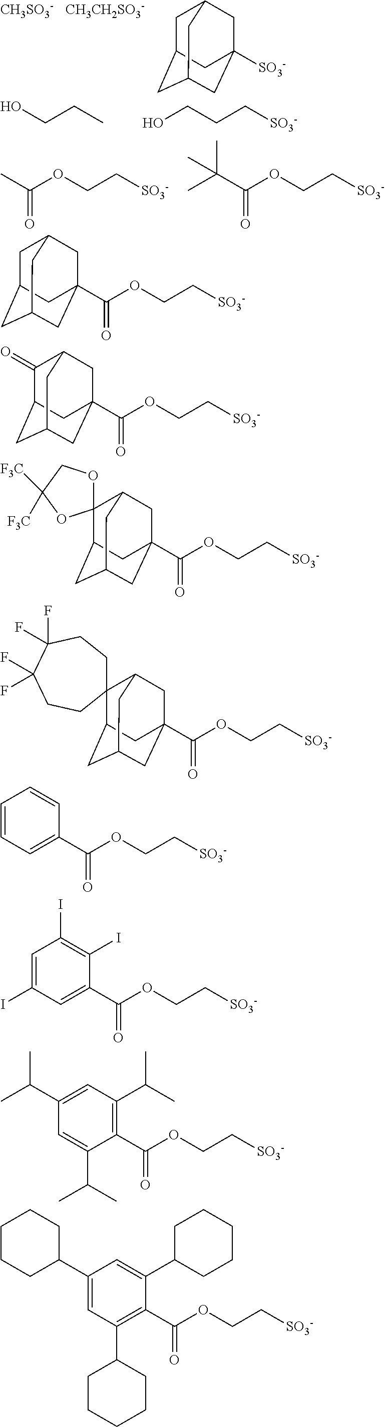

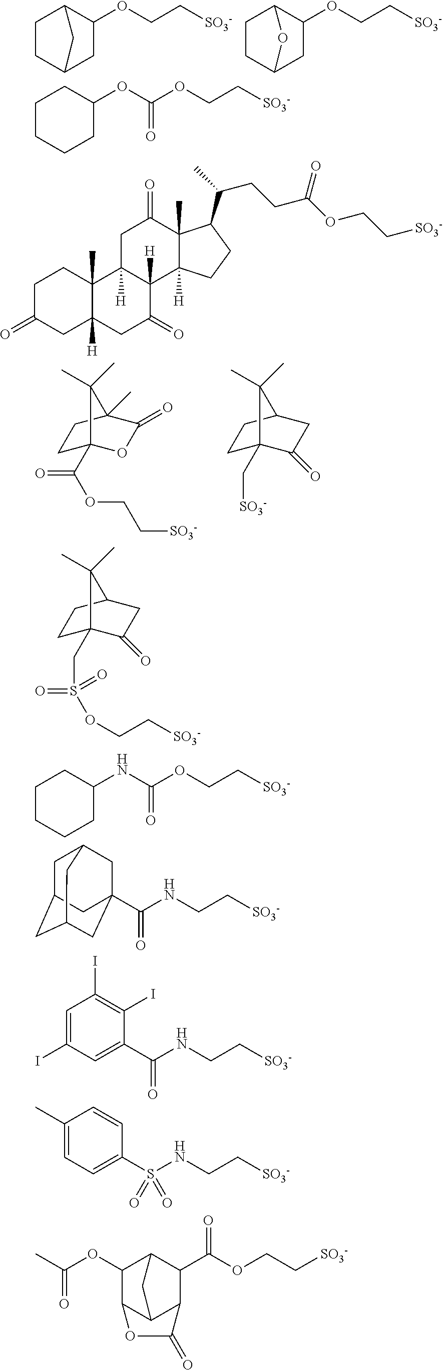

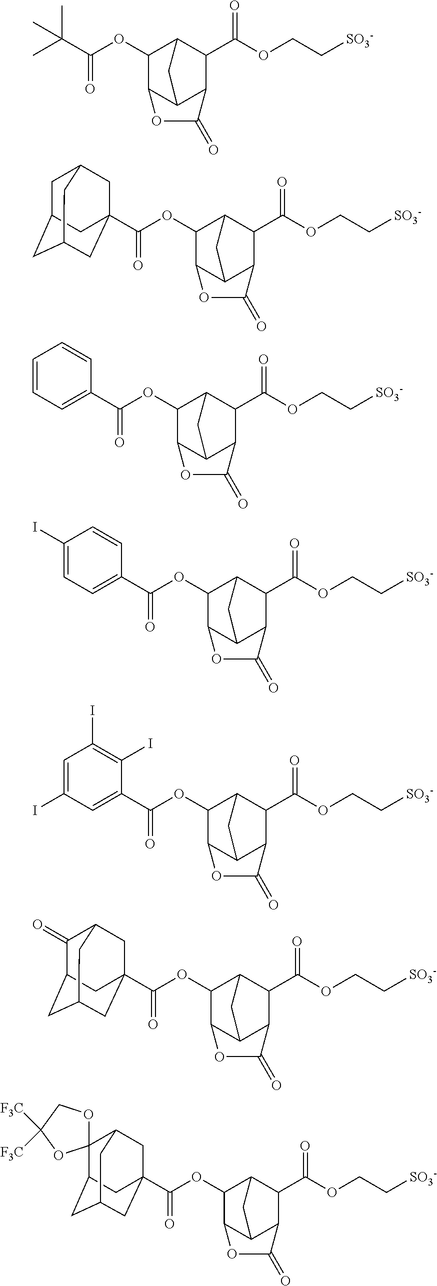

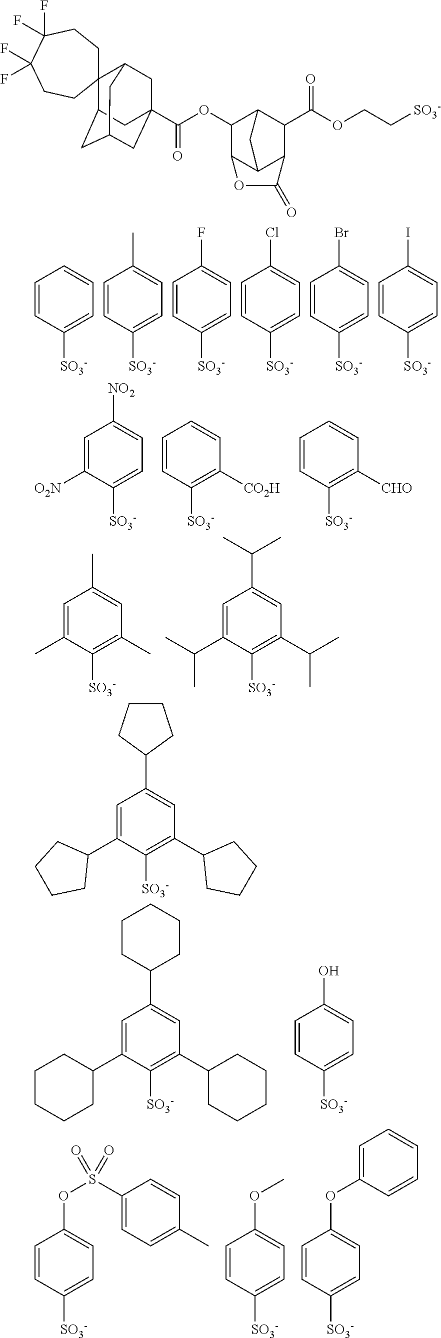

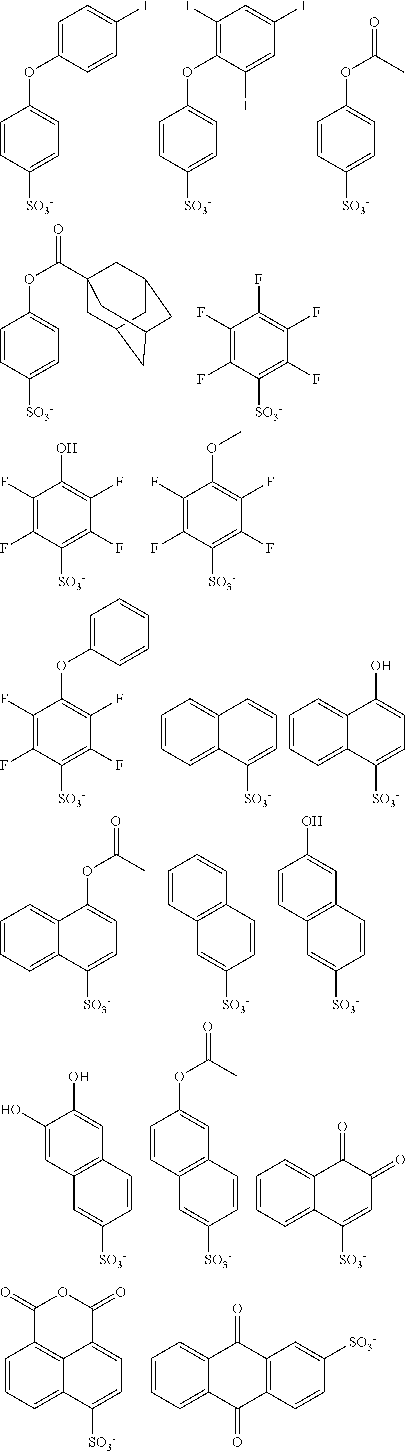

Examples of the anion in the onium salt having formula (3) are shown below, but not limited thereto.

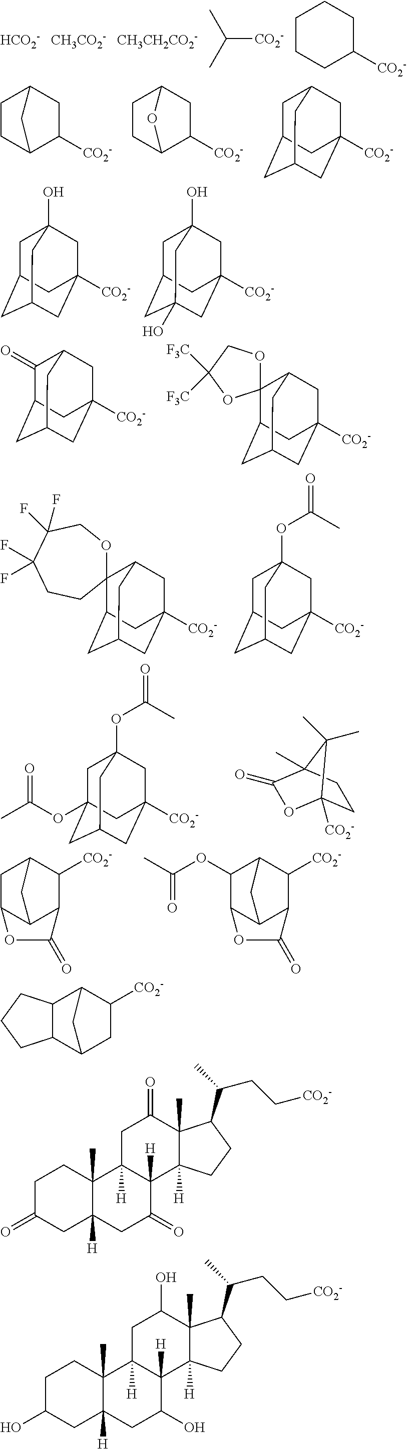

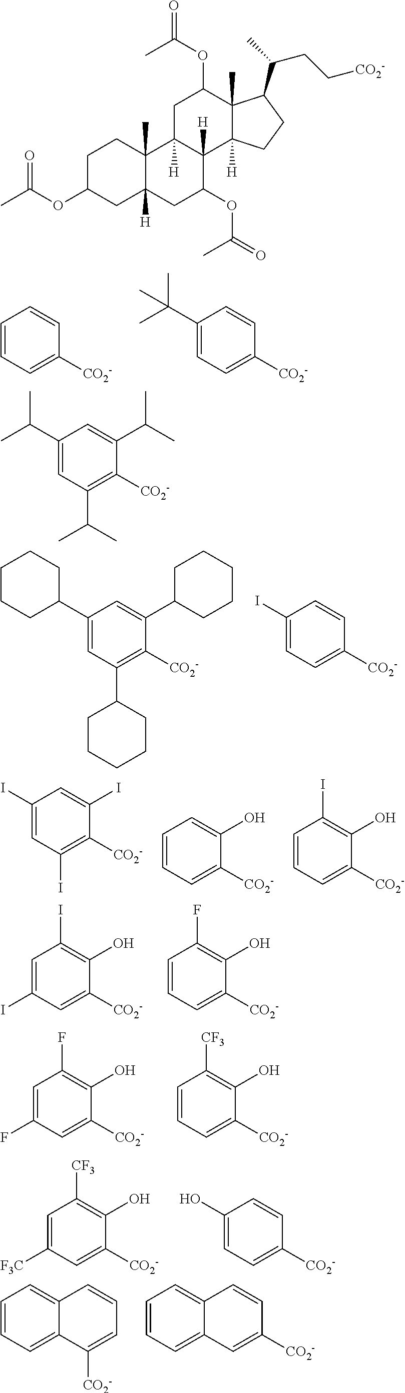

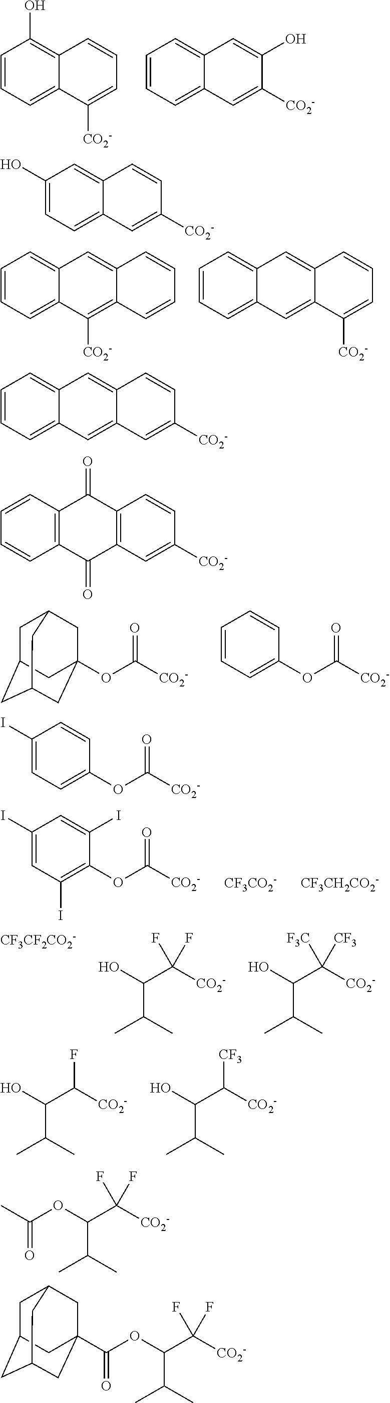

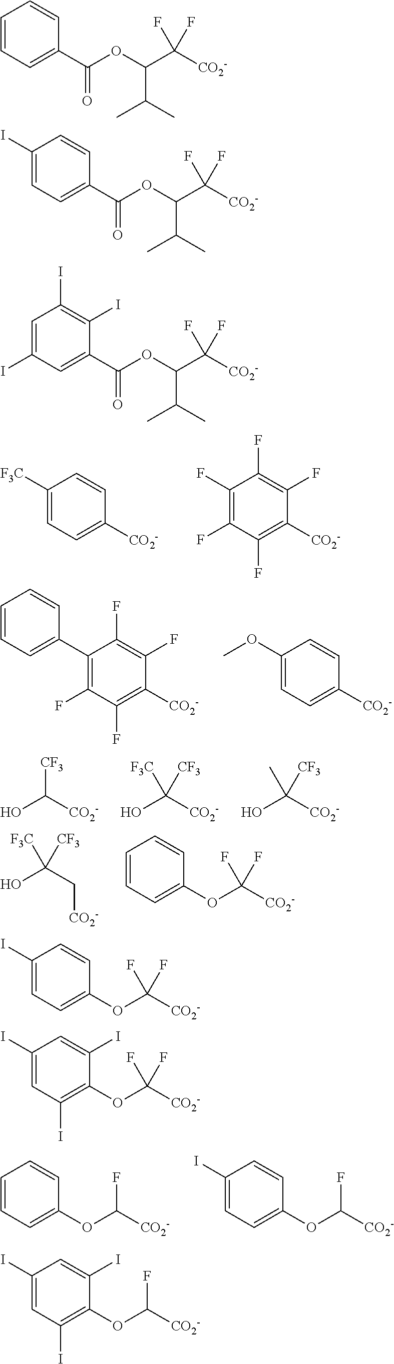

In formulae (2) and (3), Mq⁺ is an onium cation, which is preferably selected from sulfonium cations having formula (cation-1), iodonium cations having formula (cation-2), and ammonium cations having the following formula (cation-3).

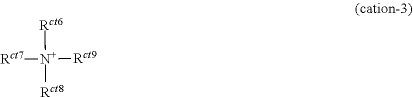

In formula (cation-3), R^{ct6} to R^{ct9} are each independently a C₁-C₄₀ hydrocarbyl group which may contain a heteroatom. A pair of R^{ct6} and R^{ct7} may bond together to form a ring with the nitrogen atom to which they are attached. Examples of the hydrocarbyl group are as exemplified above for R^{ct1} to R^{ct5} in formulae (cation-1) and (cation-2).

Examples of the ammonium cation having formula (cation-3) are shown below, but not limited thereto.

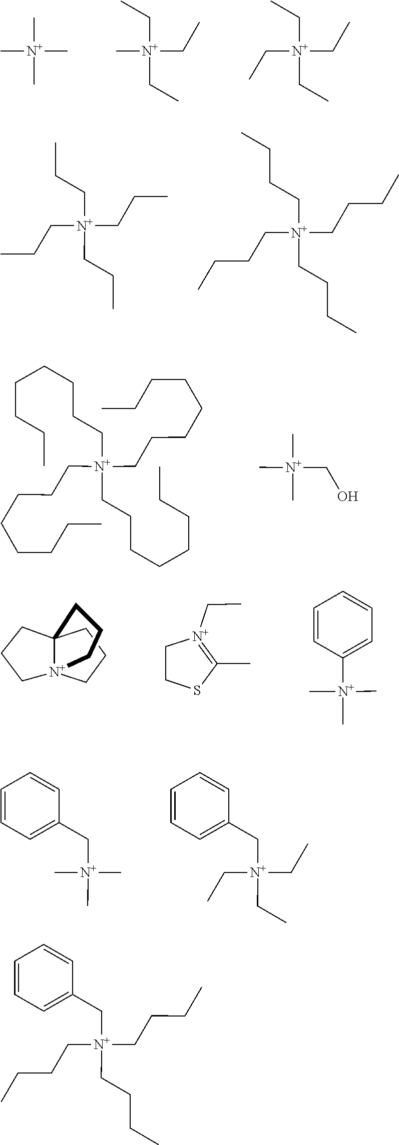

Examples of the onium salt having formula (2) or (3) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (2) or (3) functions as a quencher in the chemically amplified resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (2) or (3) functions as a quencher when used in combination with an onium salt type PAG having a conjugated base of a strong acid (typically a sulfonic acid which is fluorinated at α-position) as the counter anion. In a system using a mixture of an onium salt capable of generating a strong acid (e.g., α-position fluorinated sulfonic acid) and an onium salt capable of generating a weak acid (e.g., non-fluorinated sulfonic acid or carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

Also useful as the quencher (D) are onium salts having a sulfonium cation and a phenoxide anion site in a common molecule as described in JP 6848776, onium salts having a sulfonium cation and a carboxylate anion site in a common molecule as described in JP 6583136 and JP-A 2020-200311, and onium salts having an iodonium cation and a carboxylate anion site in a common molecule as described in JP 6274755.

If a PAG capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the onium salt having formula (2) or (3) is used as the quencher (D), the amount of the onium salt used is preferably 0.1 to 20 parts by weight, more preferably 0.1 to 10 parts by weight per 80 parts by weight of the base polymer (B). As long as the amount of component (D) is in the range, a satisfactory resolution is available without a substantial lowering of sensitivity. The onium salt having formula (2) or (3) may be used alone or in admixture.

Also nitrogen-containing compounds may be used as the quencher (D). Suitable nitrogen-containing compounds include primary, secondary and tertiary amine compounds, specifically amine compounds having a hydroxy group, ether bond, ester bond, lactone ring, cyano group or sulfonic ester bond, as described in JP-A 2008-111103, paragraphs [0146]-[0164] (U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the nitrogen-containing compound is used as the quencher (D), the amount of the nitrogen-containing compound used is preferably 0.001 to 12 parts by weight, more preferably 0.01 to 8 parts by weight per 80 parts by weight of the base polymer (B). The nitrogen-containing compound may be used alone or in admixture.

(E) Other Photoacid Generator

The chemically amplified resist composition may comprise (E) a photoacid generator other than component (A). The other PAG is not particularly limited as long as it is capable of generating an acid upon exposure to high-energy radiation. The preferred other PAG is a salt having the formula (4) or (5).

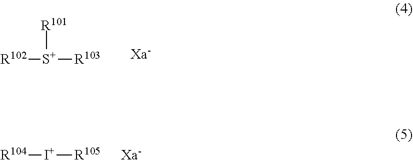

In formula (4), R¹⁰¹ to R¹⁰⁵ are each independently a C₁-C₂₀ hydrocarbyl group which may contain a heteroatom. Any two of R¹⁰¹, R¹⁰² and R¹⁰³ may bond together to form a ring with the sulfur atom to which they are attached. Examples of the hydrocarbyl group are as exemplified above for R^{ct1} to R^{ct5} in formulae (cation-1) and (cation-2).

Examples of the sulfonium cation in the salt having formula (4) are as exemplified above for the sulfonium cation having formula (cation-1). Examples of the iodonium cation in the salt having formula (5) are as exemplified above for the iodonium cation having formula (cation-2).

In formulae (4) and (5), Xa⁻ is an anion of strong acid selected from formulae (c1-1) to (c1-5).

Compounds having the formula (6) are also preferred as the other PAG (E).

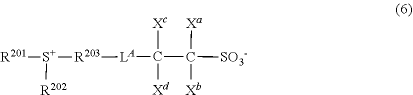

In formula (6), R²⁰¹ and R²⁰² are each independently a C₁-C₃₀ hydrocarbyl group which may contain a heteroatom. R²³ is a C₁-C₃₀ hydrocarbylene group which may contain a heteroatom. Any two of R²⁰¹, R²⁰² and R²⁰³ may bond together to form a ring with the sulfur atom to which they are attached.

The C₁-C₃₀ hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, n-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexyhnethyl, cyclohexylethyl, cyclohexylbutyl, norbornyl, oxanorbomyl, tricyclo[5.2.1.0^{2,6}]decyl, and adamantyl; and C₁-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl, and combinations thereof. In these hydrocarbyl groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl: and arylene groups such as phenylene, methylphenylene, ethylphenylene, n-propyiphenylene, isopropylphenylene, n-butylphenylene, isobutylphenylene, sec-butylphenylene, tert-butylphenylene, naphthylene, methylnaphthylene, ethylnaphthylene, n-propyinaphthylene, isopropyinaphthylene, n-butyinaphthylene, isobutylnaphthylene, sec-butylnaphthylene, and tert-butylnaplithylene. In these hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred.

In formula (6), L^{A} is a single bond, ether bond or a C₁-C₂₀ hydrocarbylene group which may contain a heteroatom. The hydrocarbylene group may be saturated or unsaturated and straight, branched or cyclic. Examples thereof are as exemplified above for the hydrocarbylene group R²⁰³.

In formula (6), X^{a}, X^{b}, X^{c} and X^{d} are each independently hydrogen, fluorine or trifluoromethyl, at least one of X^{a}, X^{b}, X^{c} and X^{d} being fluorine or trifluoromethyl.

Of the PAGs having formula (6) those having formula (6′) are preferred.

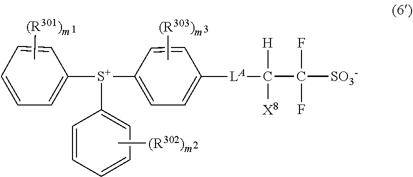

In formula (6′), L^{A} is as defined above. X° is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for R^{fa1} in formula (c1-1-1). The subscripts m¹ and m2 are each independently an integer of 0 to 5, and m³ is an integer of 0 to 4.

Examples of the PAG having formula (6) include those exemplified for the PAG having formula (2) in JP-A 2017-026980.

Of the foregoing PAGs, those having an anion of formula (c1-1-1) or (c1-4) are especially preferred because of reduced acid diffusion and high solubility in solvents. Also those having formula (6′) are especially preferred because of extremely reduced acid diffusion.

When used, the other PAG (E) is preferably added in an amount of 0.1 to 40 parts, and more preferably 0.5 to 20 parts by weight per 80 parts by weight of the base polymer (B). As long as the amount of the PAG is in the range, good resolution is achievable and the risk of foreign particles being formed after development or during stripping of resist film is avoided. The other PAG may be used alone or in admixture.

(F) Surfactant

The resist composition may further include (F) a surfactant which is insoluble or substantially insoluble in water and soluble in alkaline developer, and/or a surfactant which is insoluble or substantially insoluble in water and alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

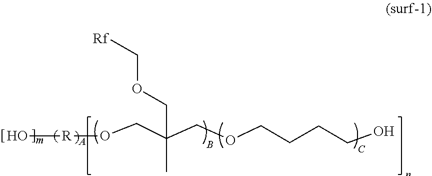

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

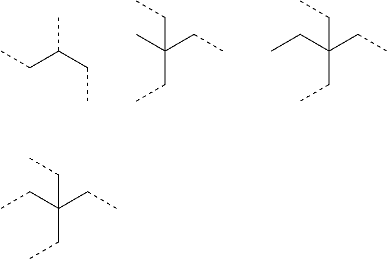

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (7A) to (7E).

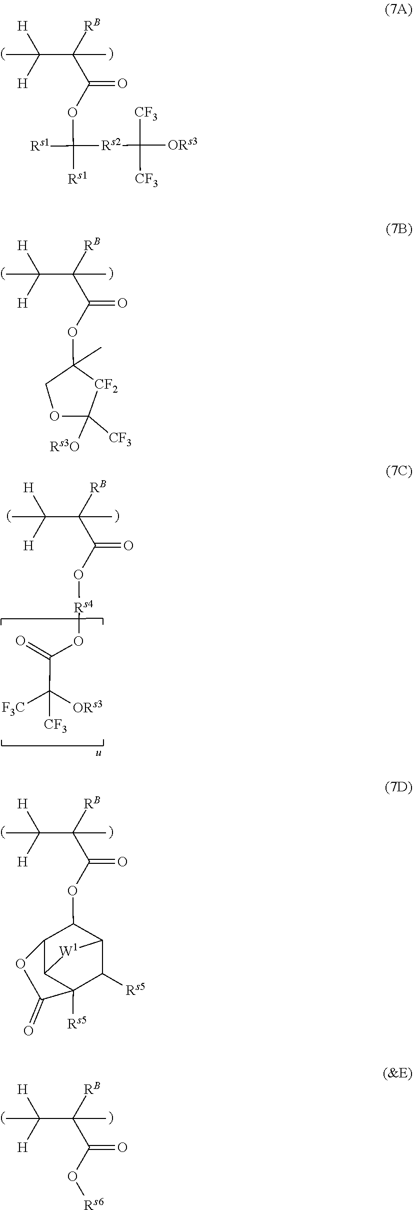

Herein, R^{B} is hydrogen, fluorine, methyl or trifluoromethyl. W¹ is —CH₂—, —CH₂CH₂— or —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R¹ is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is an integer of 1 to 3. R^{s5} is each independently hydrogen or a group: —C(═O)—O—R wherein R¹ is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group represented by R^{s1} may be straight, branched or cyclic and is preferably saturated one. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, n-decyl, and C₃-C₁₀ cyclic saturated hydrocarbyl groups such as cyclopropyl, cyclobutyl, cyclopentyl, cyclohexyl, adamantyl, and norbomyl. Inter alia, C₁-C₆ hydrocarbyl groups are preferred.

The hydrocarbylene group represented by R^{s2} may be straight, branched or cyclic. Examples thereof include methylene, ethylene, propylene, butylene and pentylene.

The hydrocarbyl group represented by R^{s3} or R^{s6} may be saturated or unsaturated and straight, branched or cyclic. Examples thereof include saturated hydrocarbyl groups, and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic and examples thereof include the foregoing hydrocarbyl or fluorinated hydrocarbyl groups from which u number of hydrogen atoms are eliminated.

The fluorinated hydrocarbyl group represented by R^{sa} may be straight, branched or cyclic and is preferably saturated. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (7A) to (7E) are shown below, but not limited thereto. Herein R^{B} is as defined above.

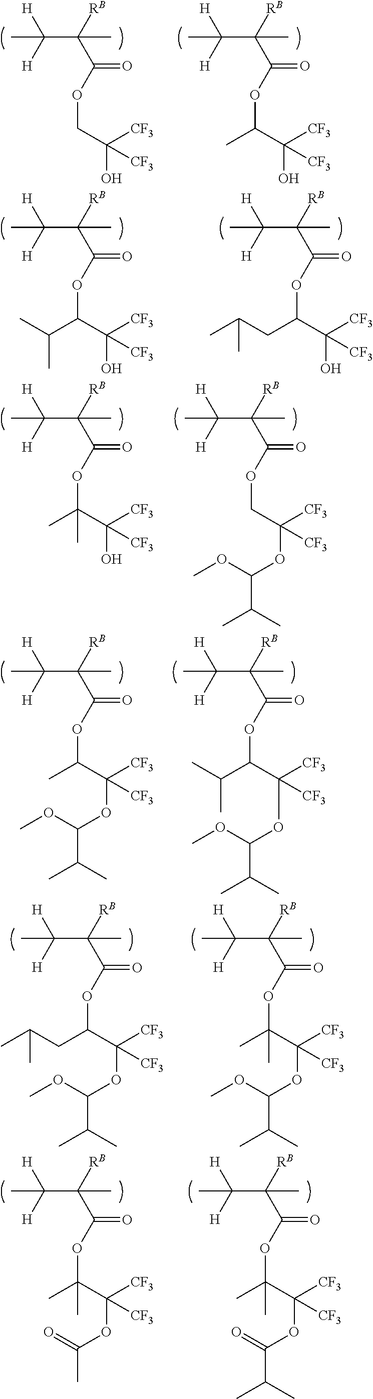

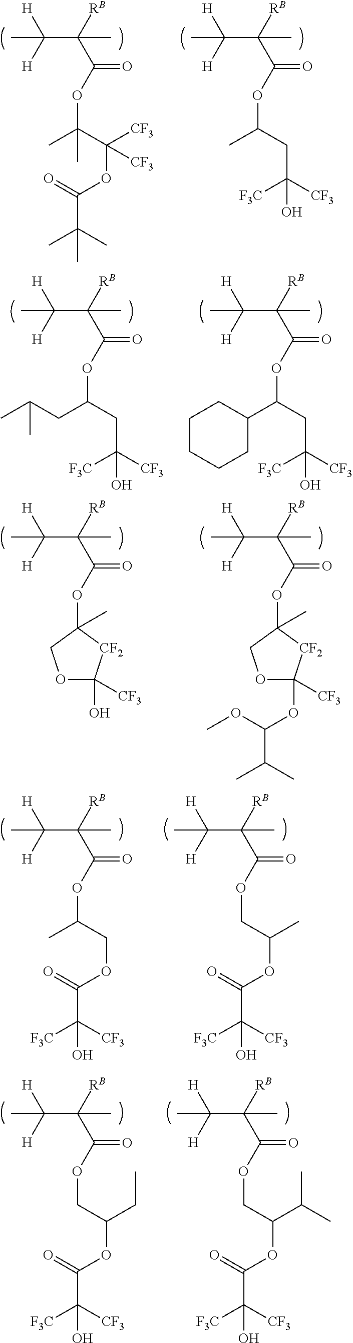

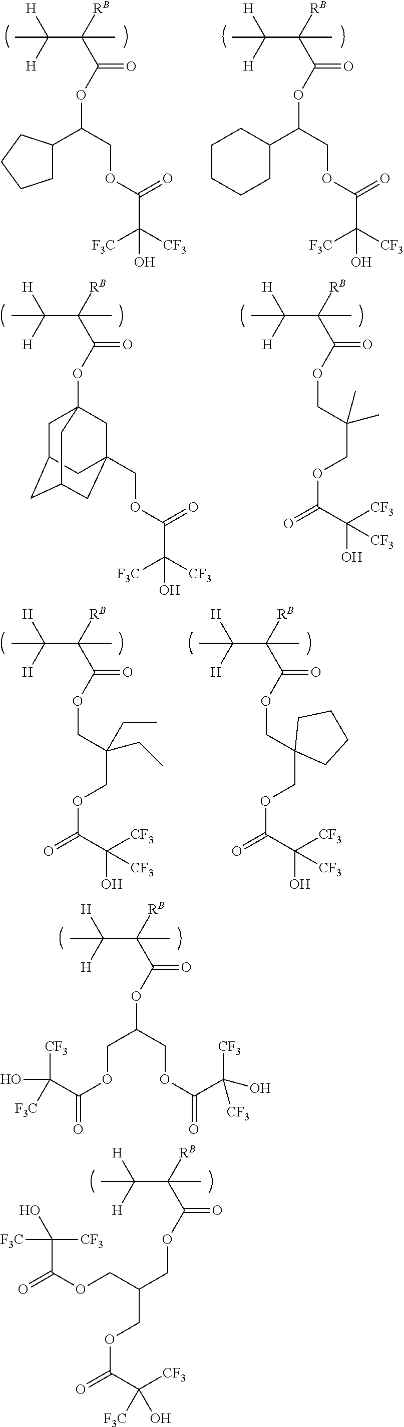

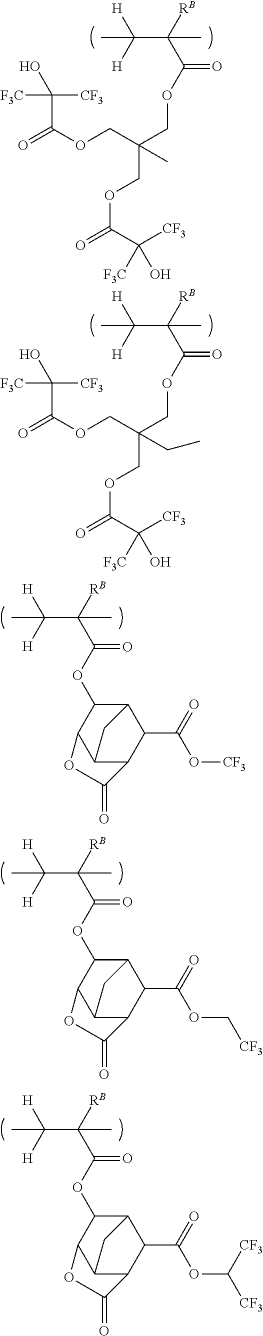

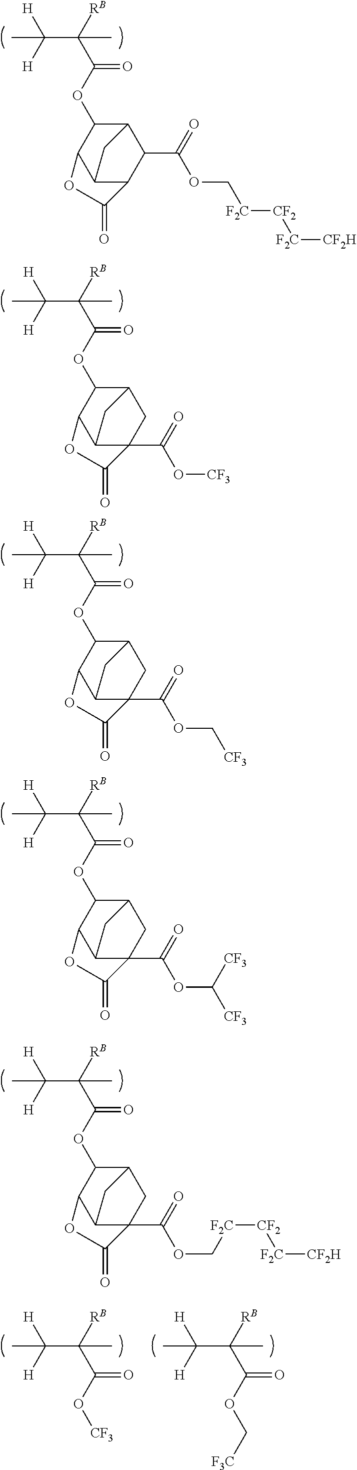

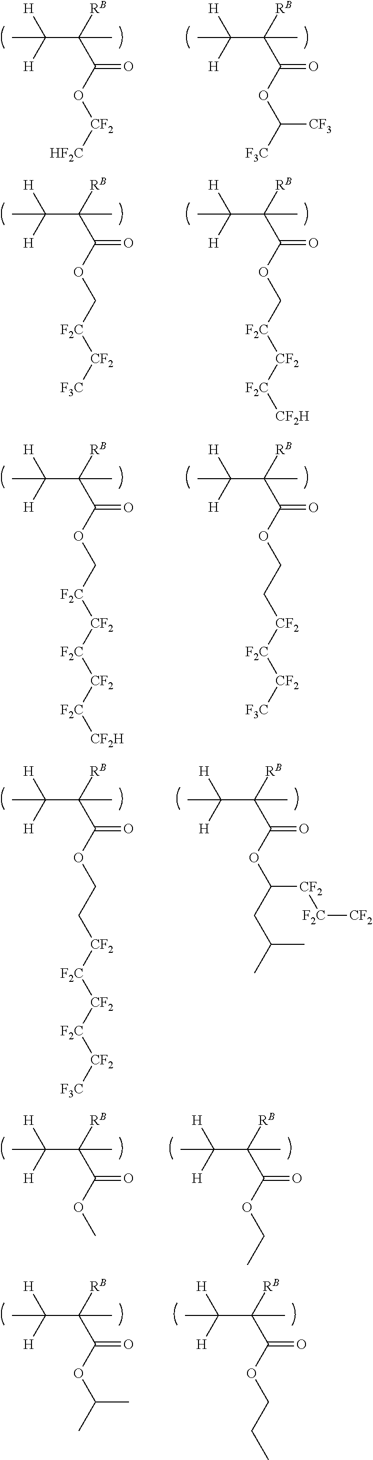

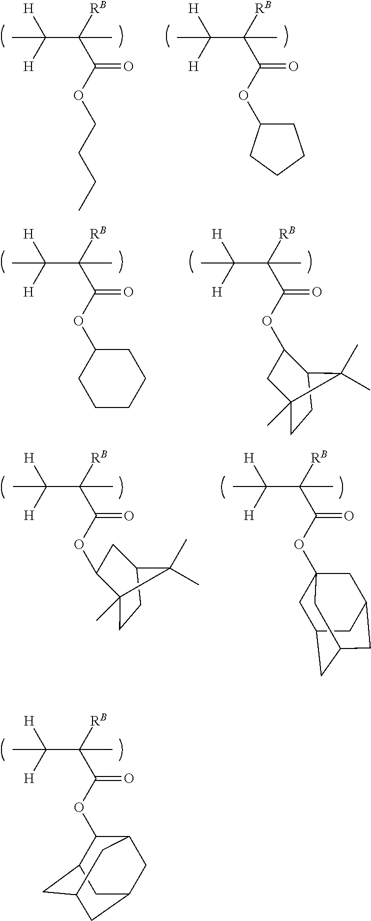

The polymeric surfactant may further contain repeat units other than the repeat units having formulae (7A) to (7E). Typical other repeat units are those derived from methacrylic acid and a-trifluoromethylacrylic acid derivatives. In the polymeric surfactant, the content of the repeat units having formulae (7A) to (7E) is preferably at least 20 mol %, more preferably at least 60 mol %, most preferably 100 mol % of the overall repeat units.

The polymeric surfactant preferably has a Mw of 1,000 to 500,000, more preferably 3,000 to 100,000 and a Mw/Mn of 1.0 to 2.0, more preferably 1.0 to 1.6.

The polymeric surfactant may be synthesized by any desired method, for example, by dissolving an unsaturated bond-containing monomer or monomers providing repeat units having formula (7A) to (7E) and optionally other repeat units in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the resist composition contains a surfactant (F), the amount thereof is preferably 0.1 to 50 parts by weight, and more preferably 0.5 to 10 parts by weight per 80 parts by weight of the base polymer (B). At least 0.1 part of the surfactant is effective in improving the receding contact angle with water of the resist film at its surface. Up to 50 parts of the surfactant is effective in forming a resist film having a low rate of dissolution in a developer and capable of maintaining the height of a small-size pattern formed therein. The surfactant (F) may be used alone or in admixture.

(G) Other Components

The resist composition may further comprise (G) another component, for example, a compound which is decomposed with an acid to generate another acid (i.e., acid amplifier compound), an organic acid derivative, a fluorinated alcohol, and a compound having a Mw ofup to 3,000 which changes its solubility in developer under the action of an acid (i.e., dissolution inhibitor). Specifically, the acid amplifier compound is described in JP-A 2009-269953 and JP-A 2010-215608 and preferably used in an amount of 0 to 5 parts, more preferably 0 to 3 parts by weight per 80 parts by weight of the base polymer (B). An extra amount of the acid amplifier compound can make the acid diffusion control difficult and cause degradations to resolution and pattern profile. With respect to the remaining additives, reference should be made to JP-A 2009-269953 and JP-A 2010-215608.

Process

A further embodiment of the invention is a process of forming a pattern from the resist composition defined above by lithography. The preferred process includes the steps of applying the resist composition onto a substrate to form a resist film thereon, exposing the resist film to high-energy radiation, and developing the exposed resist film in a developer. Any desired steps may be added to the process if necessary.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Then the resist film is exposed to a pattern of high-energy radiation, typically KrF or ArF excimer laser, EUV or EB. On use of KrF excimer laser, ArF excimer laser or EUV of wavelength 13.5 nm, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. The liquid is typically water, and in this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, the exposed region of the resist film is dissolved away, and a desired resist pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvents may be used alone or in admixture of two or more.

### EXAMPLES

Synthesis Examples, Examples and Comparative Examples are given below by way of illustration and not by way of limitation. The abbreviation “pbw” is parts by weight. Analysis is made by IR, ¹H-NMR spectroscopy and time-of-flight mass spectrometry using analytic instruments as shown below.

IR: NICOLET 6700 by Thermo Fisher Scientific Inc.

¹H-NMR: ECA-500 by JEOL Ltd.

MALDI TOF-MS: S3000 by JEOL Ltd.

### [1] Synthesis of Onium Salts

### Example 1-1 Synthesis of Onium Salt PAG-1

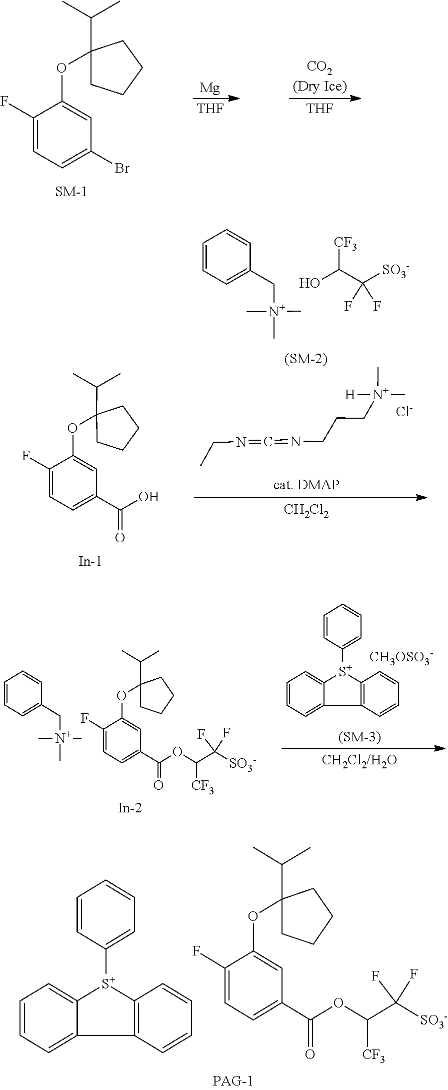

### (1) Synthesis of Intermediate In-1

In nitrogen atmosphere, a Grignard reagent was prepared using 1.5 g of magnesium, 18.1 g of reactant SM-1, and 50 mL of THF. The Grignard reagent was then added to a suspension of 30 g of dry ice in 100 mL of THF. After the addition, stirring was continued until the dry ice sublimed off. After the sublimation of dry ice was confirmed, 11.0 g of 20 wt % hydrochloric acid was added to quench the reaction. The end compound was extracted twice with 100 mL of ethyl acetate, followed by ordinary aqueous work-up. The subsequent steps of distilling off the solvent and recrystallization from hexane gave 13.1 g of Intermediate In-1 as white crystals (yield 82%).

### (2) Synthesis of Intermediate In-2

In nitrogen atmosphere, a reactor was charged with 13.1 g of Intermediate In-1, 19.0 g of reactant SM-2, 0.6 g of DMAP, and 60 g of methylene chloride and cooled in an ice bath. While the internal temperature of the reactor was kept below 20° C., 11.3 g of 1-ethyl-3-(3-dimethylaminopropyl)carbodiimide hydrochloride in powder form was added. Thereafter, the reactor was allowed to reach room temperature, at which the reaction solution was aged for 12 hours. At the end of aging, water was added to quench the reaction, followed by ordinary aqueous work-up. The subsequent steps of distilling off the solvent and recrystallization from diisopropyl ether gave 29.0 g of Intermediate In-2 as white crystals (yield 94%).

### (3) Synthesis of Onium Salt PAG-1

In nitrogen atmosphere, a reactor was charged with 12.6 g of Intermediate In-2, 8.2 g of reactant SM-3, 40 g of methylene chloride, and 30 g of water, which were stirred for 30 minutes. The organic layer was taken out, washed with water, and concentrated under reduced pressure. Diisopropyl ether was added to the concentrate for recrystallization, obtaining 13.6 g of the target compound, PAG-1 as white crystals (yield 92%).

PAG-1 was analyzed by IR spectroscopy and TOF-MS, with the data shown below. FIG. 1 is the ¹H-NMR/DMSO-d₆ spectrum of PAG-1.

IR (D-ATR): v=3061, 2972, 2877, 1743, 1607, 1591, 1509, 1481, 1450, 1423, 1373, 1334, 1274, 1257, 1246, 1203, 1185, 1167, 1122, 1102, 1070, 1057, 993, 975, 959, 895, 838, 777, 764, 755, 706, 681, 642, 620, 576, 553, 526, 500, 489, 422 cm⁻¹

MALDI TOF-MS: positive Mf 261 (corresponding to C₁₈H₁₃S⁺) negative M⁻ 477 (corresponding to C₁₈H₁₉F₆O₆S⁻)

### Examples 1-2 to 1-10 Synthesis of PAG-2 to PAG-10

Onium salts PAG-2 to PAG-10 shown below were synthesized using corresponding reactants and well-known organic synthesis reactions.

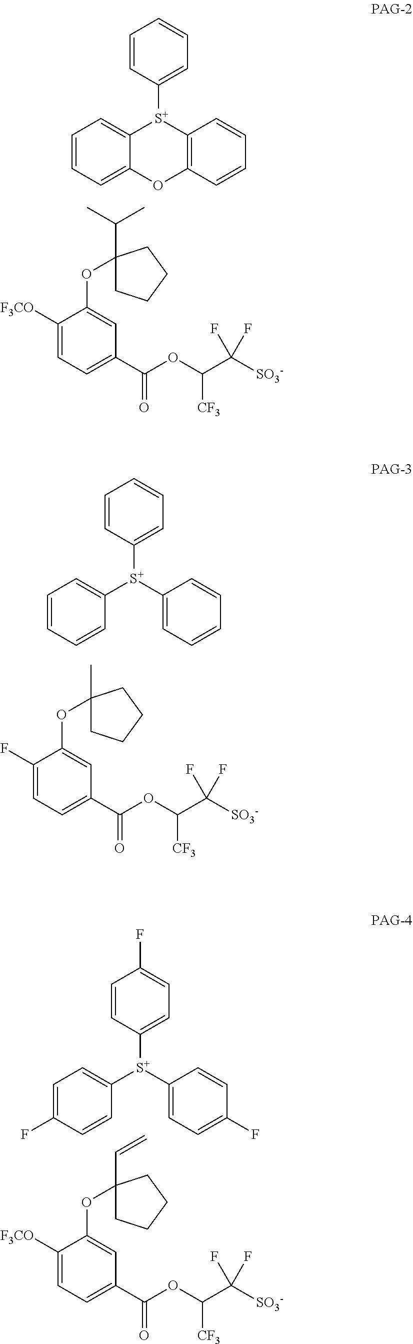

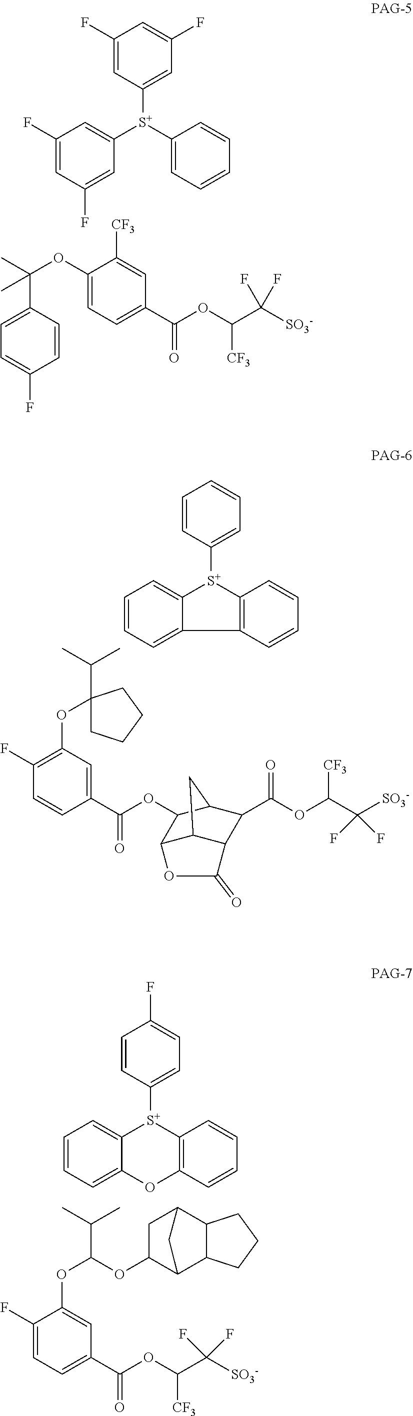

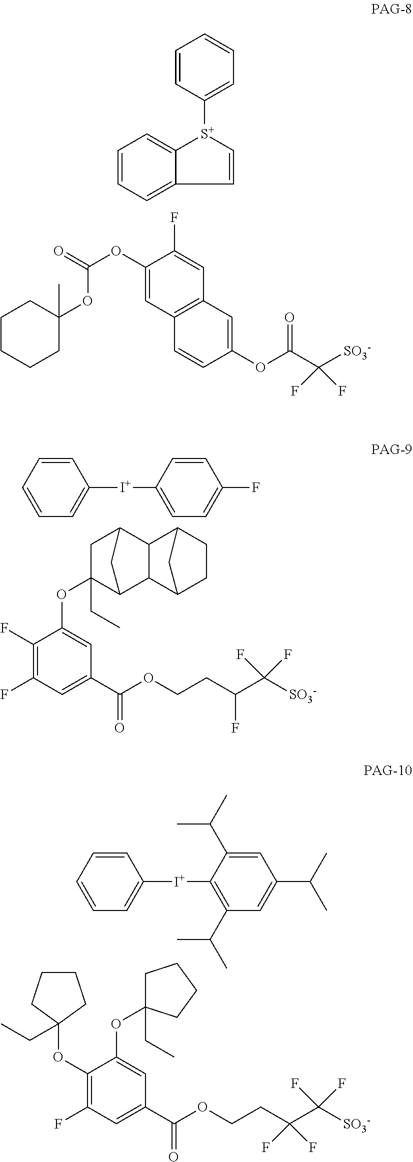

### [2] Synthesis of Base Polymers

### Synthesis Example Synthesis of Base Polymers P-1 to P-6

Base polymers P-1 to P-6 were synthesized by combining monomers, performing copolymerization reaction in MEK solvent, pouring the reaction solution to hexane for precipitation, washing the solid precipitate with hexane, isolation and drying. The polymer was analyzed for composition by H-NMR spectroscopy and for Mw and Mw/Mn by GPC versus polystyrene standards using THF solvent.

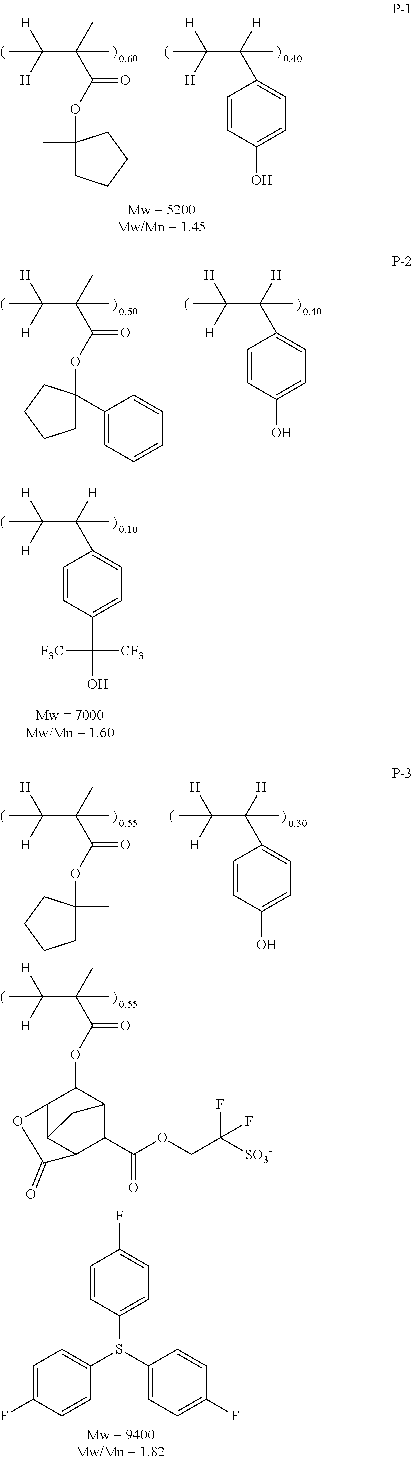

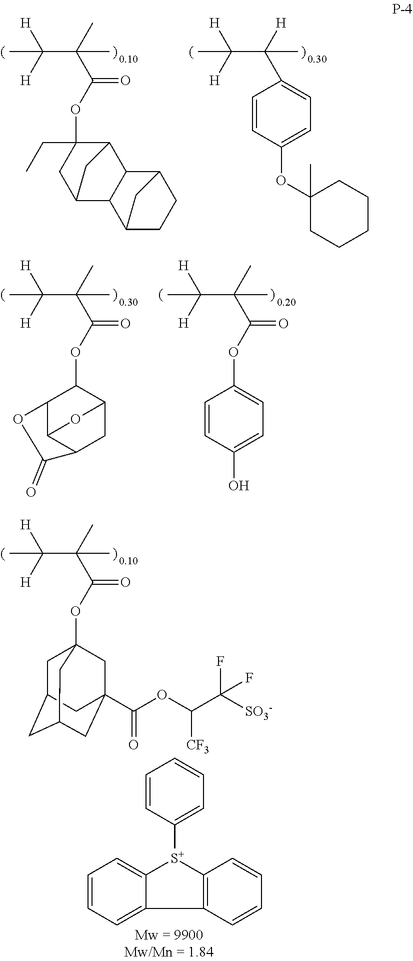

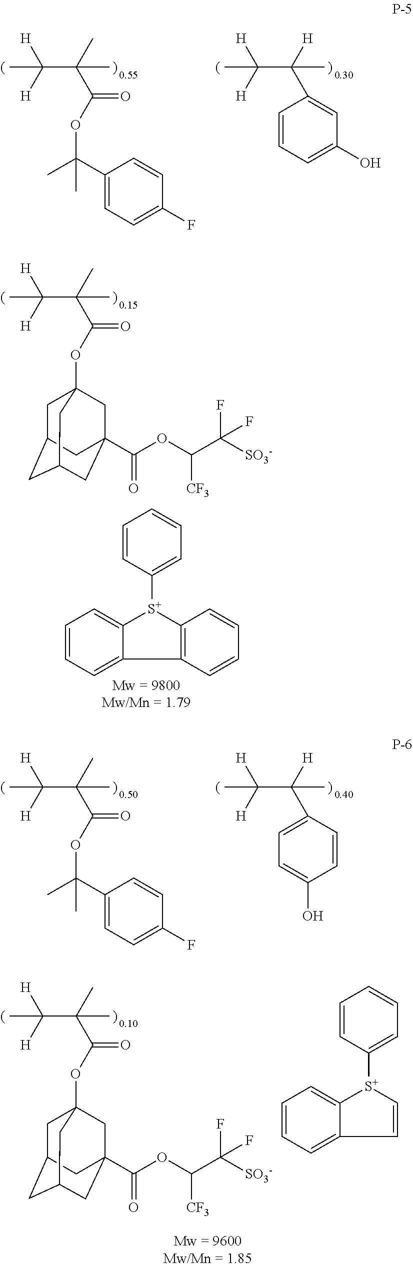

### [3] Preparation of Chemically Amplified Resist Compositions

### Examples 2-1 to 2-36 and Comparative Examples 1-1 to 1-28

Chemically amplified resist compositions (R-1 to R-36 CR-1 to CR-28) in solution form were prepared by dissolving an onium salt (PAG-1 to PAG-10) or comparative photoacid generator (PAG-A to PAG-D) other photoacid generator (PAG-X, PAG-Y), base polymer (Polymers P-1 to P-6); and quencher (Q-1 to Q-4) in a solvent containing 0.01 wt % of surfactant A in accordance with the formulation shown in Tables 1 to 3, and filtering through a Teflon® filter with a pore size of 0.2 μm.

TABLE 1

| | | | | | Other | | | |
|---|---|---|---|---|---|---|---|---|
| | | | Base | Photoacid | photoacid | | | |
| | | Resist | polymer | generator | generator | Quencher | Solvent 1 | Solvent 2 |
| | | composition | (pbw) | (pbw) | (pbw) | (pbw) | (pbw) | (pbw) |
| Example | 2-1 | R-1 | P-1 | PAG-1 | — | Q-1 | PGMEA | DAA |
| | | | (80) | (28) | | (8.0) | (2200) | (900) |
| | 2-2 | R-2 | P-1 | PAG-2 | — | Q-1 | PGMEA | DAA |
| | | | (80) | (28) | | (7.8) | (2200) | (900) |
| | 2-3 | R-3 | P-1 | PAG-3 | — | Q-1 | PGMEA | DAA |
| | | | (80) | (29) | | (7.4) | (2200) | (900) |
| | 2-4 | R-4 | P-1 | PAG-4 | — | Q-1 | PGMEA | DAA |
| | | | (80) | (27) | | (8.0) | (2200) | (900) |
| | 2-5 | R-5 | P-1 | PAG-5 | — | Q-1 | PGMEA | DAA |
| | | | (80) | (27) | | (8.0) | (2200) | (900) |
| | 2-6 | R-6 | P-1 | PAG-6 | — | Q-1 | PGMEA | DAA |
| | | | (80) | (29) | | (8.0) | (2200) | (900) |
| | 2-7 | R-7 | P-1 | PAG-7 | — | Q-1 | PGMEA | DAA |
| | | | (80) | (30) | | (7.8) | (2200) | (900) |
| | 2-8 | R-8 | P-1 | PAG-8 | — | Q-1 | PGMEA | DAA |
| | | | (80) | (28) | | (8.0) | (2200) | (900) |
| | 2-9 | R-9 | P-1 | PAG-9 | — | Q-1 | PGMEA | DAA |
| | | | (80) | (27) | | (8.0) | (2200) | (900) |
| | 2-10 | R-10 | P-1 | PAG-10 | — | Q-1 | PGMEA | DAA |
| | | | (80) | (28) | | (8.0) | (2200) | (900) |
| | 2-11 | R-11 | P-1 | PAG-1 | PAG-X | Q-2 | PGMEA | DAA |
| | | | (80) | (18) | (10) | (8.0) | (2200) | (900) |
| | 2-12 | R-12 | P-1 | PAG-2 | PAG-Y | Q-3 | PGMEA | DAA |
| | | | (80) | (18) | (10) | (7.6) | (2200) | (900) |
| | 2-13 | R-13 | P-2 | PAG-1 | — | Q-1 | PGMEA | DAA |
| | | | (80) | (24) | | (7.8) | (2200) | (900) |
| | 2-14 | R-14 | P-2 | PAG-2 | — | Q-3 | PGMEA | DAA |
| | | | (80) | (25) | | (8.0) | (2200) | (900) |
| | 2-15 | R-15 | P-2 | PAG-5 | — | Q-2 | PGMEA | DAA |
| | | | (80) | (24) | | (8.0) | (2200) | (900) |
| | 2-16 | R-16 | P-2 | PAG-6 | — | Q-1 | PGMEA | DAA |
| | | | (80) | (23) | | (8.0) | (2200) | (900) |
| | 2-17 | R-17 | P-3 | PAG-1 | — | Q-1 | PGMEA | DAA |
| | | | (80) | (10) | | (7.6) | (2200) | (900) |
| | 2-18 | R-18 | P-3 | PAG-2 | — | Q-3 | PGMEA | DAA |
| | | | (80) | (8) | | (8.0) | (2200) | (900) |
| | 2-19 | R-19 | P-3 | PAG-7 | — | Q-2 | PGMEA | DAA |
| | | | (80) | (10) | | (8.0) | (2200) | (900) |
| | 2-20 | R-20 | P-3 | PAG-8 | — | Q-2 | PGMEA | DAA |
| | | | (80) | (8) | | (7.6) | (2200) | (900) |

TABLE 2

| | | | | | Other | | | |
|---|---|---|---|---|---|---|---|---|
| | | | Base | Photoacid | photoacid | | | |
| | | Resist | polymer | generator | generator | Quencher | Solvent 1 | Solvent 2 |
| | | composition | (pbw) | (pbw) | (pbw) | (pbw) | (pbw) | (pbw) |
| Example | 2-21 | R-21 | P-4 | PAG-1 | — | Q-1 | PGMEA | DAA |
| | | | (80) | (10) | | (8.0) | (2200) | (900) |
| | 2-22 | R-22 | P-4 | PAG-2 | — | Q-2 | PGMEA | DAA |
| | | | (80) | (8) | | (8.0) | (2200) | (900) |
| | 2-23 | R-23 | P-4 | PAG-7 | — | Q-3 | PGMEA | DAA |
| | | | (80) | (8) | | (7.8) | (2200) | (900) |
| | 2-24 | R-24 | P-4 | PAG-9 | — | Q-1 (4.0) | PGMEA | DAA |
| | | | (80) | (10) | | Q-4 (4.0) | (2200) | (900) |
| | 2-25 | R-25 | P-5 | PAG-1 | — | Q-1 | PGMEA | DAA |
| | | | (80) | (10) | | (8.0) | (2200) | (900) |
| | 2-26 | R-26 | P-5 | PAG-2 | — | Q-2 | PGMEA | DAA |
| | | | (80) | (12) | | (8.0) | (2200) | (900) |
| | 2-27 | R-27 | P-5 | PAG-3 | — | Q-3 | PGMEA | DAA |
| | | | (80) | (8) | | (7.6) | (2200) | (900) |
| | 2-28 | R-28 | P-5 | PAG-4 | — | Q-2 | PGMEA | DAA |
| | | | (80) | (8) | | (8.0) | (2200) | (900) |
| | 2-29 | R-29 | P-5 | PAG-6 | PAG-X | Q-2 | PGMEA | DAA |
| | | | (80) | (6) | (4) | (8.2) | (2200) | (900) |
| | 2-30 | R-30 | P-5 | PAG-7 | — | Q-3 | PGMEA | DAA |
| | | | (80) | (10) | | (8.0) | (2200) | (900) |
| | 2-31 | R-31 | P-5 | PAG-8 | — | Q-3 (4.0) | PGMEA | DAA |
| | | | (80) | (9) | | Q-4 (4.0) | (2200) | (900) |
| | 2-32 | R-32 | P-5 | PAG-10 | — | Q-2 | PGMEA | DAA |
| | | | (80) | (10) | | (8.0) | (2200) | (900) |
| | 2-33 | R-33 | P-6 | PAG-1 | — | Q-1 | PGMEA | DAA |
| | | | (80) | (12) | | (8.4) | (2200) | (900) |
| | 2-34 | R-34 | P-6 | PAG-2 | — | Q-3 | PGMEA | DAA |
| | | | (80) | (10) | | (8.0) | (2200) | (900) |
| | 2-35 | R-35 | P-6 | PAG-5 | — | Q-2 | PGMEA | DAA |
| | | | (80) | (12) | | (7.8) | (2200) | (900) |
| | 2-36 | R-36 | P-6 | PAG-8 | — | Q-1 | PGMEA | DAA |
| | | | (80) | (12) | | (8.0) | (2200) | (900) |

TABLE 3

| | | | | | Other | | | |
|---|---|---|---|---|---|---|---|---|
| | | | Base | Photoacid | photoacid | | | |
| | | Resist | polymer | generator | generator | Quencher | Solvent 1 | Solvent 2 |
| | | composition | (pbw) | (pbw) | (pbw) | (pbw) | (pbw) | (pbw) |
| Comparative | 1-1 | CR-1 | P-1 | PAG-A | — | Q-1 | PGMEA | DAA |
| Example | | | (80) | (28) | | (8.0) | (2200) | (900) |
| | 1-2 | CR-2 | P-1 | PAG-B | — | Q-1 | PGMEA | DAA |
| | | | (80) | (28) | | (7.8) | (2200) | (900) |
| | 1-3 | CR-3 | P-1 | PAG-C | — | Q-1 | PGMEA | DAA |
| | | | (80) | (29) | | (7.8) | (2200) | (900) |
| | 1-4 | CR-4 | P-1 | PAG-D | — | Q-1 | PGMEA | DAA |
| | | | (80) | (27) | | (8.2) | (2200) | (900) |
| | 1-5 | CR-5 | P-1 | PAG-A | PAG-X | Q-2 | PGMEA | DAA |
| | | | (80) | (27) | (10) | (8.0) | (2200) | (900) |
| | 1-6 | CR-6 | P-1 | PAG-B | — | Q-3 | PGMEA | DAA |
| | | | (80) | (29) | | (8.0) | (2200) | (900) |
| | 1-7 | CR-7 | P-2 | PAG-A | — | Q-1 | PGMEA | DAA |
| | | | (80) | (28) | | (8.0) | (2200) | (900) |
| | 1-8 | CR-8 | P-2 | PAG-B | — | Q-2 | PGMEA | DAA |
| | | | (80) | (28) | | (8.0) | (2200) | (900) |
| | 1-9 | CR-9 | P-2 | PAG-C | — | Q-3 | PGMEA | DAA |
| | | | (80) | (29) | | (8.0) | (2200) | (900) |
| | 1-10 | CR-10 | P-2 | PAG-D | — | Q-1 (4.0) | PGMEA | DAA |
| | | | (80) | (27) | | Q-4 (4.0) | (2200) | (900) |
| | 1-11 | CR-11 | P-3 | PAG-A | — | Q-2 | PGMEA | DAA |
| | | | (80) | (10) | | (7.6) | (2200) | (900) |
| | 1-12 | CR-12 | P-3 | PAG-B | — | Q-1 | PGMEA | DAA |
| | | | (80) | (12) | | (7.8) | (2200) | (900) |
| | 1-13 | CR-13 | P-3 | PAG-C | — | Q-1 | PGMEA | DAA |
| | | | (80) | (8) | | (8.0) | (2200) | (900) |
| | 1-14 | CR-14 | P-3 | PAG-D | — | Q-3 | PGMEA | DAA |
| | | | (80) | (10) | | (7.6) | (2200) | (900) |
| | 1-15 | CR-15 | P-4 | PAG-A | — | Q-1 | PGMEA | DAA |
| | | | (80) | (10) | | (8.0) | (2200) | (900) |
| | 1-16 | CR-16 | P-4 | PAG-B | — | Q-1 | PGMEA | DAA |
| | | | (80) | (10) | | (7.6) | (2200) | (900) |
| | 1-17 | CR-17 | P-4 | PAG-C | — | Q-2 | PGMEA | DAA |
| | | | (80) | (8) | | (8.0) | (2200) | (900) |
| | 1-18 | CR-18 | P-4 | PAG-D | — | Q-3 | PGMEA | DAA |
| | | | (80) | (8) | | (8.0) | (2200) | (900) |
| | 1-19 | CR-19 | P-5 | PAG-A | — | Q-2 | PGMEA | DAA |
| | | | (80) | (10) | | (7.6) | (2200) | (900) |
| | 1-20 | CR-20 | P-5 | PAG-B | — | Q-1 | PGMEA | DAA |
| | | | (80) | (8) | | (8.0) | (2200) | (900) |
| | 1-21 | CR-21 | P-5 | PAG-C | — | Q-2 | PGMEA | DAA |
| | | | (80) | (10) | | (7.8) | (2200) | (900) |
| | 1-22 | CR-22 | P-5 | PAG-D | — | Q-3 | PGMEA | DAA |
| | | | (80) | (8) | | (7.8) | (2200) | (900) |
| | 1-23 | CR-23 | P-5 | PAG-B | — | Q-1 (4.0) | PGMEA | DAA |
| | | | (80) | (10) | | Q-4 (4.0) | (2200) | (900) |
| | 1-24 | CR-24 | P-5 | PAG-A | PAG-Y | Q-1 | PGMEA | DAA |
| | | | (80) | (6) | (4) | (7.08) | (2200) | (900) |
| | 1-25 | CR-25 | P-6 | PAG-A | — | Q-1 | PGMEA | DAA |
| | | | (80) | (10) | | (8.0) | (2200) | (900) |
| | 1-26 | CR-26 | P-6 | PAG-B | — | Q-3 | PGMEA | DAA |
| | | | (80) | (8) | | (7.6) | (2200) | (900) |
| | 1-27 | CR-27 | P-6 | PAG-C | — | Q-2 | PGMEA | DAA |
| | | | (80) | (10) | | (8.2) | (2200) | (900) |
| | 1-28 | CR-28 | P-6 | PAG-D | — | Q-2 | PGMEA | DAA |
| | | | (80) | (10) | | (8.2) | (2200) | (900) |

The solvents, other photoacid generators PAG-X and PAG-Y, comparative photoacid generators PAG-A to PAG-D, quenchers Q-1 to Q-4, and surfactant A in Tables 1 to 3 are identified below.

Solvent:
PGMEA (propylene glycol monomethyl ether acetate)
DAA (diacetone alcohol)

Other photoacid generators PAG-X and PAG-Y

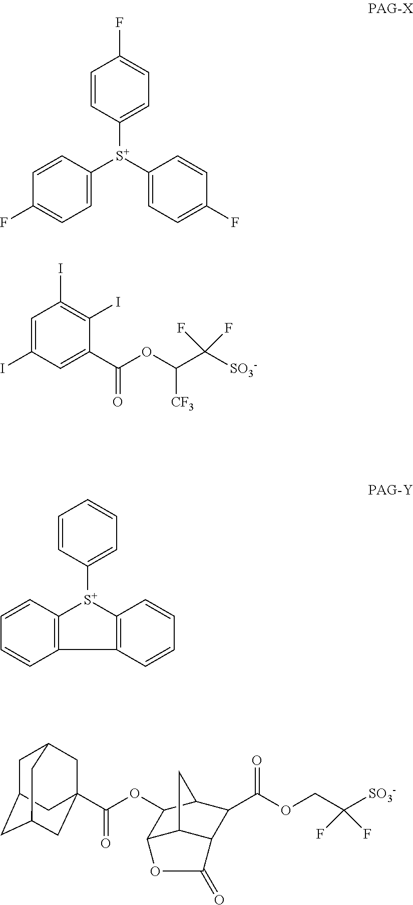

Comparative photoacid generators PAG-A to PAG-D

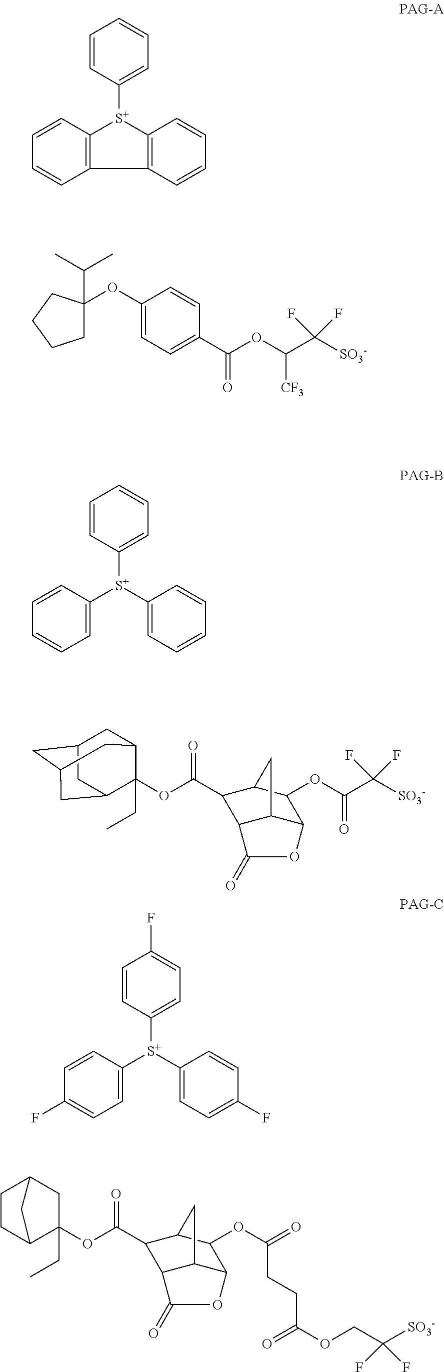

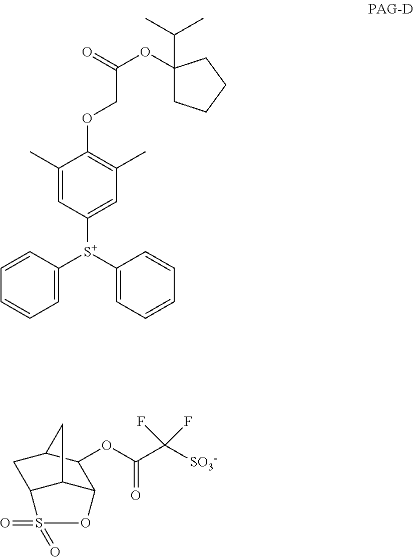

Quenchers Q-1 to Q-4

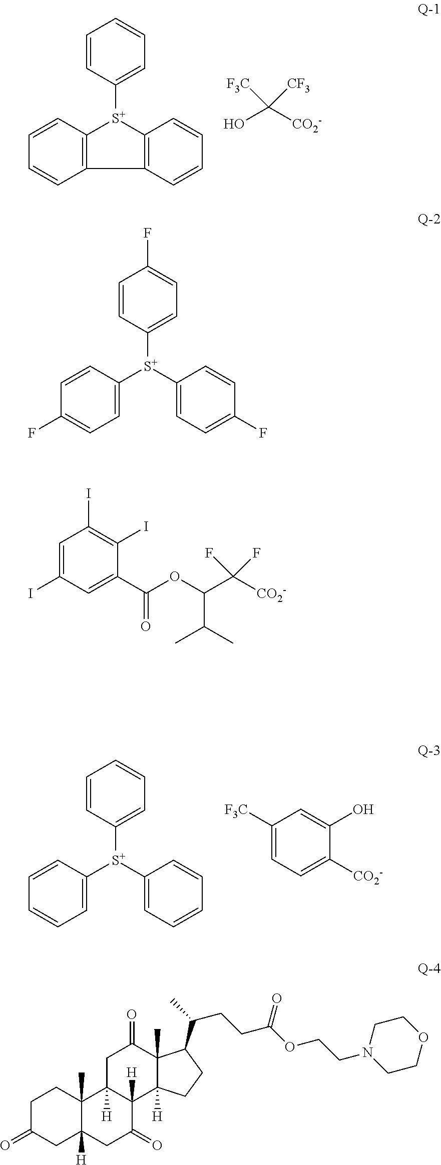

Surfactant A: 3-methyl-3-(2,2,2-trifluoroethoxymethyl)oxetane/tetrahydrofuran, 2,2-dimethyl-1,3-propane diol copolymer (Omnova Solutions, Inc.)

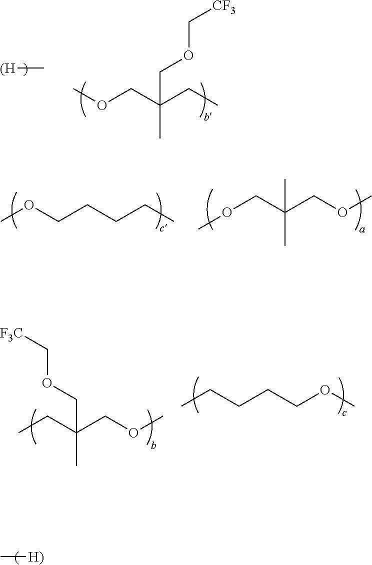

a:(b+b′):(c+c′)=1:4-7:0.01-1 (molar ratio)
Mw=1,500

### [4] EUV Lithography Test 1

### Examples 3-1 to 3-36 and Comparative Examples 2-1 to 2-28

Each of the chemically amplified resist compositions (R-1 to R-36, CR-1 to CR-28) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 (ASML, NA 0.33, σ 0.9/0.6, dipole illumination), the resist film was exposed to EUV through a mask bearing a LS pattern having a size of 18 nm and a pitch of 36 nm (on-wafer size) while varying the dose and focus (dose pitch: 1 mJ/cm², focus pitch: 0.020 μm). The resist film was baked (PEB) on a hotplate at the temperature shown in Tables 4 and 5 for 60 seconds and puddle developed in a 2.38 wt % TMAH aqueous solution for 30 seconds, rinsed with a rinse fluid containing surfactant, and spin dried to form a positive pattern.

The LS pattern as developed was observed under CD-SEM (CG6300, Hitachi High-Technologies Corp.) whereupon sensitivity, EL, LWR, DOF and collapse limit were evaluated by the following methods. The results are shown in Tables 4 and 5.

Evaluation of Sensitivity

The optimum dose Eop (mJ/cm²) which provided a LS pattern with a line width of 18 nm and a pitch of 36 nm was determined as an index of sensitivity. A smaller value indicates a higher sensitivity.

Evaluation of EL

The exposure dose which provided a LS pattern with a space width of 18 nm±10% (i.e., 16.2 to 19.8 nm) was determined. EL (%) is calculated from the exposure doses according to the following equation:

EL (%)=(|E1−E2|/Eop)×100

wherein E1 is an optimum exposure dose which provides a LS pattern with a line width of 16.2 nm and a pitch of 36 nm. E2 is an optimum exposure dose which provides a LS pattern with a line width of 19.8 nm and a pitch of 36 nm, and Eop is an optimum exposure dose which provides a LS pattern with a line width of 18 nm and a pitch of 36 nm. A greater value indicates better performance.

Evaluation of LWR

For the LS pattern formed by exposure at the optimum dose Eop, the line width was measured at 10 longitudinally spaced apart points, from which a 3-fold value (3a) of the standard deviation (a) was determined and reported as LWR. A smaller value of 3a indicates a pattern having small roughness and uniform line width.

Evaluation of DOF

As an index of DOF, a range of focus which provided a LS pattern with a size of 18 nm t 10% (i.e., 16.2 to 19.8 nm) was determined. A greater value indicates a wider DOF.

Evaluation of collapse limit of line pattern

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 4

| | | | PEB | | | | | Collapse |
|---|---|---|---|---|---|---|---|---|
| | | Resist | temp. | Eop | EL | LWR | DOF | limit |
| | | composition | (° C.) | (mJ/cm²) | (%) | (nm) | (nm) | (nm) |
| Example | 3-1 | R-1 | 100 | 41 | 19 | 2.7 | 120 | 10.8 |
| | 3-2 | R-2 | 100 | 42 | 18 | 2.7 | 120 | 10.9 |
| | 3-3 | R-3 | 100 | 41 | 17 | 2.8 | 120 | 10.5 |
| | 3-4 | R-4 | 95 | 40 | 18 | 2.9 | 110 | 10.5 |
| | 3-5 | R-5 | 100 | 42 | 18 | 2.8 | 110 | 10.8 |
| | 3-6 | R-6 | 100 | 40 | 18 | 3 | 110 | 10.5 |
| | 3-7 | R-7 | 100 | 42 | 19 | 2.9 | 110 | 10.6 |
| | 3-8 | R-8 | 95 | 41 | 19 | 2.7 | 120 | 11 |
| | 3-9 | R-9 | 100 | 42 | 17 | 2.9 | 110 | 10.9 |
| | 3-10 | R-10 | 100 | 41 | 19 | 2.9 | 120 | 10.6 |
| | 3-11 | R-11 | 95 | 41 | 18 | 2.7 | 110 | 11.1 |
| | 3-12 | R-12 | 95 | 41 | 19 | 2 | 120 | 11.2 |
| | 3-13 | R-13 | 100 | 40 | 17 | 2.9 | 120 | 10.9 |
| | 3-14 | R-14 | 100 | 39 | 17 | 2.8 | 100 | 10.7 |
| | 3-15 | R-15 | 95 | 41 | 18 | 2.8 | 120 | 11.3 |
| | 3-16 | R-16 | 100 | 40 | 19 | 2.8 | 120 | 11.2 |
| | 3-17 | R-17 | 100 | 39 | 18 | 2.7 | 110 | 11.4 |
| | 3-18 | R-18 | 95 | 38 | 18 | 2.8 | 110 | 10.8 |
| | 3-19 | R-19 | 100 | 39 | 17 | 2.8 | 120 | 10.7 |
| | 3-20 | R-20 | 100 | 39 | 19 | 2.7 | 120 | 10.9 |
| | 3-21 | R-21 | 100 | 39 | 18 | 2.7 | 110 | 10.4 |
| | 3-22 | R-22 | 95 | 40 | 19 | 2.8 | 120 | 10.8 |
| | 3-23 | R-23 | 100 | 40 | 18 | 2.7 | 100 | 11 |
| | 3-24 | R-24 | 100 | 39 | 17 | 2.9 | 120 | 10.7 |
| | 3-25 | R-25 | 95 | 39 | 18 | 2.9 | 120 | 10.6 |
| | 3-26 | R-26 | 100 | 38 | 19 | 3 | 110 | 10.8 |
| | 3-27 | R-27 | 95 | 39 | 18 | 2.8 | 120 | 10.4 |
| | 3-28 | R-28 | 100 | 40 | 18 | 2.7 | 110 | 10.9 |
| | 3-29 | R-29 | 100 | 40 | 19 | 2.9 | 120 | 11.3 |
| | 3-30 | R-30 | 95 | 38 | 18 | 2.8 | 110 | 10.8 |
| | 3-31 | R-31 | 100 | 40 | 18 | 2.8 | 110 | 11.1 |
| | 3-32 | R-32 | 100 | 40 | 17 | 2.9 | 120 | 10.4 |
| | 3-33 | R-33 | 100 | 39 | 18 | 3 | 100 | 10.8 |
| | 3-34 | R-34 | 95 | 39 | 17 | 2.8 | 120 | 10.7 |
| | 3-35 | R-35 | 100 | 38 | 18 | 2.8 | 110 | 11.4 |
| | 3-36 | R-36 | 100 | 39 | 18 | 2.7 | 120 | 11.2 |

TABLE 5

| | | | PEB | | | | | Collapse |
|---|---|---|---|---|---|---|---|---|
| | | Resist | temp. | Eop | EL | LWR | DOF | limit |
| | | composition | (° C.) | (mJ/cm²) | (%) | (nm) | (nm) | (nm) |
| Comparative | 2-1 | CR-1 | 100 | 46 | 15 | 3.8 | 70 | 14.4 |
| Example | 2-2 | CR-2 | 100 | 47 | 15 | 3.8 | 80 | 14.9 |
| | 2-3 | CR-3 | 95 | 44 | 16 | 4.1 | 70 | 14.5 |
| | 2-4 | CR-4 | 100 | 46 | 15 | 4.2 | 80 | 14.1 |
| | 2-5 | CR-5 | 100 | 45 | 14 | 4.3 | 80 | 14.5 |
| | 2-6 | CR-6 | 95 | 44 | 16 | 4 | 90 | 14.6 |
| | 2-7 | CR-7 | 100 | 46 | 16 | 3.9 | 80 | 14.8 |
| | 2-8 | CR-8 | 100 | 43 | 15 | 3.7 | 70 | 13.8 |
| | 2-9 | CR-9 | 95 | 45 | 15 | 4.1 | 80 | 14.2 |
| | 2-10 | CR-10 | 100 | 43 | 16 | 3.6 | 70 | 14.5 |
| | 2-11 | CR-11 | 100 | 43 | 15 | 3.4 | 80 | 14.9 |
| | 2-12 | CR-12 | 100 | 45 | 15 | 3.5 | 90 | 15.1 |
| | 2-13 | CR-13 | 95 | 46 | 16 | 3.8 | 80 | 14.5 |
| | 2-14 | CR-14 | 100 | 44 | 14 | 4.1 | 70 | 14.3 |
| | 2-15 | CR-15 | 100 | 45 | 15 | 3.4 | 80 | 14.5 |
| | 2-16 | CR-16 | 95 | 44 | 16 | 3.5 | 80 | 14.6 |
| | 2-17 | CR-17 | 100 | 43 | 15 | 3.7 | 90 | 13.9 |
| | 2-18 | CR-18 | 95 | 45 | 16 | 3.5 | 70 | 13.9 |
| | 2-19 | CR-19 | 100 | 44 | 14 | 4 | 70 | 14 |
| | 2-20 | CR-20 | 95 | 43 | 15 | 3.8 | 80 | 14.1 |
| | 2-21 | CR-21 | 100 | 45 | 16 | 3.5 | 90 | 13.8 |
| | 2-22 | CR-22 | 100 | 43 | 15 | 3.5 | 80 | 14.1 |
| | 2-23 | CR-23 | 95 | 44 | 15 | 3.4 | 80 | 14.6 |
| | 2-24 | CR-24 | 100 | 45 | 16 | 3.6 | 70 | 14.2 |
| | 2-25 | CR-25 | 100 | 44 | 15 | 3.4 | 80 | 14.3 |
| | 2-26 | CR-26 | 95 | 43 | 16 | 3.4 | 80 | 14.7 |
| | 2-27 | CR-27 | 100 | 42 | 14 | 3.6 | 70 | 14.2 |
| | 2-28 | CR-28 | 100 | 45 | 15 | 3.7 | 90 | 14.6 |

It is demonstrated in Tables 4 and 5 that chemically amplified resist compositions comprising PAGs within the scope of the invention exhibit a high sensitivity and improved values of EL, LWR and DOF. Small values of collapse limit attest that in forming a small-size pattern, the pattern is resistant to collapse. The resist compositions are useful in the EUV lithography process.

### [5] EUV-Lithography Test 2

### Examples 4-1 to 4-36 and Comparative Examples 3-1 to 3-28

Each of the chemically amplified resist compositions (R-1 to R-36, CR-1 to CR-28) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3300 (ASML, NA 0.33, a 0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 6 and 7 for 60 seconds and developed in a 2.38 wt % TMAH aqueous solution for 30 seconds to form a hole pattern having a size of 23 nm.

The pattern as developed was observed under CD-SEM (CG6300, Hitachi High-Technologies Corp.). The dose (mJ/cm²) at which a pattern with a hole size of 23 nm was printed was determined as an index of sensitivity. The size of 50 holes was measured, from which a 3-fold value (3a) of the standard deviation (σ) was determined as a dimensional variation (or CDU). The results are shown in Tables 6 and 7.

TABLE 6

| | | Resist | PEB | Optimum | CDU |
|---|---|---|---|---|---|
| | | composition | temp. (° C.) | dose (mJ/cm²) | (nm) |
| Example | 4-1 | R-1 | 95 | 24 | 2.2 |
| | 4-2 | R-2 | 90 | 25 | 2.4 |
| | 4-3 | R-3 | 90 | 25 | 2.3 |
| | 4-4 | R-4 | 90 | 26 | 2.4 |
| | 4-5 | R-5 | 90 | 24 | 2.5 |
| | 4-6 | R-6 | 95 | 25 | 2.6 |
| | 4-7 | R-7 | 90 | 24 | 2.4 |
| | 4-8 | R-8 | 90 | 26 | 2.5 |
| | 4-9 | R-9 | 95 | 25 | 2.3 |
| | 4-10 | R-10 | 95 | 25 | 2.4 |
| | 4-11 | R-11 | 90 | 26 | 2.5 |
| | 4-12 | R-12 | 90 | 25 | 2.3 |
| | 4-13 | R-13 | 90 | 24 | 2.5 |
| | 4-14 | R-14 | 90 | 25 | 2.4 |
| | 4-15 | R-15 | 95 | 24 | 2.4 |
| | 4-16 | R-16 | 85 | 25 | 2.2 |
| | 4-17 | R-17 | 95 | 24 | 2.4 |
| | 4-18 | R-18 | 90 | 26 | 2.4 |
| | 4-19 | R-19 | 90 | 25 | 2.5 |
| | 4-20 | R-20 | 85 | 24 | 2.4 |
| | 4-21 | R-21 | 90 | 25 | 2.6 |
| | 4-22 | R-22 | 90 | 24 | 2.6 |
| | 4-23 | R-23 | 90 | 24 | 2.3 |
| | 4-24 | R-24 | 90 | 24 | 2.6 |
| | 4-25 | R-25 | 90 | 26 | 2.4 |
| | 4-26 | R-26 | 90 | 24 | 2.6 |
| | 4-27 | R-27 | 95 | 25 | 2.3 |
| | 4-28 | R-28 | 90 | 24 | 2.3 |
| | 4-29 | R-29 | 90 | 25 | 2.4 |
| | 4-30 | R-30 | 85 | 25 | 2.5 |
| | 4-31 | R-31 | 95 | 24 | 2.3 |
| | 4-32 | R-32 | 95 | 24 | 2.5 |
| | 4-33 | R-33 | 90 | 25 | 2.5 |
| | 4-34 | R-34 | 90 | 24 | 2.4 |
| | 4-35 | R-35 | 90 | 24 | 2.2 |
| | 4-36 | R-36 | 85 | 25 | 2.3 |

TABLE 7

| | | Resist | PEB | Optimum | CDU |
|---|---|---|---|---|---|
| | | composition | temp. (° C.) | dose (mJ/cm²) | (nm) |
| Comparative | 3-1 | CR-1 | 90 | 35 | 3.5 |
| Example | 3-2 | CR-2 | 95 | 32 | 3.3 |
| | 3-3 | CR-3 | 90 | 34 | 3.3 |
| | 3-4 | CR-4 | 85 | 34 | 3.1 |
| | 3-5 | CR-5 | 90 | 33 | 3 |
| | 3-6 | CR-6 | 85 | 33 | 3.2 |
| | 3-7 | CR-7 | 90 | 34 | 2.9 |
| | 3-8 | CR-8 | 90 | 34 | 3 |
| | 3-9 | CR-9 | 90 | 35 | 3.1 |
| | 3-10 | CR-10 | 85 | 34 | 3 |
| | 3-11 | CR-11 | 95 | 33 | 3.1 |
| | 3-12 | CR-12 | 90 | 35 | 3.2 |
| | 3-13 | CR-13 | 95 | 30 | 2.8 |
| | 3-14 | CR-14 | 90 | 29 | 2.8 |
| | 3-15 | CR-15 | 90 | 30 | 2.9 |
| | 3-16 | CR-16 | 85 | 29 | 2.7 |
| | 3-17 | CR-17 | 90 | 29 | 2.9 |
| | 3-18 | CR-18 | 95 | 28 | 2.7 |
| | 3-19 | CR-19 | 90 | 29 | 2.9 |
| | 3-20 | CR-20 | 90 | 30 | 2.8 |
| | 3-21 | CR-21 | 85 | 29 | 2.9 |
| | 3-22 | CR-22 | 90 | 28 | 3 |
| | 3-23 | CR-23 | 95 | 30 | 2.9 |
| | 3-24 | CR-24 | 90 | 29 | 2.9 |
| | 3-25 | CR-25 | 95 | 29 | 2.8 |
| | 3-26 | CR-26 | 90 | 28 | 3.1 |
| | 3-27 | CR-27 | 85 | 28 | 2.7 |
| | 3-28 | CR-28 | 90 | 29 | 2.9 |

It is demonstrated in Tables 6 and 7 that chemically amplified resist compositions within the scope of the invention exhibit a high sensitivity and satisfactory CDU.

Japanese Patent Application No. 2022-129450 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An onium salt having the formula (1): 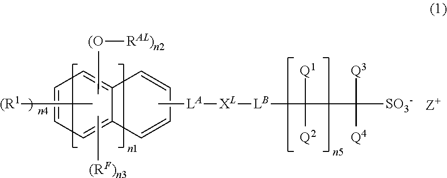
wherein n1 is 0 or 1, n2 is an integer of 1 to 3, n3 is an integer of 1 to 4, n4 is an integer of 0 to 4, meeting n2+n3+n4≤5 in case of n1=0 and n2+n3+n4≤7 in case of n1=1, n5 is an integer of 0 to 4,
R^{AL} forms an acid labile group with the adjoining oxygen atom,
R^{F} is fluorine, a C₁-C₆ fluorinated saturated hydrocarbyl group, C₁-C₆ fluorinated saturated hydrocarbyloxy group, or C₁-C₆ fluorinated saturated hydrocarbylthio group, a plurality of R^{F} may be identical or different in case of n3≥2,
R^{F} and —O—R^{AL} are attached to adjoining carbon atoms,
R¹ is a C₁-C₂₀ hydrocarbyl group which may contain a heteroatom,
L^{A} and L^{B} are each independently a single bond, ether bond, ester bond, sulfonic ester bond, carbonate bond or carbamate bond,
X^{L} is a single bond or a C₁-C₄₀ hydrocarbylene group which may contain a heteroatom,
Q¹ and Q² are each independently hydrogen, fluorine or a C₁-C₆ fluorinated saturated hydrocarbyl group,
Q³ and Q⁴ are each independently fluorine or a C₁-C₆ fluorinated saturated hydrocarbyl group, and
Z⁺ is an onium cation.

2. The onium salt of claim 1 wherein R^{A} is a group having the formula (AL-1) or (AL-2): 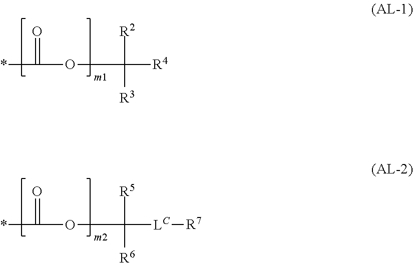
wherein R², R³ and R⁴ are each independently a C₁-C₁₂ hydrocarbyl group in which some —CH₂— may be replaced by —O— or —S—, with the proviso that when the hydrocarbyl group contains an aromatic ring, some or all of the hydrogen atoms on the aromatic ring may be substituted by halogen, cyano, nitro, optionally halogenated C₁-C₄ alkyl moiety or optionally halogenated C₁-C₄ alkoxy moiety, and R² and R³ may bond together to form a ring with the carbon atom to which they are attached, some —CH₂— in the ring may be replaced by —O— or —S—,
R⁵ and R⁶ are each independently hydrogen or a C₁-C₁₀ hydrocarbyl group, R⁷ is a C₁-C₂₀ hydrocarbyl group in which some —CH₂— may be replaced by —O— or —S—, and R⁶ and R⁷ may bond together to form a C₃-C₂₀ heterocyclic group with the carbon atom and Le to which they are attached, some —CH₂— in the heterocyclic group may be replaced by —O— or —S—,
L^{C} is —O— or —S—,
m1 is 0 or 1, m2 is 0 or 1,
* designates a point of attachment to the adjoining —O—.

3. The onium salt of claim 1, having the formula (1A): 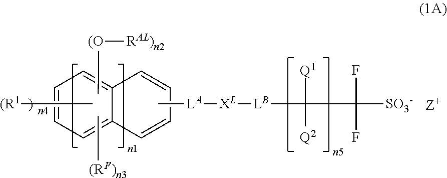
wherein R^{AL}, R^{F}, R¹, L^{A}, L^{B}, X^{L}, Q¹, Q², n1 to n5, and Z⁺ are as defined above.

4. The onium salt of claim 3, having the formula (1B): 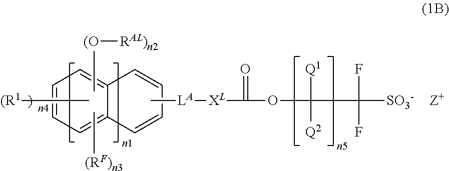
wherein R^{AL}, R^{F}, R¹, L^{A}, X^{L}, Q¹, Q², n1 to n5, and Z⁺ are as defined above.

5. The onium salt of claim 1 wherein Z⁺ is an onium cation having the formula (cation-1) or (cation-2): 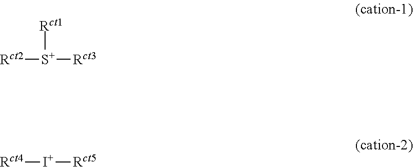
wherein R^{ct1} to R^{ct5} are each independently a C₁-C₃₀ hydrocarbyl group which may contain a heteroatom, and R^{ct1} and R^{ct2} may bond together to form a ring with the sulfur atom to which they are attached.

6. A photoacid generator comprising the onium salt of claim 1.

7. A chemically amplified resist composition comprising the photoacid generator of claim 6.

8. The resist composition of claim 7, further comprising a base polymer comprising repeat units having the formula (a1): 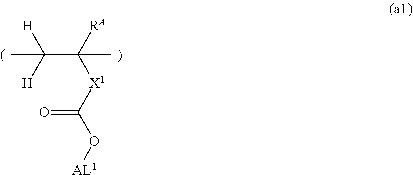
wherein R^{A} is hydrogen, fluorine, methyl or trifluoromethyl,
X¹ is a single bond, phenylene group, naphthylene group or *—C(═O)—O—X¹¹—, the phenylene or naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, or phenylene or naphthylene group, * designates a point of attachment to the carbon atom in the backbone, and
AL¹ is an acid labile group.

9. The resist composition of claim 8 wherein the base polymer further comprises repeat units having the formula (a2): 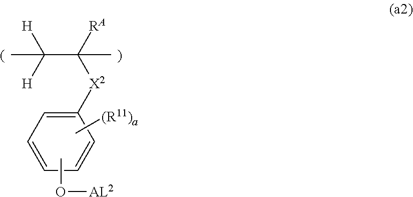
wherein R^{A} is hydrogen, fluorine, methyl or trifluoromethyl,
X² is a single bond or *—C(═O)—O—, * designates a point of attachment to the carbon atom in the backbone,
R²¹ is halogen, cyano, a C₁-C₂₀ hydrocarbyl group which may contain a heteroatom, a C1-C20 hydrocarbyloxy group which may contain a heteroatom, a C2-C20 hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
AL² is an acid labile group, and
a is an integer of 0 to 4.

10. The resist composition of claim 8 wherein the base polymer further comprises repeat units having the formula (b1) or (b2): 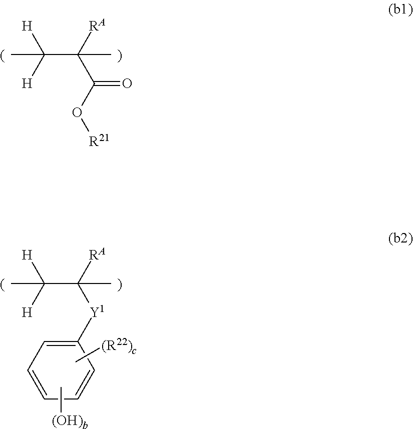
wherein R^{A} is each independently hydrogen, fluorine, methyl or trifluoromethyl,
Y¹ is a single bond or *—C(═O)—O—, * designates a point of attachment to the carbon atom in the backbone,
R²¹ is hydrogen or a C₁-C₂₀ group containing at least one structure selected from the group consisting of hydroxy other than phenolic hydroxy, cyano, carbonyl, carboxy, ether bond, ester bond, sulfonic ester bond, carbonate bond, lactone ring, sultone ring and carboxylic anhydride (—C(═O)—O—C(═O)—),
R²² is halogen, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
b is an integer of 1 to 4, c is an integer of 0 to 4, and b+c is from 1 to 5.

11. The resist composition of claim 8 wherein the base polymer further comprises repeat units of at least one type selected from repeat units having the formulae (c1) to (c4): 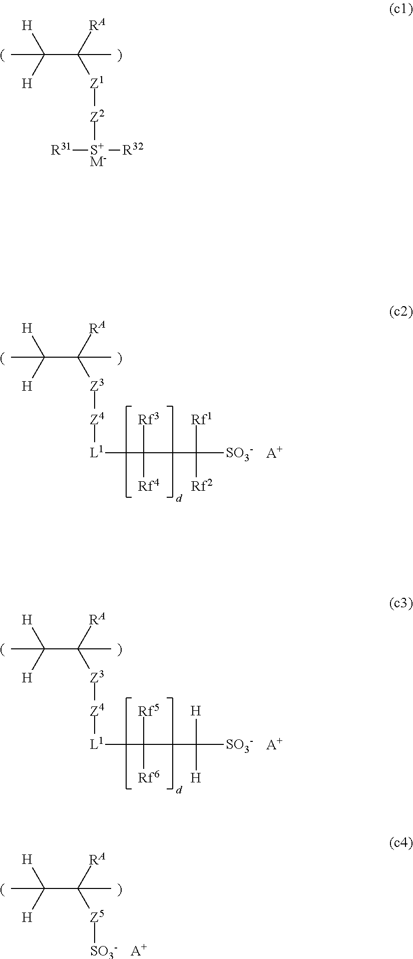
wherein R^{A} is each independently hydrogen, fluorine, methyl or trifluoromethyl,
Z¹ is a single bond or phenylene group,
Z² is *—C(═O)—O—Z²¹—, *—C(═O)—NH—Z²¹—, or *—O—Z²¹—, Z²¹ is a C₁-C₆ aliphatic hydrocarbylene group, phenylene group or a divalent group obtained by combining the foregoing, which may contain a carbonyl moiety, ester bond, ether bond or hydroxy moiety,
Z³ is a single bond, phenylene, naphthylene, or *—C(═O)—O—Z³¹—, Z³¹ is a C₁-C₁₀ aliphatic hydrocarbylene group which may contain a hydroxy moiety, ether bond, ester bond or lactone ring, or phenylene or naphthylene group,
Z⁴ is a single bond or *—Z⁴l-C(═O)—O—, Z⁴¹ is a C₁-C₂₀ hydrocarbylene group which may contain a heteroatom,
Z⁵ is a single bond, methylene, ethylene, phenylene, fluorinated phenylene, trifluoromethyl-substituted phenylene, *—C(═O)—O—Z⁵¹—, *—C(═O)—N(H)—Z⁵¹—, or *—O—Z⁵¹—, Z⁵¹ is a C₁-C₅ aliphatic hydrocarbylene group, phenylene group, fluorinated phenylene group or trifluoromethyl-substituted phenylene group, which may contain a carbonyl moiety, ester bond, ether bond or hydroxy moiety,
* designates a point of attachment to the carbon atom in the backbone,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, ether bond, ester bond, carbonyl group, sulfonic ester bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine, or a C₁-C₆ fluorinated saturated hydrocarbyl group,
Rf⁵ and Rf⁶ are each independently hydrogen, fluorine, or a C₁-C₆ fluorinated saturated hydrocarbyl group, excluding that all Rf⁵ and Rf⁶ are hydrogen at the same time,
M⁻ is a non-nucleophilic counter ion,
A⁺ is an onium cation, and
d is an integer of 0 to 3.

12. The resist composition of claim 7, further comprising an organic solvent.

13. The resist composition of claim 7, further comprising a quencher.

14. The resist composition of claim 7, further comprising another photoacid generator.

15. The resist composition of claim 7, further comprising a surfactant.

16. A pattern forming process comprising the steps of applying the chemically amplified resist composition of claim 7 onto a substrate to form a resist film thereon, exposing the resist film to high-energy radiation, and developing the exposed resist film in a developer.

17. The pattern forming process of claim 16 wherein the high-energy radiation is KrF excimer laser radiation, ArF excimer laser radiation, EB or EUV of wavelength 3 to 15 nm.
